(19) United States
(12) Patent Application Publication
(10) Pub. No.: US 2026/0356817 A1
(43) Pub. Date: Nov. 12, 2026

(54) AMINOPYRIMIDINE AND AMINOTRIAZINE DERIVATIVES AS MYC PROTEIN MODULATORS

(71) Applicant: Sanford Burnham Prebys Medical Discovery Institute, La Jolla, CA (US)

(72) Inventors: Shubhankar DUTTA, San Diego, CA (US); Christian A. HASSIG, San Diego, CA (US); Susanne HEYNEN-GENEL, Carlsbad, CA (US); Michael R. JACKSON, Del Mar, CA (US); Steven H. OLSON, San Diego, CA (US); Anthony B. PINKERTON, Rancho Santa Fe, CA (US); Brad SAVALL, San Diego, CA (US); Robert WECHSLER-REYA, San Diego, CA (US)

(21) Appl. No.: 19/105,691

(22) PCT Filed: Aug. 25, 2023
(86) PCT No.: PCT/US2023/072925

§ 371 Date: Feb. 21, 2025

Related U.S. Application Data
(60) Provisional application No. 63/401,454, filed on Aug. 26, 2022.

(51) Int. Cl. C07D 403/04; A61K 31/4418; A61K 31/4439; A61K 31/444; A61K 31/505; A61K 31/506; A61K 31/519; A61K 31/53; A61K 31/5377; A61P 35/00; C07D 213/74; C07D 239/42; C07D 401/04; C07D 401/12; C07D 401/14; C07D 403/12; C07D 403/14; C07D 405/14; C07D 409/04; C07D 409/14; C07D 413/04; C07D 413/14; C07D 417/04; C07D 417/14; C07D 471/04; C07D 487/04

(52) U.S. Cl. CPC C07D 403/04; A61K 31/4418; A61K 31/4439; A61K 31/444; A61K 31/505; A61K 31/506; A61K 31/519; A61K 31/53; A61K 31/5377; A61P 35/00; C07D 213/74; C07D 239/42; C07D 401/04; C07D 401/12; C07D 401/14; C07D 403/12; C07D 403/14; C07D 405/14; C07D 409/04; C07D 409/14; C07D 413/04; C07D 413/14; C07D 417/04; C07D 417/14; C07D 471/04; C07D 487/04

(57) ABSTRACT

The present disclosure provides compounds and compositions that are useful as MYC protein modulators and methods of using the same for treating MYC-mediated diseases or disorders.

### CROSS-REFERENCE TO RELATED APPLICATIONS

This application claims the benefit of U.S. Provisional Patent Application No. 63/401,454 filed Aug. 26, 2022, which is incorporated herein by reference in its entirety.

### BACKGROUND

### Technical Field

The present disclosure generally relates to compounds of Formula (I), (Ia), (Ib), or (II), or pharmaceutically acceptable salts thereof, and compositions comprising compounds of Formula (I), (Ia), (Ib), or (II), or pharmaceutically acceptable salts thereof, that are useful in treating various diseases and disorders, such as cancer.

### Description of the Related Technology

Oncoproteins c-Myc, N-Myc, and L-Myc, encoded by MYC proto-oncogene C-MYC, MYCN, and MYCL respectively, belong to a family of transcription factors that regulate the transcription of more than 15% of the entire genome. Recent mouse model studies have suggested that the modulation and regulation of oncogenic Myc proteins could potentially lead to the development of therapeutics useful for treating various cancers. Furthermore, amplification and overexpression of N-Myc can lead to tumorigenesis and poor health outcomes in patients, and excess amounts of N-Myc proteins are associated with a variety of tumors, such as neuroblastomas.

Transcription factors related to the oncogenic Myc proteins include the family of serine/threonine kinases known as cyclin-dependent protein kinases (CDKs). CDKs bind to a cyclin regulatory protein and are involved in regulation of the cell cycle, regulation of transcription, and mRNA processing. CDK9 is a cyclin-dependent kinase associated with P-TEFb (positive transcription elongation factor b). CDK9/P-TEFb phosphorylates the C-terminal domain of RNA polymerase II to regulate transcription elongation. Inhibition of CDK9 results in depletion of mRNA transcripts and associated proteins, including c-Myc. As such, inhibitors of CDK9 may be useful anti-cancer therapeutics.

Thus, a need exists for modulators of Myc proteins and related CDK9 proteins for the treatment of various diseases and disorders, such as cancer. The present disclosure fulfills these and other needs, as evident in reference to the following disclosure.

### SUMMARY

The present disclosure provides compounds and compositions that are useful as Myc protein modulators, and methods of using the same for treating various proliferative diseases, such as cancer. In some embodiments, the compounds are inhibitors of Myc. In some embodiments, the compounds described herein may be useful in the treatment of a Myc-mediated disease or disorder, such as cancers.

The present disclosure provides compounds and compositions that are useful as CDK protein modulators, and methods of using the same for treating various proliferative diseases, such as cancer. In some embodiments, the compounds are modulators of CDK. In some embodiments, the compounds are inhibitors of CDK. In some embodiments, the compounds described herein may be useful in the treatment of a CDK-mediated disease or disorder, such as cancer. In some embodiments, the CDK is CDK7, CDK8, CDK9, CDK11, CDK12, CDK13, CDK20. In some embodiments, the CDK is CDK9. Some embodiments provide a compound of Formula (I):

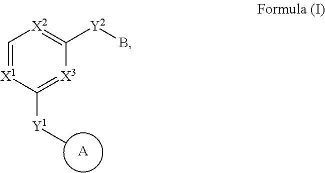

or a pharmaceutically acceptable salt thereof, for use in treating a disease or disorder mediated by a Myc protein, wherein:
X¹, X², and X³ are each independently N or CR¹, wherein at least one of X¹, X², and X₃ is N;
Y¹ is a bond, —O—, —S—, —CH₂—, or —N(R²)(CH₂)ₙ—;
Y² is —O—, —S—, —CH₂—, or —N(R²)(CH₂)ₙ—;

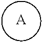

is phenyl, 5-9 membered heteroaryl,

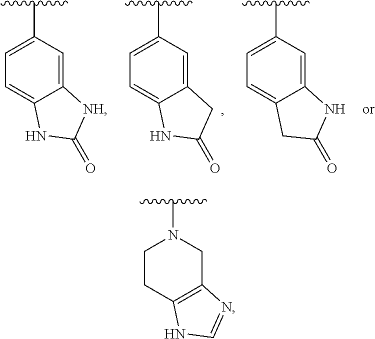

wherein the phenyl and 5-9 membered heteroaryl are each optionally substituted with 1-3 R⁹ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂;
B is C₁-C₅ alkyl, aryl, heteroaryl, 5-9 membered heterocycloalkyl, or C₃-C₁₀-cycloalkyl, wherein the aryl, heteroaryl, 5-9 membered heterocycloalkyl, and C₃-C₁₀-cycloalkyl are each optionally substituted with 1-3 R⁸ groups each independently selected from the group consisting of —(CH₂)ₙCN, —(CH₂)OH, —(CH₂)ₙN(R⁴)₂, —(CH₂)ₙC(O)N(R⁴)₂, —(CH₂)NR⁴C(O)R⁴, —(CH₂)ₙNR⁴C(O)N(R⁴)₂, —(CH₂)ₙNR⁴(C₁-C₅)alkylene(O)R^{4A}, —(CH₂)ₙC(O)R⁴, —(CH₂)ₙCO₂R⁴, —(CH₂)ₙOC(O)R⁴, —(CH₂)ₙOC(O)OR⁴, —(CH₂)ₙO(C₁-C₅-alkylene)N(R⁴)₂, —(CH₂)ₙSR⁴, —(CH₂)ₙS(O)R⁴, —(CH₂)ₙS(O)₂R⁴, —(CH₂)ₙN(R⁴)S(O)₂R⁴, —(CH₂)ₙS(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₅-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo;
R¹ is hydrogen, halogen, or C₁-C₅ alkyl;
R² and R³ are each independently hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl; and
each n is independently 0, 1, 2, 3, or 4. In some embodiments,

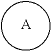

is not

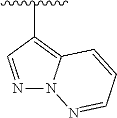 [SMILES: *c1cnn2ncccc12]

optionally substituted with 1-3 R⁹ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂.

In some embodiments, X¹ is CR¹, and X² and X³ are each independently N. In some embodiments, X¹ and X² are each independently CR¹, and X₃ is N. In some embodiments, X¹ and X³ are each independently CR¹, and X² is N. In some embodiments, X¹, X² and X³ are each independently N.

In some embodiments, Y¹ is a bond. In some embodiments, Y¹ is —O—, or —N(R²)(CH₂)ₙ—. In some embodiments, Y¹ is —N(R²)(CH₂)ₙ—. In some embodiments, Y¹ is —CH₂—, —S—, or —O—.

In some embodiments, R¹ is hydrogen, or —CH₃; and B is aryl or heteroaryl each optionally substituted with 1-3 R⁸ groups each independently selected from the group consisting of —CN, —OH, —NO₂, —N(R⁴)₂, —C(O)N(R⁴)₂, —NR⁴C(O)R⁴, —NR⁴C(O)N(R⁴)₂, —NR⁴ (C₁-C₅)alkylene(O)R⁴, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, alkylaryl, heteroaryl, 5-9 membered heterocycloalkyl, 5-9-membered cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

In some embodiments,

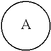

is 5-9-membered heteroaryl or phenyl each optionally substituted with 1-3 R⁹ groups each independently selected from the group consisting of halogen, C₁-C₅ alkyl, C₁-C₅ haloalkyl, —OR³, and —NH₂. In some embodiments,

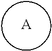

is 5-9 membered heteroaryl or phenyl each substituted with 1-3 R⁹ groups each independently selected from the group consisting of halogen, —OH, —OCH₃, —NH₂, and —CH₃.

In some embodiments,

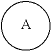

is selected from:

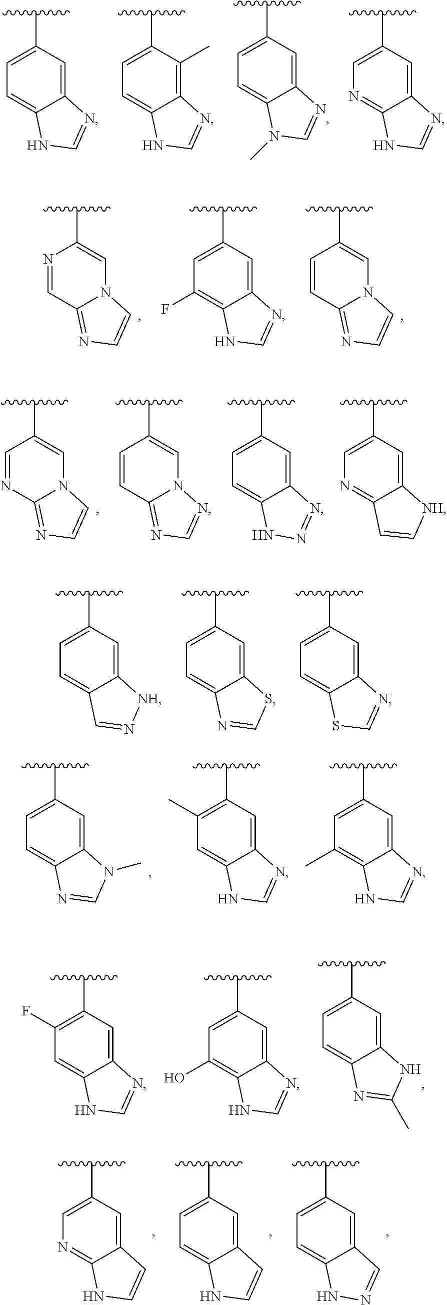

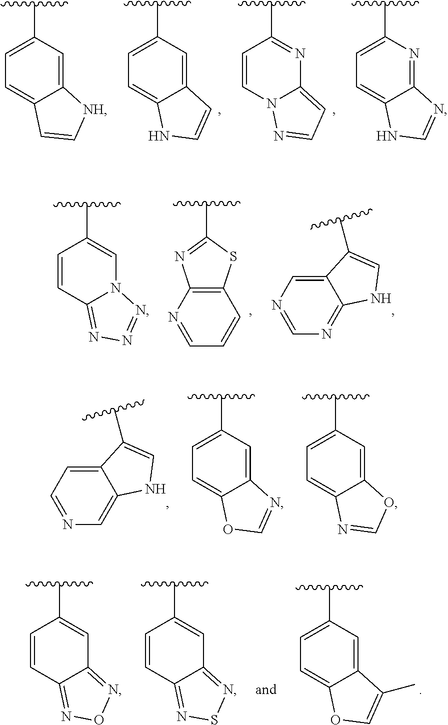

In some embodiments,

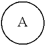

is selected from:

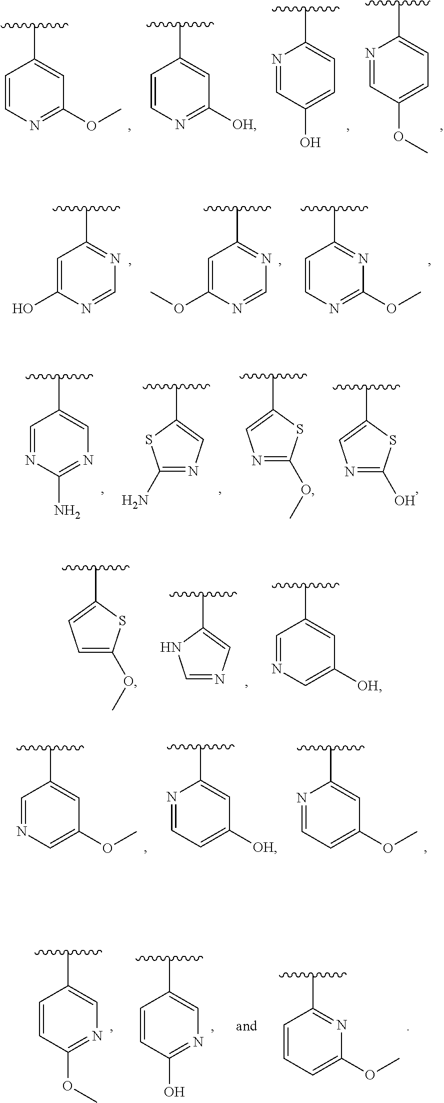

In some embodiments,

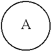

is selected from

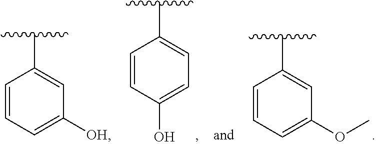

In some embodiments,

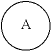

is selected from

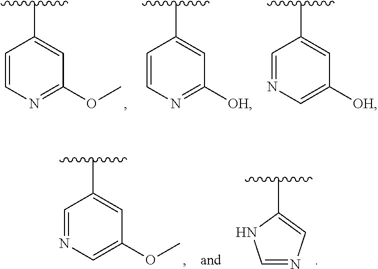

In some embodiments, B is C₁-C₅ alkyl, C₃-C₉-cycloalkyl, or 5-9 membered heterocycloalkyl. In some embodiments, B is selected from

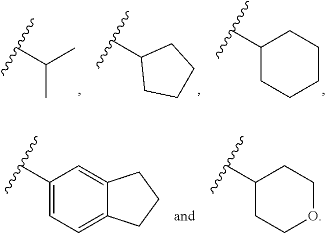

In some embodiments, B is aryl optionally substituted with 1-3 groups each independently selected from the group consisting of —CN, —OH, —NO₂, —N(R⁴)₂, —C(O)N(R⁴)₂, —NR⁴C(O)R⁴, —NR⁴C(O)N(R⁴)₂, —NR⁴ (C₁-C₅)alkylene(O)R⁴, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy.

In some embodiments, B is aryl substituted with 1 or 2 R⁸ groups each independently selected from the group consisting of halogen, C₁-C₅-alkyl, aryl, C₁-C₅-alkoxy, heteroaryl, C₃-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)₂NR⁴, —NR⁴C(O)R⁴, —NR⁴ (C₁-C₅)alkylene(O)R^{4A}, and —O(C₁-C₅-alkylene)N(R⁴)₂. In some embodiments, B is phenyl substituted with 1 or 2 R⁸ groups each independently selected from the group consisting of —F, —Cl, —Br, —CH₃, —CF₃, —CN, —CH(CH₃)₂, —C(CH₃) 3, —CH₂CH₂CH₃, —OCF₃, —OCH₃, —CONH₂, —COOH, —COOCH₃, pyridinyl, phenyl, pyrazolyl, ethynyl, cyclohexyl, cyclopentyl, morpholinyl, tetrahydropyranyl, piperidinyl, piperazinyl, —SO₂NH₂, —SO₂CH₃,

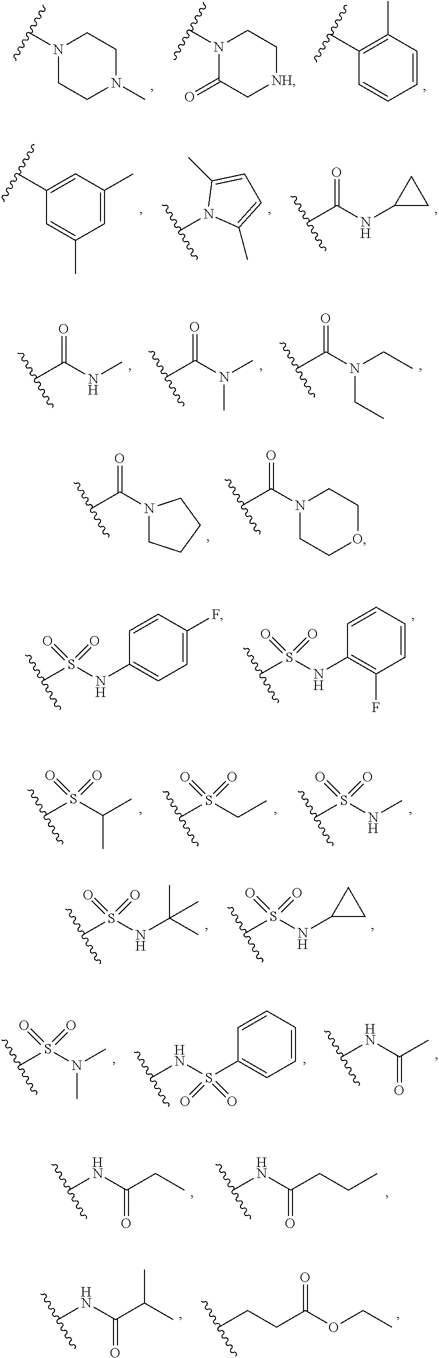

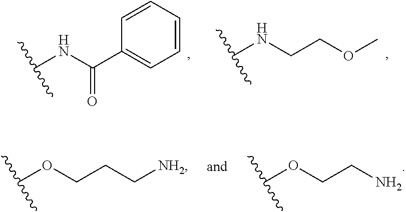

In some embodiments, B is aryl substituted with one R⁸ group selected from halogen, C₁-C₅-alkyl, aryl, C₁-C₅-alkoxy, heteroaryl, C₅-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)₂NR⁴, —NR⁴C(O)R⁴, —NR⁴ (C₁-C₅)alkylene(O)R^{4A}, and —O(C₁-C₅-alkylene)N(R⁴)₂.

In some embodiment, B is selected from

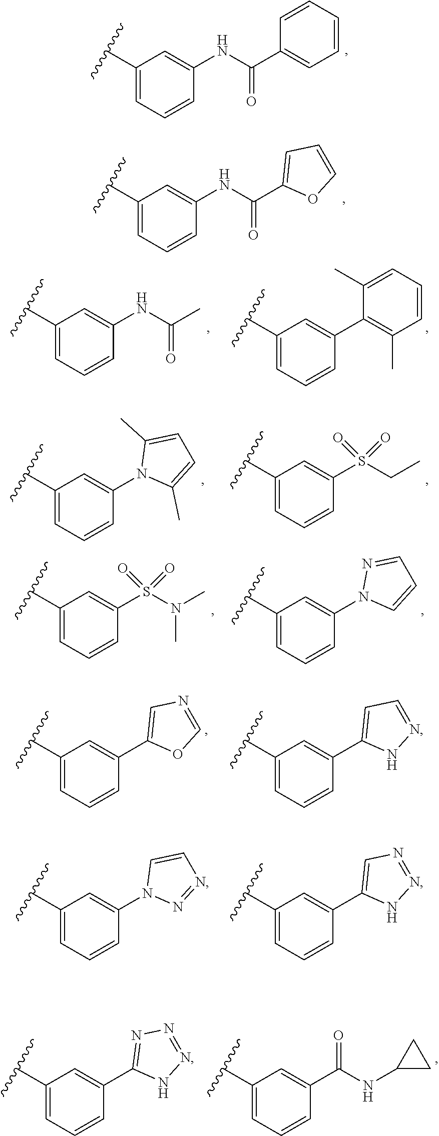

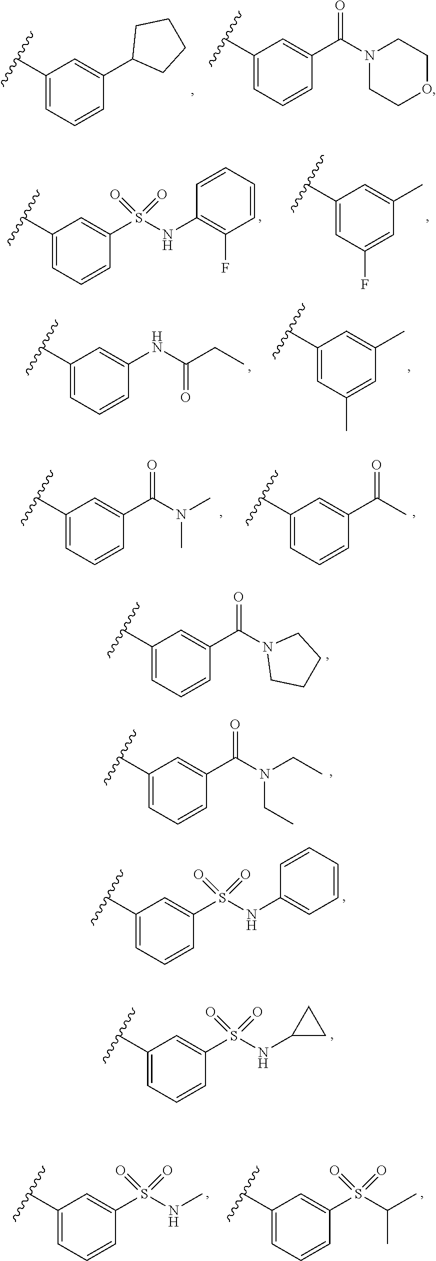

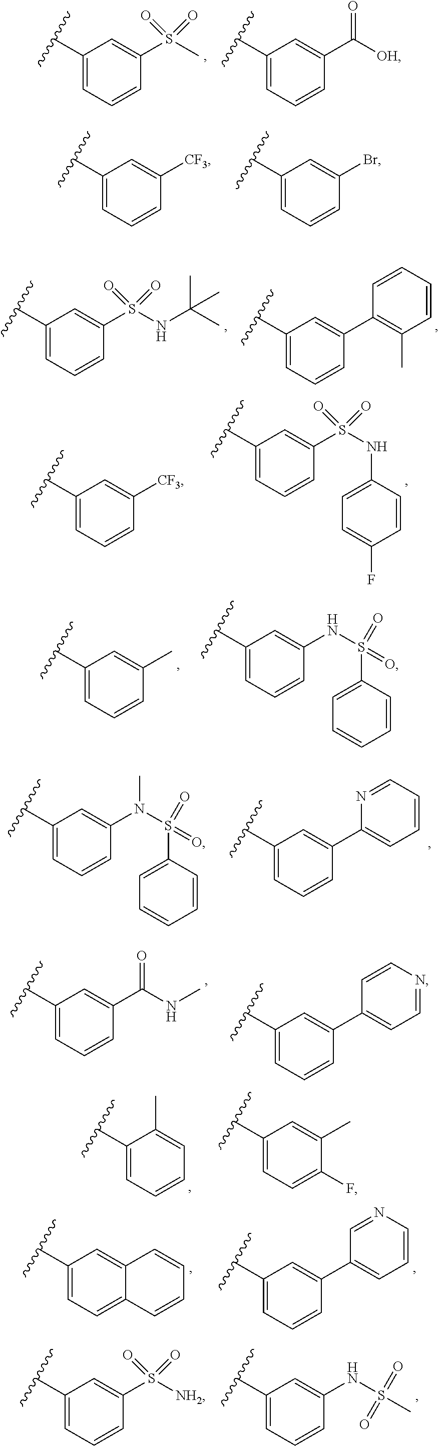

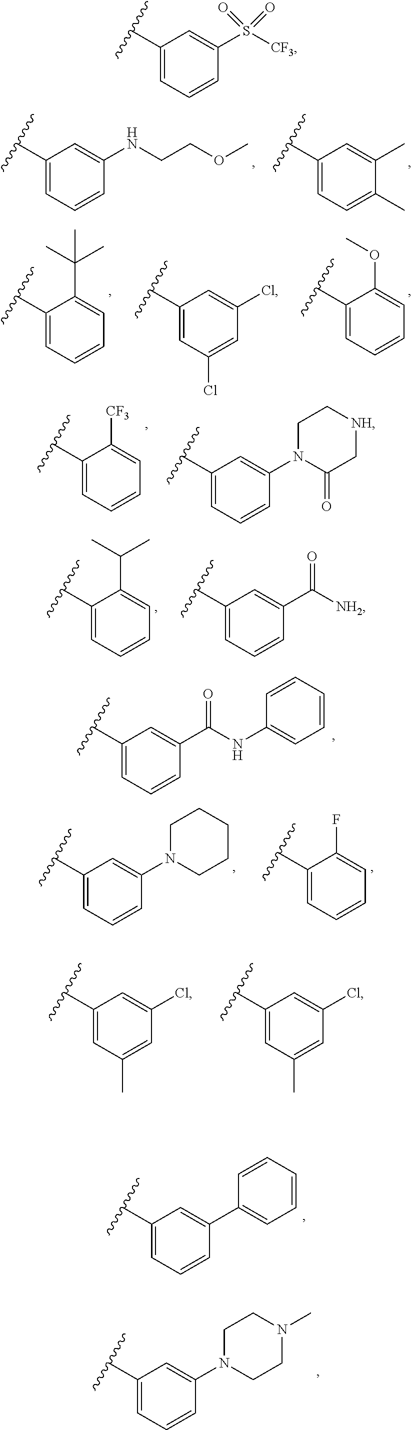

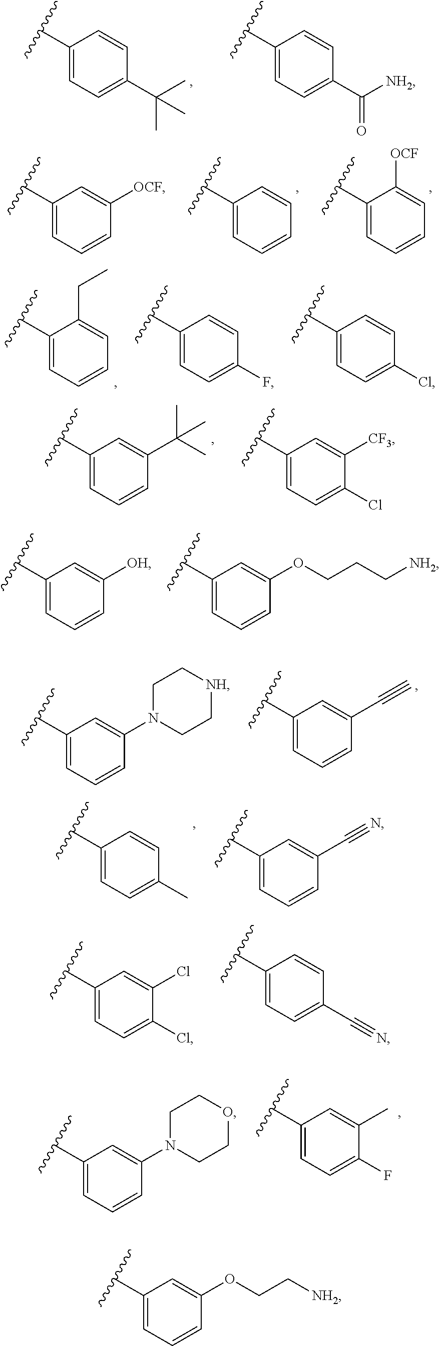

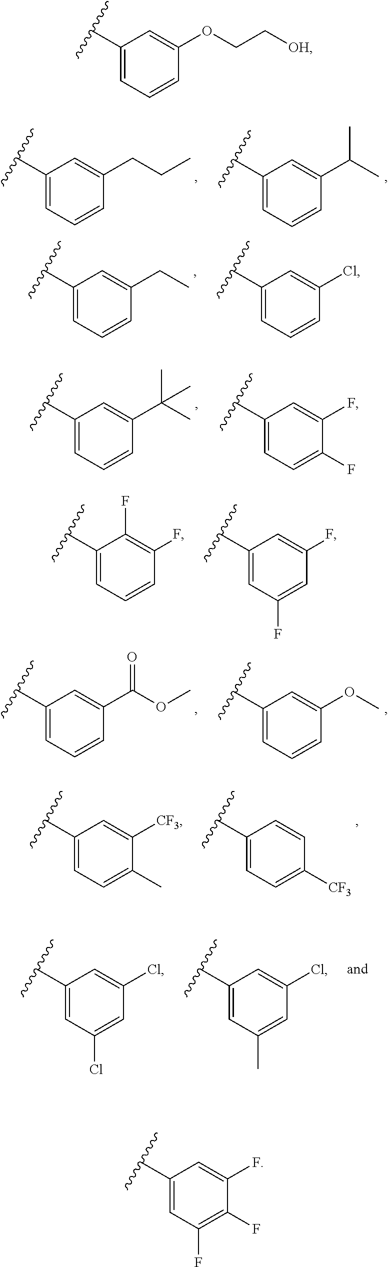

In some embodiments, B is heteroaryl optionally substituted with 1-3 R⁸ groups each independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, heteroaryl, 5-9 membered heterocycloalkyl, C₃-C₅-cycloalkyl, C₁-C₃-alkoxy, and C₁-C₃-haloalkoxy. In some embodiments, B is heteroaryl substituted with 1-3 R⁸ groups each independently selected from the group consisting of 4-9 membered heterocycloalkyl, C₁-C₅-haloalkyl, C₁-C₅-alkyl, aryl, heteroaryl, and alkylaryl. In some embodiments, B is heteroaryl substituted with 1 or 2 R⁸ groups each independently —CF₃, —OH, tetrahydropyranyl, isopropyl, pyridinyl, benzyl or phenyl.

In some embodiments, B is selected from

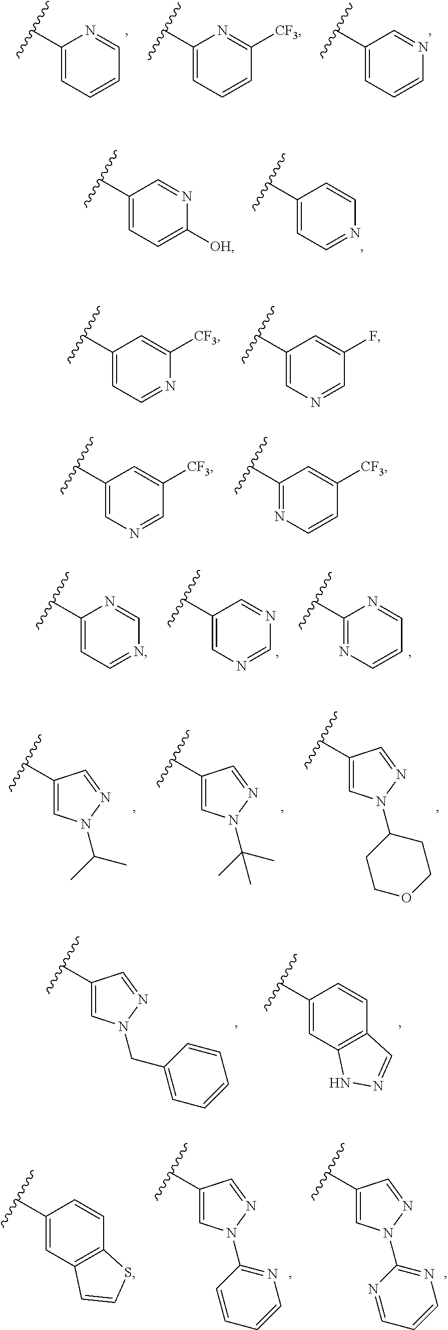

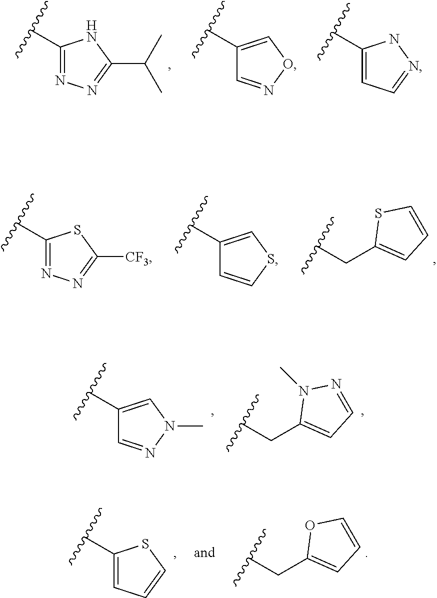

In some embodiments, B is phenyl. In some embodiments,

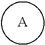

is pyridyl optionally substituted with 1-3 R⁸ groups each independently selected from the group consisting of halogen, C₁-C₃ alkyl, C₁-C₃ haloalkyl, —OR³, and —NH₂. In some embodiments,

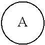

is pyridyl substituted with 1 or 2 R⁸ groups each independently selected from the group consisting of —OH and —OCH₃.

Some embodiments provide a compound of Formula (Ia):

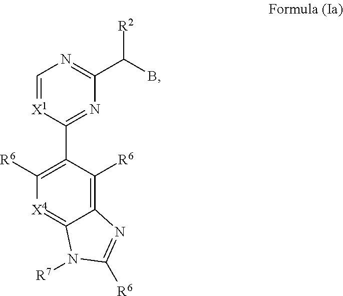

or a pharmaceutically acceptable salt thereof,
   wherein:
B is aryl or heteroaryl each optionally substituted with 1-3 R⁸ groups each independently selected from the group consisting of —(CH₂)ₘCN, —(CH₂)ₘOH, —(CH₂)ₘN(R⁴)₂, —(CH₂)ₘC(O)N(R⁴)₂, —(CH₂)ₘNR⁴C(O)R⁴, —(CH₂)ₘNR⁴C(O)N(R⁴)₂, —(CH₂)ₘNR⁴ (C₁-C₅)alkylene(O)R^{4A}, —(CH₂)ₘC(O)R⁴, —(CH₂)ₘCO₂R⁴, —(CH₂)ₘOC(O)R⁴, —(CH₂)ₘOC(O)OR⁴, —(CH₂)ₘO(C₁-C₅-alkylene)N(R⁴)₂, —(CH₂)ₘSR⁴, —(CH₂)ₘS(O)R⁴, —(CH₂)ₘS(O)₂R⁴, —(CH₂)ₘN(R⁴)S(O)₂R⁴, —(CH₂)ₘS(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy;
X¹ is N or CR¹;
X⁴ is N or CR⁵;
R¹ is hydrogen, halogen, or C₁-C₅ alkyl;
R² is hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl;
R⁵ is hydrogen, halogen, —OH, C₁-C₅ alkoxy, or C₁-C₅ alkyl;
each R⁶ is independently hydrogen, halogen, —OH, C₁-C₅ alkoxy, or C₁-C₅ alkyl;
R⁷ is hydrogen or C₁-C₅ alkyl; and
each m is independently 0, 1, or 2.

In some embodiments, X¹ is N. In some embodiments, X¹ is CH.

In some embodiments, X⁴ is N. In some embodiments, X⁴ is CH.

In some embodiments, R² is hydrogen or —CH₃. In some embodiments, R² is hydrogen. In some embodiments, R² is —CH₃.

In some embodiments, each R⁶ is independently hydrogen, halogen, or hydroxyl.

In some embodiments, R⁷ is hydrogen or —CH₃.

In some embodiments, B is phenyl substituted with 1-3 R⁸ groups each independently selected from —OH, —NR⁴C(O)R⁴, —S(O)₂R⁴, —S(O)₂N(R⁴)₂, halogen, C₁-C₅-alkyl, C₁-C₅-haloalkyl, aryl, heteroaryl, and 5-9 membered heterocycloalkyl.

In some embodiments, B is pyridyl substituted with 1-3 R⁸ groups each independently selected from halogen, C₁-C₅-alkyl, and C₁-C₅-haloalkyl.

In some embodiments, B is pyrazolyl substituted with 1-3 R⁸ groups each independently selected from halogen, C₁-C₅-alkyl, aryl, and heteroaryl.

Some embodiments provide a compound of Formula (Ib):

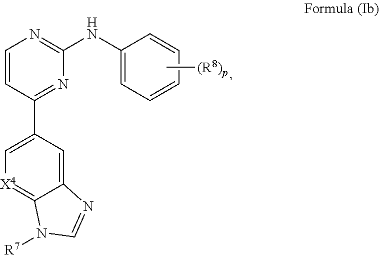

or a pharmaceutically acceptable salt thereof,
   wherein:
X⁴ is N or CR⁵;
R⁵ is hydrogen, halogen, —OH, C₁-C₅ alkoxy, or C₁-C₅ alkyl;
R⁷ is hydrogen or C₁-C₃ alkyl;
each R⁸ is independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)R⁴, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy; and
p is 0, 1, 2, or 3.

Some embodiments provide a compound of Formula (II):

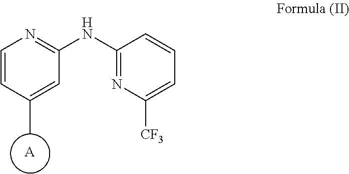

or a pharmaceutically acceptable salt thereof,
wherein:

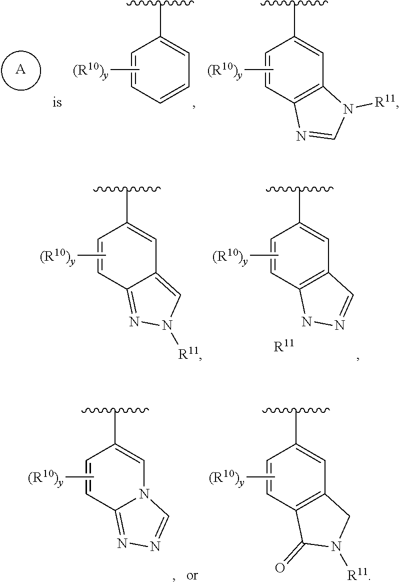

each R¹⁰ is independently selected from halogen, C₁-C₅-alkyl, C₁-C₅-alkoxy, C₁-C₅-haloalkoxy, —CN, —OH, —N(R¹²)₂, —C(O)R¹², —C(O)N(R¹²)₂, —NR¹²C(O)R¹², —CO₂R¹², —OC(O)R¹², —OC(O)OR¹², —SR¹², —S(O)R¹², or —S(O)₂R¹²;
each R¹² is independently hydrogen or C₁-C₅ alkyl;
y is 0, 1, 2, 3, or 4; and
R¹¹ is hydrogen, C₁-C₅ alkyl, C₃-C₆-cycloalkyl, or aryl.

In some embodiments, each R¹⁰ is independently selected from halogen, C₁-C₅-alkyl, or —C(O)N(R¹²)₂. In some embodiments, each R¹⁰ is independently selected from —F, —Cl, —Br, —CH₃, —CH(CH₃)₂, —C(CH₃) 3, or —CONH₂.

In some embodiments, y is 1 or 2.

In some embodiments, R¹¹ is hydrogen, C₁-C₃ alkyl, C₃-C₆-cycloalkyl, or phenyl. In some embodiments, R¹¹ is hydrogen, —CH₃, —CH(CH₃)₂, —C(CH₃) 3, cyclopropyl, or phenyl.

In some embodiments,

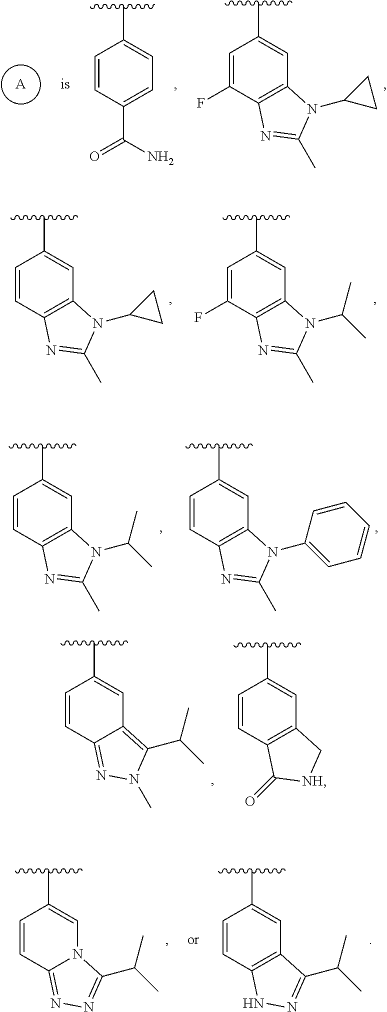

Some embodiments provide a pharmaceutical composition comprising a compound disclosed herein, or a pharmaceutically acceptable salt thereof, and at least one diluent or excipient.

Some embodiments provide a method of treating a disease or disorder, comprising administering to a subject in need thereof a therapeutically effective amount of a compound as described herein, or a pharmaceutically acceptable salt thereof.

Some embodiments provide a method of treating a disease or disorder, comprising administering to a subject in need thereof a therapeutically effective amount of a pharmaceutical composition as described herein.

In some embodiments, the disease or disorder is mediated by a Myc protein. In some embodiments, the Myc protein is selected from an N-Myc protein, a c-Myc protein, and an L-Myc protein.

Some embodiments provide a method of treating cancer, comprising administering to a subject in need thereof a therapeutically effective amount of a compound as described herein, or a pharmaceutically acceptable salt thereof.

Some embodiments provide a method of treating cancer, comprising administering to a subject in need thereof a therapeutically effective amount of a pharmaceutical composition as described herein.

In some embodiments, the cancer is mediated by a Myc protein. In some embodiments, the Myc protein is selected from an N-Myc protein, a c-Myc protein, and an L-Myc protein.

In some embodiments, the cancer is selected from head and neck cancer, nervous system cancer, brain cancer, neuroblastoma, medulloblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genitourinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site. In some embodiments, the cancer is medulloblastoma. In some embodiments, the subject is human.

Some embodiments provide a method of treating a disease or condition mediated by CDK9 in a subject comprising administering a therapeutically effective amount of a compound, or a pharmaceutically acceptable salt thereof, as described herein to the subject. In some embodiments, the compound is a CDK9 inhibitor. In some embodiments, the compound inhibits activity of CDK9.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are incorporated by reference herein to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the set-up and workflow of high content screen for inhibitors of endogenous Myc.

FIGS. 2A-2D show the compound profile for Compound A. FIG. 2A shows Myc expression inhibition in PDX511 medulloblastoma cells by Compound A with an IC₅₀ of 11.7 μM. FIG. 2B shows that Compound A had no effect on the expression of the retinoblastoma (Rb) protein in PDX511 medulloblastoma cells. FIG. 2C shows that Compound A had no effect on cell viability of PDX511 medulloblastoma cells at 4 hours. FIG. 2D shows images of immunofluorescence-based detection of Myc expression (top row), cell viability (middle row), and Rb protein expression (bottom row) of PDX511 medulloblastoma cells treated with Compound A at various concentrations.

FIGS. 3A-3D show the compound profile for Compound 171. FIG. 3A shows Myc expression inhibition in PDX511 medulloblastoma cells by Compound 171. FIG. 3B shows that Compound 171 had no effect on expression of the retinoblastoma (Rb) protein in PDX511 medulloblastoma cells. FIG. 3C shows that Compound 171 had no effect on cell viability of PDX511 medulloblastoma cells at 4 hours. FIG. 3D shows images of immunofluorescence-based detection of Myc expressions (top row), cell viability (middle row), and Rb protein expressions (bottom row) of PDX511 medulloblastoma cells treated with Compound 171 at various concentrations.

FIG. 4A shows Western Blot gel data from cultured cells treated with Compound 171 at various times. Inhibition of Myc expression was observed as early as 1 hour, and greater inhibition was seen at 2 and 4 hours. FIG. 4B and FIG. 4C show inhibition of Myc expression in cells treated with 0.18 μM of Compound 171 after 48 hours and after 96 hours respectively using CellTiter Glo assay. The data suggest that Compound 171 cased an 80% reduction in cell viability by 48 hours, and 100% reduction in viability by 96 hours.

FIGS. 5A-5D show inhibition of Myc expression in different cell lines derived from multiple types of cancer after treatment with various concentrations of Compound 171. FIG. 5A shows the inhibition result from the THP-1 cell line derived from acute monocytic leukemia.

FIG. 5B shows the inhibition result from the HL-60 cell line derived from acute promyelocytic leukemia. FIG. 5C shows the inhibition result from the BxPC-3 cell line derived from pancreatic adenocarcinoma. FIG. 5D shows the inhibition result from the H-82 cell line derived from small cell lung cancer.

FIG. 6 shows concentrations of Compound 171 at various time points in the plasma and in the brain of non-tumor-bearing mice after systemic oral administration of the compound at 100 mg/kg.

FIG. 7 shows reduction of Myc levels in intracranial tumors in mice transplanted with medulloblastoma PDX cells after oral administration of Compound 171 at 100 mg/kg.

### DETAILED DESCRIPTION

The present disclosure provides novel Myc modulator compounds as described herein. In some embodiments, the Myc modulator compounds are Myc inhibitors. As described herein, the compounds of the present disclosure exhibit potent inhibitory activities against Myc-mediated disease or disorder, such as cancers.

The present disclosure provides novel CDK9 modulator compounds as described herein. In some embodiments, the CDK9 modulator compounds are CDK9 inhibitors. As described herein, the compounds of the present disclosure exhibit potent inhibitory activities against CDK9-mediated disease or disorder, such as cancers.

### Compounds

Some embodiments provide a compound of Formula (I):

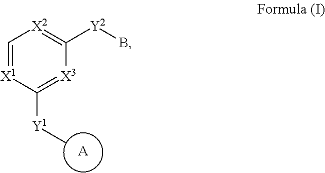

or a pharmaceutically acceptable salt thereof, for use in treating a disease or disorder mediated by a Myc protein, wherein:
X¹, X², and X³ are each independently N or CR¹, wherein at least one of X¹, X², and X₃ is N;
Y¹ is a bond, —O—, —S—, —CH₂—, or —N(R²)(CH₂)ₙ—;
Y² is —O—, —S—, —CH₂—, or —N(R²)(CH₂)ₙ—;

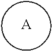

is phenyl, 5-9 membered heteroaryl,

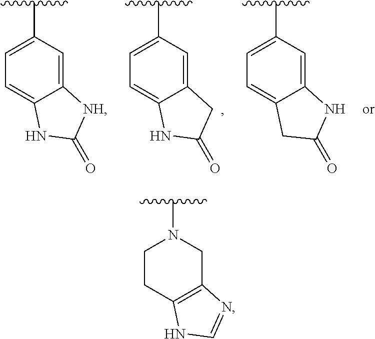

wherein the phenyl and 5-9 membered heteroaryl are each optionally substituted with 1-3 R⁹ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂;
B is C₁-C₅ alkyl, aryl, heteroaryl, 5-9 membered heterocycloalkyl, or C₃-C₁₀-cycloalkyl, wherein the aryl, heteroaryl, 5-9 membered heterocycloalkyl, and C₃-C₁₀-cycloalkyl are each optionally substituted with 1-3 R⁸ groups each independently selected from the group consisting of —(CH₂)ₙCN, —(CH₂)ₙOH, —(CH₂)ₙN(R⁴)₂, —(CH₂)ₙC(O)N(R⁴)₂, —(CH₂)ₙNR⁴C(O)R⁴, —(CH₂)ₙNR⁴C(O)N(R⁴)₂, —(CH₂)ₙNR⁴(C₁-C₅)alkylene(O)R^{4A}, —(CH₂)ₙC(O)R⁴, —(CH₂)ₙCO₂R⁴, —(CH₂)ₙOC(O)R⁴, —(CH₂)ₙOC(O)OR⁴, —(CH₂)ₙO(C₁-C₅-alkylene)N(R⁴)₂, —(CH₂)ₙSR⁴, —(CH₂)ₙS(O)R⁴, —(CH₂)ₙS(O)₂R⁴, —(CH₂)ₙN(R⁴)S(O)₂R⁴, —(CH₂)ₙS(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₅-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo;
R¹ is hydrogen, halogen, or C₁-C₅ alkyl;
R² and R³ are each independently hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl; and
each n is independently 0, 1, 2, 3, or 4. In some embodiments,

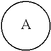

is not

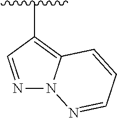 [SMILES: *c1cnn2ncccc12]

optionally substituted with 1-3 R⁹ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂.

In some embodiments, X¹ is CR¹, and X² and X³ are each independently N. In some embodiments, X¹ and X² are each independently CR¹, and X₃ is N. In some embodiments, X¹ and X³ are each independently CR¹, and X² is N. In some embodiments, X¹, X² and X³ are each independently N.

In some embodiments, Y¹ is a bond. In some embodiments, Y¹ is —O—, or —N(R²)(CH₂)ₙ—. In some embodiments, Y¹ is —N(R²)(CH₂)ₙ—. In some embodiments, Y¹ is —CH₂—, —S—, or —O—.

In some embodiments, R¹ is hydrogen, or —CH₃.

In some embodiments,

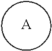

is 5-9-membered heteroaryl or phenyl each optionally substituted with 1-3 R⁹ groups each independently selected from the group consisting of halogen, C₁-C₅ alkyl, C₁-C₅ haloalkyl, —OR³, and —NH₂. In some embodiments,

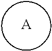

is 5-9 membered heteroaryl or phenyl each substituted with 1-3 R′ groups each independently selected from the group consisting of halogen, —OH, —OCH₃, —NH₂, and —CH₃.

In some embodiments,

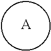

is selected from:

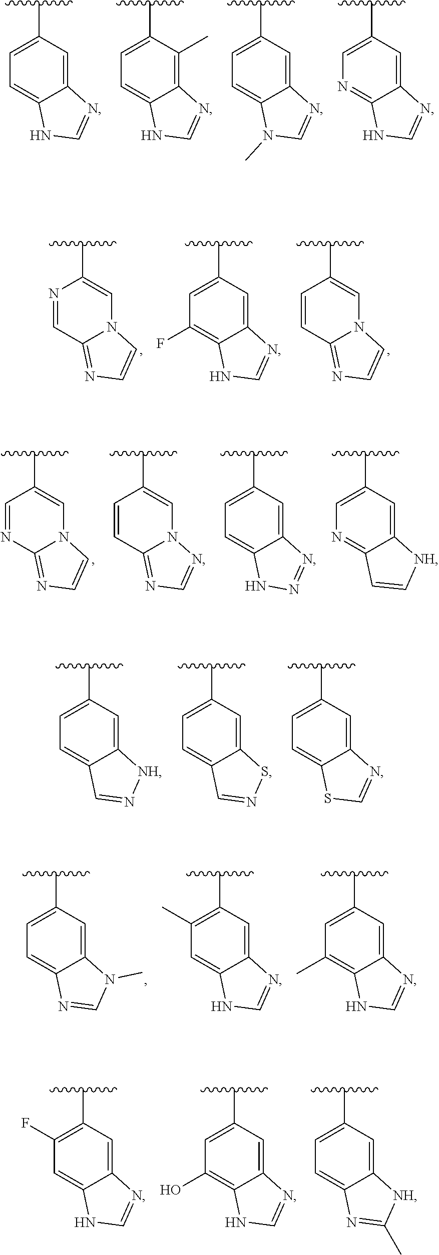

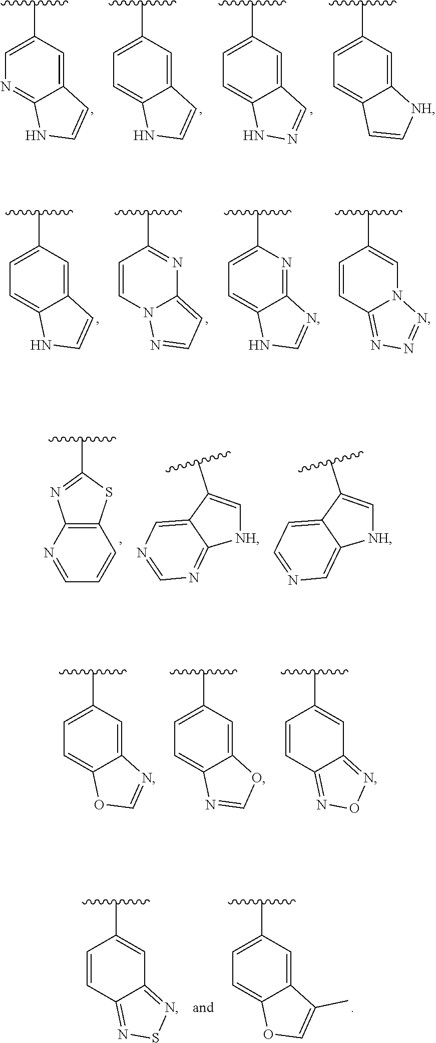

In some embodiments,

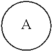

is selected from:

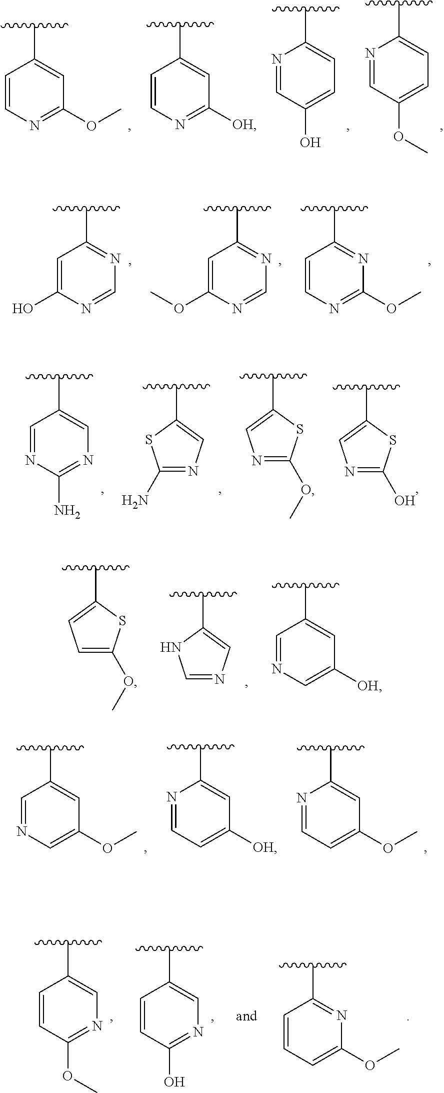

In some embodiments,

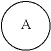

is selected from

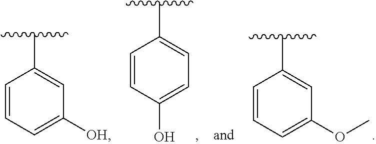

In some embodiments,

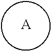

is selected from

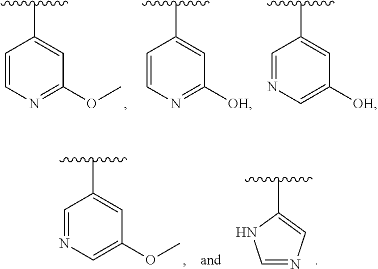

In some embodiments,

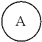

is not selected from

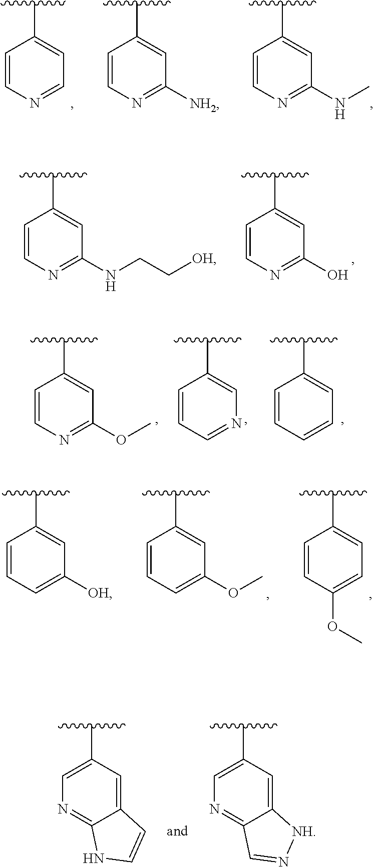

In some embodiments, B is C₁-C₅ alkyl, C₃-C₉-cycloalkyl, or 5-9 membered heterocycloalkyl. In some embodiments, B is selected from

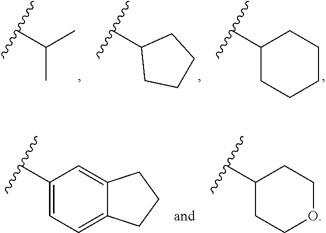

In some embodiments, B is aryl optionally substituted with 1-3 groups independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, alkylaryl, heteroaryl, 5-9 membered heterocycloalkyl, 5 C₃-C₉-cycloalkyl, C₁-C₅-alkoxy, and C₁-C₅-haloalkoxy, and wherein said aryl is optionally substituted with 1 or 2 C₁-C₅-alkyl, and said 5-9 membered heterocycloalkyl is optionally substituted with 1 or 2 substituents each independently selected from C₁-C₅-alkyl and oxo.

In some embodiments, B is aryl substituted with 1 or 2 R⁸ groups each independently selected from the group consisting of halogen, C₁-C₅-alkyl, aryl, C₁-C₅-alkoxy, heteroaryl, C₅-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)>NR⁴, —NR⁴C(O)R⁴, —NR⁴ (C₁-C₅)alkylene(O)R^{4A}, and —O(C₁-C₅-alkylene)N(R⁴)₂, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo. In some embodiments, B is phenyl substituted with 1 or 2 R⁸ groups each independently selected from the group consisting of —F, —Cl, —Br, —CH₃, —CF₃, —CN, —CH(CH₃)₂, —C(CH₃) 3, —CH₂CH₂CH₃, —OCF₃, —OCH₃, —CONH₂, —COOH, —COOCH₃, pyridinyl, phenyl, pyrazolyl, ethynyl, cyclohexyl, cyclopentyl, morpholinyl, tetrahydropyranyl, piperidinyl, piperazinyl, —SO₂NH₂, —SO₂CH₃,

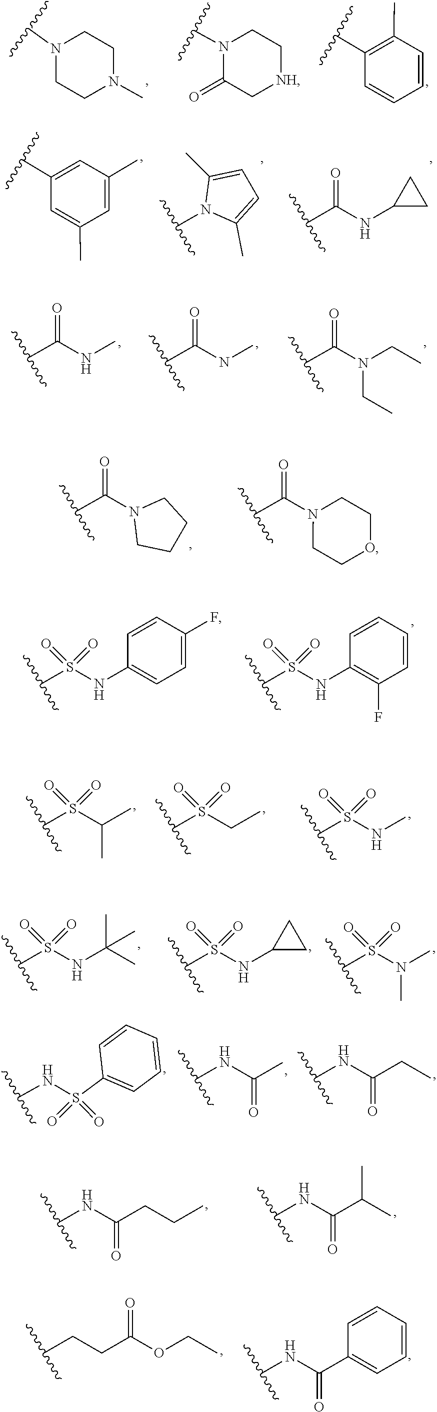

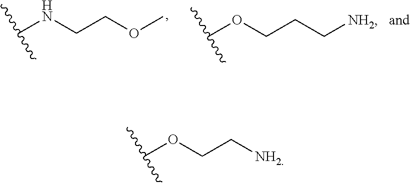

In some embodiments, aryl is substituted with one R⁸ group selected from halogen, C₁-C₅-alkyl, aryl, C₁-C₅-alkoxy, heteroaryl, C₅-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)₂NR⁴, —NR⁴C(O)R⁴, —NR⁴ (C₁-C₅)alkylene(O)R^{4A}, and —O(C₁-C₅-alkylene)N(R⁴)₂, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

In some embodiments, B is selected from

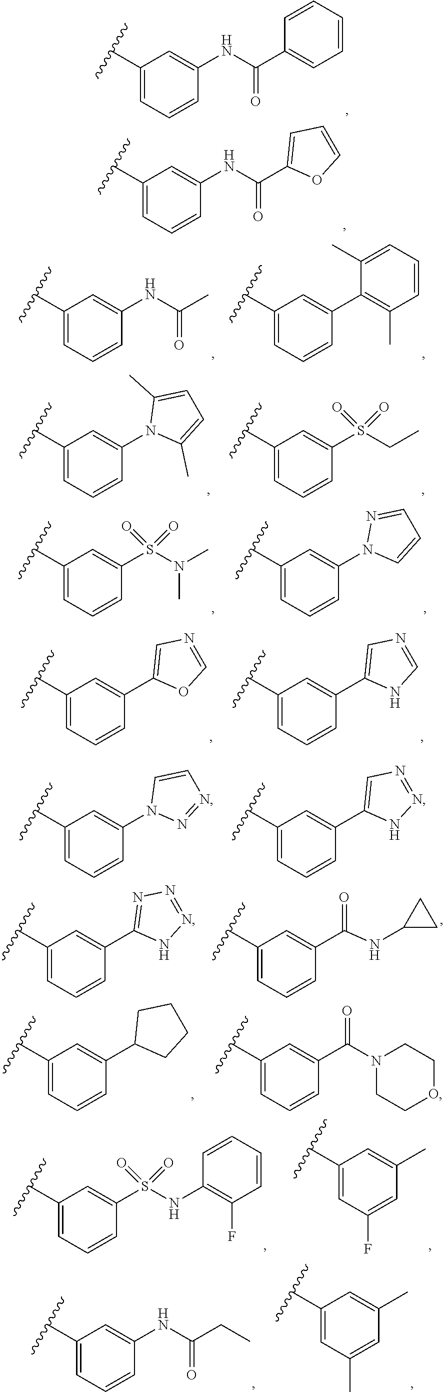

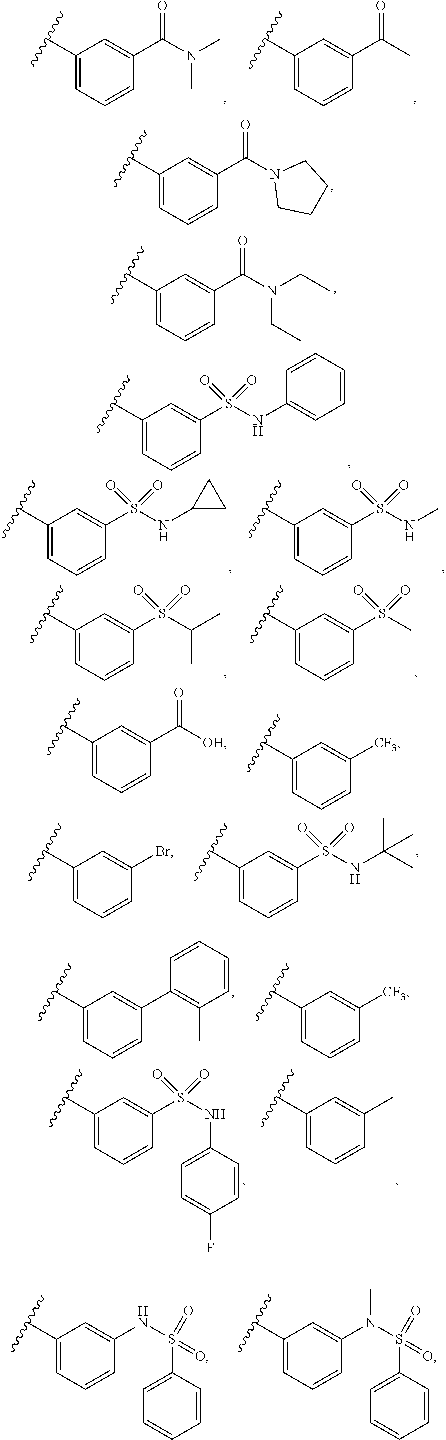

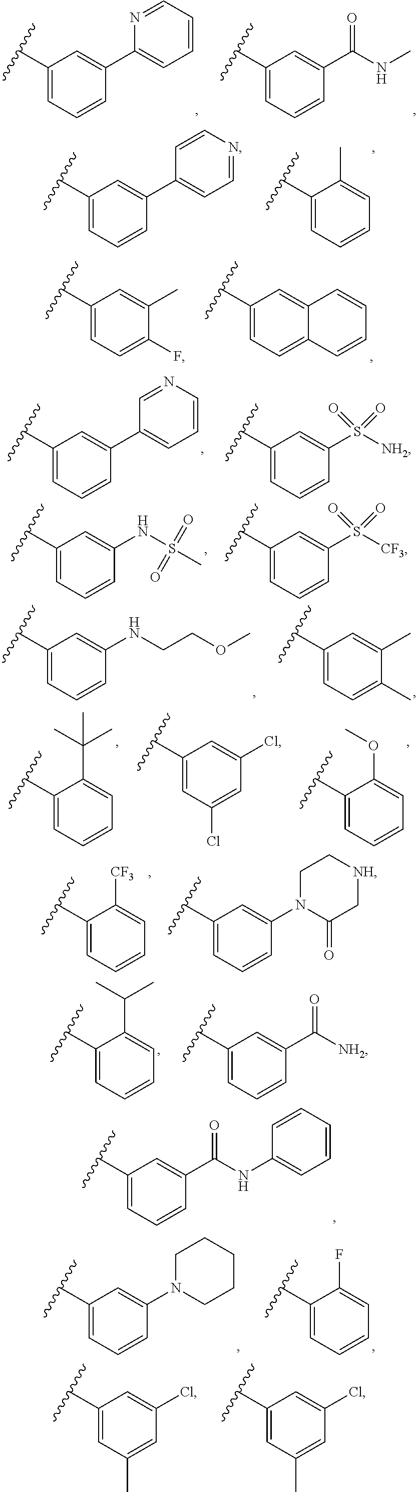

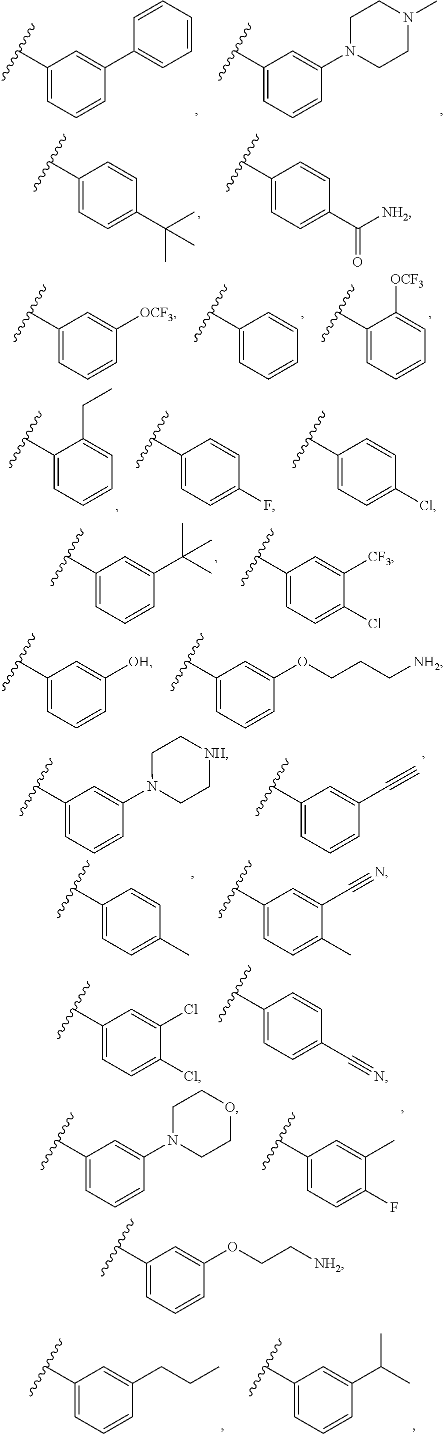

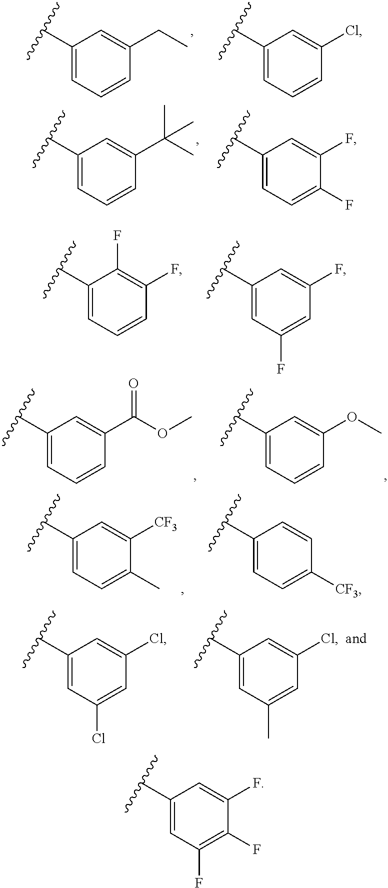

In some embodiments, B is heteroaryl optionally substituted with 1-3 R⁸ groups each independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, heteroaryl, 5-9 membered heterocycloalkyl, C₃-C₅-cycloalkyl, C₁-C₃-alkoxy, and C₁-C₃-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo. In some embodiments, B is heteroaryl substituted with 1-3 R⁸ groups each independently selected from the group consisting of 5-9 membered heterocycloalkyl, C₁-C₅-haloalkyl, C₁-C₅-alkyl, aryl, heteroaryl, and alkylaryl, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo. In some embodiments, B is heteroaryl substituted with 1 or 2 R⁸ groups each independently —CF₃, —OH, tetrahydropyranyl, isopropyl, pyridinyl, benzyl or phenyl.

In some embodiments, B is selected from

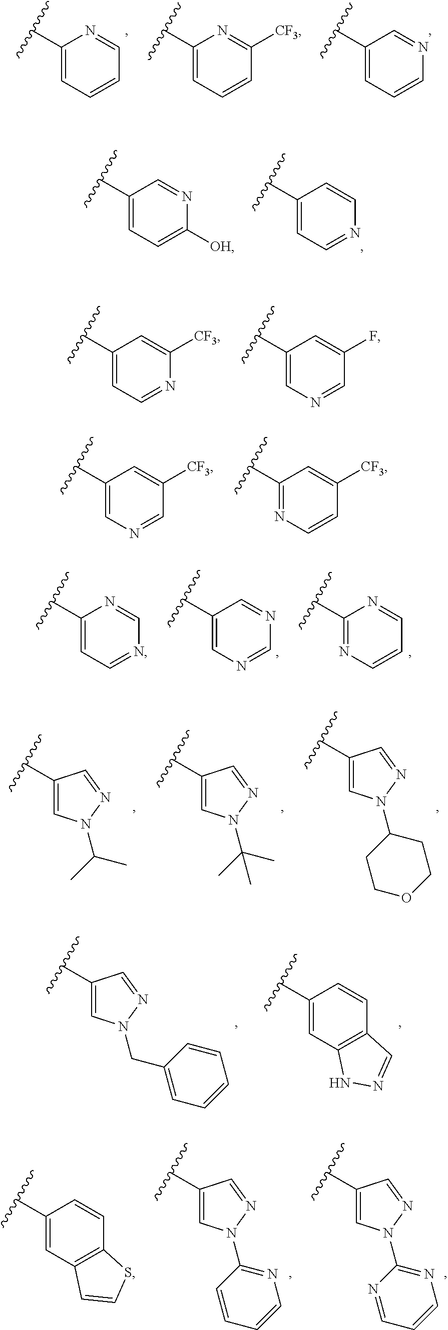

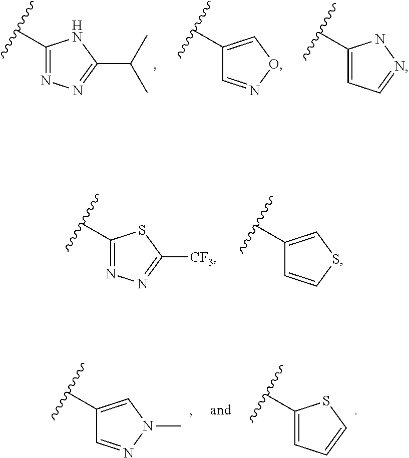

In some embodiments, B is phenyl. In some embodiments,

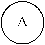

is pyridyl optionally substituted with 1-3 R⁸ groups each independently selected from the group consisting of halogen, C₁-C₃ alkyl, C₁-C₃ haloalkyl, —OR³, and —NH₂. In some embodiments,

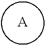

is pyridyl substituted 1 or 2 R⁸ groups each independently selected from the group consisting of —OH and —OCH₃.

Some embodiments provide a compound of to Formula (Ia):

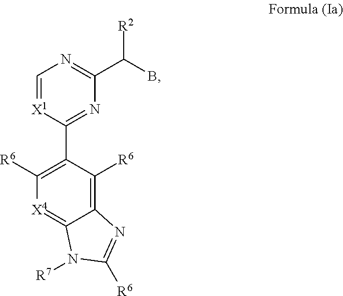

or a pharmaceutically acceptable salt thereof,
   wherein:
B is aryl or heteroaryl each optionally substituted with 1-3 R⁸ groups each independently selected from the group consisting of —(CH₂)ₘCN, —(CH₂)ₘOH, —(CH₂)ₘN(R⁴)₂, —(CH₂)ₘC(O)N(R⁴)₂, —(CH₂)ₘNR⁴C(O)R⁴, —(CH₂)ₘNR⁴C(O)N(R⁴)₂, —(CH₂)ₘNR⁴ (C₁-C₅)alkylene(O)R^{4A}, —(CH₂)ₘC(O)R⁴, —(CH₂)ₘCO₂R⁴, —(CH₂)ₘOC(O)R⁴, —(CH₂)ₘOC(O)OR⁴, —(CH₂)ₘO(C₁-C₅-alkylene)N(R⁴)₂, —(CH₂)ₘSR⁴, —(CH₂)ₘS(O)R⁴, —(CH₂)ₘS(O)₂R⁴, —(CH₂)ₘN(R⁴)S(O)₂R⁴, —(CH₂)ₘS(O)₂N(R⁴)₂, halogen, C₁-C₅-alkyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy;
X¹ is N or CR¹;
X⁴ is N or CR⁵;
R¹ is hydrogen, halogen, or C₁-C₅ alkyl;
R² is hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl;
R⁵ is hydrogen, halogen, —OH, C₁-C₅ alkoxy, or C₁-C₅ alkyl;
each R⁶ is independently hydrogen, halogen, —OH, C₁-C₅ alkoxy, or C₁-C₅ alkyl;
R⁷ is hydrogen or C₁-C₅ alkyl; and
each m is independently 0, 1, or 2.

In some embodiments, X¹ is N. In some embodiments, X¹ is CH.

In some embodiments, X⁴ is N. In some embodiments, X⁴ is CH.

In some embodiments, R² is hydrogen or —CH₃. In some embodiments, R² is hydrogen. In some embodiments, R² is —CH₃.

In some embodiments, each R⁶ is independently hydrogen, halogen, or —OH. In some embodiments, each R^{4A} is independently C₁-C₅ alkyl or C₃-C₆-cycloalkyl. In some embodiments, each R^{4A} is C₁-C₅ alkyl.

In some embodiments, R⁷ is hydrogen or —CH₃.

In some embodiments, B is phenyl substituted with 1-3 R⁸ groups each independently selected from —OH, —NR⁴C(O)R⁴, —S(O)₂R⁴, —S(O)₂N(R⁴)₂, halogen, C₁-C₅-alkyl, C₁-C₅-haloalkyl, aryl, heteroaryl, and 5-9 membered heterocycloalkyl.

In some embodiments, B is pyridyl substituted with 1-3 R⁸ groups each independently selected from halogen, C₁-C₅-alkyl, and C₁-C₅-haloalkyl.

In some embodiments, B is pyrazolyl substituted with 1-3 R⁸ groups each independently selected from halogen, C₁-C₅-alkyl, aryl, and heteroaryl.

Some embodiments provide a compound of Formula (Ib):

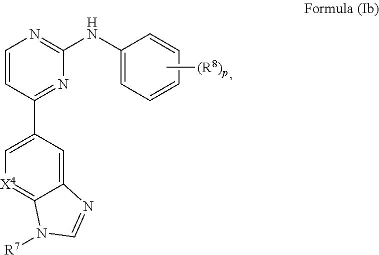

or a pharmaceutically acceptable salt thereof,
   wherein:
X⁴ is N or CR⁵;
R⁵ is hydrogen, halogen, —OH, C₁-C₅ alkoxy, or C₁-C₅ alkyl;
R⁷ is hydrogen or C₁-C₃ alkyl;
each R⁸ is independently selected from the group consisting of —CN, —OH, —NO₂, —N(R⁴)₂, —C(O)N(R⁴)₂, —NR⁴C(O)R⁴, —NR⁴C(O)N(R⁴)₂, —NR⁴ (C₁-C₅)alkylene(O)R⁴, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)R⁴, halogen, C₁-C₅-alkyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy; and
p is 0, 1, 2, or 3.

Some embodiments provide a compound of Formula (II):

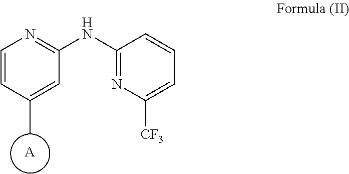

or a pharmaceutically acceptable salt thereof,
wherein:

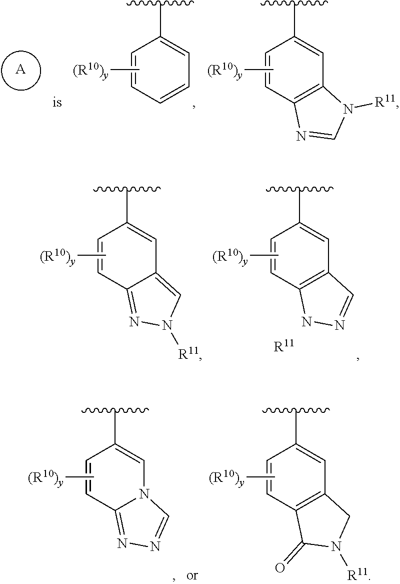

each R¹⁰ is independently selected from halogen, C₁-C₅-alkyl, C₁-C₅-alkoxy, C₁-C₅-haloalkoxy, —CN, —OH, —N(R¹²)₂, —C(O)R¹², —C(O)N(R¹²)₂, —NR¹²C(O)R¹², —CO₂R¹², —OC(O)R¹², —OC(O)OR¹², —SR¹², —S(O)R¹², or —S(O)₂R¹²;
each R¹² is independently hydrogen or C₁-C₅ alkyl;
y is 0, 1, 2, 3, or 4; and
R¹¹ is hydrogen, C₁-C₅ alkyl, C₃-C₆-cycloalkyl, or aryl.

In some embodiments, each R¹⁰ is independently selected from halogen, C₁-C₅-alkyl, or —C(O)N(R¹²)₂. In some embodiments, each R¹⁰ is independently selected from —F, —Cl, —Br, —CH₃, —CH(CH₃)₂, —C(CH₃) 3, or —CONH₂.

In some embodiments, y is 1 or 2.

In some embodiments, R¹¹ is hydrogen, C₁-C₃ alkyl, C₃-C₆-cycloalkyl, or phenyl. In some embodiments, R¹¹ is hydrogen, —CH₃, —CH(CH₃)₂, —C(CH₃) 3, cyclopropyl, or phenyl.

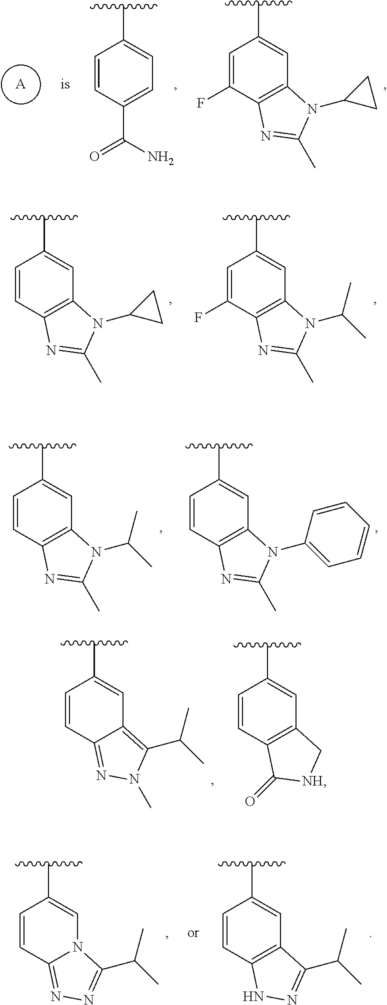

In some embodiments,

In some embodiments, the present disclosure provides a pharmaceutically acceptable salt of a compound described in Table 1.

In some embodiments, the compound or a pharmaceutically acceptable salt thereof, is selected from the group consisting of:
N-(3-((4-(1H-benzo[d]imidazol-5-yl)pyrimidin-2-yl)amino)phenyl)benzamide (Compound 1),
N-(3-((4-(1H-benzo[d]imidazol-5-yl)pyrimidin-2-yl)amino)phenyl)acetamide (Compound 2),
4-(1H-benzo[d]imidazol-5-yl)-N-(2′,6′-dimethyl-[1,1′-biphenyl]-3-yl)pyrimidin-2-amine (Compound 3),
4-(1H-benzo[d]imidazol-5-yl)-N-(3-(2,5-dimethyl-1H-pyrrol-1-yl)phenyl)pyrimidin-2-amine (Compound 4),
4-(1H-benzo[d]imidazol-5-yl)-N-(3-(ethylsulfonyl)phenyl)pyrimidin-2-amine (Compound 5),
4-(1H-benzo[d]imidazol-5-yl)-N-(1-(tetrahydro-2H-pyran-4-yl)-1H-pyrazol-4-yl)pyrimidin-2-amine (Compound 6),
3-((4-(1H-benzo[d]imidazol-5-yl)pyrimidin-2-yl)amino)-N,N-dimethylbenzenesulfonamide (Compound 7),
N-(3-(1H-pyrazol-1-yl)phenyl)-4-(1H-benzo[d]imidazol-5-yl)pyrimidin-2-amine (Compound 8),
3-((4-(1H-benzo[d]imidazol-5-yl)pyrimidin-2-yl)amino)-N-cyclopropylbenzamide (Compound 9),
4-(1H-benzo[d]imidazol-5-yl)-N-(3-cyclopentylphenyl)pyrimidin-2-amine (Compound 10),
(3-((4-(1H-benzo[d]imidazol-5-yl)pyrimidin-2-yl)amino)phenyl)(morpholino)methanone (Compound 11),
3-((4-(1H-benzo[d]imidazol-5-yl)pyrimidin-2-yl)amino)-N-(2-fluorophenyl)benzenesulfonamide (Compound 12),
4-(1H-benzo[d]imidazol-5-yl)-N-(3-fluoro-5-methylphenyl)pyrimidin-2-amine (Compound 13),
4-(1H-benzo[d]imidazol-5-yl)-N-(5-(trifluoromethyl)pyridin-3-yl)pyrimidin-2-amine (Compound 14),
N-(3-((4-(1H-benzo[d]imidazol-5-yl)pyrimidin-2-yl)amino)phenyl)propionamide (Compound 15),
4-(1H-benzo[d]imidazol-5-yl)-N-(3,5-dimethylphenyl)pyrimidin-2-amine (Compound 16),
(3-((4-(1H-benzo[d]imidazol-5-yl)pyrimidin-2-yl)amino)phenyl)(pyrrolidin-1-yl)methanone (Compound 17),
3-((4-(1H-benzo[d]imidazol-5-yl)pyrimidin-2-yl)amino)-N,N-dimethylbenzamide (Compound 18),
3-((4-(1H-benzo[d]imidazol-5-yl)pyrimidin-2-yl)amino)-N,N-diethylbenzamide (Compound 19),
4-(1H-benzo[d]imidazol-5-yl)-N-(1-(pyrimidin-2-yl)-1H-pyrazol-4-yl)pyrimidin-2-amine (Compound 20),
4-(1H-benzo[d]imidazol-5-yl)-N-(1-isopropyl-1H-pyrazol-4-yl)pyrimidin-2-amine (Compound 21),
6-((4-(1H-benzo[d]imidazol-5-yl)pyrimidin-2-yl)amino)pyridin-2-ol (Compound 22),
3-((4-(1H-benzo[d]imidazol-5-yl)pyrimidin-2-yl)amino)-N-phenylbenzenesulfonamide (Compound 23),
3-((4-(1H-benzo[d]imidazol-5-yl)pyrimidin-2-yl)amino)-N-cyclopropylbenzenesulfonamide (Compound 24),
3-((4-(1H-benzo[d]imidazol-5-yl)pyrimidin-2-yl)amino)-N-methylbenzenesulfonamide (Compound 25),
4-(1H-benzo[d]imidazol-5-yl)-N-(3-(isopropylsulfonyl)phenyl)pyrimidin-2-amine (Compound 26),
4-(1H-benzo[d]imidazol-5-yl)-N-(3-(methylsulfonyl)phenyl)pyrimidin-2-amine (Compound 27),
3-((4-(1H-benzo[d]imidazol-5-yl)pyrimidin-2-yl)amino)benzoic acid (Compound 28),
5-((4-(1H-benzo[d]imidazol-5-yl)pyrimidin-2-yl)amino)pyridin-2-ol (Compound 29),
4-(1H-pyrrolo[2,3-c]pyridin-3-yl)-N-(3-(trifluoromethyl)phenyl)pyrimidin-2-amine (Compound 30),
4-(1H-benzo[d]imidazol-5-yl)-N-(3-bromophenyl)pyrimidin-2-amine (Compound 31),
3-((4-(1H-benzo[d]imidazol-5-yl)pyrimidin-2-yl)amino)-N-(tert-butyl)benzenesulfonamide (Compound 32),
4-(1H-benzo[d]imidazol-5-yl)-N-(2′-methyl-[1,1′-biphenyl]-3-yl)pyrimidin-2-amine (Compound 33),
4-(1-methyl-1H-benzo[d]imidazol-5-yl)-N-(3-(trifluoromethyl)phenyl)pyrimidin-2-amine (Compound 34),
4-(1H-benzo[d]imidazol-5-yl)-N-(1-(pyridin-2-yl)-1H-pyrazol-4-yl)pyrimidin-2-amine (Compound 35),
3-((4-(1H-benzo[d]imidazol-5-yl)pyrimidin-2-yl)amino)-N-(4-fluorophenyl)benzenesulfonamide (Compound 36),
4-(1H-benzo[d]imidazol-5-yl)-N-(1-benzyl-1H-pyrazol-4-yl)pyrimidin-2-amine (Compound 37),
N-(3-((4-(1H-benzo[d]imidazol-5-yl)pyrimidin-2-yl)amino)phenyl)benzenesulfonamide (Compound 38),
4-(1H-benzo[d]imidazol-5-yl)-N-(1-phenyl-1H-pyrazol-4-yl)pyrimidin-2-amine (Compound 39),
4-(1H-benzo[d]imidazol-5-yl)-N-(m-tolyl)pyrimidin-2-amine (Compound 40),
4-(1-methyl-1H-benzo[d]imidazol-6-yl)-N-(3-(trifluoromethyl)phenyl)pyrimidin-2-amine (Compound 41),
3-((4-(1H-benzo[d]imidazol-5-yl)pyrimidin-2-yl)amino)-N-methylbenzamide (Compound 42),
4-(1H-benzo[d]imidazol-5-yl)-N-(3-(pyridin-4-yl)phenyl)pyrimidin-2-amine (Compound 43),
4-(1H-benzo[d]imidazol-5-yl)-N-(3-(pyridin-2-yl)phenyl)pyrimidin-2-amine (Compound 44),
4-(1H-benzo[d]imidazol-5-yl)-N-(3-(pyridin-3-yl)phenyl)pyrimidin-2-amine (Compound 45),
3-((4-(1H-benzo[d]imidazol-5-yl)pyrimidin-2-yl)amino)benzenesulfonamide (Compound 46),
N¹-(4-(1H-benzo[d]imidazol-5-yl)pyrimidin-2-yl)-N³-(2-methoxyethyl)benzene-1,3-diamine (Compound 47),
4-(1H-benzo[d]imidazol-5-yl)-N-(4-fluoro-3-methylphenyl)pyrimidin-2-amine (Compound 48),
4-(1H-benzo[d]imidazol-5-yl)-N-(3,5-dichlorophenyl)pyrimidin-2-amine (Compound 49),
6-(1H-benzo[d]imidazol-5-yl)-N-(3-(trifluoromethyl)phenyl)pyridin-2-amine (Compound 50),
4-(1H-benzo[d]imidazol-5-yl)-N-(o-tolyl)pyrimidin-2-amine (Compound 51),
4-(1H-benzo[d]imidazol-5-yl)-N-(3-(trifluoromethyl)phenyl)pyridin-2-amine (Compound 52),
4-(1H-benzo[d]imidazol-5-yl)-N-(2-(tert-butyl)phenyl)pyrimidin-2-amine (Compound 53),
4-(1H-benzo[d]imidazol-5-yl)-N-(5-isopropyl-4H-1,2,4-triazol-3-yl)pyrimidin-2-amine (Compound 54),
4-(1H-benzo[d]imidazol-5-yl)-N-(naphthalen-2-yl)pyrimidin-2-amine (Compound 55),
N-(4-(1H-benzo[d]imidazol-5-yl)pyrimidin-2-yl)isoxazol-4-amine (Compound 56),
4-(1H-benzo[d]imidazol-5-yl)-N-(1H-pyrazol-5-yl)pyrimidin-2-amine (Compound 57),
N-(4-(1H-benzo[d]imidazol-5-yl)pyrimidin-2-yl)-5-(trifluoromethyl)-1,3,4-thiadiazol-2-amine (Compound 58),
4-(benzo[d]thiazol-6-yl)-N-(3-(trifluoromethyl)phenyl)pyrimidin-2-amine (Compound 59)
4-(1H-benzo[d]imidazol-5-yl)-N-(3,4-dimethylphenyl)pyrimidin-2-amine (Compound 60),
4-(1H-benzo[d]imidazol-5-yl)-N-(2-methoxyphenyl)pyrimidin-2-amine (Compound 61),
4-(1H-benzo[d]imidazol-5-yl)-N-(2-(trifluoromethyl)phenyl)pyrimidin-2-amine (Compound 62),
1-(3-((4-(1H-benzo[d]imidazol-5-yl)pyrimidin-2-yl)amino)phenyl)piperazin-2-one (Compound 63),
4-(1H-benzo[d]imidazol-5-yl)-N-(2-isopropylphenyl)pyrimidin-2-amine (Compound 64),
4-(1H-benzo[d]imidazol-5-yl)-N-(3-(piperidin-1-yl)phenyl)pyrimidin-2-amine (Compound 65),
4-(1H-benzo[d]imidazol-5-yl)-N-(2-fluorophenyl)pyrimidin-2-amine (Compound 66),
3-((4-(1H-benzo[d]imidazol-5-yl)pyrimidin-2-yl)amino)benzamide (Compound 67),
4-(1H-benzo[d]imidazol-5-yl)-N-(thiophen-3-yl)pyrimidin-2-amine (Compound 68),
4-(1H-benzo[d]imidazol-5-yl)-N-(3-chlorophenyl)pyrimidin-2-amine (Compound 69),
N-([1,1′-biphenyl]-3-yl)-4-(1H-benzo[d]imidazol-5-yl)pyrimidin-2-amine (Compound 70),
4-(1H-benzo[d]imidazol-5-yl)-N-(3-(4-methylpiperazin-1-yl)phenyl)pyrimidin-2-amine (Compound 71),
4-(1H-benzo[d]imidazol-5-yl)-N-(4-(tert-butyl)phenyl)pyrimidin-2-amine (Compound 72),
4-((4-(1H-benzo[d]imidazol-5-yl)pyrimidin-2-yl)amino)benzamide (Compound 73),
5-(2-(4-methylpiperazin-1-yl)pyrimidin-4-yl)-1H-benzo[d]imidazole (Compound 74),
4-(1H-benzo[d]imidazol-5-yl)-N-(thiophen-2-ylmethyl)pyrimidin-2-amine (Compound 75)
4-(benzo[d]thiazol-5-yl)-N-(3-(trifluoromethyl)phenyl)pyrimidin-2-amine (Compound 76),
4-(1H-benzo[d]imidazol-5-yl)-N-(1-methyl-1H-pyrazol-4-yl)pyrimidin-2-amine (Compound 77),
4-(1H-benzo[d]imidazol-5-yl)-N-phenylpyrimidin-2-amine (Compound 78),
4-(1H-benzo[d]imidazol-5-yl)-N-(2-(trifluoromethoxy)phenyl)pyrimidin-2-amine (Compound 79),
4-(1H-benzo[d]imidazol-5-yl)-N-(2-ethylphenyl)pyrimidin-2-amine (Compound 80),
4-(1H-benzo[d]imidazol-5-yl)-N-(4-fluorophenyl)pyrimidin-2-amine (Compound 81),
4-(1H-benzo[d]imidazol-5-yl)-N-((1-methyl-1H-pyrazol-5-yl)methyl)pyrimidin-2-amine (Compound 82),
4-(1H-benzo[d]imidazol-5-yl)-N-(5-fluoropyridin-3-yl)pyrimidin-2-amine (Compound 83),
4-(1H-benzo[d]imidazol-5-yl)-N-(thiophen-2-yl)pyrimidin-2-amine (Compound 84),
4-(1H-benzo[d]imidazol-5-yl)-N-(furan-2-ylmethyl)pyrimidin-2-amine (Compound 85),
4-(1H-benzo[d]imidazol-5-yl)-N-(4-chlorophenyl)pyrimidin-2-amine (Compound 86),
4-(1H-benzo[d]imidazol-5-yl)-N-(3-(tert-butyl)phenyl)pyrimidin-2-amine (Compound 87)
4-(1H-benzo[d]imidazol-5-yl)-N-cyclopentylpyrimidin-2-amine (Compound 88),
4-(1H-benzo[d]imidazol-5-yl)-N-(4-chloro-3-(trifluoromethyl)phenyl)pyrimidin-2-amine (Compound 89),
3-((4-(1H-benzo[d]imidazol-5-yl)pyrimidin-2-yl)amino)phenol (Compound 90),
N-(3-(3-aminopropoxy)phenyl)-4-(1H-benzo[d]imidazol-5-yl)pyrimidin-2-amine (Compound 91),
4-(1H-benzo[d]imidazol-5-yl)-N-(3-(piperazin-1-yl)phenyl)pyrimidin-2-amine (Compound 92),
4-(1H-benzo[d]imidazol-5-yl)-N-(3-ethynylphenyl)pyrimidin-2-amine (Compound 93),
3-((4-(1H-benzo[d]imidazol-5-yl)pyrimidin-2-yl)amino)benzonitrile (Compound 94),
4-(1-methyl-1H-benzo[d]imidazol-5-yl)-N-(3-(trifluoromethyl)phenyl)pyrimidin-2-amine (Compound 95),
4-(1H-benzo[d]imidazol-5-yl)-N-cyclohexylpyrimidin-2-amine (Compound 96),
4-(1H-benzo[d]imidazol-5-yl)-N-(2,3-dihydro-1H-inden-5-yl)pyrimidin-2-amine (Compound 97),
4-(1H-benzo[d]imidazol-5-yl)-N-(3,4-dichlorophenyl)pyrimidin-2-amine (Compound 98)
4-((4-(1H-benzo[d]imidazol-5-yl)pyrimidin-2-yl)amino)benzonitrile (Compound 99),
4-(1H-benzo[d]imidazol-5-yl)-N-(3-(trifluoromethoxy)phenyl)pyrimidin-2-amine (Compound 100),
4-(1H-benzo[d]imidazol-5-yl)-N-(tetrahydro-2H-pyran-4-yl)pyrimidin-2-amine (Compound 101),
4-(1H-benzo[d]imidazol-5-yl)-N-(3-morpholinophenyl)pyrimidin-2-amine (Compound 102),
4-(1H-benzo[d]imidazol-5-yl)-N-(p-tolyl)pyrimidin-2-amine (Compound 103),
4-(1H-benzo[d]imidazol-5-yl)-N-isopropylpyrimidin-2-amine (Compound 104),
4-(1H-imidazo[4,5-b]pyridin-6-yl)-N-(3-(trifluoromethyl)phenyl)-1,3,5-triazin-2-amine (Compound 105),
4-(1H-imidazo[4,5-b]pyridin-6-yl)-N-(5-(trifluoromethyl)pyridin-3-yl)pyrimidin-2-amine (Compound 106),
4-(imidazo[1,2-a]pyrazin-6-yl)-N-(3-(trifluoromethyl)phenyl)pyrimidin-2-amine (Compound 107),
N²-(3-fluorophenyl)-N⁴-(6-methoxypyridin-3-yl)pyrimidine-2,4-diamine (Compound 108),
4-((6-methoxypyridin-3-yl)oxy)-N-(3-(trifluoromethyl)phenyl)pyrimidin-2-amine (Compound 109),
5-((2-((3-fluorophenyl)amino)pyrimidin-4-yl)oxy)pyridin-2-ol (Compound 110),
N⁴-(2-(1H-imidazol-5-yl)ethyl)-N²-(3-(trifluoromethyl)phenyl)pyrimidine-2,4-diamine (Compound 111),
4-(1,4,6,7-tetrahydro-5H-imidazo[4,5-c]pyridin-5-yl)-N-(3-(trifluoromethyl)phenyl)pyrimidin-2-amine (Compound 112),
4-(1H-imidazo[4,5-b]pyridin-6-yl)-5-methyl-N-(3-(trifluoromethyl)phenyl)pyrimidin-2-amine (Compound 113),
4-(5-methyl-1H-benzo[d]imidazol-6-yl)-N-(3-(trifluoromethyl)phenyl)pyrimidin-2-amine (Compound 114),
4-(7-fluoro-1H-benzo[d]imidazol-5-yl)-N-(3-(trifluoromethyl)phenyl)pyrimidin-2-amine (Compound 115),
5-(2-((3-(trifluoromethyl)phenyl)amino)pyrimidin-4-yl)-1,3-dihydro-2H-benzo[d]imidazol-2-one (Compound 116),
5-(2-((3-fluorophenyl)amino)pyrimidin-4-yl)-1,3-dihydro-2H-benzo[d]imidazol-2-one (Compound 117),
4-((2-((3-fluorophenyl)amino)pyrimidin-4-yl)amino)pyridin-2-ol (Compound 118),
5-(2-((3-(trifluoromethyl)phenyl)amino)pyrimidin-4-yl)thiazol-2-amine (Compound 119),
4-(2-((3-(2-aminoethoxy)phenyl)amino)pyrimidin-4-yl)pyridin-2-ol (Compound 120),
N-(3-(2-aminoethoxy)phenyl)-4-(2-methoxypyridin-4-yl)pyrimidin-2-amine (Compound 121),
N²-(3-fluorophenyl)-N⁴-(2-methoxypyridin-4-yl)pyrimidine-2,4-diamine (Compound 122),
5-((2-((3-fluorophenyl)amino)pyrimidin-4-yl)amino)pyridin-2-ol (Compound 123),
N²-(3-fluorophenyl)-N⁴-(6-methoxypyridin-3-yl)pyrimidine-2,4-diamine (Compound 124),
4-(4-methyl-1H-benzo[d]imidazol-5-yl)-N-(3-(trifluoromethyl)phenyl)pyrimidin-2-amine (Compound 125),
4-(6-fluoro-1H-benzo[d]imidazol-5-yl)-N-(3-(trifluoromethyl)phenyl)pyrimidin-2-amine (Compound 126),
4-(imidazo[1,2-a]pyridin-6-yl)-N-(3-(trifluoromethyl)phenyl)pyrimidin-2-amine (Compound 127),
4-(2-((3-(2-hydroxyethoxy)phenyl)amino)pyrimidin-4-yl)pyridin-2-ol (Compound 128),
2-(3-((4-(2-methoxypyridin-4-yl)pyrimidin-2-yl)amino)phenoxy)ethan-1-ol (Compound 129),
4-(2-((3-(trifluoromethyl)phenyl)amino)pyrimidin-4-yl)phenol (Compound 130),
2′-((3-(trifluoromethyl)phenyl)amino)-[4,4′-bipyrimidin]-6-ol (Compound 131),
4-(1H-pyrrolo[3,2-b]pyridin-6-yl)-N-(3-(trifluoromethyl)phenyl)pyrimidin-2-amine (Compound 132),
4-(1H-imidazo[4,5-b]pyridin-6-yl)-N-(3-propylphenyl)pyrimidin-2-amine (Compound 133),
4-(1H-imidazo[4,5-b]pyridin-6-yl)-N-(3-isopropylphenyl)pyrimidin-2-amine (Compound 134),
N-(4-fluoro-3-methylphenyl)-4-(1H-imidazo[4,5-b]pyridin-6-yl)pyrimidin-2-amine (Compound 135),
4-(1H-imidazo[4,5-b]pyridin-6-yl)-N-(m-tolyl)pyrimidin-2-amine (Compound 136),
N²-(3-(trifluoromethyl)phenyl)-[4,5′-bipyrimidine]-2,2′-diamine (Compound 137),
4-(1H-benzo[d][1,2,3]triazol-5-yl)-N-(3-fluorophenyl)pyrimidin-2-amine (Compound 138),
4-(1H-benzo[d][1,2,3]triazol-5-yl)-N-(3-(trifluoromethyl)phenyl)pyrimidin-2-amine (Compound 139),
N-(3-ethylphenyl)-4-(1H-imidazo[4,5-b]pyridin-6-yl)pyrimidin-2-amine (Compound 140),
N-(3-chlorophenyl)-4-(1H-imidazo[4,5-b]pyridin-6-yl)pyrimidin-2-amine (Compound 141),
4-(2-methyl-1H-benzo[d]imidazol-6-yl)-N-(3-(trifluoromethyl)phenyl)pyrimidin-2-amine (Compound 142),
4-(2-methoxythiazol-5-yl)-N-(3-(trifluoromethyl)phenyl)pyrimidin-2-amine (Compound 143),
5-(2-((3-(trifluoromethyl)phenyl)amino)pyrimidin-4-yl)thiazol-2-ol (Compound 144),
4-(2-((5-fluoropyridin-3-yl)amino)pyrimidin-4-yl)pyridin-2-ol (Compound 145),
N-(5-fluoropyridin-3-yl)-4-(2-methoxypyridin-4-yl)pyrimidin-2-amine (Compound 146),
4-(2-((3,5-difluorophenyl)amino)pyrimidin-4-yl)pyridin-2-ol (Compound 147),
N-(3,5-difluorophenyl)-4-(2-methoxypyridin-4-yl)pyrimidin-2-amine (Compound 148),
4-(2-((3-(tert-butyl)phenyl)amino)pyrimidin-4-yl)pyridin-2-ol (Compound 149),
N-(3-(tert-butyl)phenyl)-4-(2-methoxypyridin-4-yl)pyrimidin-2-amine (Compound 150),
N-(3-fluorophenyl)-4-(1H-imidazo[4,5-b]pyridin-6-yl)pyrimidin-2-amine (Compound 151),
4-(1H-indol-5-yl)-N-(3-(trifluoromethyl)phenyl)pyrimidin-2-amine (Compound 152),
N-(3-fluorophenyl)-4-(1H-indol-5-yl)pyrimidin-2-amine (Compound 153),
4-(2-((5-(trifluoromethyl)pyridin-3-yl)amino)pyrimidin-4-yl)pyridin-2-ol (Compound 154),
4-(2-methoxypyridin-4-yl)-N-(5-(trifluoromethyl)pyridin-3-yl)pyrimidin-2-amine (Compound 155),
4-(2-((2-(trifluoromethyl)pyridin-4-yl)amino)pyrimidin-4-yl)pyridin-2-ol (Compound 156),
4-(2-methoxypyridin-4-yl)-N-(2-(trifluoromethyl)pyridin-4-yl)pyrimidin-2-amine (Compound 157),
4-(2-((4-(trifluoromethyl)pyridin-2-yl)amino)pyrimidin-4-yl)pyridin-2-ol (Compound 158),
4-(2-(m-tolylamino)pyrimidin-4-yl)pyridin-2-ol (Compound 159),
4-(2-methoxypyridin-4-yl)-N-(m-tolyl)pyrimidin-2-amine (Compound 160),
4-(1H-imidazo[4,5-b]pyridin-6-yl)-N-(3-(trifluoromethyl)phenyl)pyrimidin-2-amine (Compound 161),
6′-methoxy-N-phenyl-[4,4′-bipyrimidin]-2-amine (Compound 162),
2′-methoxy-N-phenyl-[4,4′-bipyrimidin]-2-amine (Compound 163),
4-(1H-indazol-6-yl)-N-(3-(trifluoromethyl)phenyl)pyrimidin-2-amine (Compound 164),
N-(3-fluorophenyl)-4-(1H-indazol-5-yl)pyrimidin-2-amine (Compound 165),
4-(2-methoxypyridin-4-yl)-N-(4-(trifluoromethyl)pyridin-2-yl)pyrimidin-2-amine (Compound 166),
4-(2-((6-(trifluoromethyl)pyridin-2-yl)amino)pyrimidin-4-yl)pyridin-2-ol (Compound 167),
4-(2-methoxypyridin-4-yl)-N-(6-(trifluoromethyl)pyridin-2-yl)pyrimidin-2-amine (Compound 168),
N-(3-fluorophenyl)-4-(1H-pyrrolo[2,3-b]pyridin-5-yl)pyrimidin-2-amine (Compound 169),
6-(2-((4-fluoropyridin-2-yl)amino)pyrimidin-4-yl)pyridin-3-ol (Compound 170),
4-(1H-benzo[d]imidazol-6-yl)-N-(3-(trifluoromethyl)phenyl)pyrimidin-2-amine (Compound 171),
4-(1H-indol-6-yl)-N-(3-(trifluoromethyl)phenyl)pyrimidin-2-amine (Compound 172),
3-(2-((3-(trifluoromethyl)phenyl)amino)pyrimidin-4-yl)phenol (Compound 173),
N-(3-fluorophenyl)-4-(5-methoxypyridin-2-yl)pyrimidin-2-amine (Compound 174),
4-(2-(pyrimidin-4-ylamino)pyrimidin-4-yl)pyridin-2-ol (Compound 175),
N-(3-fluorophenyl)-4-(5-methoxythiophen-2-yl)pyrimidin-2-amine (Compound 176),
4-(2-methoxypyridin-4-yl)-N-(pyrimidin-4-yl)pyrimidin-2-amine (Compound 177),
4-(2-(pyrimidin-5-ylamino)pyrimidin-4-yl)pyridin-2-ol (Compound 178),
4-(2-methoxypyridin-4-yl)-N-(pyrimidin-5-yl)pyrimidin-2-amine (Compound 179),
N-(3-fluorophenyl)-4-(2-methoxythiazol-5-yl)pyrimidin-2-amine (Compound 180),
5-(2-((3-fluorophenyl)amino)pyrimidin-4-yl)thiazol-2-ol (Compound 181),
2-(2-((3-fluorophenyl)amino)pyrimidin-4-yl)pyridin-4-ol (Compound 182),
2-(2-(phenylamino)pyrimidin-4-yl)pyridin-4-ol (Compound 183),
4-(2-(pyrimidin-2-ylamino)pyrimidin-4-yl)pyridin-2-ol (Compound 184),
methyl 3-((4-(2-hydroxypyridin-4-yl)pyrimidin-2-yl)amino)benzoate (Compound 185),
4-(2-(pyridin-2-ylamino)pyrimidin-4-yl)pyridin-2-ol (Compound 186),
4-(2-methoxypyridin-4-yl)-N-(pyridin-2-yl)pyrimidin-2-amine (Compound 187),
4-(2-(pyridin-3-ylamino)pyrimidin-4-yl)pyridin-2-ol (Compound 188),
4-(2-methoxypyridin-4-yl)-N-(pyridin-3-yl)pyrimidin-2-amine (Compound 189),
4-(2-(pyridin-4-ylamino)pyrimidin-4-yl)pyridin-2-ol (Compound 190),
4-(2-((3-methoxyphenyl)amino)pyrimidin-4-yl)pyridin-2-ol (Compound 191),
N-(3-methoxyphenyl)-4-(2-methoxypyridin-4-yl)pyrimidin-2-amine (Compound 192),
N-(3-fluorophenyl)-4-(4-methoxypyridin-2-yl)pyrimidin-2-amine (Compound 193),
4-(2-((3-fluorophenyl)amino)pyrimidin-4-yl)phenol (Compound 194),
4-(2-methoxypyridin-4-yl)-N-(pyrimidin-2-yl)pyrimidin-2-amine (Compound 195),
3-((4-(2-hydroxypyridin-4-yl)pyrimidin-2-yl)amino)benzoic acid (Compound 196),
4-(2-methoxypyridin-4-yl)-N-(pyridin-4-yl)pyrimidin-2-amine (Compound 197),
3-((4-(2-methoxypyridin-4-yl)pyrimidin-2-yl)amino)benzamide (Compound 198),
4-(1H-benzo[d]imidazol-5-yl)-N-(3-fluorophenyl)pyrimidin-2-amine (Compound 199),
5-(2-((3-fluorophenyl)amino)pyrimidin-4-yl)pyridin-3-ol (Compound 200),
5-(2-((3-(trifluoromethyl)phenyl)amino)pyrimidin-4-yl)pyridin-3-ol (Compound 201),
N-(3-fluorophenyl)-4-(1H-indol-5-yl)pyrimidin-2-amine (Compound 202),
N-(3-fluorophenyl)-4-(5-methoxypyridin-3-yl)pyrimidin-2-amine (Compound 203),
4-(5-methoxypyridin-3-yl)-N-(3-(trifluoromethyl)phenyl)pyrimidin-2-amine (Compound 204),
4-(2-((2,3-difluorophenyl)amino)pyrimidin-4-yl)pyridin-2-ol (Compound 205),
N-(2,3-difluorophenyl)-4-(2-methoxypyridin-4-yl)pyrimidin-2-amine (Compound 206),
4-(2-((2-fluorophenyl)amino)pyrimidin-4-yl)pyridin-2-ol (Compound 207),
N-(2-fluorophenyl)-4-(2-methoxypyridin-4-yl)pyrimidin-2-amine (Compound 208),
4-(2-((3,4-difluorophenyl)amino)pyrimidin-4-yl)pyridin-2-ol (Compound 209),
N-(3,4-difluorophenyl)-4-(2-methoxypyridin-4-yl)pyrimidin-2-amine (Compound 210),
5-(2-((3-(trifluoromethyl)phenyl)amino)pyrimidin-4-yl)pyridin-2-ol (Compound 211),
4-(6-methoxypyridin-3-yl)-N-(3-(trifluoromethyl)phenyl)pyrimidin-2-amine (Compound 212),
5-(2-((3-fluorophenyl)amino)pyrimidin-4-yl)pyridin-2-ol (Compound 213),
N-(3-fluorophenyl)-4-(6-methoxypyridin-3-yl)pyrimidin-2-amine (Compound 214),
4-(4-(methyl(phenyl)amino)-1,3,5-triazin-2-yl)pyridin-2-ol (Compound 215),
4-(2-methoxypyridin-4-yl)-N-methyl-N-phenyl-1,3,5-triazin-2-amine (Compound 216),
4-(4-methoxypyridin-2-yl)-N-phenylpyrimidin-2-amine (Compound 217),
5-(2-(methyl(phenyl)amino)pyrimidin-4-yl)pyridin-3-ol (Compound 218),
5-(2-(phenylamino)pyrimidin-4-yl)pyridin-3-ol (Compound 219),
4-(2-(methyl (3-(trifluoromethyl)phenyl)amino)pyrimidin-4-yl)pyridin-2-ol (Compound 220),
4-(2-(methyl(phenyl)amino)pyrimidin-4-yl)pyridin-2-ol (Compound 221),
5-(2-(methyl(phenyl)amino)pyrimidin-4-yl)pyridin-2-ol (Compound 222),
4-(6-methoxypyridin-3-yl)-N-methyl-N-phenylpyrimidin-2-amine (Compound 223),
4-(5-methoxypyridin-3-yl)-N-methyl-N-phenylpyrimidin-2-amine (Compound 224),
6-(2-(methyl(phenyl)amino)pyrimidin-4-yl)pyridin-2-ol (Compound 225),
6-(2-(phenylamino)pyrimidin-4-yl)pyridin-2-ol (Compound 226),
4-(6-methoxypyridin-2-yl)-N-methyl-N-phenylpyrimidin-2-amine (Compound 227),
4-(5-methoxypyridin-3-yl)-N-phenylpyrimidin-2-amine (Compound 228),
4-(2-benzylpyrimidin-4-yl)pyridin-2-ol (Compound 229),
2-benzyl-4-(2-methoxypyridin-4-yl)pyrimidine (Compound 230),
4-(2-(phenylthio)pyrimidin-4-yl)pyridin-2-ol (Compound 231),
4-(2-phenoxypyrimidin-4-yl)pyridin-2-ol (Compound 232),
4-(4-(phenylamino)-1,3,5-triazin-2-yl)pyridin-2-ol (Compound 233),
4-(2-methoxypyridin-4-yl)-2-(phenylthio)pyrimidine (Compound 234),
4-(2-methoxypyridin-4-yl)-2-phenoxypyrimidine (Compound 235),
4-(6-methoxypyridin-2-yl)-N-phenylpyrimidin-2-amine (Compound 236),
4-(2-((3-fluorophenyl)(methyl)amino)pyrimidin-4-yl)pyridin-2-ol (Compound 237),
5-(2-(phenylamino)pyrimidin-4-yl)pyridin-2-ol (Compound 238),
4-(6-methoxypyridin-3-yl)-N-phenylpyrimidin-2-amine (Compound 239),
4-(2-methoxypyridin-4-yl)-N-phenyl-1,3,5-triazin-2-amine (Compound 240),
4-(2-((3-(trifluoromethyl)phenyl)amino)pyrimidin-4-yl)pyridin-2-ol (Compound 241),
4-(2-((3-fluorophenyl)amino)pyrimidin-4-yl)pyridin-2-ol (Compound 242),
N-(3-fluorophenyl)-4-(2-methoxypyridin-4-yl)pyrimidin-2-amine (Compound 243),
3-(2-(phenylamino)pyrimidin-4-yl)phenol (Compound 244),
6-(3-methoxyphenyl)-N-phenylpyridin-2-amine (Compound 245),
4-(3-methoxyphenyl)-N-phenylpyridin-2-amine (Compound 246),
4-(3-methoxyphenyl)-N-methyl-N-phenylpyrimidin-2-amine (Compound 247),
N-(2-methoxyphenyl)-4-(3-methoxyphenyl)pyrimidin-2-amine (Compound 248),
N-(3-chlorophenyl)-4-(3-methoxyphenyl)pyrimidin-2-amine (Compound 249),
N,4-bis(3-methoxyphenyl)pyrimidin-2-amine (Compound 250),
4-(2-(phenylamino)pyrimidin-4-yl)pyridin-2-ol (Compound 251),
4-(2-methoxypyridin-4-yl)-N-phenylpyrimidin-2-amine (Compound 252),
4-(3-methoxyphenyl)-N-phenylpyrimidin-2-amine (Compound 253),
N-(3-((4-(1H-benzo[d]imidazol-5-yl)pyrimidin-2-yl)amino)phenyl)furan-2-carboxamide (Compound 254),
N-(3-((4-(1H-benzo[d]imidazol-5-yl)pyrimidin-2-yl)amino)phenyl)methanesulfonamide (Compound 255),
1-(3-((4-(1H-benzo[d]imidazol-5-yl)pyrimidin-2-yl)amino)phenyl)ethan-1-one (Compound 256),
N-(3-((4-(1H-benzo[d]imidazol-5-yl)pyrimidin-2-yl)amino)phenyl)isobutyramide (Compound 257),
4-(1H-benzo[d]imidazol-5-yl)-N-(3,4,5-trifluorophenyl)pyrimidin-2-amine (Compound 258),
N-(3-(1H-1,2,3-triazol-1-yl)phenyl)-4-(1H-benzo[d]imidazol-5-yl)pyrimidin-2-amine (Compound 259),
4-(1H-benzo[d]imidazol-5-yl)-N-(3-((trifluoromethyl)sulfonyl)phenyl)pyrimidin-2-amine (Compound 260),
N-(3-(1H-pyrazol-5-yl)phenyl)-4-(1H-benzo[d]imidazol-5-yl)pyrimidin-2-amine (Compound 261),
N-(3-((4-(1H-benzo[d]imidazol-5-yl)pyrimidin-2-yl)amino)phenyl)-N-methylbenzenesulfonamide (Compound 262),
3-((4-(1H-benzo[d]imidazol-5-yl)pyrimidin-2-yl)amino)-N-phenylbenzamide (Compound 263),
4-(1H-benzo[d]imidazol-5-yl)-N-(3-(oxazol-5-yl)phenyl)pyrimidin-2-amine (Compound 264),
4-(1H-benzo[d]imidazol-5-yl)-N-(4-methyl-3-(trifluoromethyl)phenyl)pyrimidin-2-amine (Compound 265),
4-(1H-benzo[d]imidazol-5-yl)-N-(4-(trifluoromethyl)phenyl)pyrimidin-2-amine (Compound 266),
N-(3-(1H-tetrazol-5-yl)phenyl)-4-(1H-benzo[d]imidazol-5-yl)pyrimidin-2-amine (Compound 267),
N-(4-(1H-benzo[d]imidazol-5-yl)pyrimidin-2-yl)-1H-indazol-6-amine (Compound 268),
N-(benzo[b]thiophen-5-yl)-4-(1H-benzo[d]imidazol-5-yl)pyrimidin-2-amine (Compound 269),
N-(3-fluorophenyl)-4-(pyrazolo[1,5-a]pyrimidin-5-yl)pyrimidin-2-amine (Compound 270),
4-(benzo[d]oxazol-5-yl)-N-(3-fluorophenyl)pyrimidin-2-amine (Compound 271),
ethyl 3-(3-((4-(1H-benzo[d]imidazol-6-yl)pyrimidin-2-yl)amino)phenyl)propanoate (Compound 272),
N-(3-fluorophenyl)-4-(imidazo[1,2-a]pyrimidin-6-yl)pyrimidin-2-amine (Compound 273),
4-(1H-benzo[d]imidazol-6-yl)-N-(3-chloro-5-methylphenyl)pyrimidin-2-amine (Compound 274),
N1-(4-(1H-benzo[d]imidazol-6-yl)pyrimidin-2-yl)benzene-1,3-diamine (Compound 275), and
4-(1H-benzo[d]imidazol-6-yl)-N-(1-(tert-butyl)-1H-pyrazol-4-yl)pyrimidin-2-amine (Compound 276),
4-(1-cyclopropyl-4-fluoro-2-methyl-1H-benzo[d]imidazol-6-yl)-N-(6-(trifluoromethyl)pyridin-2-yl)pyridin-2-amine (Compound 282),
4-(1-cyclopropyl-2-methyl-1H-benzo[d]imidazol-6-yl)-N-(6-(trifluoromethyl)pyridin-2-yl)pyridin-2-amine (Compound 284),
4-(2-((6-(trifluoromethyl)pyridin-2-yl)amino)pyridin-4-yl)benzamide (Compound 283),
4-(3-isopropyl-[1,2,4]triazolo[4,3-a]pyridin-6-yl)-N-(6-(trifluoromethyl)pyridin-2-yl)pyridin-2-amine (Compound 289),
4-(3-isopropyl-2-methyl-2H-indazol-5-yl)-N-(6-(trifluoromethyl)pyridin-2-yl)pyridin-2-amine (Compound 285),
4-(1-isopropyl-2-methyl-1H-benzo[d]imidazol-6-yl)-N-(6-(trifluoromethyl)pyridin-2-yl)pyridin-2-amine (Compound 291),
5-(2-((6-(trifluoromethyl)pyridin-2-yl)amino)pyridin-4-yl)isoindolin-1-one (Compound 287),
4-(2-methyl-1-phenyl-1H-benzo[d]imidazol-5-yl)-N-(6-(trifluoromethyl)pyridin-2-yl)pyridin-2-amine (Compound 288),
4-(3-isopropyl-2-methyl-2H-indazol-5-yl)-N-(6-(trifluoromethyl)pyridin-2-yl)pyridin-2-amine (Compound 290), and
4-(4-fluoro-1-isopropyl-2-methyl-1H-benzo[d]imidazol-6-yl)-N-(6-(trifluoromethyl)pyridin-2-yl)pyridin-2-amine (Compound 286),
   or a pharmaceutically acceptable salt thereof.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic at the concentration or amount used, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002.

In some embodiments, pharmaceutically acceptable salts are obtained by reacting a compound of Formula (I) with an acid. In some embodiments, the compound of Formula (I) (i.e. free base form) is basic and is reacted with an organic acid or an inorganic acid.

The term “acid” refers to a compound that is an electron pair acceptor in an acid-base reaction.

The term “inorganic acid” refers to an acid that does not include a carbon bond. Inorganic acids can be a strong acid or a weak acid. Inorganic acids include, but are not limited to, hydrochloric acid, hydrobromic acid, sulfuric acid, phosphoric acid, nitric acid, sulfamic acid, perchloric acid, boric acid, fluorophosphoric acid, and metaphosphoric acid.

The term “organic acid” refers to an acid including at least one C—H bond, C—F bond, or C—C bond. Organic acids include, but are not limited to, acetic acid; benzenesulfonic acid; benzoic acid; camphor-10-sulfonic acid (+); citric acid; ethanesulfonic acid; formic acid; fumaric acid; glycolic acid; isobutyric acid; lactic acid (DL); maleic acid; malonic acid; methanesulfonic acid; oxalic acid; proprionic acid; salicylic acid; succinic acid; L-(+)-tartaric acid; p-toluenesulfonic acid; trifluoroacetic acid; and trifluoromethanesulfonic acid.

In some embodiments, a compound of Formula (I) is prepared as a chloride salt, sulfate salt, bromide salt, mesylate salt, maleate salt, citrate salt or phosphate salt.

In some embodiments, pharmaceutically acceptable salts are obtained by reacting a compound of Formula (I) with a base.

The term “base” refers to a compound that is an electron pair donor in an acid-base reaction. The base can be an inorganic base or an organic base. In some embodiments, the compound of Formula (I) is acidic and is reacted with a base.

The term “inorganic base” refers to a base that does not include at least one C—H bond and includes at least one alkali metal or alkaline earth metal. Examples of an inorganic base include, but are not limited to, sodium hydride, potassium hydride, lithium hydride, calcium hydride, barium carbonate, calcium carbonate, cesium carbonate, lithium carbonate, magnesium carbonate, potassium carbonate, sodium carbonate, cesium hydrogen carbonate, potassium hydrogen carbonate, sodium hydrogen carbonate, barium hydroxide, calcium hydroxide, cesium hydroxide, lithium hydroxide, magnesium hydroxide, potassium hydroxide, sodium hydroxide. In some embodiments, the compound of Formula (I) is acidic and is reacted with an inorganic base. In such situations, an acidic proton of the compound of Formula (I) is replaced by a metal ion, e.g., lithium, sodium, potassium, magnesium, calcium, barium, or a cesium ion.

The term “organic base” refers to a base including at least one C—H bond (e.g., an amine base). In some embodiments, the amine base can be a primary, secondary, or tertiary amine. In some cases, compounds described herein coordinate with an amine base, such as, but not limited to, DIPEA, TEA, ethanolamine, diethanolamine, triethanolamine, tromethamine, meglumine, N-methylglucamine, dicyclohexylamine, and tris(hydroxymethyl)methylamine.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Compounds described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically labeled compound. Each chemical element as represented in a compound structure may include any isotopic distribution of said element. For example, in a compound structure a hydrogen atom can be explicitly disclosed or understood to be present in the compound. At any position of the compound that a hydrogen atom can be present, the hydrogen atom can be an isotopic distribution of hydrogen, including but not limited to protium (¹H) and deuterium (²H) in proportional amounts to those usually found in nature and in proportional amounts different to those usually found in nature. Thus, reference herein to a compound encompasses all potential isotopic distributions for each atom unless the context clearly dictates otherwise. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, an atom in one position of the compound has an isotopic distribution for a chemical element in proportional amounts different to those usually found in nature (remainder atoms having an isotopic distribution for a chemical element in proportional amounts to those usually found in nature). In some embodiments, atoms in at least two positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least three positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least four positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least five positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least six positions of the compound independently have an isotopic distribution for a chemical element in proportional amounts different to those usually found in nature (remainder atoms having an isotopic distribution for a chemical element in proportional amounts to those usually found in nature).

Synthetic methods for incorporating radioisotopes into organic compounds are applicable to compound described herein. These synthetic methods, for example, incorporating activity levels of tritium into target molecules, are as follows:
A. Catalytic Reduction with Tritium Gas: This procedure normally yields high specific activity products and requires halogenated or unsaturated precursors.
B. Reduction with Sodium Borohydride [³H]: This procedure is rather inexpensive and requires precursors containing reducible functional groups such as aldehydes, ketones, lactones, esters and the like.
C. Reduction with Lithium Aluminum Hydride [³H]: This procedure offers products at almost theoretical specific activities. It also requires precursors containing reducible functional groups such as aldehydes, ketones, lactones, esters and the like.
D. Tritium Gas Exposure Labeling: This procedure involves exposing precursors containing exchangeable protons to tritium gas in the presence of a suitable catalyst.
E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:
A. A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A representative procedure was reported by Zhu, G-D. and co-workers in J. Org. Chem., 2002, 67, 943-948.
B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.
C. Aryl and heteroaryl bromide exchange with ¹²⁵I: This method is generally a two-step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm., 2001, 44, S280-S282.

A radiolabeled form of a compound described herein can be used in a screening assay to identify/evaluate compounds. In general terms, a newly synthesized or identified compound (i.e., test compound) can be evaluated for its ability to reduce binding of a radiolabeled form of a compound disclosed herein. The ability of a test compound to compete with a radiolabeled form of a compound described herein for the binding correlates to its binding affinity.

The compounds described herein can be asymmetric (e.g., having one or more stereocenters). All stereoisomers, such as enantiomers and diastereomers, are intended unless otherwise indicated. It is understood that, in any compound described herein having one or more chiral centers, if an absolute stereochemistry is not expressly indicated, then each center may independently be the (R)-configuration, or the(S)-configuration, or a mixture thereof. Thus, the compounds provided herein can be enantiomerically pure, enantiomerically enriched (i.e., a scalemic mixture), a racemic mixture, diastereomerically pure, diastereomerically enriched, or a stereoisomeric mixture. In some embodiments, the compounds of Formulae (I), (Ia), (Ib), or (II) possess one or more stereocenters and each stereocenter exists independently in either the R or S configuration. In some embodiments, the compound of Formulae (I), (Ia), (Ib), or (II) exists in the R configuration. In some embodiments, the compound of Formulae (I), (Ia), (Ib), or (II) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof. Stereochemical definitions are available in E. L. Eliel, S. H. Wilen & L. N. Mander, Stereochemistry of Organic Compounds, John Wiley & Sons, Inc., New York, NY, 1994 which is incorporated herein by reference in its entirety. In some embodiments, where the compound described herein is chiral or otherwise includes one or more stereocenters, the compound can be prepared with an enantiomeric excess or diastereomeric excess of greater than 75%, greater than 80%, greater than 85%, greater than 90%, greater than 95%, greater than 99%, or greater than 99.5%, or within a range defined by any of the preceding numbers.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formulae (I), (Ia), (Ib), or (II) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, a compound prepared by stereoselective synthesis can have at least 5%, at least 10%, at least 20%, at least 30%, at least 40%, at least 50%, at least 60%, at least 70%, at least 80%, at least 90%, at least 95%, at least 99%, or at least 99.9% enantiomeric excess, or an enantiomeric excess within a range defined by any of the preceding numbers.

The term, “compound,” as used herein is meant to include all stereoisomers, geometric isomers, and tautomers of the structures depicted. The term is also meant to refer to compounds described herein, regardless of how they are prepared, e.g., synthetically, through biological process (e.g., metabolism or enzyme conversion), or a combination thereof. All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds can be in any solid-state form, such as a polymorph or solvate, so unless clearly indicated otherwise, reference in the specification to compounds and salts thereof should be understood as encompassing any solid-state form of the compound. In some embodiments, the compounds described herein, or salts thereof, are substantially isolated. By “substantially isolated” is meant that the compound is at least partially or substantially separated from the environment in which it was formed or detected. Partial separation can include, e.g., a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salts thereof. The environment in which the compound was formed or detected can include, for example, a composition comprising one or more solvents and one or more chemical reagents.

In some instances, heterocyclic rings may exist in tautomeric forms. In such situations, it is understood that the structures of said compounds are illustrated or named in one tautomeric form but could be illustrated or named in the alternative tautomeric form. The alternative tautomeric forms are expressly included in this disclosure.

### Pharmaceutical Composition

The term “pharmaceutical composition” refers to a compound, including but not limited to, salts, solvates, and hydrates of a compound described herein, in combination with at least one additional component. Some embodiments provide a pharmaceutical composition comprising a compound as described herein, or a pharmaceutically acceptable salt thereof, and at least one pharmaceutically acceptable excipient or diluent.

As used herein, an “excipient” refers to a substance that is added to a composition to provide, without limitation, consistency, stability, binding ability, lubrication, disintegrating ability, etc., to the composition. As used herein, a “diluent” refers to an ingredient in a pharmaceutical composition that lacks pharmacological activity but can be pharmaceutically necessary or desirable. For example, a diluent can be used to increase the bulk of a potent drug substance whose mass is too small for manufacture and/or administration. It may also be a liquid for the dissolution of a drug substance to be administered by injection, ingestion, or inhalation. A pharmaceutically acceptable excipient is a physiologically and pharmaceutically suitable non-toxic and inactive material or ingredient that does not interfere with the activity of the drug substance. Pharmaceutically acceptable excipients are well known in the pharmaceutical art and described, for example, in Rowe et al., Handbook of Pharmaceutical Excipients: A

Comprehensive Guide to Uses, Properties, and Safety, 5th Ed., 2006, and in Remington: The Science and Practice of Pharmacy (Gennaro, 21st Ed. Mack Pub. Co., Easton, PA (2005)). Preservatives, stabilizers, dyes, buffers, and the like can be provided in the pharmaceutical composition. In addition, antioxidants and suspending agents may also be used. For compositions formulated as liquid solutions, acceptable carriers and/or diluents include saline and sterile water, and may optionally include antioxidants, buffers, bacteriostats and other common additives. In some embodiments, the diluents can be a buffered aqueous solution such as, without limitation, phosphate buffered saline. The compositions can also be formulated as capsules, granules, or tablets which contain, in addition to a compound as disclosed and described herein, diluents, dispersing and surface-active agents, binders, and lubricants. One skilled in this art may further formulate a compound as disclosed and described herein in an appropriate manner, and in accordance with accepted practices, such as those disclosed in Remington, supra.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington, supra; Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A compound or a pharmaceutical composition of the present disclosure is, in some embodiments, useful for the treatment of a Myc-mediated disease or disorder. In some embodiments, the pharmaceutical composition is effective at treating a disease or disorder wherein Myc is overexpressed.

In some embodiments, a compound or a pharmaceutical composition of the present disclosure is useful for the treatment of a CDK9-mediated disease, condition, or disorder. In some embodiments, the pharmaceutical composition is effective at treating a disease, condition, or disorder wherein CDK9 is overexpressed.

In some embodiments, the pharmaceutical composition is useful in the treatment of a disease or disorder. In some embodiments, the disease or disorder is mediated by a Myc protein. In some embodiments, the Myc protein is selected from a N-Myc protein, a c-Myc protein, and a L-Myc protein. In some embodiments, the disease, condition, or disorder is mediated by a CDK9 protein. In some embodiments, the disease or disorder is cancer.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes, parenteral routes, inhalational, transdermal, transmucosal, sublingual, buccal and topical administration, although the most suitable route may depend upon for example the condition and disorder of the recipient. By way of example only, compounds described herein can be administered locally to the area in need of treatment, by for example, topical application such as creams or ointments. Additional examples of local administration of the present compounds include eye drops, ocular creams, gels or hydrogels, implants, transdermal patches, or drug depots. In some embodiments, a pharmaceutical composition is administered orally (e.g., in a liquid formulation, tablet, capsule, nebulized liquid, aerosolized liquid, dry powder spray).

In some embodiments, pharmaceutical compositions suitable for oral administration are presented as discrete units such as capsules, cachets or tablets each containing a predetermined amount of the drug substance; as a powder or granules; as a solution or a suspension in an aqueous liquid or a non-aqueous liquid; or as an oil-in-water liquid emulsion or a water-in-oil liquid emulsion.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the drug substance in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the drug substance therein. All formulations for oral administration should be in dosages suitable for such administration. The push-fit capsules can contain the drug substance in admixture with filler such as lactose, binders such as starches, and/or lubricants such as talc or magnesium stearate and, optionally, stabilizers. In soft capsules, the drug substance may be dissolved or suspended in suitable liquids, such as fatty oils, liquid paraffin, or liquid polyethylene glycols. In some embodiments, stabilizers are added.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

It should be understood that in addition to the ingredients particularly mentioned above, the compounds and compositions described herein may include other agents conventional in the art having regard to the type of formulation in question, for example those suitable for oral administration may include flavoring agents.

### Method of Treatment

Some embodiments provide a method of treating a disease or disorder, comprising administering to a subject in need thereof a therapeutically effective amount of a compound, or a pharmaceutically acceptable salt thereof, as described herein.

Some embodiments provide a method of treating a disease or disorder, comprising administering to a subject in need thereof a therapeutically effective amount of a pharmaceutical composition as described herein.

In some embodiments, the disease or disorder is mediated by a Myc protein. In some embodiments, the Myc protein is selected from a N-Myc protein, a c-Myc protein, and a L-Myc protein. In some embodiments, the disease or disorder is cancer.

In some embodiments, the disease or disorder is mediated by a CDK protein. In some embodiments, the disease or disorder is mediated by a transcriptional CDK protein. In some embodiments, the disease or disorder is mediated by a CDK7, CDK8, CDK9, CDK11, CDK12, CDK13, or CDK20 protein. In some embodiments, the disease or disorder is mediated by a CDK7 or CDK9 protein. In some embodiments, the disease or disorder is mediated by a CDK9 protein.

Some embodiments provide a method of treating cancer, comprising administering to a subject in need thereof a therapeutically effective amount of a compound, or a pharmaceutically acceptable salt thereof, as described herein.

Some embodiments provide a method of treating cancer, comprising administering to a subject in need thereof a therapeutically effective amount of a pharmaceutical composition as described herein.

In some embodiments, the cancer is selected from selected from head and neck cancer, nervous system cancer, brain cancer, neuroblastoma, medulloblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site. In some embodiments, the cancer is medulloblastoma. In some embodiments, the subject is human.

Some embodiments provide a method of treating a disease, disorder, or condition mediated by CDK9 in a subject comprising administering a therapeutically effective amount of a compound, or a pharmaceutically acceptable salt thereof, as described herein to the subject. In some embodiments, the compound is a CDK9 inhibitor. In some embodiments, the compound inhibits activity of CDK9.

In various aspects, the present disclosure also provides the use of a compound, or a pharmaceutically acceptable salt thereof, as described herein, or a pharmaceutical composition as described herein, to modulate the amount and activity of a Myc protein, such as in vitro or in a subject, wherein the Myc protein is selected from a N-Myc protein, a c-Myc protein, and a L-Myc protein.

For example, the disclosure provides a method of modulating the amount (e.g., the concentration) and/or activity of a Myc protein such as (e.g., degrading a Myc protein, or modulating the rate of degradation of a Myc protein) that comprises administering an effective amount of a compound described herein, or a pharmaceutically acceptable salt thereof, including embodiments or from any examples, tables or figures as described herein.

In some embodiments, the method as described herein include methods of modulating the protein-protein interactions of the Myc family protein, or a method of decreasing the amount or decreasing the level of activity of a Myc protein.

In some embodiments, the method of modulating the amount and activity of a Myc protein may include co-administering a compound described herein, or a pharmaceutically acceptable salt thereof, and a therapeutically effective amount of a second agent, e.g., a therapeutic agent.

### Combinations of Embodiments

It is further appreciated that certain features of the present disclosure, which are, for clarity, described in the context of separate embodiments, can also be provided in combination in a single embodiment. Conversely, various features of the present disclosure which are, for brevity, described in the context of a single embodiment, can also be provided separately or in any suitable sub-combination.

### DEFINITIONS

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, Cₐ-C_{b} in which “a” and “b” are integers refer to the number of carbon atoms when indicated in an alkyl, alkenyl, alkynyl, haloalkyl, alkoxy, or haloalkoxy group, or the number of carbon atoms in the ring of a cycloalkyl, cycloalkenyl, aryl, heteroaryl or heterocyclyl group. By way of example only, a group designated as “C₁-C₆” indicates that there are one to six carbon atoms in the moiety. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. If no Cₐ-C_{b} is designated with regard to an alkyl, alkenyl, alkynyl, haloalkyl, alkoxy, haloalkoxy, cycloalkyl, cycloalkenyl, aryl, heteroaryl or heterocyclyl group, the broadest range described in these definitions is to be assumed.

An “alkyl” group refers to a fully saturated straight or branched hydrocarbon group. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, an alkyl group can be unsubstituted or substituted.

An “alkylene” group refers to a divalent alkyl radical. Any of the monovalent alkyl groups may be an alkylene by abstraction of a second hydrogen atom from the alkyl. In some embodiments, an alkylene is a C₁-C₆ alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a RO— group, where R is an alkyl as defined herein. A non-limiting list of alkoxys are methoxy, ethoxy, n-propoxy, 1-methylethoxy (iso-propoxy), n-butoxy, iso-butoxy, sec-butoxy, and tert-butoxy. The alkoxy group can be designated as “C₁-C₅-alkoxy” or similar designations. In some embodiments, an alkoxy can be unsubstituted or substituted.

The term “haloalkyl” refers to an alkyl group, as defined herein, wherein one or more hydrogen atoms of the alkyl group have been replaced by a halogen atom (e.g., mono-haloalkyl, di-haloalkyl, and tri-haloalkyl). In some embodiments, the haloalkyl group can have 1 to 5 carbons (i.e., “C₁-C₅ haloalkyl”). The C₁-C₅ haloalkyl can be fully halogen substituted in which case it can be represented by the formula CₙL₂ₙ₊₁, wherein L is a halogen and “n” is 1, 2, 3, 4, or 5. When more than one halogen is present then they can be the same or different and selected from: fluorine, chlorine, bromine, and iodine. In some embodiments, haloalkyl contains 1 to 4 carbons (i.e., C₁-C₄ haloalkyl). In some embodiments, haloalkyl contains 1 to 3 carbons (i.e., C₁-C₃ haloalkyl). In some embodiments, haloalkyl contains 1 or 2 carbons. Examples of haloalkyl groups include fluoromethyl, difluoromethyl, trifluoromethyl, chlorodifluoromethyl, 1-fluoroethyl, 2,2,2-trifluoroethyl, pentafluoroethyl, 4,4,4-trifluorobutyl, and the like.

An “haloalkoxy” refers to an alkoxy group in which one or more of the hydrogen atoms are replaced by a halogen. Such groups include but are not limited to, chloromethoxy, fluoromethoxy, difluoromethoxy, trifluoromethoxy, 1-chloro-2-fluoromethoxy and 2-fluoroisobutoxy. In some embodiments, the haloalkoxy group may have 1 to 5 carbon atoms. The haloalkoxy group can be designated as “C₁-C₅ haloalkoxy” or similar designations.

An “alkylthio” refers to a RS— group, where R is an alkyl as defined herein. A non-limiting list of alkylthios are methylsulfanyl, ethylsulfanyl, n-propylsulfanyl, 1-methylethylsulfanyl, n-butylsulfanyl, iso-butylsulfanyl, sec-butylsulfanyl, and tert-butylsulfanyl. The alkylthiogroup can be designated as “C₁-C₅-alkylthio” or similar designations. In some embodiments, an alkylthio can be unsubstituted or substituted.

The term “alkenyl” refers to a straight or branched hydrocarbon group in which at least one carbon-carbon double bond is present. In some embodiments, an alkenyl is selected from ethenyl (i.e., vinyl), propenyl (i.e., allyl), butenyl, pentenyl, pentadienyl, and the like. Non-limiting examples of an alkenyl group include —CH—CH₂, —C(CH₃)—CH₂, —CH═CHCH₃, —C(CH₃)═CHCH₃, and —CH₂CH═CH₂. The alkenyl group can be designated as “C₂-C₅-alkenyl” or similar designations.

The term “alkynyl” refers to a straight or branched hydrocarbon group in which at least one carbon-carbon triple bond is present. In some embodiments, an alkynyl is selected from ethynyl, propynyl, butynyl, pentynyl, hexynyl, and the like. Non-limiting examples of an alkynyl group include —C═CH, —C═CCH₃—C═CCH₂CH₃, —CH₂C═CH and —CH(CH₃)(C═CH). The alkynyl group can be designated as “C₂-C₅-alkynyl” or similar designations.

As used herein, the term “aryl” refers to an aromatic ring system containing 6, 10 or 14 carbon atoms that can contain a single ring, two fused rings or three fused rings, such as phenyl, naphthalenyl and phenanthrenyl. In one aspect, aryl group can have 6 or 10 carbon atoms (i.e., C₆ or C₁₀ aryl). In some embodiments, an aryl is phenyl. In some embodiments, an aryl is naphthyl. In some embodiments, an aryl is a C₆ or C₁₀aryl. In some embodiments, an aryl group can be unsubstituted or substituted.

The term “cycloalkyl” refers to a monocyclic or polycyclic hydrocarbon including at least one fully or partially saturated ring (i.e., non-aromatic ring), wherein each of the atoms forming the ring is a carbon atom. In some embodiments, cycloalkyls are spirocyclic or bridged compounds. In some embodiments, cycloalkyls include at least one partially saturated ring fused with an aromatic ring (e.g., 1,2,3,4-tetrahydronaphthalenyl), and the point of attachment is at a carbon of either ring. Cycloalkyl groups include groups having from 3 to 10 ring atoms. The cycloalkyl group can be designated as “C₃-C₁₀-cycloalkyl” or similar designations. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl, bicycle[1.1.1]pentyl, 2,3-dihydro-1H-indene and 1,2,3,4-tetrahydronaphthalenyl. In some embodiments, a cycloalkyl is a C₃-C₁₀-cycloalkyl. In some embodiments, a cycloalkyl is a C₃-C₉-cycloalkyl.

The term “halogen” or “halo” refers to a fluoro, chloro, bromo or iodo group. In some embodiments, halogen or halo is fluoro, chloro, or bromo. In some embodiments, halogen or halo is fluoro.

The terms “heteroaryl” refers to a monocyclic or fused multicyclic aromatic ring system and having at least one heteroatom in the ring system, that is, an element other than carbon, including but not limited to, nitrogen, oxygen and sulfur. Illustrative examples of heteroaryl groups include monocyclic heteroaryls and bicyclic heteroaryls. Monocyclic heteroaryls include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, 1,3,5-triazinyl, 1,3,4-oxadiazolyl, thiadiazolyl, and 1,2,5-oxadiazolyl. Bicyclic heteroaryls include, but are not limited to, indolizinyl, indolyl, benzofuranyl, benzothiophene (i.e., benzothiofuranyl), indazolyl, benzimidazolyl, purinyl, quinolinyl, isoquinolinyl, cinnolinyl, phthalazinyl, quinazolinyl, quinoxalinyl, 1,8-naphthyridinyl, and pteridinyl. In some embodiments, a heteroaryl contains 1~4 nitrogen atoms in the ring. In some embodiments, a heteroaryl has 5 to 10 ring members or 5 to 9 ring members. The heteroaryl group can be designated as “5-10 membered heteroaryl,” “5-9 membered heteroaryl,” or similar designations. In some embodiments, a heteroaryl can be an optionally substituted C₁-C₁₃ five-, six-, seven, eight-, nine-, ten-, up to 14-membered monocyclic, bicyclic, or tricyclic ring system including 1 to 5 heteroatoms selected from nitrogen, oxygen and sulfur. In some embodiments, a heteroaryl can be an optionally substituted C₁-C₈ five-, six-, seven, eight-, or nine-membered monocyclic, or bicyclic ring system including 1 to 4 heteroatoms selected from nitrogen, oxygen and sulfur. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, the C₁-C₄ 5-membered heteroaryl is furanyl, thienyl, 1,2,4-thiadiazolyl, 1,2,3-thiadiazolyl, isothiazolyl, thiazolyl, imidazolyl, pyrazolyl, isoxazolyl, oxazolyl, pyrrolyl, triazolyl, or tetrazolyl. In some embodiments, the heteroaryl is a C₃-C₅ 6-membered heteroaryl. In some embodiments, the C₃-C₅ 6-membered heteroaryl is pyridinyl, pyrimidinyl, pyrazinyl, pyridazinyl, or triazinyl. In some embodiments, a heteroaryl can be an optionally substituted C₆-C₉ ten-membered bicyclic ring system including 1 to 4 nitrogen atoms. In some embodiments, a heteroaryl can be an optionally substituted C₄-C₈ eight-, or nine-membered bicyclic ring system including 1 to 4 heteroatoms selected from nitrogen, oxygen and sulfur. In some embodiments, a heteroaryl can be an optionally substituted C₅-C₈ nine-membered bicyclic ring system including 1 to 4 heteroatoms selected from nitrogen, oxygen and sulfur. In some embodiments, a heteroaryl can be an optionally substituted C₅-C₈ nine-membered bicyclic ring system including 1 to 4 nitrogen atoms. In some embodiments, bicyclic heteroaryl is a C₆-C₉heteroaryl. In some embodiments, a heteroaryl group can be unsubstituted or substituted.

A “heterocycloalkyl” group refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system and optionally containing one or more unsaturated bonds situated in such a way, however, that a fully delocalized pi-electron system does not occur in the monocyclic ring or in at least one ring of the bicyclic or tricyclic ring system. The heteroatom(s) is an element other than carbon including, but not limited to, oxygen, sulfur, and nitrogen. When composed of two or more rings, the rings can be joined together in a fused, bridged, or spiro fashion where the heteroatom(s) can be present in any ring in the ring system. In some embodiments, a heterocycloalkyl can be an C₂-C₁₂ three-, four-, five-, six-, seven-, eight-, nine-, ten-, up to 13-membered monocyclic, bicyclic, or tricyclic ring system including 1 to 5 heteroatoms selected from nitrogen, oxygen and sulfur. In some embodiments, the heterocyclyl can be a C₂-C₆ three-, four-, five-, six-, or seven-membered monocyclic ring including 1 to 5 heteroatoms selected from nitrogen, oxygen and sulfur. In some embodiments, the heterocyclyl can be a C₂-C₁₀ four-, five-, six-, seven-, eight-, nine-, ten- or eleven-membered bicyclic ring system including 1 to 5 heteroatoms independently selected from nitrogen, oxygen and sulfur. In some embodiments, the heterocyclyl can be a C₇-C₁₂ 12- or 13-membered tricyclic ring system including 1 to 5 heteroatoms selected from nitrogen, oxygen and sulfur. In some embodiments, the heteroatom(s) of six membered monocyclic heterocyclyls are independently selected from one to three of oxygen, nitrogen and sulfur, and the heteroatom(s) of five membered monocyclic heterocyclyls are independently selected from oxygen, nitrogen and sulfur. In some embodiments, the heterocycloalkyl is pyrrolidinyl, tetrahydrofuranyl, tetrahydrothienyl, tetrahydropyranyl, tetrahydrothiopyranyl, piperidinyl, morpholinyl, thiomorpholinyl, or piperazinyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1 or 2 nitrogen atoms in the ring. In some embodiments, a heterocycloalkyl can be aziridinyl, azetidinyl, tetrahydrofuranyl, 1,3-dioxinyl, 1,3-dioxanyl, 1,4-dioxanyl, 1,2-dioxolanyl, 1,3-dioxolanyl, 1,3-oxathianyl, 1,4-oxathianyl, 1,3-oxathiolanyl, 1,3-dithiolyl, 1,3-dithiolanyl, 1,4-oxathianyl, tetrahydro-1,4-thiazinyl, imidazolinyl, imidazolidinyl, isoxazolinyl, isoxazolidinyl, isoindolinyl, indolinyl, oxazolinyl, oxazolidinyl, thiazolinyl, thiazolidinyl, morpholinyl, oxiranyl, piperidinyl, piperazinyl, pyrrolidinyl, pyrazolinyl, pyrazolidinyl, tetrahydropyranyl, tetrahydrothiopyranyl, 1,4-diazabicyclo[2.2.2]octane, 1,4-diazabicyclo[3.1.1]heptane, 2-azaspiro[3,3]heptane, 2,6-diazaspiro[3,3]heptane, tetrahydroquinolinyl, 1,2,3,4-tetrahydroisoquinolinyl, 1,2,3,4-tetrahydro-2,6-naphthyridinyl, 1,2,3,4-tetrahydro-2,7-naphthyridinyl, 1,2,3,4-tetrahydro-1,7-naphthyridinyl, 1,2,3,4-tetrahydro-1,6-naphthyridinyl, 5,6,7,8-tetrahydropyrido[2,3-d]pyrimidinyl, 5,6,7,8-tetrahydropyrido[3,4-d]pyrimidinyl, [1,3]dioxolo[4,5-c]pyridinyl, [1,3]dioxolo[4,5-b]pyridinyl, [1,3]dioxolo[4,5-d]pyrimidinyl or 3,4-methylenedioxyphenyl. In some embodiments, a substituted heterocyclyl can be oxazolidinonyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. When one or more substituents are present on the heterocyclyl group, the substituent(s) can be bonded at any available carbon atom and/or heteroatom. In some embodiments, a heterocyclyl group can be unsubstituted or substituted.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

Whenever a group is described as being “optionally substituted” that group can be unsubstituted, or can be substituted with one or more of the indicated substituents. Likewise, when a group is described as being “unsubstituted or substituted” if substituted, the substituent(s) can be selected from one or more of the indicated substituents. It is to be understood that substitution at a given atom is limited by valency. In some embodiments, substituted groups can be substituted with one or more additional group(s) individually and independently selected from halogen, —CN, —NH₂, —NH(alkyl), —N(alkyl)₂, —OH, —CO₂H, —CO₂alkyl, —C(═O) NH₂, —C(═O)NH(alkyl), —C(═O)N(alkyl)₂, —S(═O)₂NH₂, —S(═O)₂NH(alkyl), —S(═O)₂N (alkyl)₂, alkyl, cycloalkyl, alkoxy, heterocycloalkyl, aryl, heteroaryl, and alkylthio. In some embodiments, substituted groups can be substituted with substituents independently selected from halogen, —CN, —NH₂, —NH(CH₃), —N(CH₃)₂, —OH, —CO₂H, —CO₂ (C₁-C₄alkyl), —C(═O) NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄alkoxy, —SC₁-C₄alkyl, —S(═O) C₁-C₄alkyl, and —S(═O)₂C₁-C₄alkyl. In some embodiments, substituted groups can be substituted with substituents independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. In some embodiments, the compounds and compositions described herein are administered orally.

The terms “effective amount” or “therapeutically effective amount,” as used herein, refer to a sufficient amount of a compound as described herein, or a pharmaceutically acceptable salt thereof, or an amount of a pharmaceutical composition comprising the compound described herein or a pharmaceutically acceptable salt thereof, being administered, which will relieve to some extent one or more of the symptoms of the disease, disorder or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” as used herein, means to increase or prolong either in potency or duration a desired effect.

A health care practitioner can directly provide a compound described herein to a subject in the form of a sample or can indirectly provide a compound to a subject by providing an oral or written prescription for the compound. Also, for example, a subject can obtain a compound by themselves without the involvement of a health care practitioner. When the compound is administered to the subject, the body is transformed by the compound in some way. When a compound described herein is provided in combination with one or more other agents, “administration” is understood to include the compound and other agents are administered at the same time or at different times and includes both fixed and non-fixed combinations. The term “fixed combination” means that a compound as disclosed and described herein, or a pharmaceutically acceptable salt thereof, and a co-agent, are both administered to a subject simultaneously in the form of a single entity or dosage. The term “non-fixed combination” means that a compound as described herein, or a pharmaceutically acceptable salt thereof, and a co-agent, are administered to a subject as separate entities either simultaneously, concurrently or sequentially with no specific intervening time limits.

The term “subject” or “patient” refers to any animal, including mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. In the context of a clinical trial or screening or activity experiment the subject can be a healthy volunteer or healthy participant without an underlying Myc-mediated or CDK9-mediated disorder or condition or a volunteer or participant that has received a diagnosis for a disorder or condition in need of medical treatment as determined by a health care professional. In the context outside of a clinical trial a subject under the care of a health care professional who has received a diagnosis for a disorder or condition is typically described as a patient.

The terms “treat,” “treating” or “treatment,” as used herein, refer to medical management of a disease, disorder, or condition of a subject. The compounds as described herein can include beneficial or desired clinical results that comprise, but are not limited to, alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically and/or therapeutically.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:

Abbreviations:

| | |
|---|---|
| ACN | acetonitrile |
| BINAP | (±)-2,2′-Bis(diphenylphosphino)-1,1′-binaphthalene |
| Boc | tert-butyloxycarbonyl |
| Boc₂O | di-tert-butyl dicarbonate |
| Bu | butyl |
| BuLi | butyllithium |
| BuOH | butyl alcohol |
| Bu₃SnCl | tributyltin chloride |
| B₂pin₂ | bis(pinacolato)diboron |
| Cy₃P | tricyclohexylphosphine |
| Dba | dibenzylideneacetone |
| DCM | dichloromethane |
| DIPEA | N,N-diisopropylethylamine |
| DMAP | N,N-dimethylpyridin-4-amine |
| DMF | N,N-dimethylformamide |
| DMSO | dimethylsulfoxide |
| dppf | 1,1-bis(diphenylphosphino)ferrocene |
| EtOAc/EA | ethyl acetate |
| EtOH | ethyl alcohol |
| HOAc | acetic acid |
| HPLC | high pressure liquid chromatography |
| hr/hrs | hour/hours |
| IC₅₀ | half maximal inhibitory concentration |
| IPA | isopropyl alcohol |
| KOAc/AcOK | potassium acetate |
| LCMS | liquid chromatography-mass spectrometry |
| MeOH | methyl alcohol |
| mins | minutes |
| Mw | microwave |
| NaHMDS | sodium bis(trimethylsilyl)amide |
| NaOtBu | sodium tert-butoxide |
| NMR | nuclear magnetic resonance |
| O/N | overnight |
| Oxone ® | potassium hydrogen monopersulfate |
| Pd₂(dba)₃ | tris(dibenzylideneacetone)dipalladium(0) |
| Pd/C | palladium on carbon |
| Pd(dppf)Cl₂ | [1,1′-Bis(diphenylphosphino)ferrocene]palladium(II) |
| | dichloride |
| Pd(PPh₃)₄ | tetrakis(triphenylphosphine)palladium(0) |
| PE | petroleum ether |
| Ph | phenyl |
| PPh₃ | triphenylphosphine |
| prep-HPLC | preparative high pressure liquid chromatography |
| prep-TLC | preparative thin layer chromatography |
| r.t. | room temperature |
| SEM | trimethylsilylethoxymethyl |
| SEMCl | 2-(Trimethylsilyl)ethoxymethyl chloride |
| (SnBu₃)₂ | bis(tributyltin) |
| T3P | propanephosphonic acid anhydride |
| TMS-Cl | trimethylsilyl chloride |
| TEA | triethylamine |
| TFA | trifluoroacetic acid |
| THF | tetrahydrofuran |
| TLC | thin-layer chromatography |
| TMS-Cl | trimethylsilyl chloride |
| pTsOH/PTSA | p-toluenesulfonic acid |
| UPLC | ultra-performance liquid chromatography |
| Xantphos | 4,5-Bis(diphenylphosphino)-9,9-dimethylxanthene |

### General Synthetic Procedures

Unless otherwise stated, commercially available reagents and solvents were used without purification. Solvents: ACS grade. Reagents: unless otherwise noted, from Combi Blocks, Alfa Aesar, Fisher and Aldrich highest quality available. TLC: silica gel 60 F254 aluminum plates, (Whatman™, type Al Sil G/UV, 250 μm layer); visualization by UV absorption. Redisep and Biotage Flash+ systems were used for medium-pressure column chromatography. NMR: 1H spectra were obtained at Bruker 400 MHz spectrometer. ¹H NMR data are reported with chemical shifts (8) in parts-per-million (ppm) relative to the residual signal of the deuterated solvent as follows: chemical shift, multiplicity (s=singlet, d=doublet, t=triplet, q=quartet, qn=quintet, m=multiplet, and br=broad), coupling constant in Hz. Reactions were monitored by LCMS using Agilent 1260 infinity, Waters™ ACQUITY UPLC. Purity was determined by LCMS using a Waters™ ZQ Mass spectrometer equipped with an Extend-C₁₈ Rapid Resolution column (3.5 μm, 2.1 mm×50 mm). Elution was carried out with a 5-95% gradient over 4 min of ACN in water containing 0.1% formic acid at a flow rate of 0.75 mL/min at r.t. and for UPLC, the gradient was 90% water to 90% methanol with 0.1% formic acid added to both eluents, with 0.8 mL/min flow rate. The run time is 3.50 min.

### Example 1—Synthesis of Pyrimidine-Containing Compounds

### General Synthetic Scheme 1

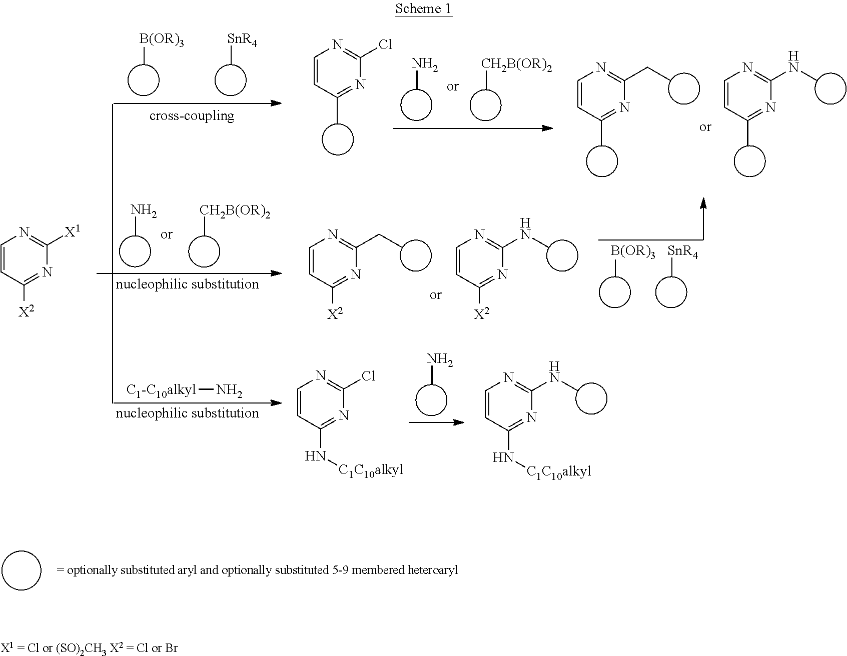

### Synthesis of 4-(1H-pyrrolo[2,3-c]pyridin-3-yl)-N-(3-(trifluoromethyl)phenyl)pyrimidin-2-amine (Compound 30)

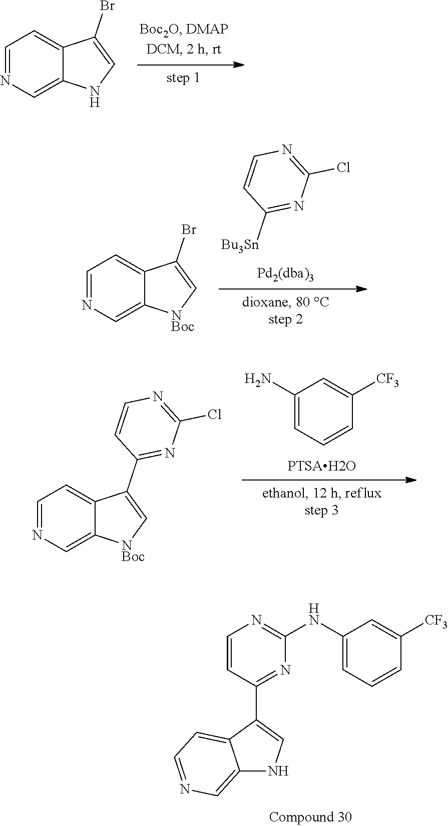

### Step 1—Synthesis of tert-butyl 3-bromo-1H-pyrrolo[2,3-c]pyridine-1-carboxylate

To a mixture of 3-bromo-1H-pyrrolo[2,3-c]pyridine (1 g, 1 Eq, 5 mmol), and di-tert-butyl dicarbonate (1 g, 1.05 Eq, 5 mmol) was added 1,4-dioxane (10 mL). Then was added N,N-dimethylpyridin-4-amine (0.6 g, 1 Eq, 5 mmol). The reaction mixture was stirred for 2 h. Concentrated under reduced pressure, and purified by 100% hexane to 100% ethyl acetate to afford the tert-butyl 3-bromo-1H-pyrrolo[2,3-c]pyridine-1-carboxylate (1.17 g, 3.94 mmol, 80%).

### Step 2—Synthesis of tert-butyl 3-(2-chloropyrimidin-4-yl)-1H-pyrrolo[2,3-b]pyridine-1-carboxylate

To a solution of tert-butyl 3-bromo-1H-pyrrolo[2,3-b]pyridine-1-carboxylate (0.1 g, 1 Eq, 0.3 mmol) and 2-chloro-4-(tributylstannyl)pyrimidine (0.1 g, 1.1 Eq, 0.4 mmol) in 1,4-dioxane (10 mL) was added Pd₂(dba)₃ (0.06 g, 0.2 Eq, 0.07 mmol), and the reaction vessel was evacuated, and then placed under N₂. The resulting mixture was treated with tert-butyl 3-bromo-1H-pyrrolo[2,3-b]pyridine-1-carboxylate (0.1 g, 1 Eq, 0.3 mmol), and 2-chloro-4-(tributylstannyl)pyrimidine (0.1 g, 1.1 Eq, 0.4 mmol) and stirred until complete. The mixture was filtered via celite and the filtrate was extracted with ethyl acetate, washed with water, then dried over brine. The crude organic layer was dried over Na₂SO₄ and the solvent was removed. Finally the remainder was purified by 10% MeOH-DCM to afford tert-butyl 3-(2-chloropyrimidin-4-yl)-1H-pyrrolo[2,3-b]pyridine-1-carboxylate (0.052 g, 0.16 mmol, 50%).

### Step 3—Synthesis of 4-(1H-pyrrolo[2,3-c]pyridin-3-yl)-N-(3-(trifluoromethyl)phenyl)pyrimidin-2-amine

To a solution of 3-(2-chloropyrimidin-4-yl)-1H-pyrrolo[2,3-c]pyridine (20 mg, 1 Eq, 87 μmol) and 3-(trifluoromethyl) aniline (28 mg, 22 μL, 2 Eq, 0.17 mmol) in ethanol (1 mL) was added pTsOH·H₂O (33 mg, 2 Eq, 0.17 mmol), and the resulting mixture was refluxed for 12 h. The reaction mixture was filtered via celite and the filtrate was extracted with ethyl acetate, washed with 10% NaHCO₃, washed with water, and brine. The crude organic layer was dried over Na₂SO₄ and the solvent was removed. Finally the remainder was purified by 10% MeOH-DCM to afford 4-(1H-pyrrolo[2,3-c]pyridin-3-yl)-N-(3-(trifluoromethyl)phenyl)pyrimidin-2-amine (9 mg, 0.03 mmol, 30%). ¹H NMR (400 MHz, CD₃OD) δ 8.63 (s, 1H), 8.54 (s, 1H), 8.43 (d, J=5.2 Hz, 1H), 8.32 (s, 1H), 8.04 (s, 1H), 7.87 (d, J=8.1 Hz, 1H), 7.70 (d, J=8.0 Hz, 2H), 7.51 (t, J=8.0 Hz, 1H), 7.31 (dd, J=12.7, 6.2 Hz, 2H), 7.23 (d, J=7.8 Hz, 2H). MS: m/z 356.1 (M+H⁺).

### Synthesis of 3-((4-(1H-benzo[d]imidazol-5-yl)pyrimidin-2-yl)amino)benzenesulfonamide (Compound 46)

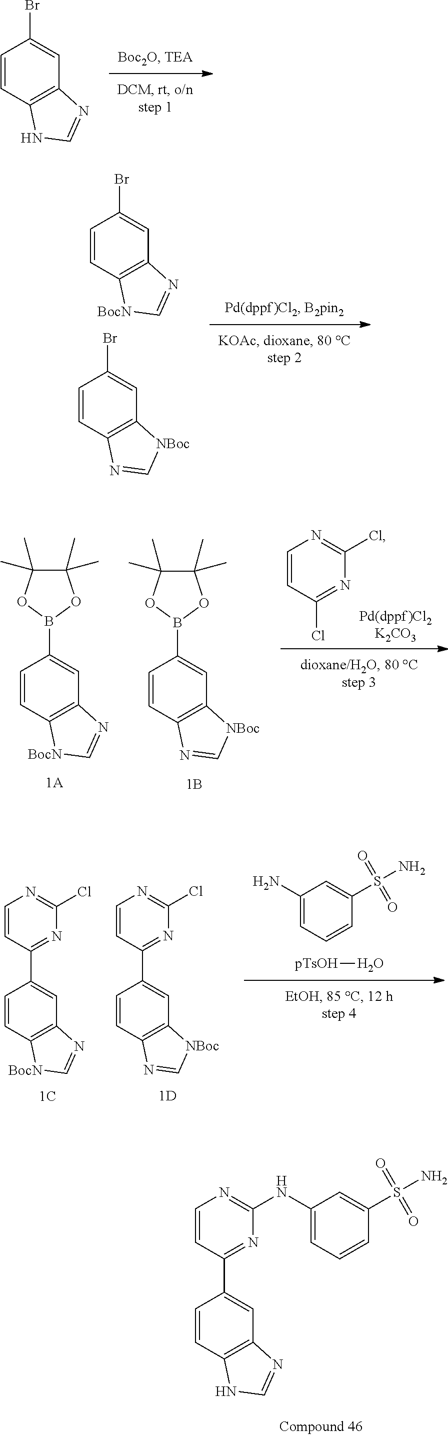

### Step 1—Synthesis of mixture of tert-butyl 5-bromo-1H-benzo[d]imidazole-1-carboxylate and tert-butyl 6-bromo-1H-benzo[d]imidazole-1-carboxylate

To mixture of 5-bromo-1H-benzo[d]imidazole (5.0 g, 1 Eq, 25 mmol) in DCM (10 mL) at 0 °C was added triethylamine (3.9 g, 5.3 mL, 1.5 Eq, 38 mmol) to afford a mixture. Di-tert-butyl decarbonate (Boc₂O, 8.3 g, 1.5 Eq, 38 mmol) dissolved in 5 mL of DCM was added slowly under nitrogen to the above mixture, and the resulting mixture was stirred for 12 h. The mixture was concentrated under reduced pressure and diluted with DCM (100 mL). The mixture was washed with water, then brine solution, and finally dried over Na₂SO₄. The organic layer was filtered, and the solvent was removed under reduced pressure. The remainder was purified on an 80 g silica column with a 100% hexane to 70% ethyl acetate gradient to furnish a mixture of tert-butyl 5-bromo-1H-benzo[d]imidazole-1-carboxylate and tert-butyl 6-bromo-1H-benzo[d]imidazole-1-carboxylate (7.1 g, 25 mmol, 94%) as a viscous liquid, which solidified on standing.

### Step 2—Synthesis of mixture of tert-butyl 5-(4,4,5,5-tetramethyl-1,3,2-dioxaborolan-2-yl)-1H-benzol 1-1-caroxylate and tert-butyl 6-(4,4,5,5-tetramethyl-1,3,2-dioxaborolan-2-yl)-1H-benzo[d]imidazole-1-carboxylate

To a mixture of potassium acetate (3.55 g, 3.0 Eq, 36.1 mmol), a mixture of tert-butyl 5-bromo-1H-benzo[d]imidazole-1-carboxylate and tert-butyl 6-bromo-1H-benzo[d]imidazole-1-carboxylate (3.58 g, 1 Eq, 12.0 mmol), and B₂pin₂ (4.59 g, 1.5 Eq, 18.1 mmol) was added 1,4-dioxane (60 mL) in a 100 mL round bottom flask. The mixture was degassed for 10 min and then was treated with Pd(dppf)Cl₂ (884 mg, 0.1 Eq, 1.20 mmol). The reaction mixture was heated for 3 h at 90 °C under N₂ and then allowed to cool to rt. The reaction mixture was diluted with ethyl acetate (100 mL) and water (100 mL), passed through a celite bed, organic layer was separated, the aqueous layer extracted with ethyl acetate (2× 100 mL). The combined organic layer was washed with water, then brine, and finally, dried over Na₂SO₄. The organic layer was filtered and dried under reduced pressure. The remainder was purified by a 120 g silica column by running a gradient of 100% hexane to 100% ethyl acetate to afford the title compound tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 4.12 g, 12 mmol, 85%).

### Step 3—Synthesis of mixture of tert-butyl 5-(2-chloropyrimidin-4-yl)-1H-benzo[d]imidazole-1-carboxylate and tert-butyl 6-(2-chloropyrimidin-4-yl)-1H-benzo[d]imidazole-1-carboxylate

To a solution of potassium carbonate (3.97 g, 2.5 Eq, 28.8 mmol) and a mixture of 2,4-dichloropyrimidine (2.40 g, 1.4 Eq, 16.1 mmol) in 1,4-dioxane (60 mL) under N₂ was added tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 3.96 g, 1 Eq, 11.5 mmol), and water (15 mL). The reaction mixture was purged with N₂ for 10 min and then was treated with Pd(dppf)Cl₂ (1.69 g, 0.2 Eq, 2.30 mmol). The resulting mixture was heated at 100 °C for 3 h under nitrogen and then allowed to cool to rt. The reaction mixture was diluted with ethyl acetate (100× 2 mL), passed through a celite bed, and then washed with water (100×2 mL), and finally with brine. The organic layer was dried over Na₂SO₄ and filtered. The organic layer was removed under reduced pressure and the remainder was loaded on a 80 g silica column to elute in 100% hexane to 100% ethyl acetate to afford a mixture of tert-butyl 5-(2-chloropyrimidin-4-yl)-1H-benzo[d]imidazole-1-carboxylate and tert-butyl 6-(2-chloropyrimidin-4-yl)-1H-benzo[d]imidazole-1-carboxylate (1C and 1D, 2.056 g, 6.216 mmol, 54.0%).

### Step 4—3-((4-(1H-benzo[d]imidazol-5-yl)pyrimidin-2-yl)amino)benzenesulfonamide (Compound 46)

To a solution of a mixture of tert-butyl 5-(2-chloropyrimidin-4-yl)-1H-benzo[d]imidazole-1-carboxylate and tert-butyl 6-(2-chloropyrimidin-4-yl)-1H-benzo[d]imidazole-1-carboxylate (1C and 1D, 50 mg, 1 Eq, 0.15 mmol) and 3-amino-N-phenylbenzenesulfonamide (56 mg, 1.5 Eq, 0.23 mmol) in ethanol (1 mL) was added pTsOH·H₂O (58 mg, 2 Eq, 0.30 mmol), and refluxed for 12 h at 85 °C and then allowed to cool to rt. The reaction mixture was diluted with ethyl acetate, and then washed with 10% NaHCO₃. The organic layer was washed with water, brine, and dried over Na₂SO₄ and the solid removed by filtration. The organic layer was removed under reduced pressure. Finally, the remainder was purified by 10% MeOH-DCM mixture to afford 3-((4-(1H-benzo[d]imidazol-6-yl)pyrimidin-2-yl)amino)benzenesulfonamide (Compound 46, 10 mg, 0.15 mmol, 18%). ¹H NMR (400 MHz, CD₃OD) δ 8.76 (s, 1H), 8.48 (s, 2H), 8.41 (dt, J=5.3, 1.4 Hz, 1H), 7.72 (s, 4H), 7.56-7.50 (m, 2H), 7.31 (dt, J=5.4, 1.4 Hz, 2H). MS: m/z 367.1 (M+H⁺).

### Synthesis of 4-(benzo[d]thiazol-5-yl)-N-(3-(trifluoromethyl)phenyl)pyrimidin-2-amine (Compound 59)

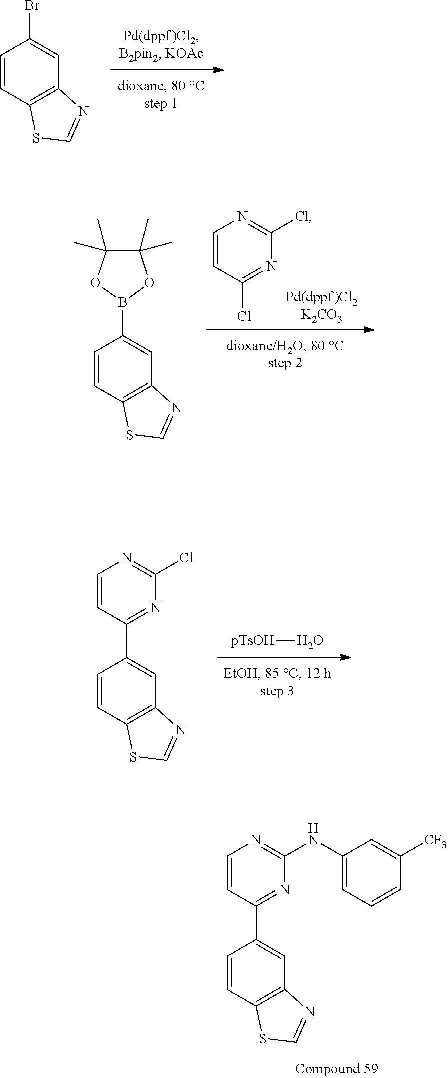

### Step 1—Synthesis of 5-(4,4,5,5-tetramethyl-1,3,2-dioxaborolan-2-yl)benzo[d]thiazole

To a mixture of potassium acetate (0.8 g, 3.6 Eq, 8 mmol) 5-bromobenzo[d]thiazole (0.5 g, 1 Eq, 2 mmol), and B₂pin₂ (1 g, 2 Eq, 5 mmol) was added 1,4-dioxane (30 mL) and the resulting mixture was degassed and placed under N₂. Subsequently, the mixture was treated with Pd(dppf)Cl₂ (0.2 g, 0.1 Eq, 0.2 mmol). The reaction mixture was heated at 90 °C for 2 h and then allowed to cool to rt. The solvent was removed under reduced pressure. The remainder was loaded on a 40 g silica column and run in 100% hexane to 100% ethyl acetate to afford 5-(4,4,5,5-tetramethyl-1,3,2-dioxaborolan-2-yl)benzo[d]thiazole (0.576 g, 2.21 mmol, 90%).

### Step 2—Synthesis of 5-(2-chloropyrimidin-4-yl)benzo[d]thiazole

To a solution of 5-(4,4,5,5-tetramethyl-1,3,2-dioxaborolan-2-yl)benzo[d]thiazole (0.575 g, 1 Eq, 2.2 mmol) and 2,4-dichloropyrimidine (394 mg, 1.2 Eq, 2.64 mmol) in 1,4-dioxane (10 mL) under nitrogen was added potassium carbonate (609 mg, 2 Eq, 4.4 mmol), and water (2.8 μL). The reaction mixture was purged with N₂ for 10 min and then was treated with Pd(dppf)Cl₂ (162 mg, 0.1 Eq, 220 μmol). The reaction mixture was heated at 90 °C for 2 h under nitrogen and then allowed to cool to rt. The reaction mixture was diluted with ethyl acetate (10×2 mL), water (10×2 mL), and finally with brine. The organic layer was removed under reduced pressure and the remainder was loaded on a 12 g silica column to elute in 100% hexane to 100% ethyl acetate to afford 5-(2-chloropyrimidin-4-yl)benzo[d]thiazole (0.027 g, 0.11 mmol, 5.0%).

### Step 3—Synthesis of 4-(benzo[d]thiazol-5-yl)-N-(3-(trifluoromethyl)phenyl)pyrimidin-2-amine (Compound 59)

To solution of 5-(2-chloropyrimidin-4-yl)benzo[d]thiazole (0.06 g, 1 Eq, 0.2 mmol) in ethanol (1 mL) was added 3-(trifluoromethyl) aniline (0.08 g, 0.06 mL, 98% wt, 2 Eq, 0.5 mmol) and 4-methylbenzenesulfonic acid monohydrate (0.08 g, 2 Eq, 0.5 mmol). The resulting mixture was heated at 80 °C for 12 h and then allowed to cool to rt. The solvent was removed under reduced pressure, diluted with ethyl acetate (20 mL), then NaHCO₃ (20 mL) and then the mixture was portioned and the organic layer was maintained. The organic layer was washed with water, dried over Na₂SO₄, and the solid removed by filtration. The solvent was removed under reduced pressure. The remainder was loaded on a 12 g silica column eluting with 10% methanol in DCM solvent mixture to afford 4-(benzo[d]thiazol-5-yl)-N-(3-(trifluoromethyl)phenyl)pyrimidin-2-amine (Compound 59, 0.017 g, 46 μmol, 20%).

### Synthesis of N⁴-(2-(1H-Imidazol-4-yl)ethyl)-N²-(3-(trifluoromethyl)phenyl)pyrimidine-2,4-diamine (Compound 111)

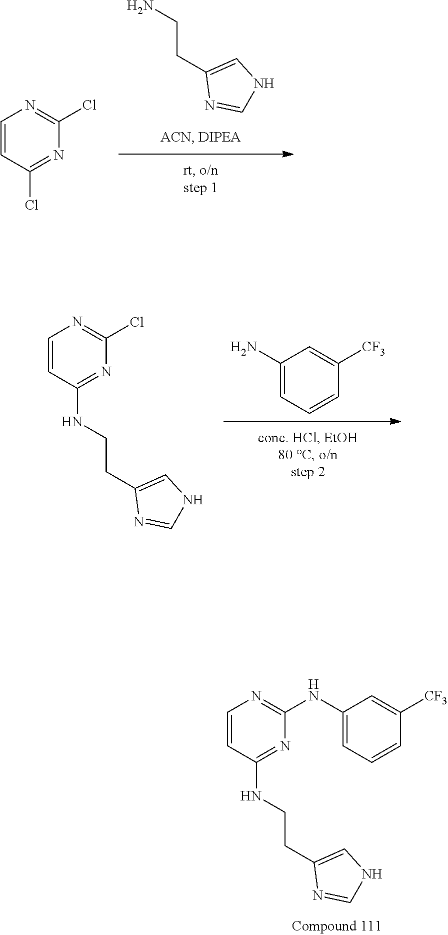 [SMILES: Clc1ccnc(Cl)n1 ; NCCc1c[nH]cn1 ; Clc1nccc(NCCc2c[nH]cn2)n1 ; Nc1cccc(C(F)(F)F)c1 ; FC(F)(F)c1cccc(Nc2nccc(NCCc3c[nH]cn3)n2)c1]

### Step 1—Synthesis of N-(2-(1H-imidazol-4-yl)ethyl)-2-chloropyrimidin-4-amine

A solution of 2,4-dichloropyrimidine (387 mg, 2.6 mmol), 2-(1H-imidazol-4-yl)ethanamine (222 mg, 2 mmol) and DIPEA (1034 mg, 8 mmol) in ACN (10 mL) was stirred at room temperature overnight under a N₂ atmosphere (balloon). The reaction mixture was concentrated under reduced pressure. The remainder was purified by silica gel column chromatography (DCM/MeOH=20/1) to afford N-(2-(1H-imidazol-4-yl)ethyl)-2-chloropyrimidin-4-amine (436 mg, yield: 97%) as a yellow oil.

### Step 2—Synthesis of N⁴-(2-(1H-imidazol-4-yl)ethyl)-N²-(3-(trifluoro methyl)phenyl)pyrimidine-2,4-diamine (Compound 111)

To a solution of N-(2-(1H-imidazol-4-yl)ethyl)-2-chloropyrimidin-4-amine (339 mg, 1.5 mmol) in EtOH (10 mL) was added 3-(trifluoromethyl) aniline (340 mg, 2.1 mmol) and conc. HCl (0.1 mL). The resulting mixture was placed under N₂ and stirred at 80 °C overnight and then allowed to cool to rt. The reaction mixture was concentrated and the remainder was purified by prep-HPLC to afford N⁴-(2-(1H-imidazol-4-yl)ethyl)-N²-(3-(trifluoro methyl)phenyl)pyrimidine-2,4-diamine (Compound 111, 35 mg, yield: 6.7%). ¹H NMR (400 MHz, DMSO-d₆): δ=11.82 (brs, 1H), 9.36 (s, 1H), 8.34 (s, 1H), 7.97 (d, J=5.6 Hz, 1H), 7.84 (s, 1H), 7.56 (s, 1H), 7.42 (t, J=7.4 Hz, 1H)), 7.35 (s, 1H), 7.17 (d, J=7.2 Hz, 1H), 6.82 (s, 1H), 6.01 (d, J=6.0 Hz, 1H), 3.59 (s, 2H), 2.80 (t, J=7.2 Hz, 2H). MS: m/z 349.1 (M+H⁺).

### Synthesis of 4-(2-((3-(Trifluoromethyl)phenyl)amino)pyrimidin-4-yl)phenol (Compound 130)

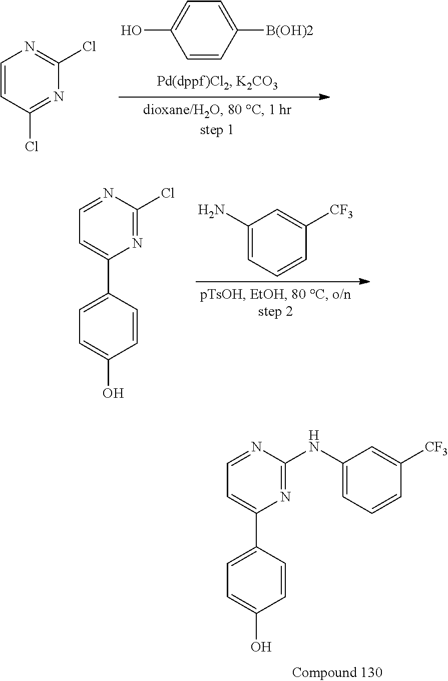 [SMILES: Clc1ccnc(Cl)n1 ; OB(O)c1ccc(O)cc1 ; Oc1ccc(-c2ccnc(Cl)n2)cc1 ; Nc1cccc(C(F)(F)F)c1 ; Oc1ccc(-c2ccnc(Nc3cccc(C(F)(F)F)c3)n2)cc1]

### Step 1—Synthesis of 4-(2-chloropyrimidin-4-yl)phenol

A solution of (4-hydroxyphenyl) boronic acid (200 mg, 1.44 mmol), 2,4-dichloropyrimidine (432 mg, 2.9 mmol), Pd(dppf)Cl₂ (109 mg, 0.15 mmol) and K₂CO₃ (400 mg, 2.9 mmol) in 1,4-dioxane/H₂O (10 mL/3 mL) was stirred at 80 °C for 3 hrs under N₂ atmosphere (balloon) and then allowed to cool to rt. The reaction mixture was filtered and the filtrate was concentrated. The remainder was purified by silica gel column to afford 4-(2-chloropyrimidin-4-yl)phenol (125 mg, yield: 43%).

### Step 2—Synthesis of 4-(2-((3-(trifluoromethyl)phenyl)amino)pyrimidin-4-yl)phenol (Compound 130)

A solution of 4-(2-chloropyrimidin-4-yl)phenol (100 mg, 0.5 mmol), 3-(trifluoromethyl) aniline (97 mg, 0.6 mmol) and pTsOH (172 mg, 1 mmol) in EtOH (10 mL) was stirred at 80 °C overnight and then allowed to cool to rt. The reaction mixture was concentrated under reduced pressure and the remainder was purified by prep-HPLC to afford 4-(2-((3-(trifluoromethyl)phenyl)amino)pyrimidin-4-yl)phenol (Compound 130, 43 mg, yield: 26%). 1H NMR (400 MHz, DMSO-d₆): δ=10.08 (brs, 1H), 9.56 (brs, 1H), 8.52-8.49 (m, 2H), 8.06 (t, J=8 Hz, 2H), 7.97 (d, J=8 Hz, 1H), 7.53 (d, J=8 Hz, 1H), 7.37 (d, J=4 Hz, 1H), 7.28 (d, J=4 Hz, 1H), 6.90 (d, J=8 Hz, 2H). MS: m/z 331.9 (M+H⁺).

### Synthesis of 2′-Methoxy-N-phenyl-[4,4′-bipyrimidin]-2-amine (Compound 163)

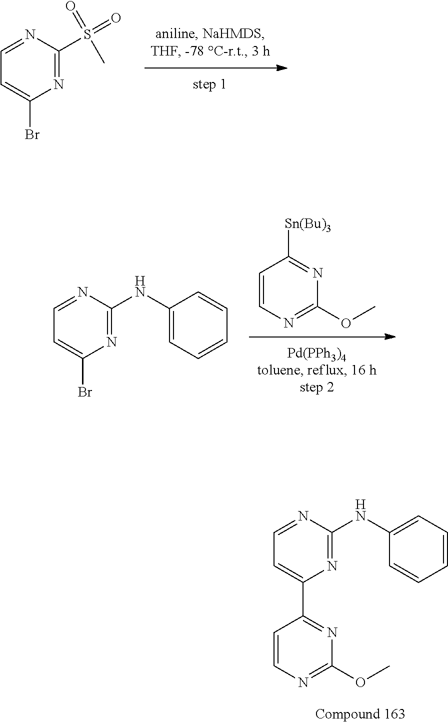

### Step 1—Synthesis of 4-bromo-N-phenylpyrimidin-2-amine

To a solution of aniline (368 mg, 4.2 mmol) in THF (20 mL) was added NaHMDS (2.5 M in hexane, 2.5 mL, 4.6 mmol) at −70 °C under nitrogen and the mixture was stirred for 1 hr, then treated with a solution of 4-bromo-2-(methylsulfonyl)pyrimidine (1.0 g, 4.2 mmol) at −70 °C and the resulting mixture was stirred at −70 °C for 2 hrs. Subsequently, the mixture was poured into ice water, the organic phase was separated and was concentrated under reduced pressure. The remainder was purified by reverse flash to afford 4-bromo-N-phenylpyrimidin-2-amine (250 mg, yield: 23.8%) as a yellow solid.

### Step 2—Synthesis of 2′-methoxy-N-phenyl-[4,4′-bipyrimidin]-2-amine (Compound 163)

A mixture of 4-bromo-N-phenylpyrimidin-2-amine (72 mg, 0.28 mmol), 2-methoxy-4-(tributylstannyl)pyrimidine (138 mg, 0.34 mmol) and Pd(PPh₃)₄ (33 mg, 0.03 mmol) in toluene (5 mL) was heated to reflux for 16 hrs. The reaction mixture was concentrated and purified by prep-HPLC to afford 2′-methoxy-N-phenyl-[4,4′-bipyrimidin]-2-amine (Compound 163, 16 mg, yield: 20.5%) as a yellow solid. ¹H NMR (400 MHz, CDCl₃): δ=8.74 (d, J=5.2 Hz, 1H), 8.63 (d, J=5.2 Hz, 1H), 7.97 (d, J=5.2 Hz, 1H), 7.81 (d, J=2.8 Hz, 1H), 7.68 (d, J=8.0 Hz, 2H), 7.37 (dd, J=7.6, 3.2 Hz, 2H), 7.07 (dd, J=7.2, 3.2 Hz, 1H), 4.11 (s, 3H). MS: m/z 280.1 (M+H⁺).

### Synthesis of 4-(Imidazo[1,2-a]pyridin-6-yl)-N-(3-(trifluoromethyl)phenyl)pyrimidin-2-amine (Compound 127)

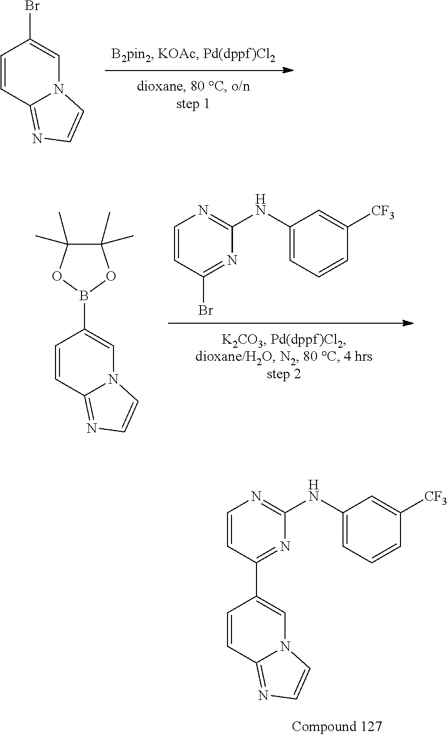

### Step 1—Synthesis of 6-(4,4,5,5-tetramethyl-1,3,2-dioxaborolan-2-yl) imidazo[1,2-a]pyridine

A solution of 6-bromoimidazo[1,2-a]pyridine (400 mg, 2.03 mmol), KOAc (600 mg, 6.09 mmol), B₂pin₂ (1.03 g, 4.06 mmol) and Pd(dppf)Cl₂ (150 mg, 0.20 mmol) in 1,4-dioxane (30 mL) was stirred at room temperature overnight under N₂ atmosphere (balloon). The reaction mixture was filtered and the filtrate was concentrated to afford 6-(4,4,5,5-tetramethyl-1,3,2-dioxaborolan-2-yl) imidazo[1,2-a]pyridine.

### Step 2—Synthesis of 4-(imidazo[1,2-a]pyridin-6-yl)-N-(3-(trifluoromethyl)phenyl)pyrimidin-2-amine

A mixture of 6-(4,4,5,5-tetramethyl-1,3,2-dioxaborolan-2-yl) imidazo[1,2-a]pyridine (100 mg, 0.41 mmol), 4-bromo-N-(3-(trifluoromethyl)phenyl)pyrimidin-2-amine (156 mg, 0.49 mmol), K₂CO₃ (170 mg, 1.23 mmol) and Pd(dppf)Cl₂ (30 mg, 0.04 mmol) in dioxane/H₂O (20 mL/4 mL) was stirred at room temperature for 4 hrs under N₂ atmosphere (balloon). The reaction mixture was filtered and the filtrate was concentrated. The remainder was purified by prep-HPLC (ACN and water with 0.05% TFA) to afford 4-(imidazo[1,2-a]pyridin-6-yl)-N-(3-(trifluoromethyl)phenyl)pyrimidin-2-amine (Compound 127, 5.5 mg, yield: 4%) as a white solid. ¹H NMR (400 MHz, DMSO-d₆): δ=10.11 (s, 1H), 9.43 (s, 1H), 8.65 (d, J=5.2 Hz, 1H), 8.41 (s, 1H), 8.04-7.98 (m, 3H), 7.70 (t, J=9.6 Hz, 2H), 7.57 (t, J=8.0 Hz, 1H), 7.51 (d, J=5.2 Hz, 1H), 7.32 (d, J=7.6 Hz, 1H). MS: m/z 355.9 (M+H⁺).

### Synthesis of 4-(Imidazo[1,2-a]pyrazin-6-yl)-N-(3-(trifluoromethyl)phenyl)pyrimidin-2-amine (Compound 107)

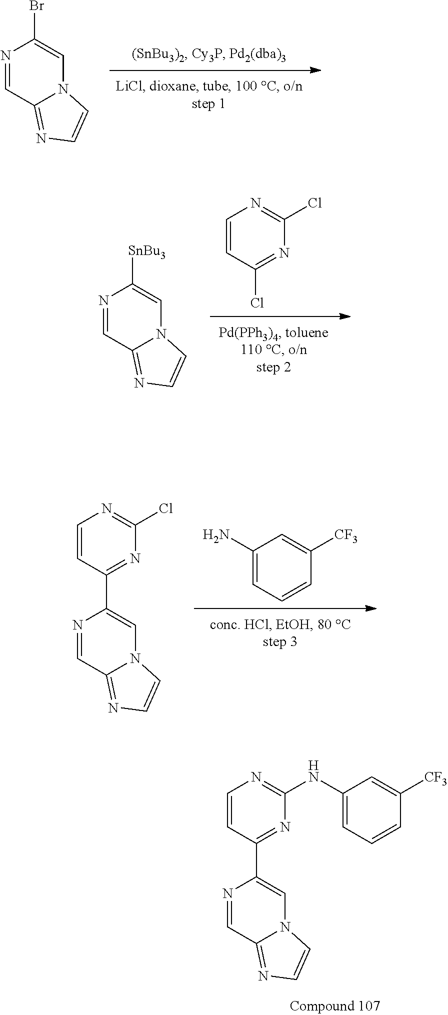

### Step 1—Synthesis of 6-(tributylstannyl) imidazo[1,2-a]pyrazine

A solution of 6-bromoimidazo[1,2-a]pyrazine (100 mg, 0.51 mmol), (SnBu₃)₂ (350 mg, 0.61 mmol), LiCl (130 mg, 3.03 mmol), Cy₃P (14 mg, 0.05 mmol) and Pd₂(dba)₃ (25 mg, 0.03 mmol) in dioxane (3 mL) was stirred at 100 °C overnight under N₂ atmosphere (balloon) and then allowed to cool to rt. The reaction mixture was filtered and the filtrate was concentrated under reduced pressure to afford 6-(tributylstannyl) imidazo[1,2-a]pyrazine.

### Step 2—Synthesis of 6-(2-chloropyrimidin-4-yl) imidazo[1,2-a]pyrazine

A solution of 6-(tributylstannyl) imidazo[1,2-a]pyrazine (140 mg, 0.34 mmol), 2,4-dichloropyrimidine (60 mg, 0.41 mmol) and Pd(PPh₃)₄, (40 mg, 0.03 mmol) in toluene (10 mL) was stirred at 110 °C overnight under N₂ atmosphere (balloon) and then allowed to cool to rt. The reaction mixture was filtered and the filtrate was concentrated under reduced pressure. The remainder was purified by prep-TLC (PE/EA=1/2) to afford 6-(2-chloropyrimidin-4-yl) imidazo[1,2-a]pyrazine (20 mg, yield: 3.5%) as a yellow solid.

### Step 3—Synthesis of 4-(imidazo[1,2-a]pyrazin-6-yl)-N-(3-(trifluoromethyl)phenyl)pyrimidin-2-amine (Compound 107)

To a solution of 6-(2-chloropyrimidin-4-yl) imidazo[1,2-a]pyrazine (20 mg, 0.09 mmol) in EtOH (10 mL) was added conc. HCl (0.25 mL), and the resulting mixture was stirred at 80 °C overnight and then allowed to cool to rt. The reaction mixture was concentrated under reduced pressure and the remainder was purified by prep-HPLC (ACN and water with 0.05% TFA) to afford 4-(imidazo[1,2-a]pyrazin-6-yl)-N-(3-(trifluoromethyl)phenyl)pyrimidin-2-amine (2.1 mg, yield: 3.5%) as a yellow solid. ¹H NMR (400 MHz, DMSO-d₆): δ=10.12 (s, 1H), 9.42 (s, 1H), 9.22 (s, 1H), 8.71 (d, J=4.8 Hz, 1H), 8.32 (s, 1H), 8.28 (s, 1H), 8.16 (d, J=8.0 Hz, 1H), 7.94 (s, 1H), 7.74 (d, J=4.8 Hz, 1H), 7.60 (t, J=3.6 Hz, 1H), 7.34 (d, J=3.6 Hz, 1H). MS: m/z 357.0 (M+H⁺).

### Synthesis of 4-(1H-Benzo[d][1,2,3]triazol-5-yl)-N-(3-(trifluoromethyl)phenyl)pyrimidin-2-amine (Compound 139)

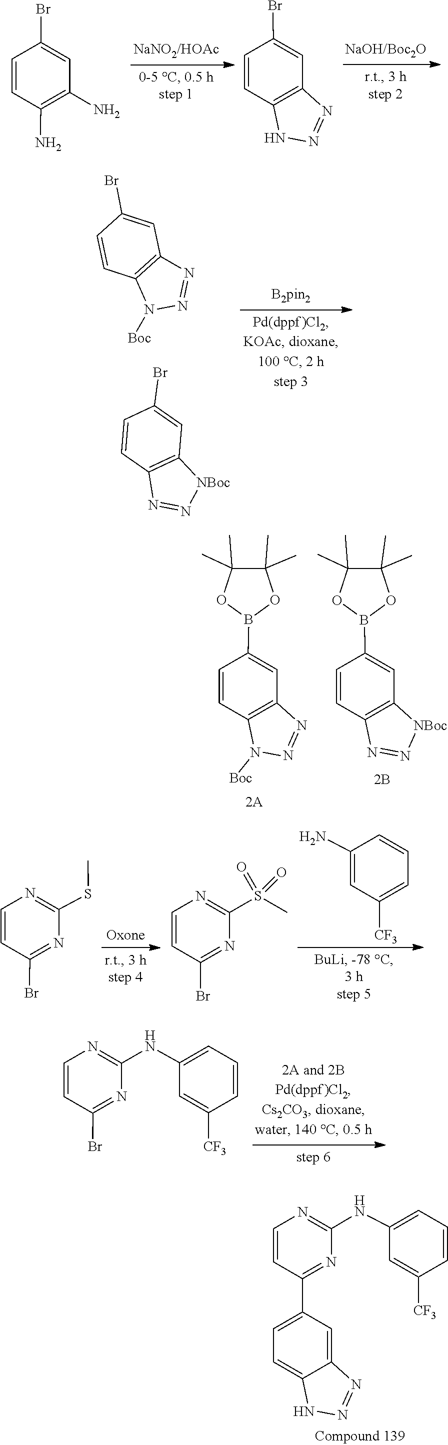

### Step 1—Synthesis of 5-bromo-1H-benzo[d][1,2,3]triazole

To a solution of 4-bromobenzene-1,2-diamine (7.5 g, 40.0 mmol) in HOAc/H₂O (50 mL/30 mL) was added a solution of NaNO₂ (2.8 g, 40.0 mmol) in water (10 mL) dropwise over 5 mins at 0 °C. Subsequently, the reaction mixture was stirred at room temperature for 30 mins. The reaction mixture was then poured into ice water (350 mL) and extracted with ethyl acetate (400 mL). The ethyl acetate layer was washed with brine (350 mL), dried over anhydrous sodium sulfate, filtered to remove solids and concentrated under reduced pressure. The remainder was purified by silica gel column (PE/EA=1/1) to afford 5-bromo-1H-benzo[d][1,2,3]triazole (4.75 g, yield: 60.1%).

### Step 2—Synthesis of mixture of tert-butyl 5-bromo-1H-benzo[d][1,2,3]triazole-1-carboxylate and tert-butyl 6-bromo-1H-benzo[d][1,2,3]triazole-1-carboxylate

A solution of 5-bromo-1H-benzo[d][1,2,3]triazole (1.0 g, 5.1 mmol), Boc₂O (1.7 g, 7.6 mmol) and NaOH (0.3 g, 7.6 mmol) in THF/water (5 mL/5 mL) was stirred at room temperature for 3 hrs. The reaction mixture was poured into H₂O (50 mL) and extracted with ethyl acetate (50 mL). The ethyl acetate layer was washed with brine (50 mL), dried over Na₂SO₄, filtered to remove solids and concentrated under reduced pressure. The remainder was purified by silica gel column (PE/EA=5/1) to afford a mixture of tert-butyl 5-bromo-1H-benzo[d][1,2,3]triazole-1-carboxylate and tert-butyl 6-bromo-1H-benzo[d][1,2,3]triazole-1-carboxylate (1.15 g, yield: 76.1%) which was carried to the next step without separation.

### Step 3—Synthesis of mixture of tert-butyl 5-(4,4,5,5-tetramethyl-1,3,2-dioxaborolan-2-yl)-1H-benzo[d][1,2,3]triazole-1-carboxylate and tert-butyl 6-(4,4,5,5-tetramethyl-1,3,2-dioxaborolan-2-yl)-1H-benzo[d][1,2,3]triazole-1-carboxylate

A solution of a mixture of tert-butyl 5-bromo-1H-benzo[d][1,2,3]triazole-1-carboxylate and tert-butyl 6-bromo-1H-benzo[d][1,2,3]triazole-1-carboxylate (915 mg, 3.08 mmol), B₂pin₂ (858 mg, 3.38 mmol), Pd(dppf)Cl₂ (220 mg, 0.31 mmol) and KOAc (604 mg, 6.16 mmol) in 1,4-dioxane (5 mL) was heated to 100 °C for 2 hrs under N₂ atmosphere (balloon) and then allowed to cool to rt. The mixture was filtered to remove solids, and the filtrate was concentrated under reduced pressure to afford a mixture of tert-butyl 5-(4,4,5,5-tetramethyl-1,3,2-dioxaborolan-2-yl)-1H-benzo[d][1,2,3]triazole-1-carboxylate and tert-butyl 6-(4,4,5,5-tetramethyl-1,3,2-dioxaborolan-2-yl)-1H-benzo[d][1,2,3]triazole-1-carboxylate (2A and 2B, 858 mg, 80.7%), which was used directly in the next step.

### Step 4—Synthesis of 4-bromo-2-(methylsulfonyl)pyrimidine

To a solution of 4-bromo-2-(methylthio)pyrimidine (1.5 g, 7.32 mmol) in methanol/water (15 mL/9 mL) was added potassium hydrogen monopersulfate (9.0 g, 14.6 mmol). The mixture was stirred at room temperature for 2 hrs. The reaction mixture was filtered to remove solids. The filtrate was poured into H₂O (100 mL) and extracted with ethyl acetate (100 mL). The ethyl acetate layer was washed with brine (100 mL), dried over anhydrous sodium sulfate, filtered to remove solids and concentrated to afford 4-bromo-2-(methylsulfonyl)pyrimidine (1.35 g, yield: 78.2%).

### Step 5—Synthesis of 4-bromo-N-(3-(trifluoromethyl)phenyl)pyrimidin-2-amine

To a solution of 3-(trifluoromethyl) aniline (680 mg, 4.2 mmol) in THF (20 mL) was added NaHMDS (2.5 M in hexane) (2.0 mL, 4.8 mmol) at −70 °C under N₂ atmosphere (balloon). The mixture was stirred at −70 °C for 1 hr, then added a solution of 4-bromo-2-(methylsulfonyl)pyrimidine (1.0 g, 4.2 mmol) and stirred at −70 °C for 2 hrs. The mixture was poured into ice water and the layers were allowed to partition. The organic phase was maintained and concentrated under reduced pressure. The remainder was purified by reverse flash to afford 4-bromo-N-(3-(trifluoromethyl)phenyl)pyrimidin-2-amine (360 mg, yield: 35.6%).

### Step 6—Synthesis of 4-(1H-benzo[d][1,2,3]triazol-5-yl)-N-(3-(trifluoromethyl)phenyl)pyrimidin-2-amine (Compound 139)

A solution of a mixture of tert-butyl 5-(4,4,5,5-tetramethyl-1,3,2-dioxaborolan-2-yl)-1H-benzo[d][1,2,3]triazole-1-carboxylate and tert-butyl 6-(4,4,5,5-tetramethyl-1,3,2-dioxaborolan-2-yl)-1H-benzo[d][1,2,3]triazole-1-carboxylate (2A and 2B, 90 mg, 0.26 mmol), 4-bromo-N-(3-(trifluoromethyl)phenyl)pyrimidin-2-amine (83 mg, 0.26 mmol), Pd(dppf)Cl₂ (22 mg, 0.03 mmol) and Cs₂CO₃ (163 mg, 0.52 mmol) in dioxane/water (3 mL/1 mL) was heated to 140 °C for 0.5 hr under nitrogen and then allowed to cool to rt. The reaction mixture was concentrated under reduced pressure and the remainder was purified by prep-HPLC to afford 4-(1H-benzo[d][1,2,3]triazol-5-yl)-N-(3-(trifluoromethyl)phenyl)pyrimidin-2-amine (Compound 139, 3.6 mg, yield: 4.0%). ¹H NMR (300 MHz, CD₃OD): δ=8.73 (s, 1H), 8.54 (d, J=4.8 Hz, 1H), 8.29 (d, J=4.8 Hz, 1H), 7.97 (d, J=3.6 Hz, 1H), 7.81 (d, J=3.6 Hz, 1H), 7.61 (dd, J=4.8, 3.6 Hz, 1H), 7.55 (d, J=8.0 Hz, 2H), 7.45 (dd, J=3.6, 1.2 Hz, 1H), 7.27 (dd, J=4.8, 1.2 Hz, 1H), 6.84 (dd, J=2.4, 1.2 Hz, 1H). MS: m/z 357.0 (M+H⁺).

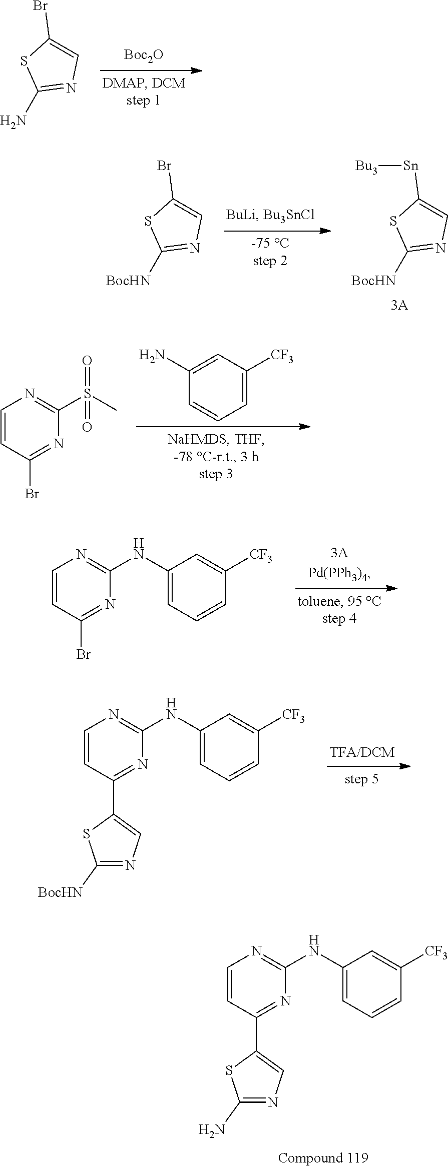

### Synthesis of 5-(2-((3-(Trifluoromethyl)phenyl)amino)pyrimidin-4-yl)thiazol-2-amine (Compound 119)

### Step 1—Synthesis of tert-butyl (5-bromothiazol-2-yl) carbamate

A solution of 5-bromothiazol-2-amine (554 mg, 3.13 mmol), Boc₂O (758 mg, 3.56 mmol), TEA (631 mg, 6.26 mmol) and DMAP (50 mg) in DCM (5 mL) was stirred at room temperature for 2 hrs. The reaction mixture was concentrated and the residue was purified by silica gel column (PE/EA=10/1) to afford tert-butyl (5-bromothiazol-2-yl) carbamate (722 mg, yield: 83.2%).

### Step 2—Synthesis of tert-butyl (5-(tributylstannyl)thiazol-2-yl) carbamate

To a solution of tert-butyl (5-bromothiazol-2-yl) carbamate (200 mg, 0.72 mmol) in THF (5 mL) was treated with BuLi (2.5 M in hexane, 0.64 mL, 1.58 mmol) at −75 °C under N₂ atmosphere (balloon). The resulting mixture was stirred at −75 °C for 30 mins, then treated with Bu₃SnCl (0.3 mL, 1.08 mmol) and the resulting mixture was stirred at −75 °C for 1 hr. The reaction mixture was poured into ice/water (30 mL) and extracted with ethyl acetate (30 mL×2). The combined ethyl acetate layers were concentrated under reduced pressure to afford an isolate of tert-butyl (5-(tributylstannyl)thiazol-2-yl) carbamate (3A, 300 mg, yield: 95.8%), which was used directly in next step.

### Step 3—Synthesis of 4-bromo-N-(3-(trifluoromethyl)phenyl)pyrimidin-2-amine

To a solution of 3-(trifluoromethyl) aniline (680 mg, 4.2 mmol) in THF (20 mL) was added NaHMDS (2.5 M in hexane) (2.0 mL, 4.8 mmol) at −70 °C under N₂ atmosphere (balloon). The mixture was stirred at −70 °C for 1 hr, then added a solution of 4-bromo-2-(methylsulfonyl)pyrimidine (1.0 g, 4.2 mmol) and the new mixture was stirred at −70 °C for 2 hrs. The reaction mixture was poured into ice/water (40 mL) and extracted with ethyl acetate (40 mL). The ethyl acetate layer was concentrated under reduced pressure and the remainder was purified by reverse flash to afford 4-bromo-N-(3-(trifluoromethyl)phenyl)pyrimidin-2-amine (360 mg, yield: 35.6%).

### Step 4—Synthesis of tert-butyl (5-(2-((3-(trifluoromethyl)phenyl)amino)pyrimidin-4-yl)thiazol-2-yl) carbamate

A mixture of 4-bromo-N-(3-(trifluoromethyl)phenyl)pyrimidin-2-amine (191 mg, 0.60 mmol), tert-butyl (5-(tributylstannyl)thiazol-2-yl) carbamate (3A, 313 mg, 0.72 mmol) and Pd(PPh₃)₄ (70 mg, 0.06 mmol) in toluene (5 mL) was heated to reflux for 16 hrs and then allowed to cool to rt. The reaction mixture was concentrated under reduced pressure and the remainder was purified by prep-HPLC to afford tert-butyl (5-(2-((3-(trifluoromethyl)phenyl)amino)pyrimidin-4-yl)thiazol-2-yl) carbamate (45 mg, yield: 16.8%).

### Step 5—Synthesis of 5-(2-((3-(trifluoromethyl)phenyl)amino)pyrimidin-4-yl)thiazol-2-amine (Compound 119)

A solution of tert-butyl (5-(2-((3-(trifluoromethyl)phenyl)amino)pyrimidin-4-yl)thiazol-2-yl) carbamate (25 mg, 0.06 mmol) and TFA (1 mL) in DCM (2 mL) was stirred at 25 °C for 1 hr. The reaction mixture was concentrated under reduced pressure and the remainder was purified by prep-HPLC to afford 5-(2-((3-(trifluoromethyl)phenyl)amino)pyrimidin-4-yl)thiazol-2-amine (Compound 119, 12.5 mg, yield: 65.1%). ¹H NMR (400 MHz, DMSO-d₆): δ=9.82 (s, 1H), 8.35 (s, 1H), 8.33 (s, 1H), 7.99 (s, 1H), 7.94 (d, J=8.8 Hz, 1H), 7.68 (s, 2H), 7.47 (t, J=7.6, 3.6 Hz, 1H), 7.24 (d, J=7.6 Hz, 1H), 7.21 (d, J=5.6 Hz, 1H). MS: m/z 338.0 (M+H⁺).

### Synthesis of 5-(2-((3-Fluorophenyl)amino)pyrimidin-4-yl)-1H-benzo[d]imidazol-2(3H)-one (Compound 116)

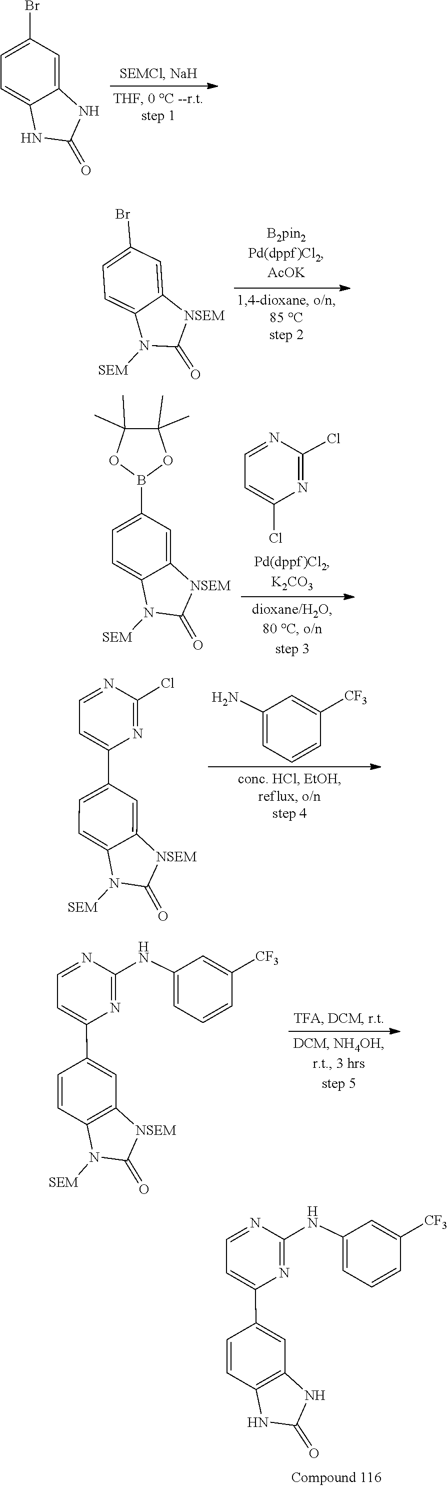

### Step 1—Synthesis of 5-bromo-1,3-bis((2-(trimethylsilyl)ethoxy)methyl)-1H-benzo[d]imidazol-2(3H)-one

To a mixture of 5-bromo-1H-benzo[d]imidazol-2(3H)-one (426 mg, 2.0 mmol) in dry THF was added NaH (60% in oil, 320 mg, 8.0 mmol) at 0 °C. After 15 mins, the mixture was treated with a solution of SEMCl (1 g, 6.0 mmol) in dry THF (10 mL) by dropwise addition. The resulting mixture was stirred at room temperature for 3 hrs. The mixture was poured into water (20 mL) and extracted with ethyl acetate (10 mL×3). The combined organic layers were concentrated under reduced pressure and the remainder was purified by silica gel column (PE/EA=8/1) to give 5-bromo-1,3-bis((2-(trimethylsilyl)ethoxy)methyl)-1H-benzo[d]imidazol-2(3H)-one (342 mg, yield: 36%). ¹H NMR (400 MHz, CDCl₃): δ=7.35 (d, J=1.8 Hz, 1H), 7.30-7.25 (m, 1H), 7.09-7.04 (m, 1H), 5.31 (d, J=3.7 Hz, 4H), 3.66-3.57 (m, 4H), 0.99-0.90 (m, 4H), 0.03-0.07 (m, 18H).

### Step 2—Synthesis of 5-(4,4,5,5-tetramethyl-1,3,2-dioxaborolan-2-yl)-1,3-bis((2-(trimethylsilyl)ethoxy)methyl)-1H-benzo[d]imidazol-2(3H)-one

A solution of 5-bromo-1,3-bis((2-(trimethylsilyl)ethoxy)methyl)-1H-benzo[d]imidazol-2(3H)-one (342 mg, 0.192 mmol), B₂pin₂ (369 mg, 1.45 mmol), Pd(dppf)Cl₂ (52.7 mg, 0.072 mmol) and AcOK (213 mg, 2.17 mmol) in 1,4-dioxane (6 mL) was stirred at 85 °C for 3 hrs under N₂ atmosphere (balloon) and then allowed to cool to rt. The mixture was concentrated under reduced pressure and the remainder was purified by silica gel column (PE/EA=8/1) to afford 5-(4,4,5,5-tetramethyl-1,3,2-dioxaborolan-2-yl)-1,3-bis((2-(trimethylsilyl)ethoxy)methyl)-1H-benzo[d]imidazol-2(3H)-one (424 mg, yield: 99%) as colorless oil. ¹H NMR (400 MHz, CDCl₃): δ=7.69-7.63 (m, 2H), 7.22 (d, J=8.0 Hz, 1H), 5.37 (d, J=5.1 Hz, 4H), 3.71-3.59 (m, 4H), 1.39 (s, 12H), 1.01-0.91 (m, 4H), 0.00 (d, J=3.8 Hz, 18H).

### Step 3—5-(2-chloropyrimidin-4-yl)-1,3-bis((2-(trimethylsilyl)ethoxy)methyl)-1H-benzo[d]imidazol-2(3H)-one

A solution of 5-(4,4,5,5-tetramethyl-1,3,2-dioxaborolan-2-yl)-1,3-bis((2-(trimethylsilyl)ethoxy)methyl)-1H-benzo[d]imidazol-2(3H)-one (424 mg, 0.8 mmol), 2,4-dichloropyrimidine (133 mg, 0.892 mmol), Pd(dppf)Cl₂ (52.7 mg, 0.072 mmoL) and K₂CO₃ (213 mg, 2.17 mmol) in 1,4-dioxane/H₂O (10 mL/2 mL) was stirred at 85 °C for 4 hrs and then allowed to cool to rt. The reaction mixture was concentrated under reduced pressure and the remainder was purified by silica gel column (PE/EA=10/1) to afford 5-(2-chloropyrimidin-4-yl)-1,3-bis((2-(trimethylsilyl)ethoxy)methyl)-1H-benzo[d]imidazol-2(3H)-one (280 mg, yield: 68.2%). ¹H NMR (400 MHz, CDCl₃): δ=8.63 (d, J=5.3 Hz, 1H), 8.00 (d, J=1.5 Hz, 1H), 7.96 (dd, J=8.3, 1.7 Hz, 1H), 7.67 (d, J=5.3 Hz, 1H), 7.32 (d, J=8.3 Hz, 1H), 5.42 (d, J=18.4 Hz, 4H), 3.67 (dd, J=16.2, 7.6 Hz, 4H), 0.98 (dd, J=16.4, 9.0 Hz, 4H), 0.00 (d, J=1.8 Hz, 18H).

### Step 4—Synthesis of 5-(2-((3-(trifluoromethyl)phenyl)amino)pyrimidin-4-yl)-1,3-bis((2-(trimethylsilyl)ethoxy)methyl)-1H-benzo[d]imidazol-2(3H)-one

A solution of 5-(2-chloropyrimidin-4-yl)-1,3-bis((2-(trimethylsilyl)ethoxy)methyl)-1H-benzo[d]imidazol-2(3H)-one (150 mg, 0.296 mmol), conc. HCl (0.2 mL) and 3-(trifluoromethyl) aniline (52.4 mg, 0.325 mmol) in EtOH was stirred at 80 °C overnight and then allowed to cool to rt. The mixture was concentrated under reduced pressure and the remainder was purified by silica gel column (PE/EA=10/1) to afford 5-(2-((3-(trifluoromethyl)phenyl)amino)pyrimidin-4-yl)-1,3-bis((2-(trimethylsilyl)ethoxy)methyl)-1H-benzo[d]imidazol-2(3H)-one (67 mg, yield: 36%) as yellow oil. ¹H NMR (400 MHz, CDCl₃): δ=8.51 (d, J=5.3 Hz, 1H), 8.33 (s, 1H), 7.98-7.96 (m, 1H), 7.75 (d, J=8.1 Hz, 1H), 7.49 (d, J=7.9 Hz, 1H), 7.46 (s, 1H), 7.32 (d, J=8.2 Hz, 2H), 7.26 (d, J=5.3 Hz, 1H), 5.44-5.39 (m, 4H), 3.66 (dd, J=11.6, 4.6 Hz, 4H), 0.99-0.95 (m, 4H), 0.01-0.02 (m, 18H).

### Step 5—Synthesis of 5-(2-((3-(trifluoromethyl)phenyl)amino)pyrimidin-4-yl)-1H-benzo[d]imidazol-2(3H)-one (Compound 116)

A solution of 5-(2-((3-(trifluoromethyl)phenyl)amino)pyrimidin-4-yl)-1,3-bis((2-(trimethylsilyl)ethoxy)methyl)-1H-benzo[d]imidazol-2(3H)-one (44 mg, 0.0696 mmol) and TFA (1 mL) in DCM (5 mL) was stirred at room temperature overnight. The reaction mixture was concentrated under reduced pressure, and then a portion of DCM (2 mL) and NH₄OH (5 mL) were combined with the remainder. The resulting mixture was stirred at room temperature for 3 hrs. The mixture was filtered to collect the solid which was the desired component from the phase interface. Purification by prep-TLC afforded 5-(2-((3-(trifluoromethyl)phenyl)amino)pyrimidin-4-yl)-1H-benzo[d]imidazol-2(3H)-one as a white solid (Compound 116, 7.7 mg, yield: 30%). ¹H NMR (400 MHz, DMSO-d₆): δ=10.92 (s, 2H), 9.98 (s, 1H), 8.54 (d, J=5.3 Hz, 1H), 8.38 (s, 1H), 8.06 (d, J=8.2 Hz, 1H), 7.84 (d, J=8.2 Hz, 1H), 7.76 (s, 1H), 7.54 (t, J=8.0 Hz, 1H), 7.42 (d, J=5.3 Hz, 1H), 7.29 (d, J=7.6 Hz, 1H), 7.06 (d, J=8.2 Hz, 1H). MS: m/z 372.0 (M+H⁺).

### Synthesis of N-(3-Fluorophenyl)-4-(1H-indazol-5-yl)pyrimidin-2-amine (Compound 165)

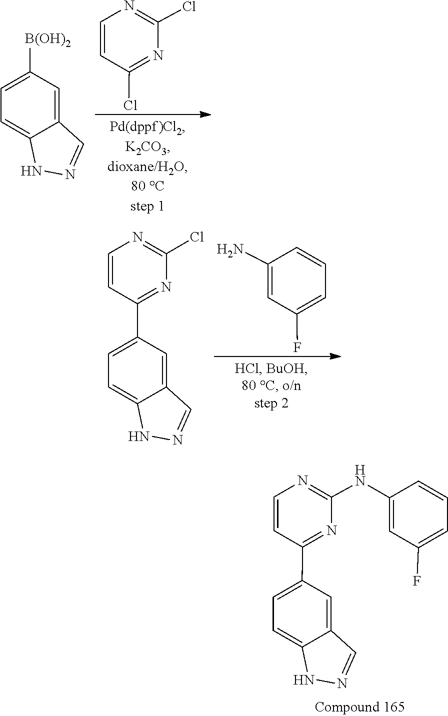

### Step 1—Synthesis of 5-(2-chloropyrimidin-4-yl)-1H-indazole

A solution of (1H-indazol-5-yl) boronic acid (162 mg, 1 mmol), Pd(dppf)Cl₂ (73.1 mg, 0.1 mmol), K₂CO₃ (276 mg, 2 mmol) and 2,4-dichloropyrimidine (178.8 mg, 1.2 mmol) in 1,4-dioxane/H₂O (10 mL/1 mL) was stirred at 80 °C overnight under N₂ atmosphere (balloon) and then allowed to cool to rt. The reaction mixture was treated with water (10 mL), extracted with ethyl acetate (10 mL×3), dried over anhydrous Na₂SO₄, filtered to remove solids and concentrated under reduced pressure. The remainder was purified by flash column chromatography (ACN in water=30%-60%, 30 mins) to afford 5-(2-chloropyrimidin-4-yl)-1H-indazole (157 mg, yield: 68.3%).

### Step 2—Synthesis of N-(3-fluorophenyl)-4-(1H-indazol-5-yl)pyrimidin-2-amine (Compound 165)

To a solution of 5-(2-chloropyrimidin-4-yl)-1H-indazole (157 mg, 0.683 mmol) in BuOH (5 mL) was added conc. HCl (0.3 mL) and 3-fluoroaniline (90.9 mg, 0.82 mmol). The mixture was stirred at 80 °C overnight and then allowed to cool to rt. The reaction mixture was concentrated under reduced pressure. The remainder was purified by prep-HPLC to afford N-(3-fluorophenyl)-4-(1H-indazol-5-yl)pyrimidin-2-amine (7.5 mg, yield: 3.6%). ¹H NMR (400 MHz, CD₃OD): δ=12.33 (s, 1H), 9.91 (s, 1H), 8.65 (s, 1H), 8.57 (d, J=5.2 Hz, 1H), 8.25-8.19 (m, 2H), 7.90 (d, J=12.8 Hz, 1H), 7.70 (d, J=8.8 Hz, 1H), 7.60 (d, J=8.4 Hz, 1H), 7.53 (d, J=5.2 Hz, 1H), 7.38-7.32 (m, 1H), 6.80-6.75 (m, 1H). MS: m/z 306.1 (M+H⁺).

### Synthesis of 2-Benzyl-4-(2-methoxypyridin-4-yl)pyrimidine (Compound 230)

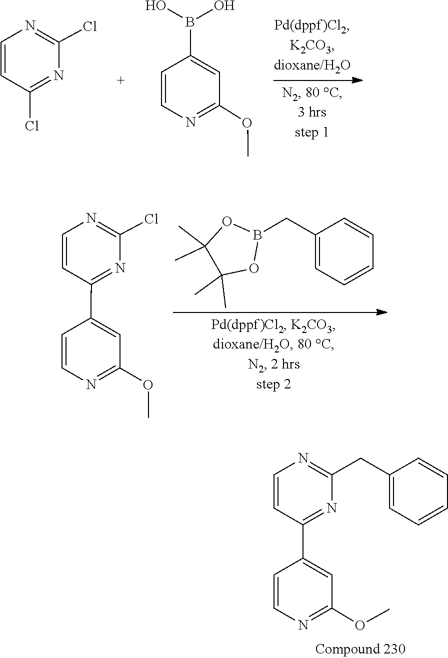

### Step 1—Synthesis of 2-chloro-4-(3-methoxyphenyl)pyrimidine

A solution of 2,4-dichloropyrimidine (596 mg, 4 mmol), (3-methoxyphenyl) boronic acid (608 mg, 4 mmol), Pd(dppf)Cl₂ (293 mg, 0.4 mmol) and K₂CO₃ (1.1 g, 8 mmol) in dioxane/H₂O (30 mL/6 mL) was stirred at 80 °C overnight under N₂ atmosphere (balloon) and then allowed to cool to rt. The reaction mixture was filtered and the filtrate was concentrated under reduced pressure. The remainder was purified by silica gel column (PE/EA=10/1) to afford 2-chloro-4-(3-methoxyphenyl)pyrimidine (754 mg, yield: 56%).

### Step 2—Synthesis of 2-benzyl-4-(2-methoxypyridin-4-yl)pyrimidine (Compound 230)

A solution of 2-chloro-4-(2-methoxypyridin-4-yl)pyrimidine (111 mg, 0.5 mmol), 2-benzyl-4,4,5,5-tetramethyl-1,3,2-dioxaborolane (120 mg, 0.55 mmol), Pd(dppf)Cl₂ (37 mg, 0.05 mmol) and K₂CO₃ (138 mg, 1 mmol) in dioxane/H₂O (10 mL/2 mL) was stirred at 80 °C overnight under N₂ atmosphere (balloon) and then allowed to cool to rt. The reaction mixture was filtered to remove solids and the filtrate was concentrated under reduced pressure. The remainder was purified by prep-HPLC to afford 2-benzyl-4-(2-methoxypyridin-4-yl)pyrimidine (Compound 230, 4 mg, yield: 3%). ¹H NMR (400 MHz, DMSO-d₆): δ=8.89 (d, J=5.2 Hz, 1H), 8.35 (dd, J=5.2, 1.2 Hz, 1H), 8.02 (d, J=5.2 Hz, 1H), 7.71 (dd, J=5.2, 1.2 Hz, 1H), 7.55 (s, 1H), 7.38-7.36 (m, 2H), 7.31 (t, J=7.6 Hz, 2H), 7.24-7.20 (m, 1H), 4.30 (s, 2H), 3.92 (s, 3H). MS: m/z 278.1 (M+H⁺).

### Synthesis of 4-(2-Benzylpyrimidin-4-yl)pyridin-2-ol (Compound 229)

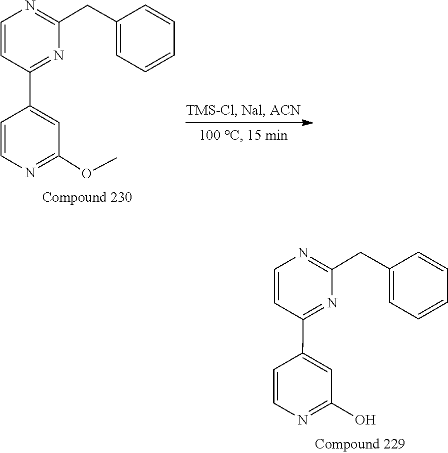 [SMILES: COc1cc(-c2ccnc(Cc3ccccc3)n2)ccn1 ; Oc1cc(-c2ccnc(Cc3ccccc3)n2)ccn1]

A solution of 2-benzyl-4-(2-methoxypyridin-4-yl)pyrimidine (Compound 230, 65 mg, 0.23 mmol) and NaI (141 mg, 0.94 mmol) in ACN (5 mL) was stirred at room temperature for 5 mins, then treated with TMS-Cl (102 mg, 0.94 mmol). The resulting mixture was stirred at 100 °C for 15 mins and then allowed to cool to rt. The resulting mixture was concentrated under reduced pressure and purified by prep-HPLC to afford 4-(2-benzylpyrimidin-4-yl)pyridin-2-ol (Compound 229, 5.9 mg, yield: 10%). ¹H NMR (400 MHz, DMSO-d₆): δ=11.79 (brs, 1H), 8.86 (d, J=5.2 Hz, 1H), 7.93 (d, J=5.2 Hz, 1H), 7.53 (d, J=6.4 Hz, 1H), 7.36-7.28 (m, 4H), 7.23-7.19 (m, 1H), 7.12 (d, J=1.2 Hz, 1H), 6.87 (dd, J=6.8, 1.6 Hz, 1H), 4.28 (s, 2H). MS: m/z 264.1 (M+H⁺).

### Synthesis of N²′-(3-(Trifluoromethyl)phenyl)-[2,4′-bipyrimidine]-2′,5-diamine (Compound 137)

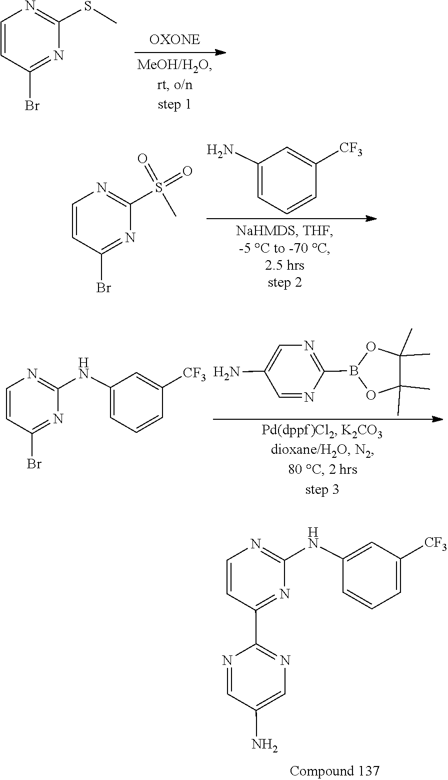

### Step 1—Synthesis of 4-bromo-2-(methylsulfonyl)pyrimidine

To a solution of 4-bromo-2-(methylthio)pyrimidine (3.0 g, 14.63 mmol) in MeOH/H₂O (100 mL/50 mL) was added potassium hydrogen monopersulfate (18 g, 29.26 mmol). The mixture was stirred at room temperature for 16 hrs. The reaction mixture was filtered to remove solids. The filtrate was concentrated under reduced pressure to remove MeOH. The mixture was extracted with ethyl acetate (150 mL). The organic layer was washed with water (30 mL), brine (30 mL), dried over Na₂SO₄, filtered to remove solids and concentrated under reduced pressure to afford 4-bromo-2-(methylsulfonyl)pyrimidine (2.99 g, yield: 86%). ¹H NMR (300 MHz, DMSO-d₆): δ=8.96 (d, J=6.8 Hz, 1H), 8.24 (d, J=7.2 Hz, 1H), 3.46 (s, 3H).

### Step 2—Synthesis of 4-bromo-N-(3-(trifluoromethyl)phenyl)pyrimidin-2-amine

To a solution of 3-(trifluoromethyl) aniline (1.08 g, 6.75 mmol) in THF (15 mL) was added NaHMDS (6.8 mL, 13.5 mmol) with ice-brine. The mixture was stirred at −5 °C for 30 mins under N₂ (balloon). The mixture was cooled to −70 °C and then treated with a solution of 4-bromo-2-(methylsulfonyl)pyrimidine (1.6 g, 6.75 mmol) in THF (20 mL). The resulting mixture was stirred at −70 °C for 2 hrs under N₂ (balloon). The reaction mixture was quenched by sat. NH₄Cl solution, extracted with ethyl acetate (150 mL). The organic layer was dried over Na₂SO₄, filtered to remove solids and concentrated under reduced pressure. The isolate was purified by silica gel column (PE/EA=20/1) to give a second isolate. The second isolate was purified by reverse flash to afford 4-bromo-N-(3-(trifluoromethyl)phenyl)pyrimidin-2-amine (0.98 g, yield: 45%). ¹H NMR (300 MHz, DMSO-d₆): δ=10.41 (s, 1H), 8.41 (d, J=5.4 Hz, 1H), 8.22 (s, 1H), 7.96 (d, J=8.4 Hz, 1H), 7.56 (t, J=7.8 Hz, 1H), 7.35 (d, J=7.5 Hz, 1H), 7.96 (d, J=5.4 Hz, 1H).

### Step 3—Synthesis of N2′-(3-(trifluoromethyl)phenyl)-[2,4′-bipyrimidine]-2′,5-diamine (Compound 137)

A mixture of 4-bromo-N-(3-(trifluoromethyl)phenyl)pyrimidin-2-amine (50 mg, 0.16 mmol), 2-(4,4,5,5-tetramethyl-1,3,2-dioxaborolan-2-yl)pyrimidin-5-amine (61 mg, 0.27 mmol), Pd(dppf)Cl₂ (14 mg, 0.016 mmol) and K₂CO₃ (44 mg, 0.32 mmol) in 1,4-dioxane/H₂O (5 mL/1 mL) was stirred at 80 °C for 2 hrs overnight under N₂ atmosphere (balloon) and then allowed to cool to rt. The reaction mixture was filtered and the filtrate was concentrated under reduced pressure. The remainder was purified by silica gel column (DCM-DCM/MeOH=50/1) and prep-HPLC to afford N²′-(3-(trifluoromethyl)phenyl)-[2,4′-bipyrimidine]-2′,5-diamine (Compound 137, 4.7 mg, yield: 9%). ¹H NMR (400 MHz, DMSO-d₆): δ=10.17 (brs, 1H), 9.15 (brs, 2H), 8.59 (d, J=5.2 Hz, 1H), 8.41 (s, 1H), 7.95 (d, J=8.0 Hz, 1H), 7.56 (t, J=8.0 Hz, 1H), 7.49 (d, J=5.2 Hz, 1H), 7.46-7.20 (m, 2H). MS: m/z 333.0 (M+H⁺).

### Synthesis of 4-(2-Methyl-1H-benzo[d]imidazol-6-yl)-N-(3-(trifluoromethyl)phenyl)pyrimidin-2-amine (Compound 142)

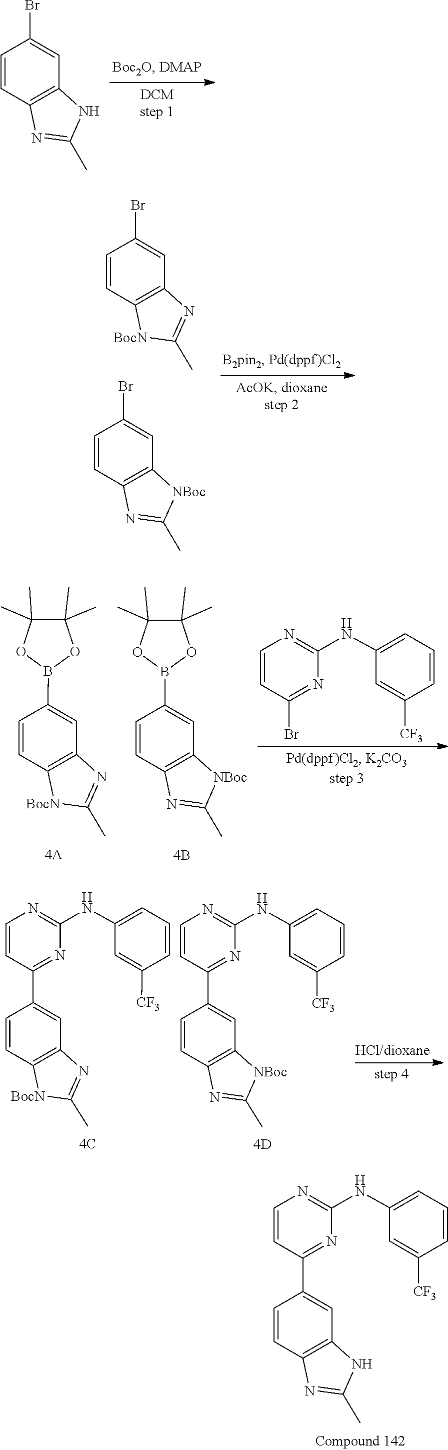

### Step 1—Synthesis of mixture of tert-butyl 6-bromo-2-methyl-1H-benzo[d]imidazole-1-carboxylate and tert-butyl 5-bromo-2-methyl-1H-benzo[d]imidazole-1-carboxylate

A solution of 6-bromo-2-methyl-1H-benzo[d]imidazole (1 g, 4.7 mmol), Boc₂O (2 g, 9.5 mmol) and DMAP (1.2 g, 9.5 mmol) in DCM (10 mL) was stirred at room temperature overnight. The reaction mixture was concentrated and the remainder was purified by silica gel column chromatography to give a mixture of tert-butyl 6-bromo-2-methyl-1H-benzo[d]imidazole-1-carboxylate and tert-butyl 5-bromo-2-methyl-1H-benzo[d]imidazole-1-carboxylate (1.2 g, yield: 80%).

### Step 2—Synthesis of mixture of tert-butyl 2-methyl-6-(4,4,5,5-tetramethyl-1,3,2-dioxaborolan-2-yl)-1H-benzo[d]imidazole-1-carboxylate and tert-butyl 2-methyl-5-(4,4,5,5-tetramethyl-1,3,2-dioxaborolan-2-yl)-1H-benzo[d]imidazole-1-carboxylate

A solution of a mixture of tert-butyl 6-bromo-2-methyl-1H-benzo[d]imidazole-1-carboxylate and tert-butyl 5-bromo-2-methyl-1H-benzo[d]imidazole-1-carboxylate (100 mg, 0.32 mmol), B₂pin₂ (163 mg, 0.64 mmol), AcOK (63 mg, 0.64 mmol) and Pd(dppf)Cl₂ (23 mg, 0.032 mmol) in 1,4-dioxane (1 mL) was stirred at 80 °C for 16 hrs under N₂ atmosphere (balloon). The resulting mixture containing a mixture of tert-butyl 2-methyl-6-(4,4,5,5-tetramethyl-1,3,2-dioxaborolan-2-yl)-1H-benzo[d]imidazole-1-carboxylate and tert-butyl 2-methyl-5-(4,4,5,5-tetramethyl-1,3,2-dioxaborolan-2-yl)-1H-benzo[d]imidazole-1-carboxylate (4A and 4B) was cooled to room temperature and used directly for the next step. MS: m/z 359.2 (M+H⁺).

### Step 3—Synthesis of mixture of tert-butyl 2-methyl-6-(2-((3-(trifluoromethyl)phenyl)amino)pyrimidin-4-yl)-1H-benzo[d]imidazole-1-carboxylate and tert-butyl 2-methyl-6-(2-((3-(trifluoromethyl)phenyl)amino)pyrimidin-4-yl)-1H-benzo[d]imidazole-1-carboxylate

A mixture of tert-butyl 2-methyl-6-(4,4,5,5-tetramethyl-1,3,2-dioxaborolan-2-yl)-1H-benzo[d]imidazole-1-carboxylate and tert-butyl 2-methyl-5-(4,4,5,5-tetramethyl-1,3,2-dioxaborolan-2-yl)-1H-benzo[d]imidazole-1-carboxylate (4A and 4B, 115 mg, 0.32 mmol), 4-bromo-N-(3-(trifluoromethyl)phenyl)pyrimidin-2-amine (102 mg, 0.32 mmol), K₂CO₃ (90 mg, 0.64 mmol) and Pd(dppf)Cl₂ (23 mg, 0.032 mmol) in 1,4-dioxane/H₂O (10 mL/2 mL) was stirred at 80 °C overnight under N₂ atmosphere (balloon) and then allowed to cool to rt. The mixture was poured into water (40 mL) and extracted with DCM (40 mL). The organic layer was dried over Na₂SO₄, filtered to remove solids and concentrated under reduced pressure to afford tert-butyl 2-methyl-6-(2-((3-(trifluoromethyl)phenyl)amino)pyrimidin-4-yl)-1H-benzo[d]imidazole-1-carboxylate and tert-butyl 2-methyl-6-(2-((3-(trifluoromethyl)phenyl)amino)pyrimidin-4-yl)-1H-benzo[d]imidazole-1-carboxylate (4C and 4D, 150 mg) as crude mixture.

### Step 4—Synthesis of 4-(2-methyl-1H-benzo[d]imidazol-6-yl)-N-(3-(trifluoromethyl)phenyl)pyrimidin-2-amine (Compound 142)

A solution of tert-butyl 2-methyl-6-(2-((3-(trifluoromethyl)phenyl)amino)pyrimidin-4-yl)-1H-benzo[d]imidazole-1-carboxylate and tert-butyl 2-methyl-6-(2-((3-(trifluoromethyl)phenyl)amino)pyrimidin-4-yl)-1H-benzo[d]imidazole-1-carboxylate (4C and 4D, 150 mg, 0.32 mmol) in HCl/1,4-dioxane (1 mL, 4 N) was stirred at room temperature for 3 hrs. The reaction mixture was concentrated under reduced pressure and purified by prep-HPLC to afford 4-(2-methyl-1H-benzo[d]imidazol-6-yl)-N-(3-(trifluoromethyl)phenyl)pyrimidin-2-amine (Compound 142, 27 mg, yield: 23%) as a white solid. ¹H NMR (400 MHz, DMSO-d₆): δ=12.45 (brs, 1H), 9.99 (s, 1H), 8.56 (t, J=5.2 Hz, 1H), 8.50 (s, 1H), 8.31 (s, 1H), 8.02 (t, J=7.6 Hz, 2H), 7.58-7.52 (m, 3H), 7.29 (d, J=7.6 Hz, 1H), 2.54 (s, 3H). MS: m/z 370.1 (M+H⁺).

### Synthesis of 4-(1H-Benzo[d]imidazol-6-yl)-N-(3-(trifluoromethyl)phenyl)pyrimidin-2-amine 2HCl (Compound 171)

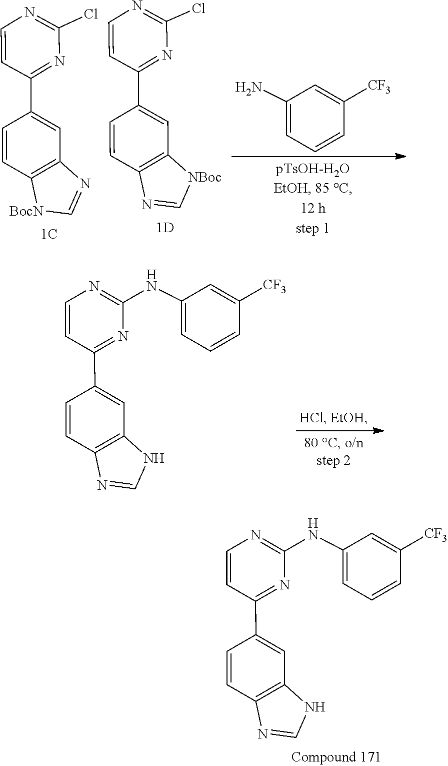

### Step 1: Synthesis of 4-(1H-benzo[d]imidazol-5-yl)-N-(3-(trifluoromethyl)phenyl)pyrimidin-2-amine

To a solution of a mixture of tert-butyl 5-(2-chloropyrimidin-4-yl)-1H-benzo[d]imidazole-1-carboxylate and tert-butyl 6-(2-chloropyrimidin-4-yl)-1H-benzo[d]imidazole-1-carboxylate (1C and 1D, 0.6 g, 1 Eq, 2 mmol) in ethanol (10 mL) was added 3-(trifluoromethyl) aniline (0.6 g, 0.5 mL, 2 Eq, 4 mmol) and 4-methylbenzenesulfonic acid (0.6 g, 2 Eq, 4 mmol). The resulting mixture was heated at 80 °C for 12 hrs. The mixture was allowed to cool to rt and then the organic solvent removed under reduced pressure and the remainder was combined with ethyl acetate (100 mL). The organic layer was washed with a saturated solution of sodium bicarbonate, water, and brine. The organic layer was dried over Na₂SO₄ and the solid removed by filtration. The organic later was removed under reduced pressure. Finally, the remainder was purified by 10% MeOH-DCM mixture on a 80 g column to afford 4-(1H-benzo[d]imidazol-5-yl)-N-(3-(trifluoromethyl)phenyl)pyrimidin-2-amine (0.289 g, 813 μmol, 40%).

### Step 2—Synthesis of 5-(2-((3-(trifluoromethyl)phenyl)amino)pyrimidin-1-ium-4-yl)-1H-benzo[d]imidazol-3-ium chloride (Compound 171)

To a solution of 4-(1H-benzo[d]imidazol-5-yl)-N-(3-(trifluoromethyl)phenyl)pyrimidin-2-amine (0.497 g, 95% Wt, 1 Eq, 1.33 mmol) in THF (10 mL) was added hydrogen chloride (107 mg, 731 μL, 4 molar, 2.2 Eq, 2.92 mmol) and stirred for 1 h. The reaction mixture was dried under reduced pressure and then was triturated with ethyl ether (2×10 mL). Dried again under high vacuum for 24 hr at 50 °C to afford 5-(2-((3-(trifluoromethyl)phenyl)amino)pyrimidin-1-ium-4-yl)-1H-benzo[d]imidazol-3-ium chloride (Compound 171). ¹H NMR (400 MHz, DMSO-d₆): δ=12.74 (brs, 1H), 106.01 (s, 1H), 8.58 (d, J=5.6 Hz, 1H), 8.49-8.33 (m, 2H), 8.35 (s, 1H), 8.09 (d, J=8.0 Hz, 1H), 8.03 (d, J=8.0 Hz, 1H), 7.71-7.70 (m, 1H), 7.57-7.53 (m, 2H), 7.29 (d, J=7.8 Hz, 1H). MS: m/z 356.1 (M+H⁺).

### Synthesis of 4-(1H-benzo[d]imidazol-5-yl)-N-(3-(difluoromethyl)phenyl)pyrimidin-2-amine (Compound 280)

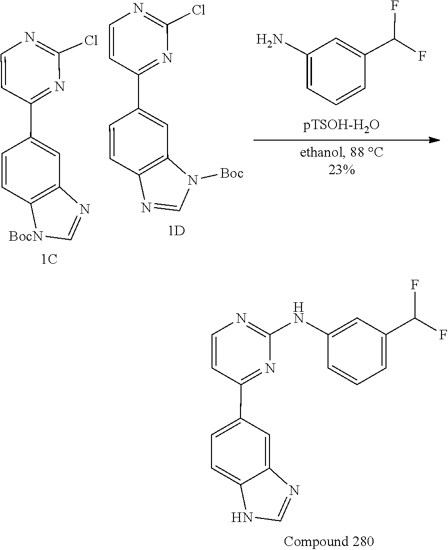

To a solution of tert-butyl 5-(2-chloropyrimidin-4-yl)-1H-benzo[d]imidazole-1-carboxylate and tert-butyl 6-(2-chloropyrimidin-4-yl)-1H-benzo[d]imidazole-1-carboxylate (1C and 1D, 0.050 g, 1 Eq, 0.15 mmol) and 3-(difluoromethyl) aniline (43 mg, 2 Eq, 0.30 mmol) in 4-methylbenzenesulfonic acid hydrate (58 mg, 2 Eq, 0.30 mmol) was added in a microwave 2-5 mL vial. The via was capped. The reaction mixture was heated for 12 h at 88 °C and then was cooled. The mixture was diluted with ethyl acetate, washed with NaHCO₃, dried over Na₂SO₄, and the solid removed by filtration. The organic layer was removed under the reduced pressure and the remainder was loaded on a 24 g silica column. The crude was purified with 10% MeOH in DCM to furnish the final compound 4-(1H-benzo[d]imidazol-6-yl)-N-(3-(difluoromethyl)phenyl)pyrimidin-2-amine (Compound 280, 11.8 mg, 35.0 μmol, 23%) 1H NMR (400 MHz, CDCl₃) δ 7.66 (d, J=5.4 Hz, 1H), 7.36 (d, J=37.5 Hz, 2H), 7.05 (d, J=8.2 Hz, 1H), 6.95 (s, 2H), 6.69-6.48 (m, 2H), 6.36 (d, J=7.6 Hz, 1H), 5.94 (s, 1H), 3.70 (s, 1H). MS: m/z 339.0 (M+H).

### Synthesis of (Compound 277A and Compound 277B)

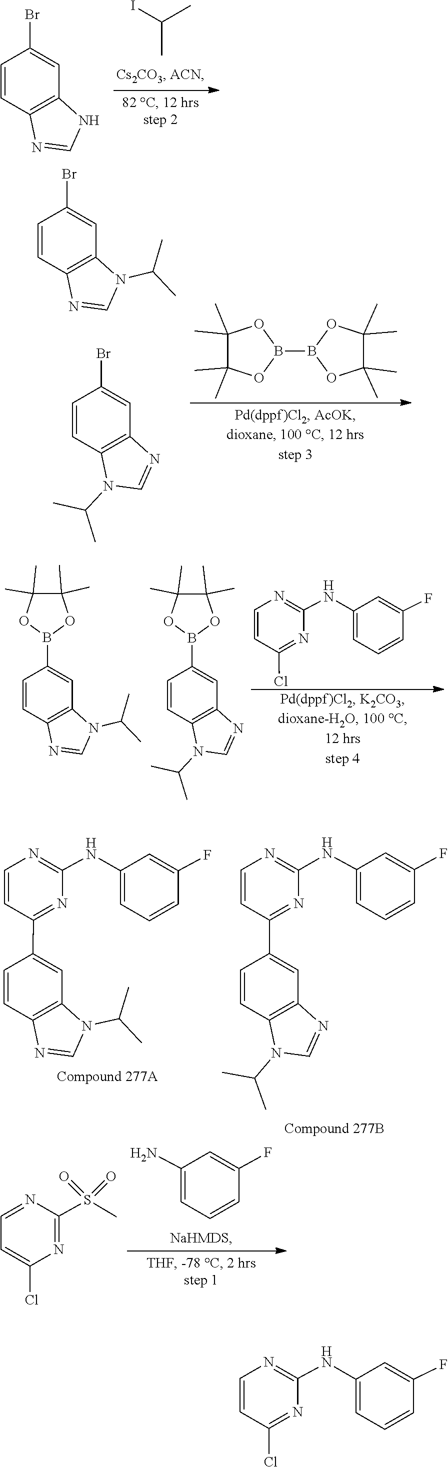

### Step 1—Synthesis of 4-chloro-N-(3-fluorophenyl)pyrimidin-2-amine

A mixture of 3-fluoroaniline (0.5 g, 0.5 mL, 1 Eq, 4 mmol) and THF (10 mL) under N₂ was treated with NaHMDS (0.9 g, 5 mL, 1 molar, 1.1 Eq, 5 mmol). The resulting mixture was stirred for 1 h at −70 °C, and then treated with 4-chloro-2-(methylsulfonyl)pyrimidine (0.9 g, 1 Eq, 4 mmol) in THF (20 mL). The resulting mixture was stirred for an additional 2 hr at −70 °C. The mixture was treated with ammonium chloride solution (20 mL). The resulting mixture was extracted with ethyl acetate, washed with water, and then washed with brine. The organic layer was dried over Na₂SO₄, solid removed by filtration and solvent removed under reduced pressure. The remainder was purified by 10% ethyl acetate in hexane to afford 4-chloro-N-(3-fluorophenyl)pyrimidin-2-amine (543 mg, 2.43 mmol, 50%). ¹H NMR (400 MHz, CDCl₃) δ 8.26 (d, J=5.4 Hz, 1H), 7.60 (dt, J=11.5, 2.2 Hz, 1H), 7.32-7.21 (m, 1H), 7.18 (d, J=6.2 Hz, 1H), 7.11 (d, J=8.0 Hz, 1H), 6.78-6.67 (m, 2H).

### Step 2—Synthesis of mixture of 5-bromo-1-isopropyl-1H-benzo[d]imidazole and 6-bromo-1-isopropyl-1H-benzo[d]imidazole

5-bromo-1H-benzo[d]imidazole (3 g, 1 Eq, 0.02 mol) and cesium carbonate (5 g, 1 Eq, 0.02 mol) were added to a 50 mL round bottom flask. Acetonitrile (15 mL) was added to the flask, followed by the addition of 2-iodopropane (5 g, 3 mL, 1.8 Eq, 0.03 mol). The resulting mixture was heated to reflux at 82 °C under a nitrogen atmosphere overnight. The volatiles were then removed in vacuo and the residual solid was extracted three times with ethyl acetate (3×20 mL). The ethyl acetate solution was then washed three times with water (3×25 mL) and a final time with brine solution (25 mL). The organic layer was then dried over anhydrous magnesium sulfate and then filtered over a medium porosity frit to remove solid and concentrated to dryness under a vacuum to afford a mixture of 5-bromo-1-isopropyl-1H-benzo[d]imidazole and 6-bromo-1-isopropyl-1H-benzo[d]imidazole (3.5 g, 15 mmol, 100%), a tacky light pink solid which was used in the next step without further purification as a mixture of two isomers. ¹H NMR (400 MHz, CDCl₃) δ 8.01-7.91 (m, 2H), 7.65 (d, J=8.6 Hz, 1H), 7.57 (s, 1H), 7.41-7.35 (m, 1H), 7.28 (d, J=8.4 Hz, 1H), 4.58 (dq, J=17.3, 6.8 Hz, 1H), 1.60 (d, J=6.4 Hz, 6H).

### Step 3—Synthesis of mixture of 1-isopropyl-6-(4,4,5,5-tetramethyl-1,3,2-dioxaborolan-2-yl)-1H-benzo[d]imidazole and 1-isopropyl-5-(4,4,5,5-tetramethyl-1,3,2-dioxaborolan-2-yl)-1H-benzo[d]imidazole

To a mixture of potassium acetate (294 mg, 3 Eq, 3.00 mmol), a mixture of 5-bromo-1-isopropyl-1H-benzo[d]imidazole and 6-bromo-1-isopropyl-1H-benzo[d]imidazole (239 mg, 1 Eq, 999 μmol), and B₂pin₂ (279 mg, 1.1 Eq, 1.10 mmol) was added toluene (10 mL) and degassed with N₂ for 10 min. Then was added Pd(dppf)Cl₂ (73.3 mg, 0.1 Eq, 99.9 μmol). And degassed again for 5 min. The reaction mixture was heated at 90 °C for 4 h. Then cooled. then passed via celite and purified by 100% hexane to 100% ethyl acetate to afford a mixture of 1-isopropyl-6-(4,4,5,5-tetramethyl-1,3,2-dioxaborolan-2-yl)-1H-benzo[d]imidazole and 1-isopropyl-5-(4,4,5,5-tetramethyl-1,3,2-dioxaborolan-2-yl)-1H-benzo[d]imidazole (254 mg, 888 μmol, 88.8%). ¹H NMR (400 MHz, CDCl₃) δ 8.25 (s, 1H), 7.98 (d, J=15.2 Hz, 2H), 7.88 (s, 1H), 7.77 (s, 1H), 7.77-7.66 (m, 3H), 7.38 (d, J=8.1 Hz, 1H), 4.64 (dp, J=29.9, 6.8 Hz, 2H), 1.34 (d, J=3.6 Hz, 12H).

### Step 4—Synthesis of N-(3-fluorophenyl)-4-(1-isopropyl-1H-benzo[d]imidazol-6-yl)pyrimidin-2-amine and N-(3-fluorophenyl)-4-(1-isopropyl-1H-benzo[d]imidazol-5-yl)pyrimidin-2-amine (Compound 277A and Compound 277B)

To a mixture of 4-chloro-N-(3-fluorophenyl)pyrimidin-2-amine (109.9 mg, 1.1 Eq, 491.6 μmol), a mixture of 1-isopropyl-6-(4,4,5,5-tetramethyl-1,3,2-dioxaborolan-2-yl)-1H-benzo[d]imidazole and 1-isopropyl-5-(4,4,5,5-tetramethyl-1,3,2-dioxaborolan-2-yl)-1H-benzo[d]imidazole (0.1279 g, 1.0 Eq, 446.9 μmol), and potassium carbonate (185.3 mg, 3 Eq, 1.341 mmol) was added 1,4-dioxane (10 mL) and water (2.0 mL) degassed with N₂. Then was added Pd(dppf)Cl₂ (32.79 mg, 0.1 Eq, 44.69 μmol). The reaction mixture was heated at 90 °C for 4 h. Then cooled. then passed via celite and purified by 100% hexane to 100% ethyl acetate. LCMS did not show any parent ion peak. The crude was dried under reduced pressure and loaded in a column, purified by 10% MeOH in CH₃Cl, and finally, with reverse HPLC using 100% water to 100% MeOH added 0.05% HCOOH to afford a mixture of N-(3-fluorophenyl)-4-(1-isopropyl-1H-benzo[d]imidazol-6-yl)pyrimidin-2-amine and N-(3-fluorophenyl)-4-(1-isopropyl-1H-benzo[d]imidazol-5-yl)pyrimidin-2-amine (Compound 277A and Compound 277B, 1.8 mg, 5.2 μmol, 1.2%) as a 88:12 mixture of isomers. ¹H NMR (400 MHz, CD₃OD) δ 8.58-8.46 (m, 2H), 8.40 (s, 1H), 8.29 (s, 1H), 8.19 (dd, J=8.5, 1.8 Hz, 1H), 7.89-7.73 (m, 2H), 7.51-7.39 (m, 2H), 7.35-7.25 (m, 1H), 6.71 (td, J=8.2, 2.5 Hz, 1H), 1.74-1.63 (m, 6H). MS: m/z 347.8 (M+).

### Example 2—Synthesis of 1,3,5-Triazine-Containing Compounds

### General Synthetic Scheme 2

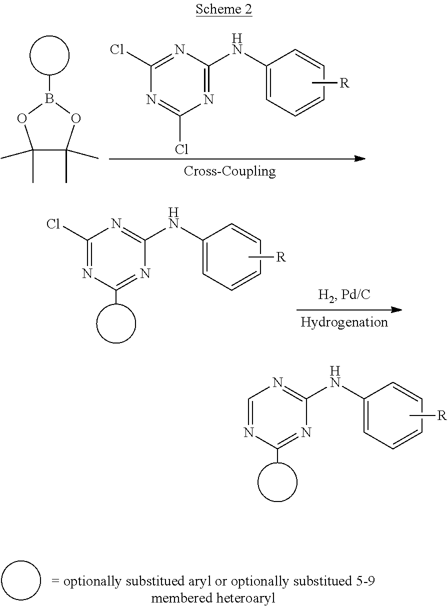

### Synthesis of 4-(1H-Imidazo[4,5-b]pyridin-6-yl)-N-(3-(trifluoromethyl)phenyl)-1,3,5-triazin-2-amine (Compound 105)

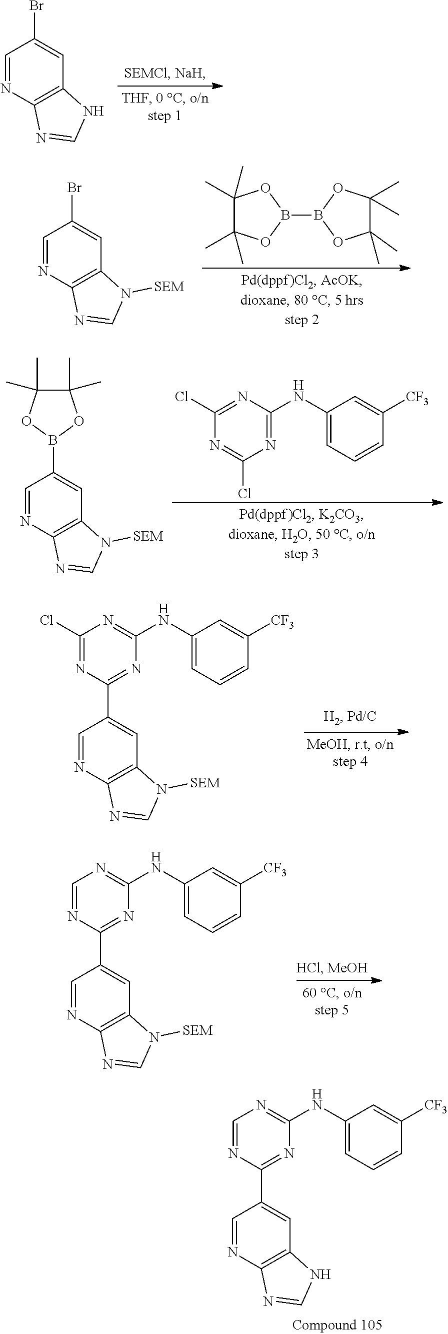

### Step 1—Synthesis of 6-bromo-1-((2-(trimethylsilyl)ethoxy)methyl)-1H-imidazo[4,5-b]pyridine

To a solution of 6-bromo-1H-imidazo[4,5-b]pyridine (1 g, 5.1 mmol) in THF (40 mL) was added SEMCl (1 g, 6.1 mmol) and NaH (122 mg, 5.1 mmol) at 0 °C. The reaction mixture was stirred at room temperature overnight. The mixture was concentrated under reduced pressure and the remainder was purified by silica gel column (DCM/MeOH=30/1) to afford 6-bromo-1-((2-(trimethylsilyl)ethoxy)methyl)-1H-imidazo[4,5-b]pyridine (500 mg, yield: 31%).

### Step 2—Synthesis of 6-(4,4,5,5-tetramethyl-1,3,2-dioxaborolan-2-yl)-1-((2-(trimethylsilyl)ethoxy)methyl)-1H-imidazo[4,5-b]pyridine

A solution of 6-bromo-1-((2-(trimethylsilyl)ethoxy)methyl)-1H-imidazo[4,5-b]pyridine (500 mg, 1.53 mmol), B₂pin₂ (777 mg, 3.06 mmol), Pd(dppf)Cl₂ (110 mg, 0.15 mmol) and AcOK (300 mg, 3.06 mmol) in 1,4-dioxane (40 mL) was stirred at 80 °C overnight under N₂ atmosphere (balloon) and then allowed to cool to rt. The reaction mixture was filtered to remove solids and the filtrate was concentrated under reduced pressure. The remainder was purified by silica gel column (DCM/MeOH=30/1) to afford 6-(4,4,5,5-tetramethyl-1,3,2-dioxaborolan-2-yl)-1-((2-(trimethylsilyl)ethoxy)methyl)-1H-imidazo[4,5-b]pyridine (500 mg, crude).

### Step 3—Synthesis of 4-chloro-N-(3-(trifluoromethyl)phenyl)-6-(1-((2-(trimethylsilyl)ethoxy)methyl)-1H-imidazo[4,5-b]pyridin-6-yl)-1,3,5-triazin-2-amine

A solution of 6-(4,4,5,5-tetramethyl-1,3,2-dioxaborolan-2-yl)-1-((2-(trimethylsilyl)ethoxy)methyl)-1H-imidazo[4,5-b]pyridine (100 mg, 0.27 mmol), 4,6-dichloro-N-(3-(trifluoromethyl)phenyl)-1,3,5-triazin-2-amine (99 mg, 0.32 mmol), Pd(dppf)Cl₂ (23 mg, 0.1 mmol) and K₂CO₃ (75 mg, 0.54 mmol) in 1,4-dioxane/H₂O (16 mL/4 mL) was stirred at 50 °C overnight under N₂ atmosphere (balloon) and then allowed to cool to rt. The mixture was filtered to remove solids and the filtrate was concentrated under reduced pressure. The remainder was purified by silica gel column (DCM/MeOH=30/1). The isolate was triturated with ethyl acetate (10 mL) to afford 4-chloro-N-(3-(trifluoromethyl)phenyl)-6-(1-((2-(trimethylsilyl)ethoxy)methyl)-1H-imidazo[4,5-b]pyridin-6-yl)-1,3,5-triazin-2-amine (20 mg, yield: 14%).

### Step 4—Synthesis of N-(3-(trifluoromethyl)phenyl)-4-(1-((2-(trimethylsilyl)ethoxy)methyl)-1H-imidazo[4,5-b]pyridin-6-yl)-1,3,5-triazin-2-amine

To a solution of 4-chloro-N-(3-(trifluoromethyl)phenyl)-6-(1-((2-(trimethylsilyl)ethoxy)methyl)-1H-imidazo[4,5-b]pyridin-6-yl)-1,3,5-triazin-2-amine (20 mg, 0.04 mmol) in MeOH (10 mL) was added Pd/C (20 mg). The mixture was stirred at room temperature under H₂ atmosphere (balloon) overnight and then the H₂ was replaced with N₂. The reaction mixture was filtered to remove solids and the filtrate was concentrated under reduced pressure to afford N-(3-(trifluoromethyl)phenyl)-4-(1-((2-(trimethylsilyl)ethoxy)methyl)-1H-imidazo[4,5-b]pyridin-6-yl)-1,3,5-triazin-2-amine (17 mg, yield: 90%).

### Step 5—Synthesis of 4-(1H-imidazo[4,5-b]pyridin-6-yl)-N-(3-(trifluoromethyl)phenyl)-1,3,5-triazin-2-amine (Compound 105)

To a solution of N-(3-(trifluoromethyl)phenyl)-4-(1-((2-(trimethylsilyl)ethoxy)methyl)-1H-imidazo[4,5-b]pyridin-6-yl)-1,3,5-triazin-2-amine (17 mg, 0.03 mmol) in MeOH (3 mL) was added conc. HCl (3 mL). The mixture was stirred at room temperature overnight under N₂ atmosphere. The reaction mixture was filtered to remove solids and the filtrate was concentrated under reduced pressure to afford 4-(1H-imidazo[4,5-b]pyridin-6-yl)-N-(3-(trifluoromethyl)phenyl)-1,3,5-triazin-2-amine (Compound 105, 0.6 mg, yield: 6%). ¹H NMR (400 MHz, DMSO-d₆): δ=10.67 (brs, 1H), 9.39 (s, 1H), 8.93 (s, 1H), 8.86 (s, 1H), 8.59 (s, 1H), 8.40 (brs, 1H), 8.04-8.02 (m, 1H), 7.65-7.63 (m, 1H), 7.47-7.45 (m, 1H). MS: m/z 358.0 (M+H⁺).

### Synthesis of 4-(2-methoxypyridin-4-yl)-N-phenyl-1,3,5-triazin-2-amine (Compound 240)

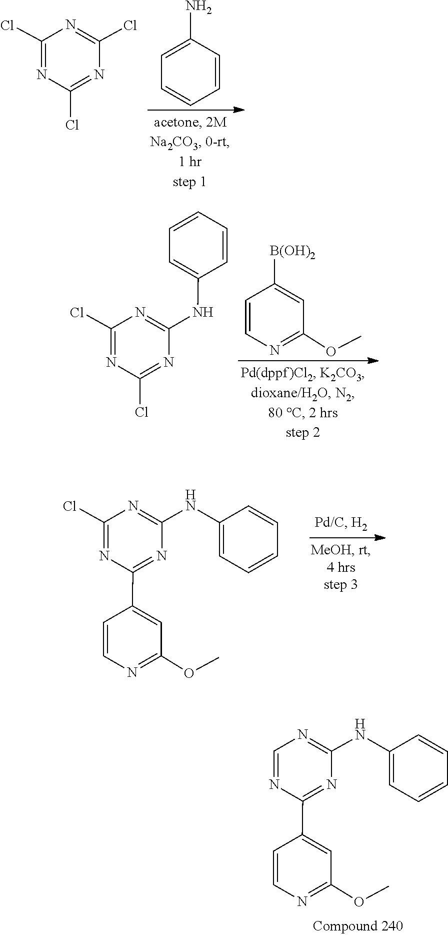

### Step 1—Synthesis of 4,6-dichloro-N-phenyl-1,3,5-triazin-2-amine

To a solution of 2,4,6-trichloro-1,3,5-triazine (0.92 g, 5.0 mmol) in acetone (40 mL) was added a solution of aniline (465 mg, 5.0 mmol) in acetone (5 mL) dropwise at 0 °C. The resulting mixture was stirred at 0 °C for 1 hr. Then the mixture was mixed with Na₂CO₃ (5 mL, 2 M), adjusted to pH=7, and extracted with ethyl acetate (30 mL×2). The combined organic layers were concentrated under reduced pressure to afford 4,6-dichloro-N-phenyl-1,3,5-triazin-2-amine (1.12 g, yield: 93%).

### Step 2—Synthesis of 4-chloro-6-(2-methoxypyridin-4-yl)-N-phenyl-1,3,5-triazin-2-amine

To a solution of 4,6-dichloro-N-phenyl-1,3,5-triazin-2-amine (240 mg, 1.0 mmol) in 1,4-dioxane/H₂O (20 mL/5 mL) were added 2,4-dichloropyrimidine (153 mg, 1.0 mmol), Pd(dppf)Cl₂ (73 mg, 0.1 mmol), and K₂CO₃ (414 g, 3.0 mmol). The mixture was placed under N₂ and stirred at 80 °C for 2 hrs and then allowed to cool to rt. The resulting mixture was filtered to remove solids and the filtrate was concentrated under reduced pressure to give an isolate, which was purified by silica gel column (DCM) to afford 4-chloro-6-(2-methoxypyridin-4-yl)-N-phenyl-1,3,5-triazin-2-amine (167 mg, yield: 53%).

### Step 3—Synthesis of 4-(2-methoxypyridin-4-yl)-N-phenyl-1,3,5-triazin-2-amine (Compound 240)

To a solution of 4-chloro-6-(2-methoxypyridin-4-yl)-N-phenyl-1,3,5-triazin-2-amine (167 mg, 0.53 mmol) in MeOH (20 mL) was added Pd/C (50 mg). The resulting mixture was stirred for 4 hrs under H₂ atmosphere (balloon) and then the H₂ was replaced with N₂. The mixture was filtered to remove solids and the filtrate was concentrated under reduced pressure to give an isolate, which was purified by prep-HPLC to afford 4-(2-methoxypyridin-4-yl)-N-phenyl-1,3,5-triazin-2-amine (Compound 240, 72.2 mg, yield: 48%). ¹H NMR (400 MHz, DMSO-d₆): δ=10.48 (brs, 1H), 8.90 (s, 1H), 8.40 (d, J=5.6 Hz, 1H), 7.81 (d, J=5.2 Hz, 1H), 7.77 (d, J=8.0 Hz, 2H), 7.62 (s, 1H), 7.46-7.34 (m, 2H), 7.13 (t, J=7.6 Hz, 1H), 3.93 (s, 3H). MS: m/z 280.1 (M+H⁺).

### Synthesis of 4-(4-(phenylamino)-1,3,5-triazin-2-yl)pyridin-2-ol (Compound 233)

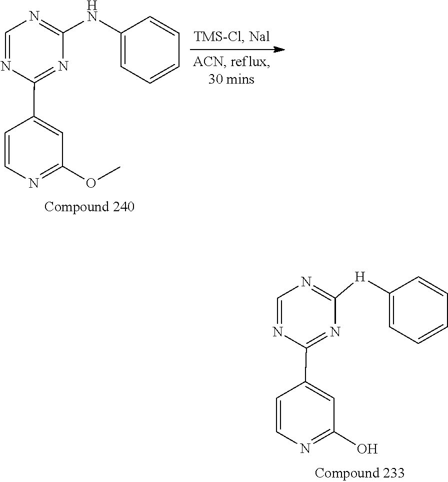

To a solution of 4-(2-methoxypyridin-4-yl)-N-phenyl-1,3,5-triazin-2-amine (Compound 240, 70 mg, 0.25 mmol) in ACN (10 mL) was added TMS-Cl (54 mg, 0.50 mmol), followed by NaI (75 mg, 0.50 mmol). The resulting mixture was stirred to reflux for 30 mins and then allowed to cool to rt. Then the mixture was filtered to remove solids and the filtrate was concentrated under reduced pressure to afford an isolate, which was purified by prep-HPLC to afford 4-(4-(phenylamino)-1,3,5-triazin-2-yl)pyridin-2-ol (Compound 233, 11.6 mg, yield: 17%). ¹H NMR (400 MHz, DMSO-d₆): δ=11.80 (brs, 1H), 10.45 (brs, 1H), 8.88 (s, 1H), 7.75 (d, J=7.6 Hz, 2H), 7.55 (d, J=6.8 Hz, 1H), 7.39 (t, J=7.2 Hz, 2H), 7.27 (s, 1H), 7.12 (t, J=7.2 Hz, 1H), 6.96 (t, J=6.4 Hz, 1H). MS: m/z 266.0 (M+H⁺).

### Example 3—Synthesis of Pyridine-Containing Compounds

### General Synthetic Scheme 3

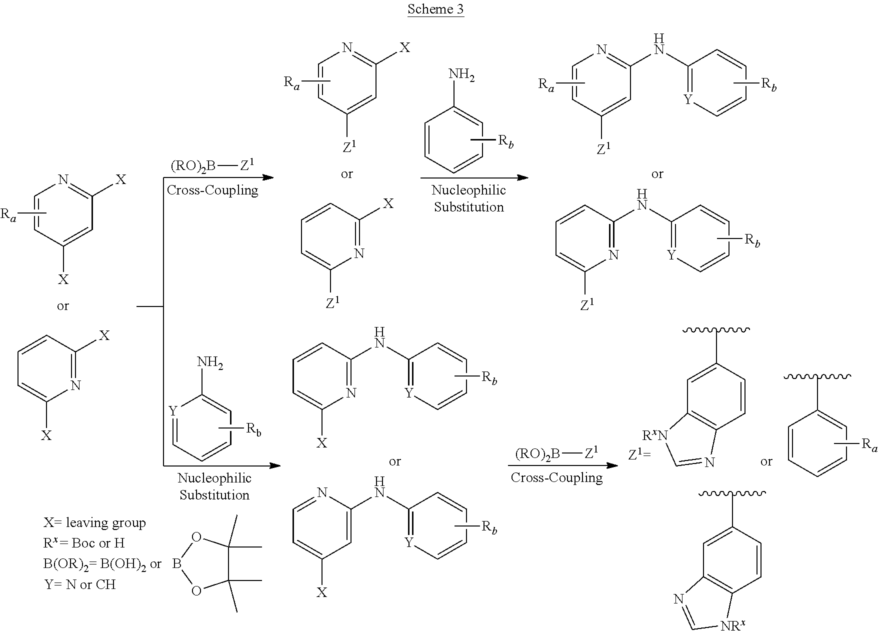

### Synthesis of 6-(1H-benzo[d]imidazol-5-yl)-N-(3-(trifluoromethyl)phenyl)pyridin-2-amine (Compound 50)

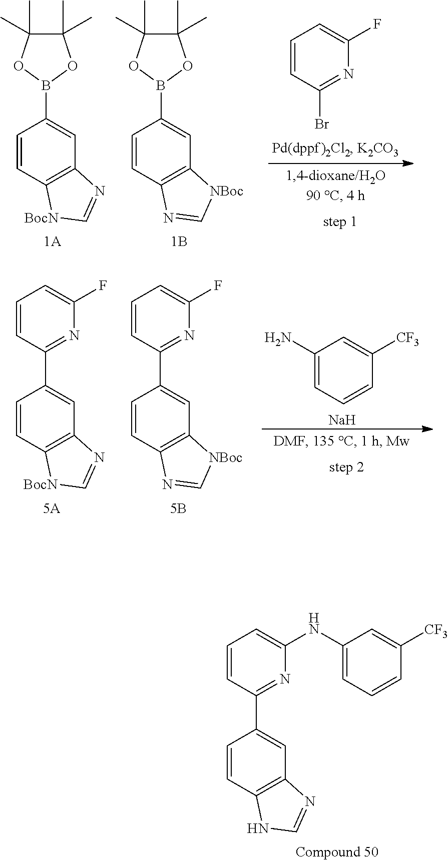

### Step 1—Synthesis of mixture of tert-butyl 6-(6-fluoropyridin-2-yl)-1H-benzo[d]imidazole-1-carboxylate and tert-butyl 6-(6-fluoropyridin-2-yl)-1H-benzo[d]imidazole-1-carboxylate

To a solution of tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 1.38 g, 1 Eq, 4 mmol) in 1,4-dioxane (10 mL) and 2-bromo-6-fluoropyridine (704 mg, 1 Eq, 4 mmol) under nitrogen was added potassium carbonate (1.11 g, 2 Eq, 8 mmol), and water (2.8 mL). The resulting mixture was purged with N₂ for 10 min, and then treated with Pd(dppf)Cl₂ (294 mg, 0.1 Eq, 400 μmol). The resulting mixture was heated at 90 °C for 4 h under nitrogen and then allowed to cool to rt. Subsequently, the mixture was diluted with ethyl acetate (50 mL) and the organic layer separated. The aqueous layer was extracted with ethyl acetate (10×2 mL). The combined organic layer was washed with water (10×2 mL), and brine (20 mL). The organic layer was dried over Na₂SO₄ and filtered to remove solid. The organic layer was removed under reduced pressure and the remainder was purified by silica column chromatography running 100% hexane to 100% ethyl acetate to afford a mixture of tert-butyl 5-(6-fluoropyridin-2-yl)-1H-benzo[d]imidazole-1-carboxylate and tert-butyl 6-(6-fluoropyridin-2-yl)-1H-benzo[d]imidazole-1-carboxylate (5A and 5B, 0.495 g, 1.58 mmol, 39.5%).

### Step 2—Synthesis of 6-(1H-benzo[d]imidazol-5-yl)-N-(3-(trifluoromethyl)phenyl)pyridin-2-amine (Compound 50)

To a solution of a mixture of tert-butyl 5-(6-fluoropyridin-2-yl)-1H-benzo[d]imidazole-1-carboxylate and tert-butyl 6-(6-fluoropyridin-2-yl)-1H-benzo[d]imidazole-1-carboxylate (5A and 5B, 0.11 g, 1 Eq, 0.35 mmol) and 3-(trifluoromethyl) aniline (0.11 g, 88 μL, 2 Eq, 0.70 mmol) in DMF (3 mL) and NaH (42 mg, 2 eq, 1.8 mmol, 60% wet) was heated at 135 °C for 1 h in microwave. The mixture was cooled and diluted with ethyl acetate followed by water. The layers were allowed to partition, and the aqueous layer was extracted with ethyl acetate. The combined ethyl acetate layer was washed with water and then concentrated under reduced pressure to afford an isolate, which was purified by silica column chromatography running 100% hexane to 100% EtOAc, then by the reverse phase HPLC to purify the compound in 20 min run, with 100% water to 100% MeOH eluant to afford 6-(1H-benzo[d]imidazol-5-yl)-N-(3-(trifluoromethyl)phenyl)pyridin-2-amine (Compound 50, 3.5 mg, 9.9 μmol, 2.8%). MS: m/z 356.3 (M+H⁺).

### Synthesis of 4-(1H-benzo[d]imidazol-5-yl)-N-(3-(trifluoromethyl)phenyl)pyridin-2-amine (Compound 52)

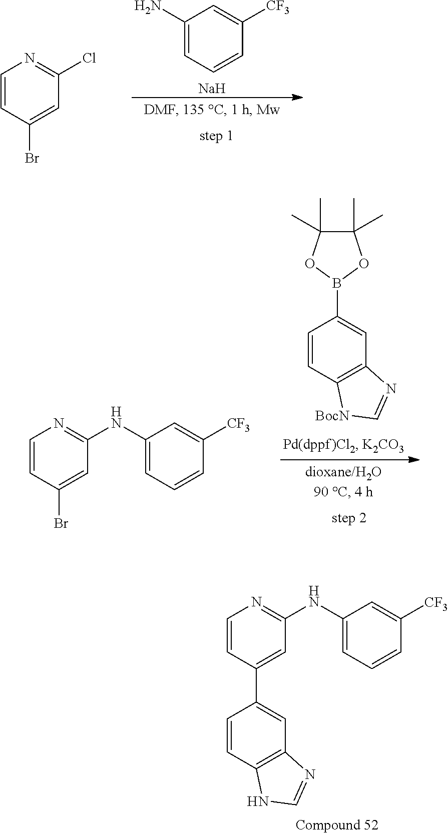

### Step 1—Synthesis of 4-bromo-N-(3-(trifluoromethyl)phenyl)pyridin-2-amine

A solution of 4-bromo-2-fluoropyridine (0.2 g, 1 Eq, 1 mmol) and 3-(trifluoromethyl) aniline (0.4 g, 0.3 mL, 2 Eq, 2 mmol) in DMF (2 mL) was added NaH (50 mg, 2 eq, 2 mmol, 60% wet) heated at 135 °C for 1 h in a microwave. The mixture was allowed to cool to rt, and then diluted with ethyl acetate, followed by water. The aqueous layer was extracted with ethyl acetate, and combined ethyl acetate layer was washed with water and then concentrated under reduced pressure to afford an isolate, which was purified by silica column chromatography running 100% hexane to 100% EtOAc to afford 4-bromo-N-(3-(trifluoromethyl)phenyl)pyridin-2-amine (0.080 g, 0.25 mmol, 20%).

### Step 2—Synthesis of 4-(1H-benzo[d]imidazol-5-yl)-N-(3-(trifluoromethyl)phenyl)pyridin-2-amine (Compound 52)

To a solution of tert-butyl 5-(4,4,5,5-tetramethyl-1,3,2-dioxaborolan-2-yl)-1H-benzo[d]imidazole-1-carboxylate (0.07 g, 1 Eq, 0.2 mmol) and 4-bromo-N-(3-(trifluoromethyl)phenyl)pyridin-2-amine (0.08 g, 1.2 Eq, 0.2 mmol) under nitrogen in 1,4-dioxane (5 mL) was added potassium carbonate (0.06 g, 2 Eq, 0.4 mmol), and water (1.25 mL). The reaction mixture was purged with N₂ for 10 min and then was treated with Pd(dppf)Cl₂ (0.01 g, 0.1 Eq, 0.02 mmol). The resulting mixture was heated at 90 °C for 4 h under nitrogen and then allowed to cool to rt. The reaction mixture was diluted with ethyl acetate (2× 10 mL), washed with water (2×10 mL), and brine (1× 10 mL). The organic layer was dried over anhydrous Na₂SO₄, filtered to remove solids, and concentrated under reduced pressure. The remainder was loaded in a silica column (12 g) to elute in 100% hexane to 100% EtOAc to afford 4-(1H-benzo[d]imidazol-5-yl)-N-(3-(trifluoromethyl)phenyl)pyridin-2-amine (Compound 52, 35 mg, 99 μmol, 50%). ¹H NMR (400 MHz, CDCl₃) δ 8.27 (m, 2H), 7.62 (m, 4H), 7.33-6.75 (m, 3H), 6.68-6.63 (m, 2H). MS: m/z 355.2 (M+H⁺).

### Synthesis of 6-(3-Methoxyphenyl)-N-phenylpyridin-2-amine (Compound 245)

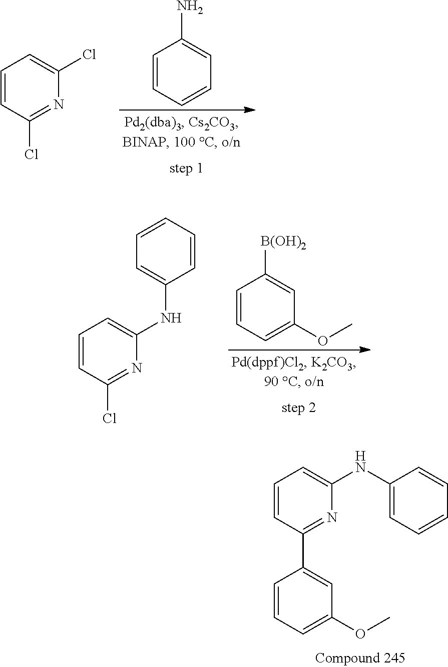 [SMILES: Clc1cccc(Cl)n1 ; Nc1ccccc1 ; Clc1cccc(Nc2ccccc2)n1 ; COc1cccc(B(O)O)c1 ; COc1cccc(-c2cccc(Nc3ccccc3)n2)c1]

### Step 1—Synthesis of 3-chloro-N-phenylaniline

A solution of 2,6-dichloropyridine (500 mg, 3.38 mmol), Pd₂(dba)₃ (194.4 mg, 0.34 mmol), BINAP (210.5 mg, 0.34 mmol), aniline (317.7 mg, 3.38 mmol) and Cs₂CO₃ (2.2 g, 6.7 mmol) in toluene (10 mL) was stirred at 100 °C overnight under N₂ atmosphere (balloon) and then allowed to cool to rt. The reaction mixture was combined with water (10 mL) and extracted with ethyl acetate (10 mL×3). The combined ethyl acetate phases were dried over anhydrous Na₂SO₄, filtered to remove solids and concentrated under reduced pressure. The remainder was purified by silica gel column (PE/EA=1/1) to afford 3-chloro-N-phenylaniline (562 mg, yield: 81.3%).

### Step 2—Synthesis of 6-(3-methoxyphenyl)-N-phenylpyridin-2-amine (Compound 245)

A solution of 3-chloro-N-phenylaniline (562 mg, 2.75 mmol), Pd(dppf)Cl₂ (201.2 mg, 0.28 mmol), (3-methoxyphenyl) boronic acid (501 mg, 3.3 mmol) and K₂CO₃ (759 mg, 5.5 mmol) in 1,4-dioxane/H₂O (10 mL/1 mL) was stirred at 90 °C overnight under N₂ atmosphere (balloon) and then allowed to cool to rt. The reaction mixture was combined with H₂O (10 mL) and extracted with ethyl acetate (10 mL×3). The ethyl acetate phases were dried over anhydrous Na₂SO₄, filtered to remove solids and concentrated under reduced pressure. The remainder was purified by prep-HPLC to afford 6-(3-methoxyphenyl)-N-phenylpyridin-2-amine (Compound 245, 28.2 mg, yield: 3.7%). ¹H NMR (400 MHz, CD₃OD): δ=9.11 (s, 1H), 7.80 (d, J=7.6 Hz, 2H), 7.68-7.61 (m, 3H), 7.41-7.28 (m, 4H), 6.97-6.99 (m, 1H), 6.91 (t, J=7.2 Hz, 1H), 6.80 (d, J=8.0 Hz, 1H), 3.85 (s, 3H). MS: m/z 277.1 (M+H⁺).

### Synthesis of 4-(3-Methoxyphenyl)-N-phenylpyridin-2-amine (Compound 246)

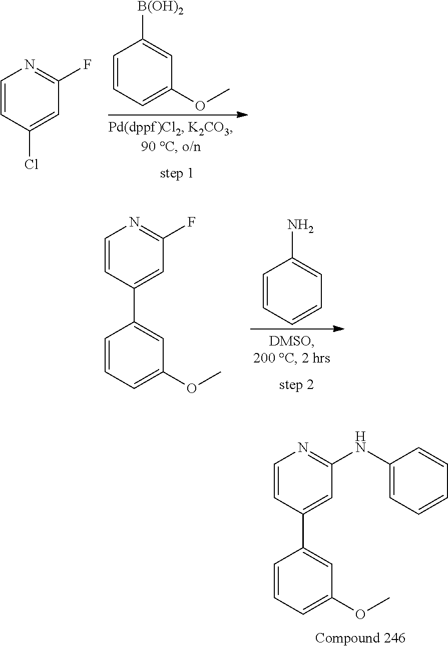

### Step 1—Synthesis of 2-fluoro-4-(3-methoxyphenyl)pyridine

A mixture of 4-chloro-2-fluoropyridine (150 mg, 1.14 mmol), Pd(dppf)Cl₂ (83.4 mg, 0.11 mmol), (3-methoxyphenyl) boronic acid (208 mg, 1.37 mmol) and K₂CO₃ (314.6 mg, 2.28 mmol) in 1,4-dioxane/H₂O (10 mL/1 mL) was stirred at 90 °C overnight under N₂ atmosphere and then allowed to cool to rt. The reaction mixture was combined with H₂O (10 mL) and extracted with ethyl acetate (10 mL×3). The combined ethyl acetate phases were dried over anhydrous Na₂SO₄, filtered to remove solids and concentrated under reduced pressure. The remainder was purified by prep-TLC (PE/EA=4/1) to afford 2-fluoro-4-(3-methoxyphenyl)pyridine (182 mg, yield: 78.8%).

### Step 2—Synthesis of 4-(3-methoxyphenyl)-N-phenylpyridin-2-amine (Compound 246)

A mixture of 2-fluoro-4-(3-methoxyphenyl)pyridine (182 mg, 0.897 mmol) and aniline (100.1 mg, 1.08 mmol) in DMSO (3 mL) was stirred at 200 °C under microwave irradiation for 2 hrs and then allowed to cool to rt. The reaction mixture was combined with H₂O (10 mL) and extracted with ethyl acetate (10 mL×3). The combined ethyl acetate phases were dried over anhydrous Na₂SO₄, filtered to remove solids and concentrated under reduced pressure. The remainder was purified by prep-HPLC to afford 4-(3-methoxyphenyl)-N-phenylpyridin-2-amine (Compound 246, 1.5 mg, yield: 0.92%). ¹H NMR (400 MHz, CD₃OD): δ=8.19 (d, J=5.6 Hz, 1H), 7.58-7.56 (m, 2H), 7.46 (t, J=8.0 Hz, 1H), 7.38-7.34 (m, 2H), 7.31-7.29 (m, 1H), 7.26-7.25 (m, 1H), 7.12 (s, 1H), 7.11-7.03 (m, 3H), 3.93 (s, 3H). MS: m/z 277.1 (M+H⁺).

### Synthesis of N-(4-(1H-benzo[d]imidazol-5-yl)pyridin-2-yl)-6-(difluoromethyl)pyridin-2-amine (Compound 278)

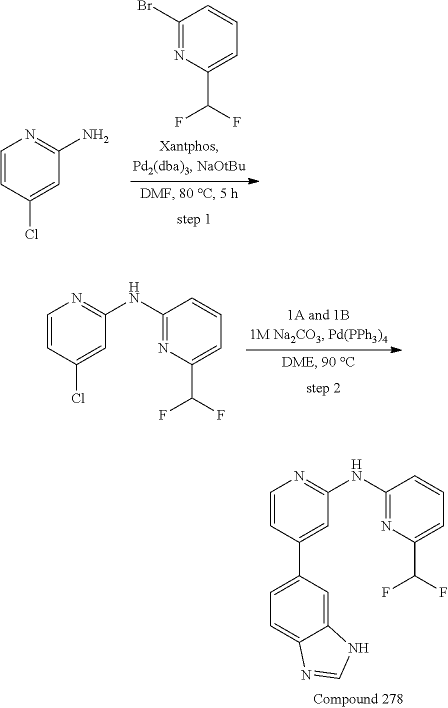 [SMILES: FC(F)c1cccc(Br)n1 ; Nc1cc(Cl)ccn1 ; FC(F)c1cccc(Nc2cc(Cl)ccn2)n1 ; FC(F)c1cccc(Nc2cc(-c3ccc4nc[nH]c4c3)ccn2)n1]

### Step 1—Synthesis of 4-chloro-N-(6-(difluoromethyl)pyridin-2-yl)pyridin-2-amine

To a mixture of NaOtBu (748 mg, 10 Eq, 7.78 mmol) 4-chloropyridin-2-amine (100 mg, 1 Eq, 778 μmol), and 2-bromo-6-(difluoromethyl)pyridine (324 mg, 2 Eq, 1.56 mmol) was added DMF (5 mL) and Xantphos (45.0 mg, 0.1 Eq, 77.8 μmol) degassed with N₂ for 3 min. Then was added Pd₂(dba)₃ (35.6 mg, 0.05 Eq, 38.9 μmol). Degassed again for 2 min. Capped. The reaction mixture was heated at 80 °C for 60 min in a microwave. The crude was passed via celite and dried under reduced pressure. Then purified by 100% hexane to 100% ethyl acetate to furnish 4-chloro-N-(6-(difluoromethyl)pyridin-2-yl)pyridin-2-amine (0.0313 g, 122 μmol, 15.7%).

### Step 2—Synthesis of N-(4-(1H-benzo[d]imidazol-5-yl)pyridin-2-yl)-6-(difluoromethyl)pyridin-2-amine

A mixture of sodium carbonate (0.3 mL, 1 molar, 3 Eq, 0.3 mmol) tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 0.03 g, 1.0 Eq, 0.09 mmol), and 4-bromo-N-(6-(difluoromethyl)pyridin-2-yl)pyridin-2-amine (0.03 g, 1.1 Eq, 0.1 mmol) was treated with DME (4 mL) and sodium carbonate (0.3 mL, 1 molar, 3 Eq, 0.3 mmol) degassed with N₂. The resulting mixture was treated with Pd(PPh₃)₄ (5 mg, 0.05 Eq, 4 μmol). The resulting mixture was heated at 100 °C for 12 h. The mixture was concentrated and the remainder was purified using silica gel chromatography with 10% methanol in DCM to afford N-(4-(1H-benzo[d]imidazol-5-yl)pyridin-2-yl)-6-(difluoromethyl)pyridin-2-amine (Compound 278, 6.8 mg, 20 μmol, 20%). ¹H NMR (400 MHz, CD₃OD) δ 8.25 (d, J=5.7 Hz, 3H), 7.99 (s, 1H), 7.83-7.65 (m, 3H), 7.62 (d, J=8.6 Hz, 2H), 7.28 (d, J=5.9 Hz, 2H), 7.15 (d, J=7.2 Hz, 1H). MS: m/z 338 (M+H).

### Synthesis of N-(4-(1H-benzo[d]imidazol-6-yl)pyridin-2-yl)-6-(trifluoromethyl)pyridin-2-amine (Compound 281)

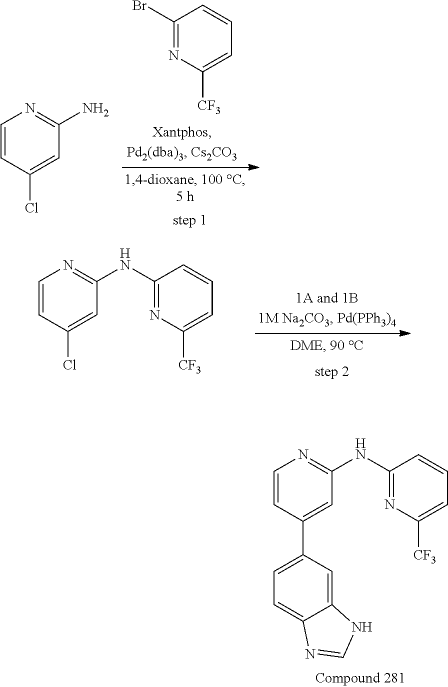 [SMILES: FC(F)(F)c1cccc(Br)n1 ; Nc1cc(Cl)ccn1 ; FC(F)(F)c1cccc(Nc2cc(Cl)ccn2)n1 ; FC(F)(F)c1cccc(Nc2cc(-c3ccc4nc[nH]c4c3)ccn2)n1]

### Step 1—Synthesis of 4-chloro-N-(6-(trifluoromethyl)pyridin-2-yl)pyridin-2-amine

A mixture of 4-chloropyridin-2-amine (0.85 g, 1.5 Eq, 6.6 mmol), 2-bromo-6-(trifluoromethyl)pyridine (2.0 g, 50% Wt, 1 Eq, 4.4 mmol), Cs₂CO₃ (2.9 g, 2 Eq, 8.8 mmol), and Xantphos (0.31 g, 0.12 Eq, 0.53 mmol) was treated with 1,4-dioxane (15 mL). The resulting mixture was purged with N₂ and treated with added Pd₂(dba)₃ (0.24 g, 0.06 Eq, 0.27 mmol). The vessel was then purged with N₂ for 10 min and then capped. The mixture was heated at 100 °C for 5 h. The mixture was passed via celite, diluted with ethyl acetate, and the dilutant washed with water and brine, and dried over Na₂SO₄. The solid was removed by filtration and the solvent was removed under reduced pressure. The remainder was purified using silica gel chromatography with 100% hexane to 100% ethyl acetate to afford 4-chloro-N-(6-(trifluoromethyl)pyridin-2-yl)pyridin-2-amine (1.1 g, 4.0 mmol, 91%). ¹H NMR (400 MHz, DMSO-d₆) δ 10.41 (s, 1H), 8.25 (d, J=5.3 Hz, 1H), 8.03-7.92 (m, 2H), 7.89 (s, 1H), 7.39 (d, J=6.8 Hz, 1H), 7.07 (d, J=5.6 Hz, 1H). MS: m/z 274 (M+H).

### Step 2—Synthesis of N-(4-(1H-benzo[d]imidazol-6-yl)pyridin-2-yl)-6-(trifluoromethyl)pyridin-2-amine

A mixture of sodium carbonate (5.13 mL, 1 molar, 3 Eq, 5.13 mmol), tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 589 mg, 1 Eq, 1.71 mmol), and 4-chloro-N-(6-(trifluoromethyl)pyridin-2-yl)pyridin-2-amine (515 mg, 1.1 Eq, 1.88 mmol) was treated with DME (15 mL) and sodium carbonate (5.13 mL, 1 molar, 3 Eq, 5.13 mmol) and degassed with N₂ for 3 min. The resulting mixture was treated with Pd(PPh₃)₄ (198 mg, 0.1 Eq, 171 μmol). The mixture was heated at 100 °C for 12 h. The mixture was concentrated and the remainder was purified using silica gel chromatography with ethyl acetate-hexane to afford a material which was then repurified with reverse phase HPLC using 100% water to 100% methanol (0.05% formic acid) to afford N-(4-(1H-benzo[d]imidazol-6-yl)pyridin-2-yl)-6-(trifluoromethyl)pyridin-2-amine (Compound 281, 166 mg, 468 μmol, 27%). ¹H NMR (400 MHz, CD₃OD) δ 6.71-6.62 (m, 3H), 6.50 (s, 1H), 6.37 (s, 1H), 6.21 (t, J=8.0 Hz, 1H), 6.07 (dd, J=12.5, 7.1 Hz, 3H), 5.66 (dd, J=20.2, 6.6 Hz, 2H). MS: m/z 356.1 (M+H).

### Synthesis of 4-(1H-benzo[d]imidazol-6-yl)-5-fluoro-N-(6-(trifluoromethyl)pyridin-2-yl)pyridin-2-amine (Compound 279)

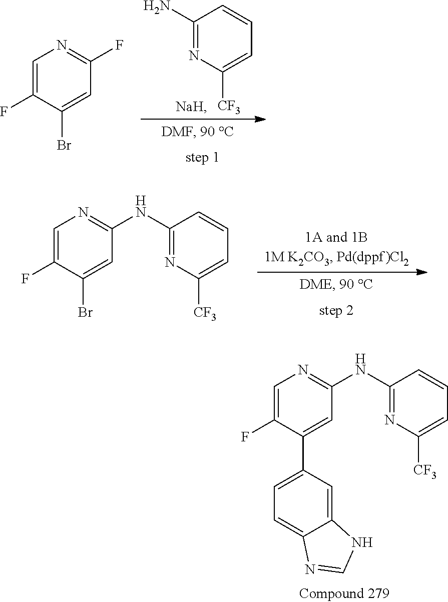 [SMILES: Fc1cc(Br)c(F)cn1 ; Nc1cccc(C(F)(F)F)n1 ; Fc1cnc(Nc2cccc(C(F)(F)F)n2)cc1Br ; Fc1cnc(Nc2cccc(C(F)(F)F)n2)cc1-c1ccc2nc[nH]c2c1]

### Step 1—Synthesis of 4-bromo-5-fluoro-N-(6-(trifluoromethyl)pyridin-2-yl)pyridin-2-amine

To a mixture of 6-(trifluoromethyl)pyridin-2-amine (350 mg, 1.76 Eq, 2.16 mmol), 4-bromo-2,5-difluoropyridine (0.238 g, 1 Eq, 1.23 mmol), and sodium hydride (98 mg, 60% Wt, 2 Eq, 2.45 mmol) was added DMF (10 mL). The reaction mixture was heated at 90 °C for 12 hr. The crude was removed under reduced pressure, then diluted with ethyl acetate and brine, and finally dried over Na₂SO₄ to afford the title compound 4-bromo-5-fluoro-N-(6-(trifluoromethyl)pyridin-2-yl)pyridin-2-amine (67 mg, 0.20 mmol, 16%). ¹H NMR (400 MHz, CDCl₃) δ 12.03 (s, 1H), 8.48 (s, 1H), 8.12 (s, 2H), 7.75 (d, J=12.0 Hz, 1H), 7.50 (s, 1H).

### Step 2—Synthesis of 4-(1H-benzo[d]imidazol-6-yl)-5-fluoro-N-(6-(trifluoromethyl)pyridin-2-yl)pyridin-2-amine

A mixture of 4-bromo-5-fluoro-N-(6-(trifluoromethyl)pyridin-2-yl)pyridin-2-amine (0.09 g, 1.1 Eq, 0.3 mmol) tert-butyl 6-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (1A and 1B, 0.08 g, 1.0 Eq, 0.2 mmol), and potassium carbonate (0.1 g, 3 Eq, 0.7 mmol) was treated with 1,4-dioxane (10 mL) and water (2.0 mL) degassed with N₂. The resulting mixture was treated with Pd(dppf)Cl₂ (0.02 g, 0.1 Eq, 0.02 mmol). The reaction mixture was heated at 90 °C for 4 h and then allowed to cool to rt. Then the solvent was removed under reduced pressure and the crude was loaded in a reverse phase HPLC as a DMF solution and purified using 10% MeOH to 90% MeOH in water with 0.05% formic acid to afford 4-(1H-benzo[d]imidazol-6-yl)-5-fluoro-N-(6-(trifluoromethyl)pyridin-2-yl)pyridin-2-amine (Compound 279, 9.9 mg, 27 μmol, 10%). ¹H NMR (400 MHz, CD₃OD) δ 8.32-8.24 (m, 2H), 8.19 (s, 1H), 7.96 (s, 1H), 7.84-7.70 (m, 2H), 7.61 (t, J=7.8 Hz, 2H), 7.22 (d, J=7.4 Hz, 1H). MS: m/z 373.6 (M+).

### Synthesis of 4-(1-cyclopropyl-4-fluoro-2-methyl-1H-benzo[d]imidazol-6-yl)-N-(6-(trifluoromethyl)pyridin-2-yl)pyridin-2-amine (Compound 282)

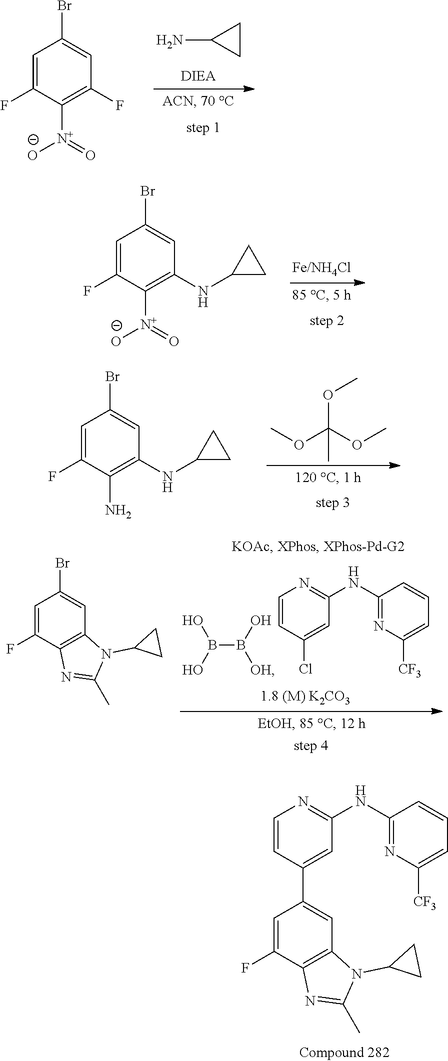

### Step 1—Synthesis of 5-bromo-N-cyclopropyl-3-fluoro-2-nitroaniline

To a solution of 5-bromo-1,3-difluoro-2-nitrobenzene (2 g, 1 Eq, 8 mmol) in ACN (15 mL) was added cyclopropanamine (0.5 g, 1 Eq, 8 mmol). The reaction was heated at 50 °C for 2 h to afford 5-bromo-N-cyclopropyl-3-fluoro-2-nitroaniline (2.2 g, 8.0 mmol, 100%) as the major product. The reaction mixture was dried under reduced pressure and loaded on a silica column, and purified with 100% hexane to 100% ethyl acetate to furnish 5-bromo-N-cyclopropyl-3-fluoro-2-nitroaniline (2.2 g, 8.0 mmol, 100%).

### Step 2—Synthesis of 5-bromo-N¹-cyclopropyl-3-fluorobenzene-1,2-diamine

To a solution of 5-bromo-N-cyclopropyl-3-fluoro-2-nitroaniline (2.21 g, 1 Eq, 8.03 mmol) in EtOH/water (10 mL/2 mL) was added ammonium chloride (2.15 g, 5 Eq, 40.2 mmol) and iron (2.24 g, 5 Eq, 40.2 mmol). The reaction was heated for 12 h at 80 °C and cooled. The reaction mixture was passed through a celite pad, then extracted with ethyl acetate and washed with water. The combined organic layer was washed with brine and dried over Na₂SO₄, and dried under reduced pressure. The organic layer was purified via column chromatography with 10% MeOH in DCM to afford the final compound 5-bromo-N¹-cyclopropyl-3-fluorobenzene-1,2-diamine (1.1 g, 4.5 mmol, 56%).

### Step 3—Synthesis of 6-bromo-1-cyclopropyl-4-fluoro-2-methyl-1H-benzo[d]imidazole

1,1,1-trimethoxymethane (0.54 g, 10 mL, 1 Eq, 4.5 mmol) was added to 5-bromo-N1-cyclopropyl-3-fluorobenzene-1,2-diamine (1.1 g, 1 Eq, 4.5 mmol) and heated for 2 h at 100 °C. The reaction was cooled and dried under reduced pressure. The crude was purified via column chromatography with a gradient of 100% hexane to 100% ethyl acetate to afford the product 6-bromo-1-cyclopropyl-4-fluoro-2-methyl-1H-benzo[d]imidazole (66 mg, 0.25 mmol, 5.5%).

### Step 4—Synthesis of 4-(1-cyclopropyl-4-fluoro-2-methyl-1H-benzo[d]imidazol-6-yl)-N-(6-(trifluoromethyl)pyridin-2-yl)pyridin-2-amine

To a solution of potassium acetate (71 mg, 3 Eq, 0.72 mmol) in EtOH (2 mL) was added hypodiboric acid (65 mg, 3 Eq, 0.72 mmol), 6-bromo-1-cyclopropyl-4-fluoro-2-methyl-1H-benzo[d]imidazole (65 mg, 1 Eq, 0.24 mmol), XPhos (3.5 mg, 0.03 Eq, 7.2 μmol), and XPhos-Pd_G2 (5.7 mg, 0.03 Eq, 7.2 μmol). The reaction mixture was degassed for 4 minutes and heated at 85 °C until the color turned orange. The reaction mixture was cooled, and 4-chloro-N-(6-(trifluoromethyl)pyridin-2-yl)pyridin-2-amine (73 mg, 1.1 Eq, 0.27 mmol) and K₂CO₃ (0.10 g, 0.40 mL, 1.8 molar, 3 Eq, 0.72 mmol) were added under nitrogen. The reaction mixture was heated at 85 °C overnight. The reaction mixture was cooled and passed through celite, then dried under reduced pressure. The crude was loaded in a reverse phase column to elute with 10% MeOH to 100% MeOH to afford 4-(1-cyclopropyl-4-fluoro-2-methyl-1H-benzo[d]imidazol-6-yl)-N-(6-(trifluoromethyl)pyridin-2-yl)pyridin-2-amine (Compound 282, 12.7 mg, 29.7 μmol, 12%). ¹H NMR (400 MHz, CD₃OD) δ 7.64 (s, 1H), 7.44 (s, 1H), 7.15-6.86 (m, 2H), 6.75 (d, J=8.3 Hz, 1H), 6.62-6.31 (m, 3H), 1.87 (s, 3H), 0.50 (d, J=6.7 Hz, 2H), 0.31 (d, J=3.5 Hz, 2H). M+H=429.2

### Synthesis of 4-(1-cyclopropyl-2-methyl-1H-benzo[d]imidazol-6-yl)-N-(6-(trifluoromethyl)pyridin-2-yl)pyridin-2-amine (Compound 284)

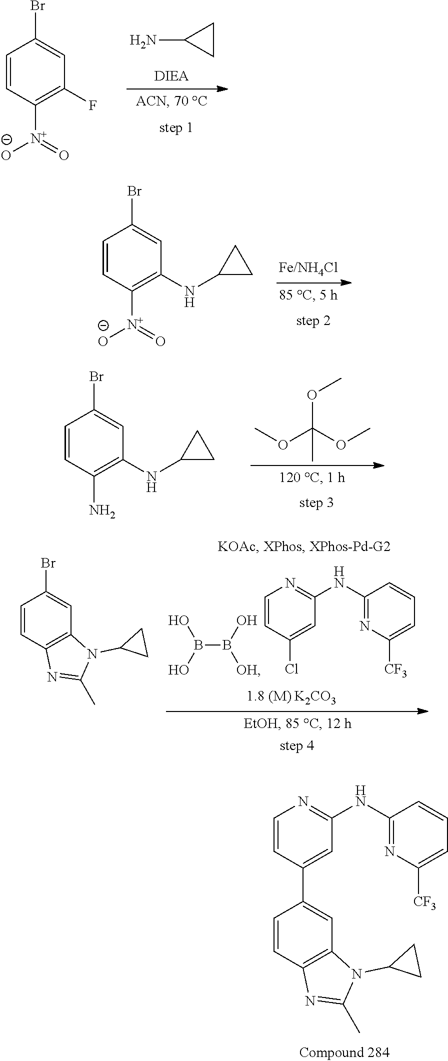

### Step 1—Synthesis of 5-bromo-N-cyclopropyl-2-nitroaniline

To a solution of 4-bromo-2-fluoro-1-nitrobenzene (2 g, 1 Eq, 9 mmol) in ACN (10 mL) was added cyclopropanamine (2 g, 4 Eq, 0.04 mol) and DIEA (1 g, 2 mL, 1 Eq, 9 mmol). The reaction mixture was heated at 80 °C for 2 h, dried under reduced pressure, and purified via column chromatography with a gradient 20% DCM in methanol to afford 5-bromo-N-cyclopropyl-2-nitroaniline (2 g, 8 mmol, 90%).

### Step 2—Synthesis of 5-bromo-N¹-cyclopropylbenzene-1,2-diamine

To a solution of 5-bromo-N-cyclopropyl-2-nitroaniline (2 g, 1 Eq, 8 mmol) in ethanol/water (15 mL/3 mL) was added ammonium chloride (2 g, 5 Eq, 0.04 mol) and iron (2 g, 5 Eq, 0.04 mol). The reaction was heated at 85 °C for 2 h. The reaction was passed through celite and extracted with DCM and water. The organic layer was washed with water, dried over Na₂SO₄, and concentrated under reduced pressure to afford 5-bromo-N¹-cyclopropylbenzene-1,2-diamine (2 g, 9 mmol, 100%).

### Step 3—Synthesis of 6-bromo-1-cyclopropyl-2-methyl-1H-benzo[d]imidazole

5-bromo-N1-cyclopropylbenzene-1,2-diamine (355 mg, 1 Eq, 1.56 mmol) and 1,1,1-triethoxyethane (254 mg, 6 mL, 1 Eq, 1.56 mmol) were mixed and heated at 110 °C for 2 h. The crude was dried under reduced pressure and loaded in a silica column. The crude was purified with 100% hexane to 100% ethyl acetate to afford 6-bromo-1-cyclopropyl-2-methyl-1H-benzo[d]imidazole (292 mg, 1.16 mmol, 74.4%).

### Step 4—Synthesis of 4-(1-cyclopropyl-2-methyl-1H-benzo[d]imidazol-6-yl)-N-(6-(trifluoromethyl)pyridin-2-yl)pyridin-2-amine

To a solution of potassium acetate (151 mg, 3 Eq, 1.54 mmol) in EtOH (2 mL) was added hypodiboric acid (138 mg, 3 Eq, 1.54 mmol), 6-bromo-1-cyclopropyl-2-methyl-1H-benzo[d]imidazole (129 mg, 1 Eq, 514 μmol) XPhos (7 mg, 0.03 Eq, 15 μmol), and XPhos-Pd_G2 (12 mg, 0.03 Eq, 15 μmol). The reaction mixture was degassed for 4 minutes and heated at 85 °C until the color turned orange. The reaction mixture was cooled, and 4-chloro-N-(6-(trifluoromethyl)pyridin-2-yl)pyridin-2-amine (155 mg, 1.1 Eq, 565 μmol) and K₂CO₃ (213 mg, 856 μL, 1.8 molar, 3 Eq, 1.54 mmol) were added under nitrogen. The reaction mixture was heated at 85 °C overnight. The reaction mixture was cooled and passed through celite, then dried under reduced pressure. The crude was loaded in a reverse phase column and purified with 10% MeOH to 100% MeOH to afford the final compound 4-(1-cyclopropyl-2-methyl-1H-benzo[d]imidazol-6-yl)-N-(6-(trifluoromethyl)pyridin-2-yl)pyridin-2-amine (Compound 284, 8.1 mg, 20 μmol, 3.9%). ¹H NMR (400 MHz, DMSO-d₆) δ 10.23 (s, 1H), 8.36-8.25 (m, 3H), 8.03-7.88 (m, 2H), 7.84 (s, 1H), 7.63 (d, J=8.2 Hz, 1H), 7.54 (d, J=8.4 Hz, 1H), 7.34 (d, J=7.6 Hz, 2H), 2.62 (s, 3H), 1.23 (t, J=6.3 Hz, 2H), 1.10 (q, J=3.9 Hz, 2H). M+H=410

### Synthesis of 4-(2-((6-(trifluoromethyl)pyridin-2-yl)amino)pyridin-4-yl)benzamide (Compound 283)

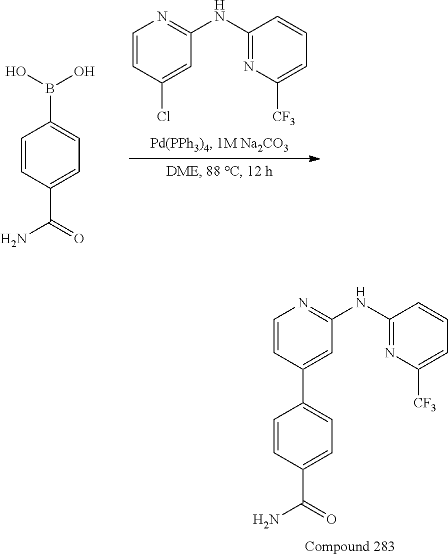 [SMILES: NC(=O)c1ccc(B(O)O)cc1 ; FC(F)(F)c1cccc(Nc2cc(Cl)ccn2)n1 ; NC(=O)c1ccc(-c2ccnc(Nc3cccc(C(F)(F)F)n3)c2)cc1]

To solution of 4-chloro-N-(6-(trifluoromethyl)pyridin-2-yl)pyridin-2-amine (182 mg, 1.1 Eq, 667 μmol) and (4-carbamoylphenyl) boronic acid (100 mg, 1 Eq, 606 μmol) was added Na₂CO₃ (193 mg, 1.82 mL, 1 molar, 3 Eq, 1.82 mmol). The reaction mixture was degassed with N₂ and Pd(PPh₃)₄ (70 mg, 0.1 Eq, 61 μmol) was added followed by DME (10 mL). The reaction mixture was degassed again and heated at 88 °C for 12 h. The reaction was cooled and dried under reduced pressure and purified by prep HPLC to afford 4-(2-((6-(trifluoromethyl)pyridin-2-yl)amino)pyridin-4-yl)benzamide (Compound 283, 92 mg, 0.26 mmol, 42%). ¹H NMR (400 MHz, DMSO-d₆) δ 10.30 (s, 1H), 8.25 (d, J=15.7 Hz, 2H), 8.09 (s, 1H), 8.02 (t, J=9.1 Hz, 3H), 7.94 (t, J=7.9 Hz, 1H), 7.81 (d, J=8.0 Hz, 2H), 7.48 (s, 1H), 7.35 (t, J=6.4 Hz, 2H). M+H=359

### Synthesis of 4-(3-isopropyl-[1,2,4]triazolo[4,3-a]pyridin-6-yl)-N-(6-(trifluoromethyl)pyridin-2-yl)pyridin-2-amine (Compound 289)

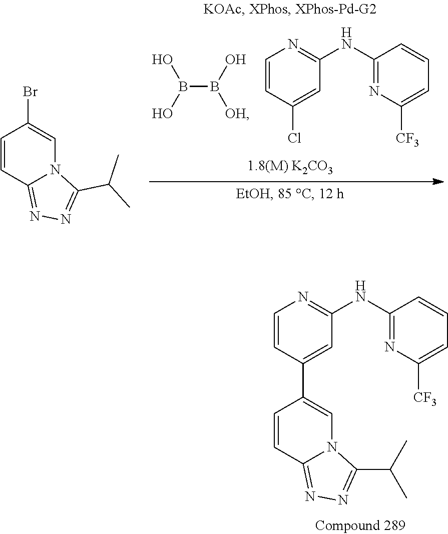

To a pressure tube was added XPhos (5.96 mg, 0.03 Eq, 12.5 μmol), XPhos-Pd-G2 (9.84 mg, 0.03 Eq, 12.5 μmol), 6-bromo-3-isopropyl-[1,2,4]triazolo[4,3-a]pyridine (100 mg, 1 Eq, 416 μmol), hypodiboric acid (112 mg, 3 Eq, 1.25 mmol), potassium acetate (123 mg, 3 Eq, 1.25 mmol), and EtOH (4 mL). The reaction was degassed, sealed, and heated at 85 °C until the color changed from orange to yellow. The reaction mixture was cooled, and 4-chloro-N-(6-(trifluoromethyl)pyridin-2-yl)pyridin-2-amine (125 mg, 1.1 Eq, 458 μmol) and potassium carbonate (173 mg, 694 μL, 1.8 molar, 3 Eq, 1.25 mmol) were added under nitrogen. The reaction was degassed and heated 15 h at 85 °C. The reaction mixture was passed via celite, dried under reduced pressure, loaded in a reverse phase HPLC column, and purified by 10% MeOH to 100% MeOH in a 22 min run to afford the final compound 4-(3-isopropyl-[1,2,4]triazolo[4,3-a]pyridin-6-yl)-N-(6-(trifluoromethyl)pyridin-2-yl)pyridin-2-amine (Compound 289, 29 mg, 73 μmol, 17%). M+H=400.

### Synthesis of 4-(3-isopropyl-2-methyl-2H-indazol-5-yl)-N-(6-(trifluoromethyl)pyridin-2-yl)pyridin-2-amine (Compound 285)

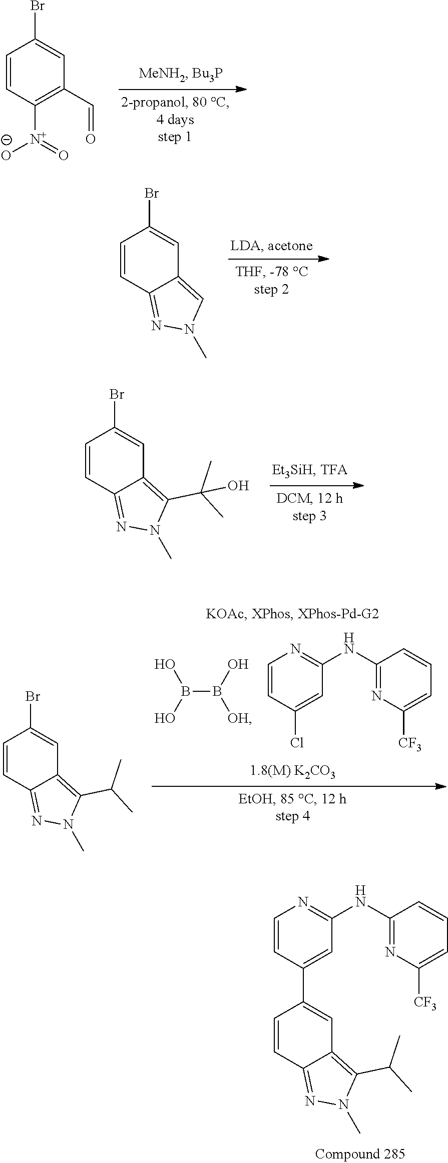

### Step 1—Synthesis of 5-bromo-2-methyl-2H-indazole

To a mixture of 5-bromo-2-nitrobenzaldehyde (564 mg, 1 Eq, 2.45 mmol) and methylamine solution (209 mg, 0.34 mL, 40% Wt in water, 1.1 Eq, 2.70 mmol), was added 2-propanol (10 mL). The reaction mixture was heated at 80 °C for 4 h. Then, the reaction was cooled and tributylphosphane (1.49 g, 1.80 mL, 3 Eq, 7.36 mmol) was added under nitrogen. The reaction was heated to reflux at 80 °C for 4 days. The reaction mixture was dried under reduced pressure and loaded in a column and purified with a gradient of 100% hexane to 100% ethyl acetate to afford the compound 5-bromo-2-methyl-2H-indazole (327 mg, 1.55 mmol, 63.2%).

### Step 2—Synthesis of 2-(5-bromo-2-methyl-2H-indazol-3-yl) propan-2-ol

To solution of 5-bromo-2-methyl-2H-indazole (5 g, 1 Eq, 0.02 mol) in THF (100 mL) was added LDA (4 g, 0.04 L, 1 molar, 1.5 Eq, 0.04 mol) at 0-5 °C. The resulting mixture was stirred for 1 h and then was cooled to −78 °C. The cooled mixture was treated with acetone (2 g, 3 mL, 1.5 Eq, 0.04 mol) and then stirred for 12 h under N₂. The mixture was then treated with water, extracted with ethyl acetate, washed with brine, dried over Na₂SO₄, and filtered to remove solid. The solvent was removed under reduced pressure, and used directly in the next step.

### Step 3—Synthesis of 5-bromo-3-isopropyl-2-methyl-2H-indazole

To a solution of 2-(5-bromo-2-methyl-2H-indazol-3-yl) propan-2-ol (84 mg, 1 Eq, 0.31 mmol) in DCM (6 mL) was added triethylsilane (0.36 g, 0.50 mL, 10 Eq, 3.1 mmol) and trifluoroacetic acid (0.36 g, 0.24 mL, 10 Eq, 3.1 mmol). The reaction mixture was stirred for 12 h at 25 °C. The reaction mixture was diluted with NaHCO₃, extracted with ethyl acetate, and the combined organic layer was dried over Na₂SO₄. The crude was loaded on silica gel and purified with 10% MeOH in DCM to afford the compound 5-bromo-3-isopropyl-2-methyl-2H-indazole (61.4 mg, 243 μmol, 78%).

### Step 4—Synthesis of 4-(3-isopropyl-2-methyl-2H-indazol-5-yl)-N-(6-(trifluoromethyl)pyridin-2-yl)pyridin-2-amine

To a solution of potassium acetate (72 mg, 3 Eq, 0.73 mmol) in EtOH (2 mL) was added hypodiboric acid (66 mg, 3 Eq, 0.73 mmol), 5-bromo-3-isopropyl-2-methyl-2H-indazole (62 mg, 1 Eq, 0.24 mmol), XPhos (3.5 mg, 0.03 Eq, 7.3 μmol), and XPhos-Pd_G2 (5.8 mg, 0.03 Eq, 7.3 μmol). The reaction mixture was degassed for 4 minutes and heated at 85 °C until color turned orange. The reaction mixture was cooled, and 4-chloro-N-(6-(trifluoromethyl)pyridin-2-yl)pyridin-2-amine (74 mg, 1.1 Eq, 0.27 mmol) and K₂CO₃ (0.10 g, 0.41 mL, 1.8 molar, 3 Eq, 0.73 mmol) were added under nitrogen. The reaction mixture was heated at 85 °C o/n. The reaction mixture was cooled and passed through a celite pad, then dried under reduced pressure. The crude was loaded in a reverse phase column and purified with 10% MeOH to 100% MeOH to afford 4-(3-isopropyl-2-methyl-2H-indazol-5-yl)-N-(6-(trifluoromethyl)pyridin-2-yl)pyridin-2-amine (Compound 285, 13 mg, 32 μmol, 13%). M+H=412

### Synthesis of 4-(1-isopropyl-2-methyl-1H-benzo[d]imidazol-6-yl)-N-(6-(trifluoromethyl)pyridin-2-yl)pyridin-2-amine (Compound 291)

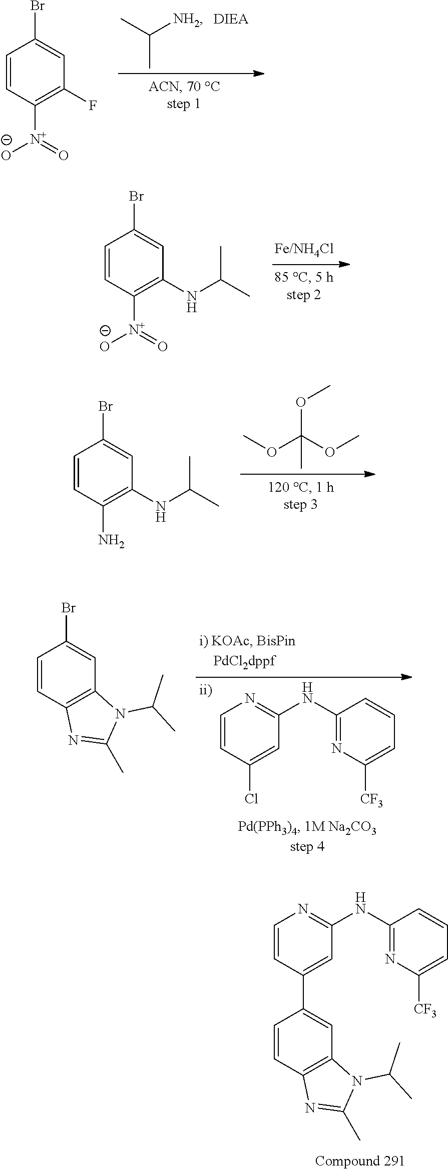

### Step 1—Synthesis of 5-bromo-N-isopropyl-2-nitroaniline

To a mixture of 4-bromo-2-fluoro-1-nitrobenzene (2 g, 1 Eq, 9 mmol), propan-2-amine (2 g, 3 mL, 4 Eq, 0.04 mol), and DIPEA (5 g, 6 mL, 4 Eq, 0.04 mol) was added ACN (10 mL). The reaction mixture was heated at 50 °C for 2 h. The crude was dried under reduced pressure, loaded in a column, and purified by 100% hexane to 100% ethyl acetate to furnish the product 5-bromo-N-isopropyl-2-nitroaniline (2 g, 8 mmol, 80%).

### Step 2—Synthesis of 5-bromo-N1-isopropylbenzene-1,2-diamine

To a mixture of 5-bromo-N-isopropyl-2-nitroaniline (4 g, 1 Eq, 0.02 mol) dissolved in ethanol (20 mL), iron (3 g, 4 Eq, 0.06 mol), and ammonium chloride (3 g, 4 Eq, 0.06 mol), was added water (4 mL). The reaction mixture was heated at 80 °C for 3 h. The solution turned deep brown and was filtered, washed with water, and dried over Na₂SO₄. The crude was passed through a silica column to afford 5-bromo-N1-isopropylbenzene-1,2-diamine (1.75 g, 7.64 mmol, 50%).

### Step 3—Synthesis of 6-bromo-1-isopropyl-2-methyl-1H-benzo[d]imidazole

The mixture of 5-bromo-N1-isopropylbenzene-1,2-diamine (2 g, 1 Eq, 9 mmol) and 1,1,1-triethoxyethane (1 g, 15 mL, 1 Eq, 9 mmol) was heated at 100 °C for 2 h. The reaction mixture was then concentrated under reduced pressure. The crude was purified with 100% hexane to 100% ethyl acetate to furnish the product 6-bromo-1-isopropyl-2-methyl-1H-benzo[d]imidazole (1 g, 4 mmol, 50%).

### Step 4—Synthesis of 4-(1-isopropyl-2-methyl-1H-benzo[d]imidazol-6-yl)-N-(6-(trifluoromethyl)pyridin-2-yl)pyridin-2-amine

To a mixture of sodium carbonate (116 mg, 1.10 mL, 1 molar, 3 Eq, 1.10 mmol), 1-isopropyl-2-methyl-6-(4,4,5,5-tetramethyl-1,3,2-dioxaborolan-2-yl)-1H-benzo[d]imidazole (121 mg, 1.1 Eq, 402 μmol), and 4-chloro-N-(6-(trifluoromethyl)pyridin-2-yl)pyridin-2-amine (100 mg, 1.0 Eq, 365 μmol) was added degassed DME (10 mL) and sodium carbonate (116 mg, 1.10 mL, 1 molar, 3 Eq, 1.10 mmol) followed by Pd(PPh₃)₄ (21 mg, 0.05 Eq, 18 μmol). The reaction mixture was heated at 100 °C for 12 h. The reaction mixture was dried under reduced pressure and loaded with reverse HPLC to afford 4-(1-isopropyl-2-methyl-1H-benzo[d]imidazol-6-yl)-N-(6-(trifluoromethyl)pyridin-2-yl)pyridin-2-amine (Compound 291, 22.3 mg, 54.2 μmol, 14.8%). ¹H NMR (400 MHz, DMSO-d₆) δ 10.20 (s, 1H), 8.32 (d, J=5.4 Hz, 1H), 8.25 (d, J=4.9 Hz, 2H), 8.00 (d, J=8.6 Hz, 1H), 7.96-7.88 (m, 2H), 7.64 (d, J=8.5 Hz, 1H), 7.53 (d, J=8.6 Hz, 1H), 7.36 (dd, J=9.8, 6.3 Hz, 2H), 4.82 (p, J=7.0 Hz, 1H), 2.60 (s, 3H), 1.62 (d, J=6.8 Hz, 6H). M+H=412

### Synthesis of 5-(2-((6-(trifluoromethyl)pyridin-2-yl)amino)pyridin-4-yl)isoindolin-1-one (Compound 287)

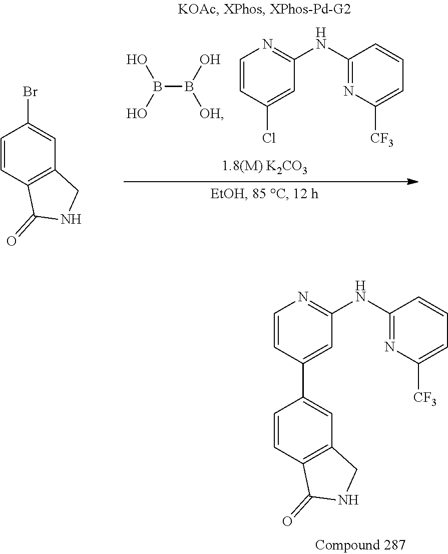

To a solution of potassium acetate (139 mg, 3 Eq, 1.41 mmol) in EtOH (4 mL) was added hypodiboric acid (127 mg, 3 Eq, 1.41 mmol), 4-chloro-N-(6-(trifluoromethyl)pyridin-2-yl)pyridin-2-amine (142 mg, 1.1 Eq, 519 μmol), XPhos (7 mg, 0.03 Eq, 14.1 μmol), and XPhos-Pd_G2 (11.2 mg, 0.03 Eq, 14.1 μmol). The reaction was degassed for 4 minutes and heated at 85 °C until the color turned orange. The reaction mixture was cooled, and 4-chloro-N-(6-(trifluoromethyl)pyridin-2-yl)pyridin-2-amine (142 mg, 1.1 Eq, 519 μmol) and K₂CO₃ (196 mg, 786 μL, 1.8 molar, 3 Eq, 1.41 mmol) were added under nitrogen. The reaction was heated at 85 °C o/n. The reaction mixture was cooled, passed through celite, and dried under reduced pressure. The crude was loaded in a reverse phase column, and purified with 10% MeOH to 100% MeOH to afford 5-(2-((6-(trifluoromethyl)pyridin-2-yl)amino)pyridin-4-yl)isoindolin-1-one (Compound 287, 1.3 mg, 3.5 μmol, 0.74%). M+H=371

### Synthesis of 4-(2-methyl-1-phenyl-1H-benzo[d]imidazol-5-yl)-N-(6-(trifluoromethyl)pyridin-2-yl)pyridin-2-amine (Compound 288)

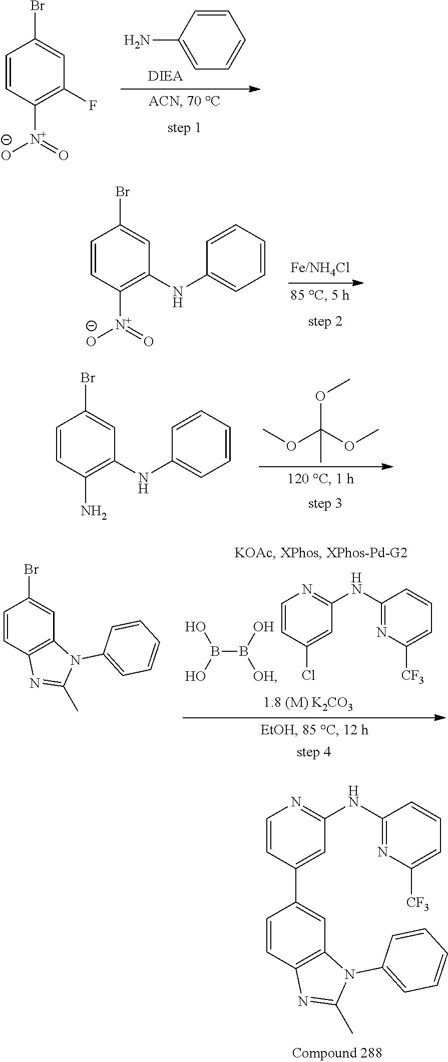

### Step 1—Synthesis of 5-bromo-2-nitro-N-phenylaniline

To a mixture of 4-bromo-2-fluoro-1-nitrobenzene (2 g, 1 Eq, 9 mmol), aniline (3 g, 2 mL, 4 Eq, 0.04 mol) and DIPEA (5 g, 6 mL, 4 Eq, 0.04 mol) was added ACN (10 mL). The reaction mixture was heated at 50 °C for 2 h. The crude was dried under reduced pressure, loaded in a column, and purified by 100% hexane to 100% ethyl acetate to furnish 5-bromo-2-nitro-N-phenylaniline (2 g, 7 mmol, 80%).

### Step 2—Synthesis of 5-bromo-N1-phenylbenzene-1,2-diamine

To a mixture of 5-bromo-2-nitro-N-phenylaniline (3 g, 1 Eq, 0.01 mol), iron (3 g, 5 Eq, 0.05 mol), and ammonium chloride (3 g, 5 Eq, 0.05 mol) was added EtOH (30 mL), and water (12 mL). The reaction mixture was heated at 80 °C for 12 h. The crude was dried under reduced pressure, loaded in a column, and purified by 100% hexane to 100% ethyl acetate to furnish the product 5-bromo-N1-phenylbenzene-1,2-diamine (2.5 g, 9.5 mmol, 90%).

### Step 3—Synthesis of 6-bromo-2-methyl-1-phenyl-1H-benzo[d]imidazole

The mixture of 5-bromo-N1-phenylbenzene-1,2-diamine (2.5 g, 1 Eq, 9.5 mmol) and 1,1,1-triethoxyethane (1.5 g, 15 mL, 1 Eq, 9.5 mmol) heated at 100 °C for 2 h. The reaction mixture was then concentrated under reduced pressure. The crude was loaded in a column and purified with 100% hexane to 100% ethyl acetate to furnish the product 6-bromo-2-methyl-1-phenyl-1H-benzo[d]imidazole (126 mg, 439 μmol, 4.6%).

### Step 4—Synthesis of 4-(2-methyl-1-phenyl-1H-benzo[d]imidazol-5-yl)-N-(6-(trifluoromethyl)pyridin-2-yl)pyridin-2-amine

To a mixture of potassium acetate (175 mg, 3 Eq, 1.79 mmol), Pd(dppf)Cl₂ (43.6 mg, 0.1 Eq, 59.5 μmol), and 5-bromo-2-methyl-1-phenyl-1H-benzo[d]imidazole (171 mg, 1 Eq, 595 μmol) was added 1,4-dioxane (10 mL) under nitrogen. The mixture was degassed and Pd(dppf)Cl₂ (43.6 mg, 0.1 Eq, 59.5 μmol) was added. The reaction mixture was degassed and heated to reflux at 90 °C for 12 h under nitrogen. The mixture was passed via celite and purified by column chromatography with 100% hexane to 100% ethyl acetate to furnish 2-methyl-1-phenyl-5-(4,4,5,5-tetramethyl-1,3,2-dioxaborolan-2-yl)-1H-benzo[d]imidazole (190 mg, 568 μmol, 95.5%). To a mixture of sodium carbonate (225 mg, 2.13 mL, 1 molar, 3 Eq, 2.13 mmol), 2-methyl-1-phenyl-5-(4,4,5,5-tetramethyl-1,3,2-dioxaborolan-2-yl)-1H-benzo[d]imidazole (188 mg, 0.793 Eq, 562 μmol), and 4-chloro-N-(6-(trifluoromethyl)pyridin-2-yl)pyridin-2-amine (194 mg, 1.0 Eq, 709 μmol) was added DME (8 mL). The mixture was degassed, and Pd(PPh₃)₄ was added (41 mg, 0.05 Eq, 35 μmol). The reaction mixture was heated at 100 °C for 12 h. The crude was cooled, extracted with ethyl acetate, washed with water, and dried over Na₂SO₄. The crude was loaded in a reverse phase column and purified with 100% water to 100% methanol to afford 4-(2-methyl-1-phenyl-1H-benzo[d]imidazol-5-yl)-N-(6-(trifluoromethyl)pyridin-2-yl)pyridin-2-amine (Compound 288, 3.48 mg, 7.81 μmol, 1%). M+H=446

### Synthesis of 4-(3-isopropyl-2-methyl-2H-indazol-5-yl)-N-(6-(trifluoromethyl)pyridin-2-yl)pyridin-2-amine (Compound 290)

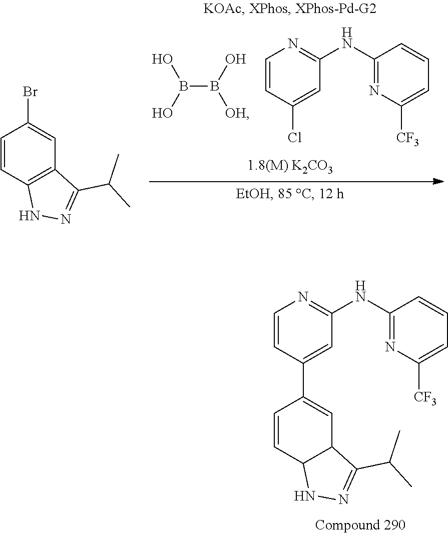

To a solution of potassium acetate (72 mg, 3 Eq, 0.73 mmol) in EtOH (2 mL) was added hypodiboric acid (66 mg, 3 Eq, 0.73 mmol), 4-chloro-N-(6-(trifluoromethyl)pyridin-2-yl)pyridin-2-amine (74 mg, 1.1 Eq, 0.27 mmol), XPhos (3.5 mg, 0.03 Eq, 7.3 μmol), and XPhos-Pd_G2 (5.8 mg, 0.03 Eq, 7.3 μmol). The mixture was degassed and heated at 85 °C until the color turned orange. The mixture was cooled, and 5-bromo-3-isopropyl-2-methyl-2H-indazole (62 mg, 1 Eq, 0.24 mmol) and K₂CO₃ (0.10 g, 0.41 mL, 1.8 molar, 3 Eq, 0.73 mmol) were added under nitrogen. The mixture was heated to 85 °C o/n. The reaction mixture was cooled and passed via celite pad, and dried under reduced pressure. The crude was loaded in a reverse phase column and purified with 10% MeOH to 100% MeOH to afford 4-(3-isopropyl-2-methyl-2H-indazol-5-yl)-N-(6-(trifluoromethyl)pyridin-2-yl)pyridin-2-amine (Compound 290, 13 mg, 32 μmol, 13%). ¹H NMR (400 MHz, DMSO-d₆) δ 12.82 (s, 1H), 10.22 (s, 1H), 8.52 (s, 1H), 8.38-8.30 (m, 2H), 8.15 (s, 1H), 7.93 (d, J=4.3 Hz, 2H), 7.75 (dd, J=19.0, 8.7 Hz, 1H), 7.61 (d, J=8.7 Hz, 1H), 7.40-7.35 (m, 2H), 3.45 (p, J=6.9 Hz, 1H), 1.43 (d, J=6.8 Hz, 6H). M+H=397.9

### Synthesis of 4-(4-fluoro-1-isopropyl-2-methyl-1H-benzo[d]imidazol-6-yl)-N-(6-(trifluoromethyl)pyridin-2-yl)pyridin-2-amine (Compound 286)

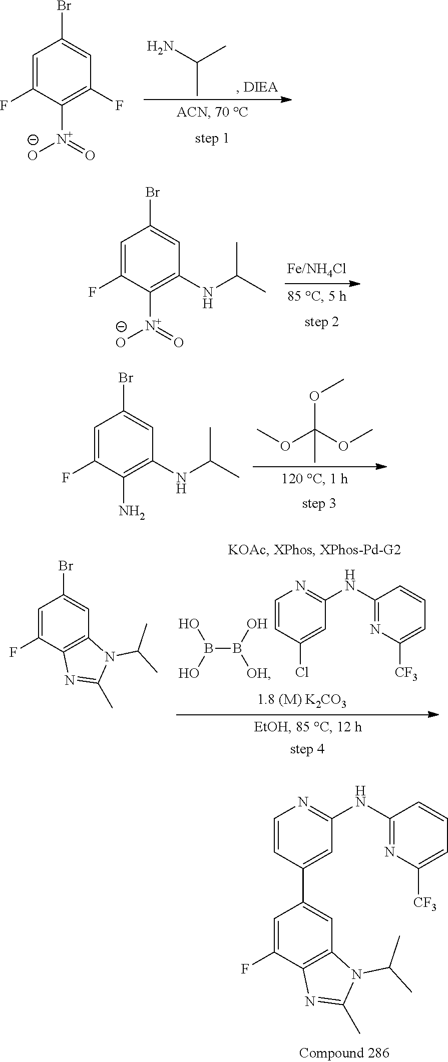

### Step 1—Synthesis of 5-bromo-N1,N3-diisopropyl-2-nitrobenzene-1,3-diamine

To a solution of 5-bromo-1,3-difluoro-2-nitrobenzene (2 g, 1 Eq, 8 mmol) in ACN (15 mL) was added propan-2-amine (1 g, 2 mL, 3 Eq, 0.03 mol). The reaction mixture was heated at 70 °C for 2 h. The reaction mixture was dried under reduced pressure and loaded in a silica column and purified with 100% hexane to 100% ethyl acetate to furnish 5-bromo-3-fluoro-N-isopropyl-2-nitroaniline.

### Step 2—Synthesis of 5-bromo-3-fluoro-N1-isopropylbenzene-1,2-diamine

To a solution of 5-bromo-3-fluoro-N-isopropyl-2-nitroaniline (2 g, 1 Eq, 7 mmol) in ethanol (40 mL)/water (10 mL) was added iron (2 g, 5 Eq, 0.04 mol) and ammonium chloride (2 g, 5 Eq, 0.04 mol). Then the reaction mixture was heated at 85 °C for 4 h. The reaction mixture was cooled and passed through a pad of celite, then was diluted with ethyl acetate and washed with water. The combined organic layer was washed with a saturated solution of NaCl, dried over Na₂SO₄, filtered to remove solid, and concentrated under reduced pressure. The crude material was purified with 100% hexane to 45% hexane in ethyl acetate to furnish the desired product 5-bromo-3-fluoro-N1-isopropylbenzene-1,2-diamine (503 mg, 2.04 mmol, 30%).

### Step 3—Synthesis of 6-bromo-4-fluoro-1-isopropyl-2-methyl-1H-benzo[d]imidazole

5-bromo-3-fluoro-N1-isopropylbenzene-1,2-diamine (503 mg, 1 Eq, 2.04 mmol) and 1,1,1-triethoxyethane (330 mg, 6 mL, 1 Eq, 2.04 mmol) were mixed and heated at 110 °C for 2 h. The crude was dried under reduced pressure and loaded in a silica column. The crude was purified with 100% hexane to 100% ethyl acetate to afford the final compound 6-bromo-4-fluoro-1-isopropyl-2-methyl-1H-benzo[d]imidazole (28 mg, 0.10 mmol, 5%).

### Step 4—Synthesis of 4-(4-fluoro-1-isopropyl-2-methyl-1H-benzo[d]imidazol-6-yl)-N-(6-(trifluoromethyl)pyridin-2-yl)pyridin-2-amine

To a solution of potassium acetate (30 mg, 3 Eq, 0.31 mmol) in EtOH (2 mL) was added hypodiboric acid (28 mg, 3 Eq, 0.31 mmol), XPhos (1.5 mg, 0.03 Eq, 3.1 μmol), XPhos (1.5 mg, 0.03 Eq, 3.1 μmol), XPhos-Pd_G2 (2.4 mg, 0.03 Eq, 3.1 μmol), and 6-bromo-4-fluoro-1-isopropyl-2-methyl-1H-benzo[d]imidazole (28 mg, 1 Eq, 0.10 mmol). The mixture was degassed for 4 minutes and heated at 85 °C until color turned orange. The reaction mixture was cooled, and 4-chloro-N-(6-(trifluoromethyl)pyridin-2-yl)pyridin-2-amine (31 mg, 1.1 Eq, 0.11 mmol) and potassium carbonate (43 mg, 0.17 mL, 1.8 molar, 3 Eq, 0.31 mmol) were added under nitrogen. The reaction was heated at 85 °C o/n. The reaction mixture was cooled, passed through celite, and concentrated. The crude was loaded in a reverse phase column and purified with 10% MeOH to 100% MeOH to afford the title compound in 20% yield. ¹H NMR (400 MHz, DMSO-d₆) δ 10.22 (s, 1H), 8.37-8.27 (m, 2H), 7.94 (d, J=6.1 Hz, 2H), 7.80 (s, 1H), 7.51-7.29 (m, 3H), 4.84 (p, J=7.0 Hz, 1H), 2.63 (s, 3H), 1.63 (d, J=6.8 Hz, 6H). M+H=430.99

Table 1 below shows the characterization data for the compounds as described herein and the IC₅₀ value ranges for Myc inhibition assays of compounds described herein, with compounds having *=IC₅₀ less than 0.2 μM; A=IC₅₀ greater than or equal to 0.2 μM and less than 1 μM; B=IC₅₀ greater than or equal to 1 μM and less than 10 μM; and C=IC₅₀ greater than or equal to 10 μM and less than 100 μM.

TABLE 1
Compound Structures and Characterization Data

| Cmpd No. | Structure | IC₅₀ | NMR; MS (m/z) |
|---|---|---|---|
| 1 | 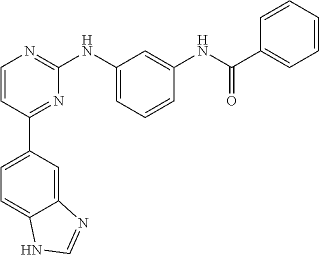 [SMILES: O=C(Nc1cccc(Nc2nccc(-c3ccc4[nH]cnc4c3)n2)c1)c1ccccc1] | * | 407.1 (M + H⁺) |
| 2 | 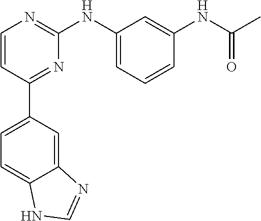 [SMILES: CC(=O)Nc1cccc(Nc2nccc(-c3ccc4[nH]cnc4c3)n2)c1] | A | 345.1 (M + H⁺) |
| 3 | 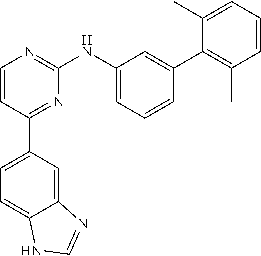 [SMILES: Cc1cccc(C)c1-c1cccc(Nc2nccc(-c3ccc4[nH]cnc4c3)n2)c1] | B | 392.1 (M + H⁺) |
| 4 | 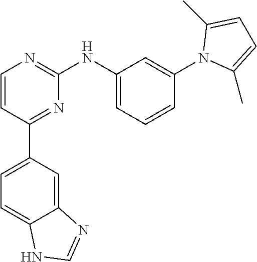 [SMILES: Cc1ccc(C)n1-c1cccc(Nc2nccc(-c3ccc4[nH]cnc4c3)n2)c1] | B | 381.1 (M + H⁺) |
| 5 | 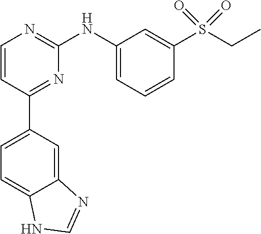 [SMILES: CCS(=O)(=O)c1cccc(Nc2nccc(-c3ccc4[nH]cnc4c3)n2)c1] | * | ¹H NMR (400 MHz, CD₃OD) δ 8.51-8.40 (m, 1H), 8.27 (s, 0H), 8.09 (d, J = 8.5 Hz, 1H), 8.00 (s, 0H), 7.84-7.77 (m, 1H), 7.70 (d, J = 8.5 Hz, 1H), 7.45-7.33 (m, 1H), 6.98 (d, J = 7.9 Hz, 1H), 3.56 (q, J = 7.1 Hz, 1H), 3.35 (q, J = 7.7 Hz, 1H), 1.25 (t, J = 7.1 Hz, 2H), 1.14 (t, J = 7.3 Hz, 2H); 380.1 (M + H⁺) |
| 6 | 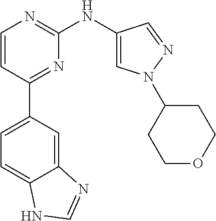 [SMILES: c1cc(-c2ccc3[nH]cnc3c2)nc(Nc2cnn(C3CCOCC3)c2)n1] | B | 362.1 (M + H⁺) |
| 7 | 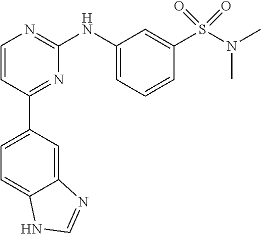 [SMILES: CN(C)S(=O)(=O)c1cccc(Nc2nccc(-c3ccc4[nH]cnc4c3)n2)c1] | * | 395.1 (M + H⁺) |
| 8 | 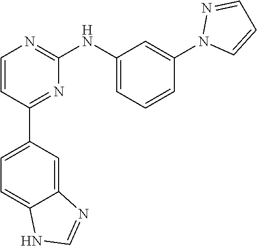 [SMILES: c1cc(Nc2nccc(-c3ccc4[nH]cnc4c3)n2)cc(-n2cccn2)c1] | * | ¹H NMR (400 MHz, CD₃OD) δ 8.48 (d, J = 5.7 Hz, 2H), 8.45- 8.39 (m, 1H), 8.27 (s, 1H), 8.17 (m, 2H), 7.76-7.66 (m, 3H), 7.47-7.37 (m, 3H), 6.53 (d, J = 2.5 Hz, 1H); 354.1 (M + H⁺). |
| 9 | 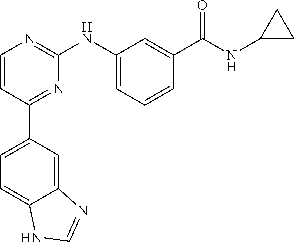 [SMILES: O=C(NC1CC1)c1cccc(Nc2nccc(-c3ccc4[nH]cnc4c3)n2)c1] | A | ¹H NMR (400 MHz, CD₃OD) δ 8.47-8.37 (m, 2H), 8.27 (m, 2H), 8.18-8.06 (m, 1H), 7.89 (td, J = 4.5, 2.3 Hz, 1H), 7.70 (d, J = 8.5 Hz, 3H), 7.43-7.35 (m, 1H), 2.86 (tt, J = 7.4, 3.9 Hz, 1H), 0.87-0.72 (m, 2H), 0.68-0.55 (m, 2H); 371.1 (M + H⁺). |
| 10 | 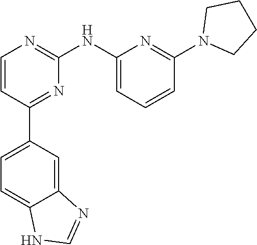 [SMILES: c1cc(Nc2nccc(-c3ccc4[nH]cnc4c3)n2)nc(N2CCCC2)c1] | B | ¹H NMR (400 MHz, CD₃OD) δ 9.47 (s, 1H), 8.66 (d, J = 1.6 Hz, 1H), 8.53 (d, J = 5.7 Hz, 1H), 8.41 (dd, J = 8.8, 1.5 Hz, 1H), 8.00-7.93 (m, 2H), 7.59 (d, J = 5.7 Hz, 1H), 7.54-7.45 (m, 1H), 3.71 (d, J = 6.6 Hz, 4H), 2.27-2.17 (m, 4H); 357.1 (M + H⁺). |
| 11 | 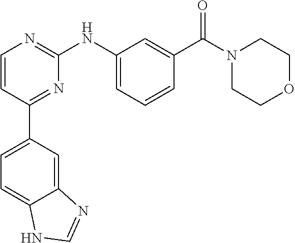 [SMILES: O=C(c1cccc(Nc2nccc(-c3ccc4[nH]cnc4c3)n2)c1)N1CCOCC1] | A | ¹H NMR (400 MHz, CD₃OD) δ 8.49-8.41 (m, 2H), 8.27 (d, J = 0.9 Hz, 1H), 8.12-8.05 (m, 2H), 8.01 (t, J = 1.8 Hz, 1H), 7.84 (ddd, J = 8.2, 2.3, 1.1 Hz, 1H), 7.71 (d, J = 8.5 Hz, 1H), 7.46-7.33 (m, 1H), 7.05 (ddt, J = 7.5, 1.7, 1.0 Hz, 1H), 3.60 (m, 8H); 401.1 (M + H⁺). |
| 12 | 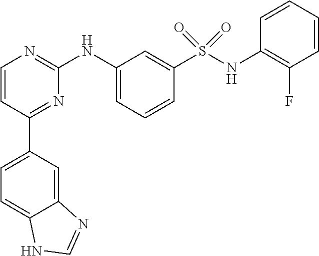 [SMILES: O=S(=O)(Nc1ccccc1F)c1cccc(Nc2nccc(-c3ccc4[nH]cnc4c3)n2)c1] | A | 461.1 (M + H⁺) |
| 13 | 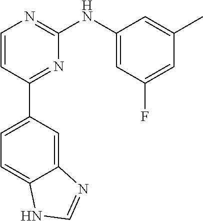 [SMILES: Cc1cc(F)cc(Nc2nccc(-c3ccc4[nH]cnc4c3)n2)c1] | * | 320.1 (M + H⁺) |
| 14 | 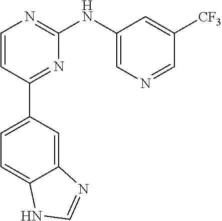 [SMILES: FC(F)(F)c1cncc(Nc2nccc(-c3ccc4[nH]cnc4c3)n2)c1] | C | ¹H NMR (400 MHz, DMSO- D₆) δ 10.29 (s, 1H), 9.21 (s, 1H), 8.87 (s, 1H), 8.63 (d, J = 5.4 Hz, 1H), 8.54 (s, 1H), 8.36 (s, 1H), 8.20 (s, 1H), 8.07 (s, 1H), 7.72 (s, 1H), 7.63 (d, J = 5.4 Hz, 1H); 357.0 (M + H⁺). |
| 15 | 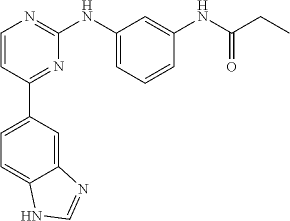 [SMILES: CCC(=O)Nc1cccc(Nc2nccc(-c3ccc4[nH]cnc4c3)n2)c1] | A | 359.1 (M + H⁺) |
| 16 | 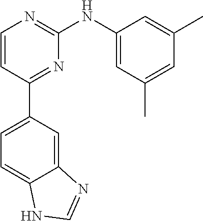 [SMILES: Cc1cc(C)cc(Nc2nccc(-c3ccc4[nH]cnc4c3)n2)c1] | * | ¹H NMR (400 MHz, CD₃OD) δ 8.46-8.36 (m, 2H), 8.25 (m, 1H), 8.12-8.05 (m, 1H), 7.69 (m, 3H), 7.37 (m, 3H), 6.66 (s, 1H), 3.32 (s, 6H); 316.1 (M + H⁺) |
| 17 | 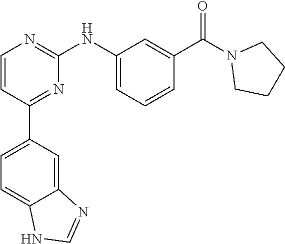 [SMILES: O=C(c1cccc(Nc2nccc(-c3ccc4[nH]cnc4c3)n2)c1)N1CCCC1] | C | 385.16 (M + H⁺). |
| 18 | 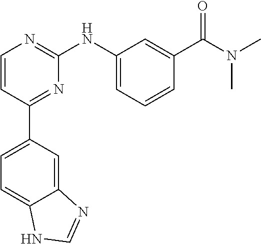 [SMILES: CN(C)C(=O)c1cccc(Nc2nccc(-c3ccc4[nH]cnc4c3)n2)c1] | B | 359.19 (M + H⁺). |
| 19 | 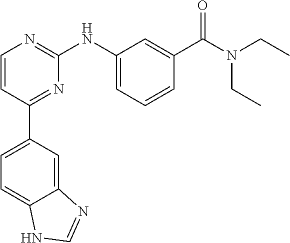 [SMILES: CCN(CC)C(=O)c1cccc(Nc2nccc(-c3ccc4[nH]cnc4c3)n2)c1] | A | 387.25 (M + H⁺). |
| 20 | 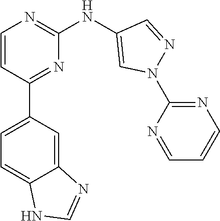 [SMILES: c1cnc(-n2cc(Nc3nccc(-c4ccc5[nH]cnc5c4)n3)cn2)nc1] | A | ¹H NMR (400 MHz, CD₃OD) δ 9.10 (s, 1H), 8.79 (t, J = 5.2 Hz, 2H), 8.56-8.46 (m, 1H), 8.28 (d, J = 5.1 Hz, 1H), 8.16-8.04 (m, 3H), 7.73 (s, 3H), 7.34 (t, J = 5.0 Hz, 2H); 356.1 (M + H⁺). |
| 21 | 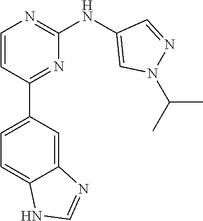 [SMILES: CC(C)n1cc(Nc2nccc(-c3ccc4[nH]cnc4c3)n2)cn1] | A | ¹H NMR (400 MHz, CD₃OD) δ 8.36-8.28 (m, 2H), 8.21 (d, J = 1.1 Hz, 1H), 8.02-7.94 (m, 2H), 7.63 (d, J = 9.1 Hz, 2H), 7.15 (dd, J = 5.3, 1.1 Hz, 1H), 1.45 (dd, J = 6.7, 1.1 Hz, 6H); 320.1 (M + H⁺). |
| 22 | 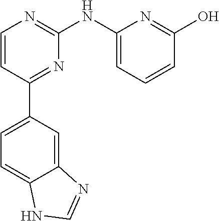 [SMILES: Oc1cccc(Nc2nccc(-c3ccc4[nH]cnc4c3)n2)n1] | C | ¹H NMR (400 MHz, CD₃OD) δ 9.29 (d, J = 8.2 Hz, 1H), 8.69 (dt, J = 5.3, 0.9 Hz, 1H), 8.61- 8.52 (m, 1H), 8.34-8.27 (m, 1H), 7.96-7.85 (m, 2H), 7.72- 7.59 (m, 2H), 7.21 (d, J = 7.5 Hz, 1H), 6.52 (dt, J = 8.2, 0.8 Hz, 1H), 6.43 (dt, J = 7.7, 0.7 Hz, 1H); 305.1 (M + H⁺). |
| 23 | 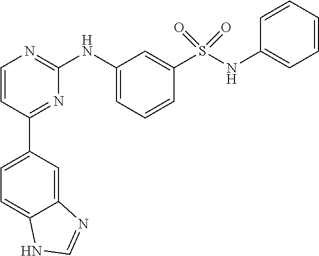 [SMILES: O=S(=O)(Nc1ccccc1)c1cccc(Nc2nccc(-c3ccc4[nH]cnc4c3)n2)c1] | B | ¹H NMR (400 MHz, CD₃OD) δ 8.69 (s, 1H), 8.52-8.42 (m, 2H), 8.28 (s, 1H), 8.21-8.14 (m, 1H), 7.81-7.67 (m, 2H), 7.46-7.31 (m, 3H), 7.23 (ddq, J = 8.4, 1.9, 0.9 Hz, 3H), 7.16- 7.03 (m, 4H), 7.02-6.93 (m, 1H); 443.1 (M + H⁺). |
| 24 | 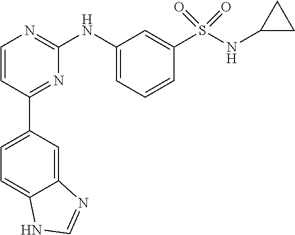 [SMILES: O=S(=O)(NC1CC1)c1cccc(Nc2nccc(-c3ccc4[nH]cnc4c3)n2)c1] | A | ¹H NMR (400 MHz, CD₃OD) δ 9.43 (s, 1H), 8.72 (m, 2H), 8.55- 8.42 (m, 2H), 7.91 (d, J = 8.8 Hz, 1H), 7.75-7.62 (m, 1H), 7.60-7.48 (m, 3H), 3.30 (m, 5H); 407.1 (M + H⁺). |
| 25 | 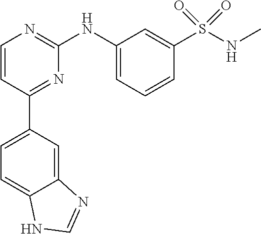 [SMILES: CNS(=O)(=O)c1cccc(Nc2nccc(-c3ccc4[nH]cnc4c3)n2)c1] | * | ¹H NMR (400 MHz, CD₃OD) δ 8.72 (s, 1H), 8.53-8.46 (m, 2H), 8.28 (m, 2H), 7.87 (d, J = 8.1 Hz, 1H), 7.73 (d, J = 8.5 Hz, 1H), 7.56-7.40 (m, 4H), 2.58 (d, J = 1.4 Hz, 3H); 381.1 (M + H⁺). |
| 26 | 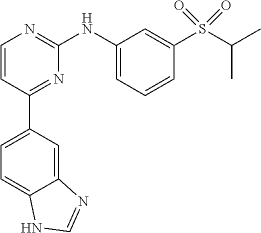 [SMILES: CC(C)S(=O)(=O)c1cccc(Nc2nccc(-c3ccc4[nH]cnc4c3)n2)c1] | * | ¹H NMR (400 MHz, CD₃OD) δ 8.80 (s, 1H), 8.52-8.42 (m, 2H), 8.26 (d, J = 0.9 Hz, 1H), 8.16 (d, J = 8.6 Hz, 1H), 7.91 (d, J = 8.2 Hz, 1H), 7.70 (d, J = 8.5 Hz, 1H), 7.60-7.49 (m, 1H), 7.49-7.37 (m, 2H), 1.27 (dd, J = 6.8, 0.9 Hz, 7H); 394.1 (M + H⁺). |
| 27 | 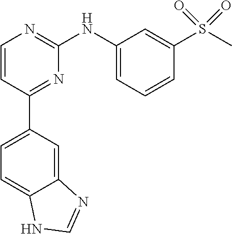 [SMILES: CS(=O)(=O)c1cccc(Nc2nccc(-c3ccc4[nH]cnc4c3)n2)c1] | * | ¹H NMR (400 MHz, CD₃OD) δ 8.82 (s, 1H), 8.51-8.43 (m, 2H), 8.23 (d, J = 1.0 Hz, 1H), 8.14 (d, J = 8.6 Hz, 1H), 7.88 (s, 1H), 7.69 (d, J = 8.6 Hz, 1H), 7.60-7.37 (m, 2H), 3.25 (s, 3H); 366.1 (M + H⁺). |
| 28 | 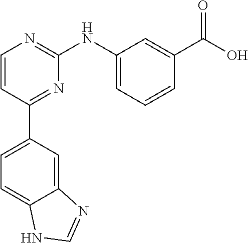 [SMILES: O=C(O)c1cccc(Nc2nccc(-c3ccc4[nH]cnc4c3)n2)c1] | B | ¹H NMR (400 MHz CD₃OD) δ 9.51 (s, 1H), 8.71 (d, J = 5.6 Hz, 1H), 8.51 (dt, J = 11.5, 6.2 Hz, 3H), 8.00 (d, J = 8.8 Hz, 1H), 7.85 (dd, J = 12.9, 7.9 Hz, 2H), 7.54 (t, J = 8.0 Hz, 1H). 360.1 (M + CH₃OH⁺). |
| 29 | 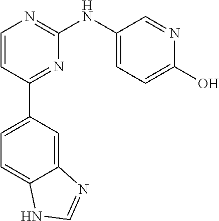 [SMILES: Oc1ccc(Nc2nccc(-c3ccc4[nH]cnc4c3)n2)cn1] | B | ¹H NMR (400 MHz, CD₃OD) δ 7.74-7.67 (m, 2H), 7.62-7.45 (m, 2H), 7.30-7.19 (m, 4H), 6.61 (ddd, J = 9.5, 5.1, 0.9 Hz, 2H); 305.18 (M + H⁺). |
| 30 | 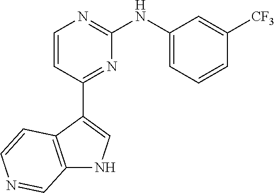 [SMILES: FC(F)(F)c1cccc(Nc2nccc(-c3c[nH]c4cnccc34)n2)c1] | B | ¹H NMR (400 MHz, CD₃OD) δ 8.63 (s, 1H), 8.54 (s, 1H), 8.43 (d, J = 5.2 Hz, 1H), 8.32 (s, 1H), 8.04 (s, 1H), 7.87 (d, J = 8.1 Hz, 1H), 7.70 (d, J = 8.0 Hz, 2H), 7.51 (t, J = 8.0 Hz, 1H), 7.31 (dd, J = 12.7, 6.2 Hz, 2H), 7.23 (d, J = 7.8 Hz, 2H); 356.1 (M + H⁺). |
| 31 | 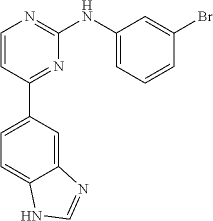 [SMILES: Brc1cccc(Nc2nccc(-c3ccc4[nH]cnc4c3)n2)c1] | * | 367.9 (M + H⁺) |
| 32 | 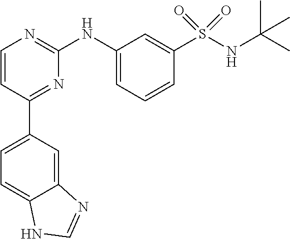 [SMILES: CC(C)(C)NS(=O)(=O)c1cccc(Nc2nccc(-c3ccc4[nH]cnc4c3)n2)c1] | A | ¹H NMR (400 MHz, CD₃OD) δ 8.64-8.40 (m, 2H), 8.28 (s, 2H), 7.20-7.64 (m, 5H), 7.61- 7.37 (m, 3H). 1.21 (9H, s); 423.1 (M + H⁺). |
| 33 | 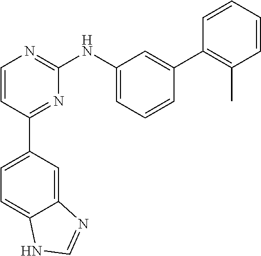 [SMILES: Cc1ccccc1-c1cccc(Nc2nccc(-c3ccc4[nH]cnc4c3)n2)c1] | A | 378.15 (M + H⁺) |
| 34 | 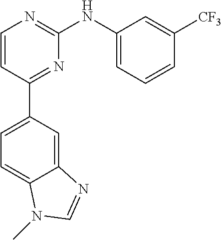 [SMILES: Cn1cnc2cc(-c3ccnc(Nc4cccc(C(F)(F)F)c4)n3)ccc21] | B | ¹H NMR (400 MHz, CD₃OD) δ 8.53-8.43 (m, 2H), 8.35 (t, J = 2.1 Hz, 1H), 8.22-8.15 (m, 2H), 7.94 (dd, J = 8.2, 2.2 Hz, 1H), 7.63 (dd, J = 8.6, 0.7 Hz, 1H), 7.52-7.44 (m, 1H), 7.40 (d, J = 5.3 Hz, 1H), 7.25 (ddt, J = 7.7, 1.7, 0.8 Hz, 1H), 3.92 (s, 3H); 370.1 (M + H⁺). |
| 35 | 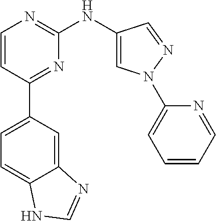 [SMILES: c1ccc(-n2cc(Nc3nccc(-c4ccc5[nH]cnc5c4)n3)cn2)nc1] | * | ¹H NMR (400 MHz, DMSO-d₆) δ 9.81 (d, J = 14.7 Hz, 1H), 8.95 (d, J = 9.6 Hz, 1H), 8.54- 8.46 (m, 2H), 8.36 (m, 3H), 7.95 (d, J = 16.5 Hz, 2H), 7.81 (m, 2H), 7.68 (m, 1H), 7.45 (m, 1H), 7.31 (m, 1H); 355.1 (M + H⁺). |
| 36 | 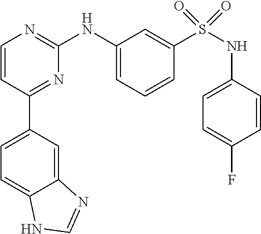 [SMILES: O=S(=O)(Nc1ccc(F)cc1)c1cccc(Nc2nccc(-c3ccc4[nH]cnc4c3)n2)c1] | A | ¹H NMR (400 MHz, DMSO-d₆) δ 10.21 (s, 1H), 10.00 (d, J = 13.7 Hz, 1H), 8.54 (m, 2H), 8.36 (d, J = 8.6 Hz, 2H), 8.17 (m, 1H), 8.06 (m, 1H), 7.92 (m, 1H), 7.79 (m, 1H), 7.67 (m, 1H), 7.57 (m, 1H), 7.47 (t, J = 8.0 Hz, 1H), 7.29 (d, J = 7.8 Hz, 1H), 7.14 (s, 2H), 7.03 (t, J = 8.8 Hz, 2H); 461.1 (M + H⁺). |
| 37 | 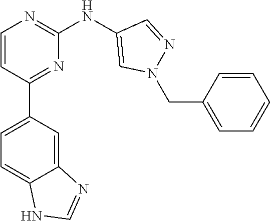 [SMILES: c1ccc(Cn2cc(Nc3nccc(-c4ccc5[nH]cnc5c4)n3)cn2)cc1] | A | ¹H NMR (400 MHz, CD₃OD) δ 8.54 (d, J = 1.4 Hz, 1H), 8.45- 8.38 (m, 2H), 8.25 (d, J = 1.5 Hz, 1H), 8.06 (dt, J = 8.6, 1.6 Hz, 1H), 7.91 (s, 1H), 7.73- 7.65 (m, 3H), 7.46 (ddt, J = 8.6, 7.5, 1.3 Hz, 2H), 7.33-7.22 (m, 2H), 3.37-3.29 (m, 2H); 369.1 (M + H⁺). |
| 38 | 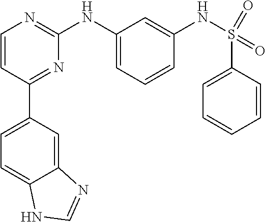 [SMILES: O=S(=O)(Nc1cccc(Nc2nccc(-c3ccc4[nH]cnc4c3)n2)c1)c1ccccc1] | A | ¹H NMR (400 MHz, CD₃OD) δ 8.48-8.35 (m, 2H), 8.25 (d, J = 6.2 Hz, 1H), 8.10 (s, 1H), 7.83-7.74 (m, 3H), 7.70 (s, 1H), 7.52-7.25 (m, 6H), 7.17- 7.03 (m, 1H), 6.69 (d, J = 7.9 Hz, 1H); 443.19 (M + H⁺). |
| 39 | 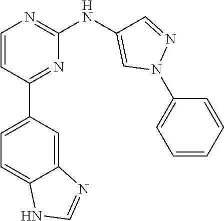 [SMILES: c1ccc(-n2cc(Nc3nccc(-c4ccc5[nH]cnc5c4)n3)cn2)cc1] | * | ¹H NMR (400 MHz, CD₃OD) δ 8.49 (d, J = 1.4 Hz, 1H), 8.41- 8.33 (m, 2H), 8.21 (d, J = 1.5 Hz, 1H), 8.01 (dt, J = 8.6, 1.6 Hz, 1H), 7.87 (s, 1H), 7.68- 7.60 (m, 3H), 7.42 (ddt, J = 8.6, 7.5, 1.3 Hz, 2H), 7.29-7.17 (m, 2H); 354.2 (M + H⁺). |
| 40 | 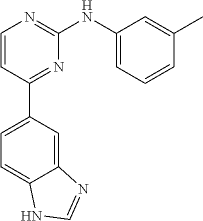 [SMILES: Cc1cccc(Nc2nccc(-c3ccc4[nH]cnc4c3)n2)c1] | A | ¹H NMR (400 MHz, CD₃OD) δ 8.41 (d, J = 5.2 Hz, 2H), 8.25 (m, 2H), 8.09 (m, 2H), 7.58 (d, J = 12.3 Hz, 2H), 7.33 (d, J = 5.3 Hz, 3H), 7.19 (t, J = 7.7 Hz, 2H), 6.83 (d, J = 7.6 Hz, 2H), 3.35 (s, 3H); 302.1 (M + H⁺). |
| 41 | 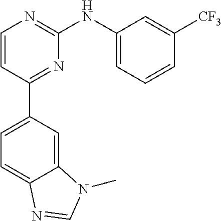 [SMILES: Cn1cnc2ccc(-c3ccnc(Nc4cccc(C(F)(F)F)c4)n3)cc21] | * | ¹H NMR (400 MHz, CD₃OD) δ 8.58-8.49 (m, 2H), 8.44 (dd, J = 1.7, 0.7 Hz, 1H), 8.25 (s, 1H), 8.13 (dd, J = 8.6, 1.7 Hz, 1H), 7.84-7.74 (m, 2H), 7.54-7.46 (m, 2H), 7.31-7.25 (m, 1H), 3.99 (s, 3H); 370.7 (M + H⁺). |
| 42 | 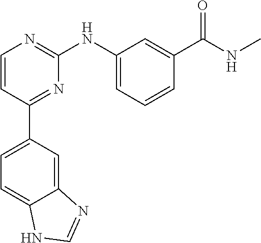 [SMILES: CNC(=O)c1cccc(Nc2nccc(-c3ccc4[nH]cnc4c3)n2)c1] | A | 345.1 (M + H⁺) |
| 43 | 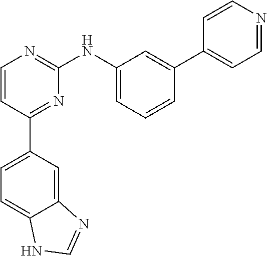 [SMILES: c1cc(Nc2nccc(-c3ccc4[nH]cnc4c3)n2)cc(-c2ccncc2)c1] | * | 365.1 (M + H⁺) |
| 44 | 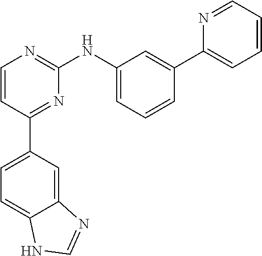 [SMILES: c1ccc(-c2cccc(Nc3nccc(-c4ccc5[nH]cnc5c4)n3)c2)nc1] | * | 365.1 (M + H⁺) |
| 45 | 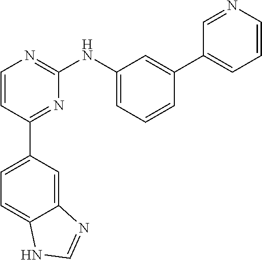 [SMILES: c1cncc(-c2cccc(Nc3nccc(-c4ccc5[nH]cnc5c4)n3)c2)c1] | * | 365.1 (M + H⁺) |
| 46 | 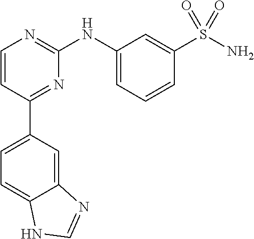 [SMILES: NS(=O)(=O)c1cccc(Nc2nccc(-c3ccc4[nH]cnc4c3)n2)c1] | * | ¹H NMR (400 MHz, CD₃OD) δ 8.76 (s, 1H), 8.48 (s, 2H), 8.41 (dt, J = 5.3, 1.4 Hz, 1H), 7.72 (s, 4H), 7.56-7.50 (m, 2H), 7.31 (dt, J = 5.4, 1.4 Hz, 2H); 367.1 (M + H⁺). |
| 47 | 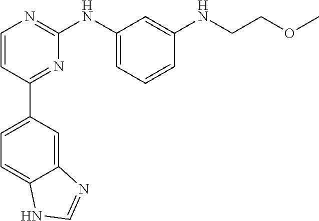 [SMILES: COCCNc1cccc(Nc2nccc(-c3ccc4[nH]cnc4c3)n2)c1] | A | 361.2 (M + H⁺) |
| 48 | 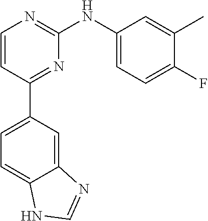 [SMILES: Cc1cc(Nc2nccc(-c3ccc4[nH]cnc4c3)n2)ccc1F] | A | 320.1 (M + H⁺) |
| 49 | 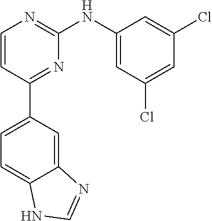 [SMILES: Clc1cc(Cl)cc(Nc2nccc(-c3ccc4[nH]cnc4c3)n2)c1] | B | 356.0 (M + H⁺) |
| 50 | 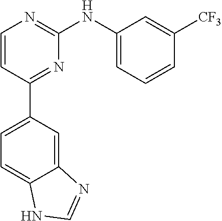 [SMILES: FC(F)(F)c1cccc(Nc2nccc(-c3ccc4[nH]cnc4c3)n2)c1] | B | 356.3 (M + H⁺) |
| 51 | 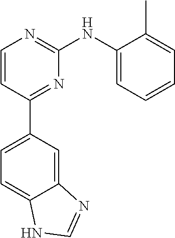 [SMILES: Cc1ccccc1Nc1nccc(-c2ccc3[nH]cnc3c2)n1] | B | ¹H NMR (400 MHz, CDCl₃) δ 8.44 (dd, J = 5.3, 1.6 Hz, 2H), 8.17-8.10 (m, 3H), 7.27-7.22 (m, 3H), 7.04 (t, J = 7.5 Hz, 2H), 2.36 (s, 3H); 302.2 (M + H⁺). |
| 52 | 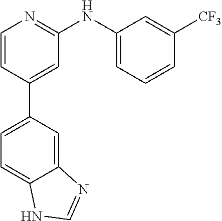 [SMILES: FC(F)(F)c1cccc(Nc2cc(-c3ccc4[nH]cnc4c3)ccn2)c1] | B | ¹H NMR (400 MHz, CDCl₃) δ 8.27 (m, 2H), 7.62 (m, 4H), 7.33-6.75 (m, 3H), 6.68-6.63 (m, 2H); 355.2 (M + H⁺). |
| 53 | 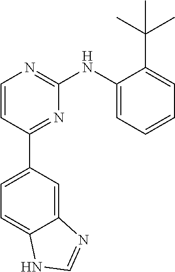 [SMILES: CC(C)(C)c1ccccc1Nc1nccc(-c2ccc3[nH]cnc3c2)n1] | B | ¹H NMR (400 MHz, CDCl₃) δ 8.39 (dd, J = 5.0, 2.8 Hz, 2H), 8.09 (d, J = 2.9 Hz, 1H), 8.00 (m, 2H), 7.77 (d, J = 8.1 Hz, 1H), 7.44 (d, J = 8.0 Hz, 2H), 7.25 (dd, J = 3.6, 1.3 Hz, 2H), 7.16 (dt, J = 5.3, 2.2 Hz, 2H), 6.94 (s, 1H), 1.49-1.44 (s, 9H); 344.3 (M + H⁺). |
| 54 | 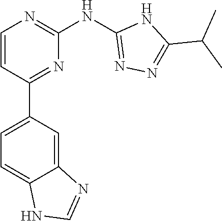 [SMILES: CC(C)c1nnc(Nc2nccc(-c3ccc4[nH]cnc4c3)n2)[nH]1] | B | ¹H NMR (400 MHz, CD₃OD) δ 8.86-8.77 (m, 1H), 8.64 (dd, J = 9.3, 5.6 Hz, 1H), 8.52 (d, J = 17.5 Hz, 2H), 8.31 (q, J = 4.7 Hz, 1H), 8.18-8.08 (m, 1H), 8.02-7.94 (m, 2H), 7.91-7.83 (m, 1H), 7.74 (d, J = 10.7 Hz, 1H), 3.48 (m, 1H), 1.38-1.23 (m, 6H); 321.1 (M + H⁺). |
| 55 | 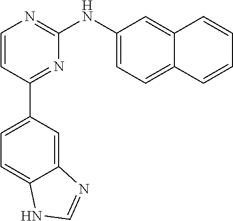 [SMILES: c1ccc2cc(Nc3nccc(-c4ccc5[nH]cnc5c4)n3)ccc2c1] | A | ¹H NMR (400 MHz, DMSO-d₆) δ 10.29 (s, 1H), 9.91 (s, 1H), 8.94-8.87 (m, 2H), 8.82 (d, J = 2.0 Hz, 1H), 8.65 (dd, J = 8.7, 1.6 Hz, 1H), 8.31-8.15 (m, 2H), 7.86-7.74 (m, 2H), 7.59 (ddd, J = 7.9, 6.7, 1.2 Hz, 1H), 7.33 (d, J = 7.9 Hz, 3H); 338.2 (M + H⁺). |
| 56 | 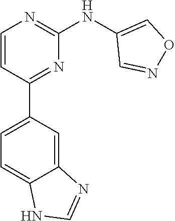 [SMILES: c1cc(-c2ccc3[nH]cnc3c2)nc(Nc2cnoc2)n1] | A | ¹H NMR (400 MHz, CD₃OD) δ 8.52-8.42 (m, 2H), 8.28 (m, 1H), 8.11 (d, J = 8.5 Hz, 1H), 7.73 (dd, J = 3.2, 1.4 Hz, 2H), 7.38-7.29 (m, 2H), 7.21 (ddd, J = 5.2, 1.4, 0.5 Hz, 1H); 279.1 (M + H⁺). |
| 57 | 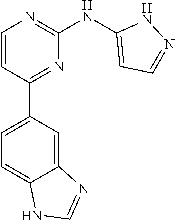 [SMILES: c1cc(Nc2nccc(-c3ccc4[nH]cnc4c3)n2)[nH]n1] | B | 278.1 (M + H⁺) |
| 58 | 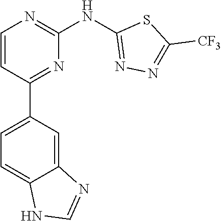 [SMILES: FC(F)(F)c1nnc(Nc2nccc(-c3ccc4[nH]cnc4c3)n2)s1] | B | ¹H NMR (400 MHz, DMSO-d₆) δ 8.77 (s, 1H), 8.57 (s, 1H), 8.40 (d, J = 15.8 Hz, 2H), 8.30- 7.96 (m, 2H), 7.88-7.61 (m, 1H). |
| 59 | 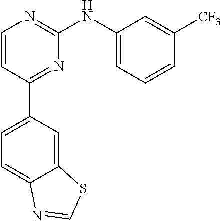 [SMILES: FC(F)(F)c1cccc(Nc2nccc(-c3ccc4ncsc4c3)n2)c1] | A | ¹H NMR (400 MHz, DMSO-d₆) δ 10.58-9.91 (m, 1H), 9.76- 9.27 (m, 1H), 9.19-8.82 (m, 1H), 8.79-8.11 (m, 4H), 7.73- 7.36 (m, 2H), 7.30 (d, J = 17.4 Hz, 1H); 373.1 (M + H⁺). |
| 60 | 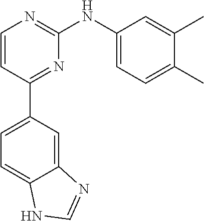 [SMILES: Cc1ccc(Nc2nccc(-c3ccc4[nH]cnc4c3)n2)cc1C] | A | ¹H NMR (400 MHz, CDCl₃) δ 8.26-8.18 (m, 2H), 7.53 (d, J = 8.1 Hz, 2H), 7.32-7.23 (m, 1H), 7.20 (d, J = 3.0 Hz, 1H), 7.10-6.86 (m, 4H), 2.04 (dd, J = 17.0, 3.4 Hz, 6H); 316.1 (M + H⁺). |
| 61 | 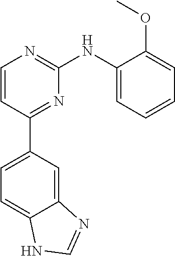 [SMILES: COc1ccccc1Nc1nccc(-c2ccc3[nH]cnc3c2)n1] | B | ¹H NMR (400 MHz, CD₃OD) δ 8.57-8.43 (m, 2H), 8.26 (s, 1H), 8.06 (dd, J = 8.6, 1.7 Hz, 1H), 7.71 (dd, J = 8.6, 0.7 Hz, 1H), 7.34 (dd, J = 5.3, 1.5 Hz, 1H), 7.06-6.96 (m, 3H), 3.93 (s, J = 1.5 Hz, 3H); 318.2 (M + H⁺). |
| 62 | 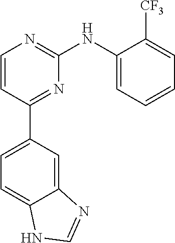 [SMILES: FC(F)(F)c1ccccc1Nc1nccc(-c2ccc3[nH]cnc3c2)n1] | B | ¹H NMR (400 MHz, CDCl₃) δ 8.58 (d, J = 13.1 Hz, 2H), 8.15 (m, 1H), 7.69 (m, 3H), 7.41 (m, 1H), 7.26 (m, 4H); 356.1 (M + H⁺). |
| 63 | 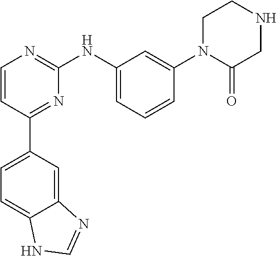 [SMILES: O=C1CNCCN1c1cccc(Nc2nccc(-c3ccc4[nH]cnc4c3)n2)c1] | B | ¹H NMR (400 MHz, CDCl₃) δ 8.41 (dd, J = 5.3, 0.5 Hz, 1H), 7.25 (d, J = 0.5 Hz, 1H), 6.72- 6.56 (m, 2H), 4.27 (t, J = 5.4 Hz, 2H), 3.87-3.62 (m, 2H), 1.24 (m, 2H), 0.83 (m, 1H); 386.1 (M + H⁺). |
| 64 | 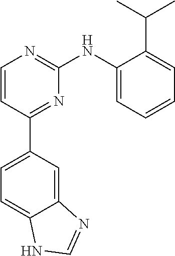 [SMILES: CC(C)c1ccccc1Nc1nccc(-c2ccc3[nH]cnc3c2)n1] | B | ¹H NMR (400 MHz, CDCl₃) δ 8.43-8.33 (m, 2H), 8.05 (s, 1H), 7.98 (dd, J = 8.5, 1.6 Hz, 1H), 7.88 (dd, J = 8.0, 1.4 Hz, 1H), 7.71-7.62 (m, 1H), 7.30 (dd, J = 7.7, 1.6 Hz, H), 7.26- 7.08 (m, 2H), 6.97 (s, 1H), 3.21 (hept, J = 6.7 Hz, 1H), 1.23 (d, J = 6.8 Hz, 6H); 330.28 (M + H⁺). |
| 65 | 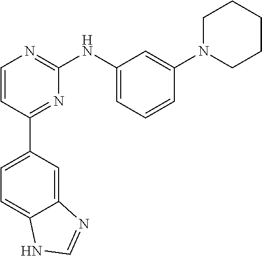 [SMILES: c1cc(Nc2nccc(-c3ccc4[nH]cnc4c3)n2)cc(N2CCCCC2)c1] | A | ¹H NMR (400 MHz, CDCl₃) δ 8.58-8.44 (m, 2H), 8.28-8.10 (m, 2H), 7.82 (t, J = 9.1 Hz, 1H), 7.69-7.59 (m, 1H), 7.53 (d, J = 12.2 Hz, 1H), 7.43- 7.15 (m, 4H), 6.78 (ddd, J = 13.0, 8.7, 4.8 Hz, 1H), 1.83 (d, J = 10.2 Hz, 4H), 1.70 (d, J = 7.8 Hz, 2H), 0.99 (m, 2H); 371.4 (M + H⁺). |
| 66 | 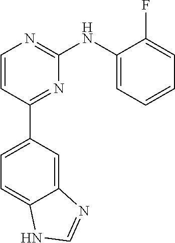 [SMILES: Fc1ccccc1Nc1nccc(-c2ccc3[nH]cnc3c2)n1] | B | ¹H NMR (400 MHz, CD₃OD) δ 8.32-8.23 (m, 2H), 8.18-8.08 (m, 2H), 7.54 (dt, J = 8.6, 1.1 Hz, 1H), 7.23 (dd, J = 5.4, 1.7 Hz, 1H), 7.09-6.86 (m, 3H); 306.2 (M + H⁺). |
| 67 | 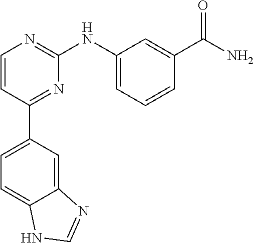 [SMILES: NC(=O)c1cccc(Nc2nccc(-c3ccc4[nH]cnc4c3)n2)c1] | A | ¹H NMR (400 MHz, CD₃OD) δ 8.55-8.44 (m, 3H), 8.28 (s, 1H), 8.20-8.10 (m, 1H), 8.05- 7.95 (m, 2H), 7.78-7.76 (m, 2H), 7.54-7.38 (m, 3H); 331.2 (M + H⁺). |
| 68 | 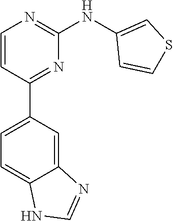 [SMILES: c1cc(-c2ccc3[nH]cnc3c2)nc(Nc2ccsc2)n1] | A | ¹H NMR (400 MHz, CD₃OD) δ 8.36-8.27 (m, 2H), 8.12 (m, 1H), 7.95 (dd, J = 8.5, 1.7 Hz, 2H), 7.61-7.52 (m, 2H), 7.21- 7.14 (m, 1H); 294.1 (M + H⁺). |
| 69 | 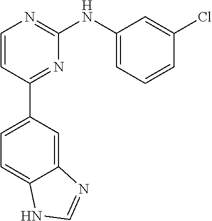 [SMILES: Clc1cccc(Nc2nccc(-c3ccc4[nH]cnc4c3)n2)c1] | * | ¹H NMR (400 MHz, CD₃OD) δ 8.43 (dd, J = 5.4, 2.1 Hz, 2H), 8.27-8.21 (m, 1H), 8.08 (dq, J = 8.6, 1.9 Hz, 1H), 8.00 (p, J = 2.1 Hz, 1H), 7.69 (dt, J = 8.5, 2.1 Hz, 1H), 7.61 (dtt, J = 8.2, 2.1, 1.1 Hz, 1H), 7.35 (dt, J = 5.2, 2.1 Hz, 1H), 7.30-7.20 (m, 1H), 6.95 (dtt, J = 7.9, 2.1, 1.3 Hz, 1H); 322.2 (M + H⁺). |
| 70 | 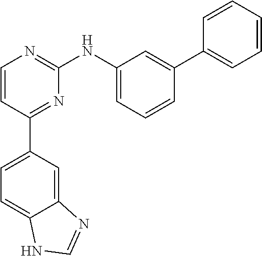 [SMILES: c1ccc(-c2cccc(Nc3nccc(-c4ccc5[nH]cnc5c4)n3)c2)cc1] | * | ¹H NMR (400 MHz, CD₃OD) δ 8.49-8.34 (m, 2H), 8.22 (s, 1H), 8.19-8.04 (m, 2H), 7.77- 7.56 (m, 4H), 7.47-7.15 (m, 6H); 364.1 (M + H⁺). |
| 71 | 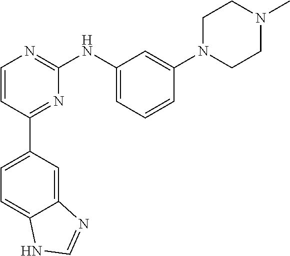 [SMILES: CN1CCN(c2cccc(Nc3nccc(-c4ccc5[nH]cnc5c4)n3)c2)CC1] | A | 386.4 (M + H⁺) |
| 72 | 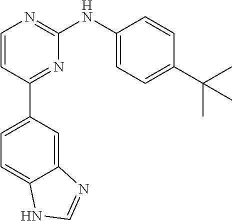 [SMILES: CC(C)(C)c1ccc(Nc2nccc(-c3ccc4[nH]cnc4c3)n2)cc1] | B | ¹H NMR (400 MHz, CD₃OD) δ 8.45 (s, 1H), 8.39-8.32 (m, 1H), 8.24 (s, 1H), 8.05 (dt, J = 8.5, 1.5 Hz, 1H), 7.71-7.61 (m, 3H), 7.39-7.30 (m, 2H), 7.26 (dt, J = 5.3, 1.0 Hz, 1H), 1.31 (d, J = 1.3 Hz, 9H); 344.2 (M + H⁺). |
| 73 | 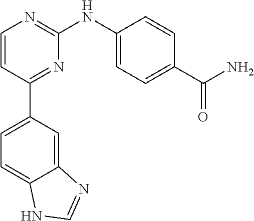 [SMILES: NC(=O)c1ccc(Nc2nccc(-c3ccc4[nH]cnc4c3)n2)cc1] | B | ¹H NMR (400 MHz, CD₃OD) δ 9.32 (s, 1H), 8.67 (dd, J = 1.5, 0.7 Hz, 2H), 8.60 (dd, J = 5.2, 0.7 Hz, 1H), 8.41 (dd, J = 8.7, 1.5 Hz, 1H), 7.99-7.86 (m, 2H), 7.69 (dd, J = 10.6, 8.4 Hz, 2H), 7.50 (dd, J = 5.3, 0.7 Hz, 1H), 7.26-7.19 (m, 2H); 331.2 (M + H⁺). |
| 74 | 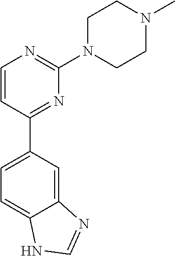 [SMILES: CN1CCN(c2nccc(-c3ccc4[nH]cnc4c3)n2)CC1] | B | ¹H NMR (400 MHz, CD₃OD) δ 8.35 (d, J = 3.8 Hz, 2H), 8.20 (d, J = 4.7 Hz, 1H), 8.04-7.96 (m, 1H), 7.63 (dd, J = 8.7, 3.9 Hz, 1H), 7.12 (t, J = 5.1 Hz, 1H), 3.89 (q, J = 4.8 Hz, 4H), 2.50 (q, J = 4.8 Hz, 4H), 2.31 (d, J = 4.8 Hz, 3H); 295.2 (M + H⁺). |
| 75 | 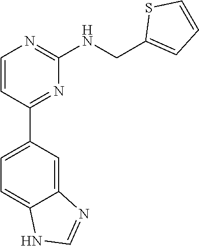 [SMILES: c1csc(CNc2nccc(-c3ccc4[nH]cnc4c3)n2)c1] | B | ¹H NMR (400 MHz, CD₃OD) δ 8.47 (s, 1H), 8.34-8.23 (m, 2H), 8.12-8.04 (m, 1H), 7.69 (dd, J = 8.6, 2.8 Hz, 1H), 7.33- 7.18 (m, 2H), 7.07 (ddq, J = 3.4, 2.1, 1.0 Hz, 1H), 7.03- 6.90 (m, 3H), 4.02-3.96 (m, 2H); 308.1 (M + H⁺). |
| 76 | 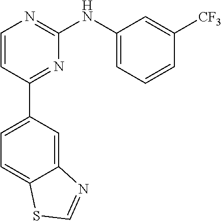 [SMILES: FC(F)(F)c1cccc(Nc2nccc(-c3ccc4scnc4c3)n2)c1] | B | ¹H NMR (400 MHz, CD₃OD) δ 9.31 (d, J = 6.6 Hz, 1H), 8.91- 8.83 (m, 1H), 8.62-8.53 (m, 1H), 8.36 (d, J = 2.3 Hz, 1H), 8.34-8.25 (m, 1H), 8.24-8.14 (m, 1H), 8.03-7.91 (m, 1H), 7.76-7.67 (m, 1H), 7.54-7.43 (m, 2H), 7.42-7.31 (m, 1H), 7.26 (d, J = 7.7 Hz, 1H); 373.4 (M + H⁺). |
| 77 | 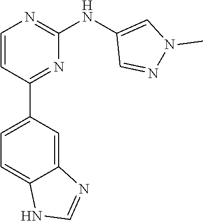 [SMILES: Cn1cc(Nc2nccc(-c3ccc4[nH]cnc4c3)n2)cn1] | A | ¹H NMR (400 MHz, CD₃OD) δ 8.47-8.37 (m, 2H), 8.28 (d, J = 4.3 Hz, 1H), 8.08 (m, 1H), 7.73 (m, 1H), 7.65 (m, 1H), 7.28 (t, J = 5.0 Hz, 2H), 3.92 (d, J = 4.4 Hz, 3H); 292.2 (M + H⁺). |
| 78 | 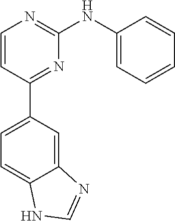 [SMILES: c1ccc(Nc2nccc(-c3ccc4[nH]cnc4c3)n2)cc1] | B | ¹H NMR (400 MHz, CD₃OD) δ 8.37 (m, 1H), 8.31 (dd, J = 5.3, 3.2 Hz, 1H), 8.22 (d, J = 2.9 Hz, 1H), 8.00 (dq, J = 8.6, 1.7 Hz, 1H), 7.72-7.62 (m, 2H), 7.31- 7.15 (m, 3H), 7.06 (tq, J = 7.5, 1.4 Hz, 1H); 288.2 (M + H⁺). |
| 79 | 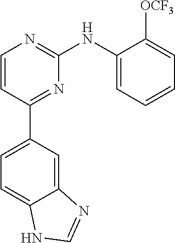 [SMILES: FC(F)(F)Oc1ccccc1Nc1nccc(-c2ccc3[nH]cnc3c2)n1] | A | ¹H NMR (400 MHz, CD₃OD) δ 8.47-8.38 (m, 3H), 8.22 (d, J = 1.3 Hz, 1H), 8.02 (dt, J = 8.6, 1.5 Hz, 1H), 7.67 (d, J = 8.5 Hz, 1H), 7.42-7.27 (m, 3H), 7.09 (ddt, J = 8.2, 7.4, 1.6 Hz, 1H); 372.2 (M + H⁺). |
| 80 | 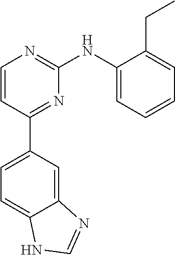 [SMILES: CCc1ccccc1Nc1nccc(-c2ccc3[nH]cnc3c2)n1] | B | ¹H NMR (400 MHz, CDCl₃) δ 8.44-8.34 (m, 2H), 8.08-7.95 (m, 3H), 7.69 (d, J = 8.5 Hz, 2H), 7.29-7.19 (m, 1H), 7.16 (dd, J = 5.3, 0.8 Hz, 1H), 7.07 (td, J = 7.5, 1.2 Hz, 1H), 6.98 (s, 1H), 2.70 (q, J = 7.6 Hz, 2H), 1.24 (t, J = 7.6, 3H); 316.2 (M + H⁺). |
| 81 | 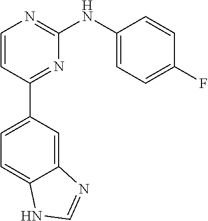 [SMILES: Fc1ccc(Nc2nccc(-c3ccc4[nH]cnc4c3)n2)cc1] | A | ¹H NMR (400 MHz, CD₃OD) δ 8.44 (s, 2H), 8.36-8.16 (m, 1H), 8.08 (d, J = 15.1 Hz, 1H), 7.73 (m, 4H), 7.34 (d, J = 17.1 Hz, 1H), 7.06 (m, 2H); 306.2 (M + H⁺). |
| 82 | 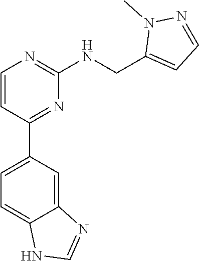 [SMILES: Cn1nccc1CNc1nccc(-c2ccc3[nH]cnc3c2)n1] | B | ¹H NMR (400 MHz, CD₃OD) δ 8.38 (s, 1H), 8.33-8.23 (m, 2H), 8.05-7.98 (m, 1H), 7.68 (d, J = 8.6 Hz, 1H), 7.35 (dd, J = 2.0, 0.5 Hz, 1H), 7.20 (dd, J = 5.4, 0.6 Hz, 1H), 6.31-6.24 (m, 1H), 4.74 (s, 2H), 3.91 (d, J = 0.5 Hz, 3H); 306.2 (M + H⁺). |
| 83 | 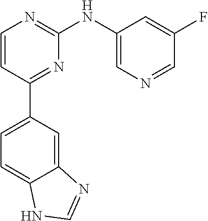 [SMILES: Fc1cncc(Nc2nccc(-c3ccc4[nH]cnc4c3)n2)c1] | B | ¹H NMR (400 MHz, CD₃OD) δ 8.70-8.60 (m, 2H), 8.47 (s, 2H), 8.38-8.19 (m, 2H), 8.13 (m, 4H), 8.04-7.93 (m, 2H), 7.73 (m, 1H); 306.27 (M+). |
| 84 | 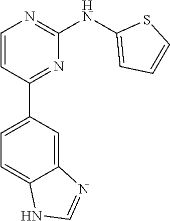 [SMILES: c1csc(Nc2nccc(-c3ccc4[nH]cnc4c3)n2)c1] | B | ¹H NMR (400 MHz, CD₃OD) δ 8.80-8.65 (m, 3H), 8.60 (m, 1H), 8.35-8.19 (m, 2H), 8.18- 8.13 (m, 3H), 8.04-7.03 (m, 1H), 7.95 (m, 1H); 316.1 (M + Na⁺). |
| 85 | 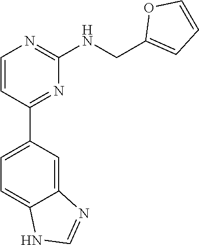 [SMILES: c1coc(CNc2nccc(-c3ccc4[nH]cnc4c3)n2)c1] | B | ¹H NMR (400 MHz, DMSO-d₆) δ 8.28 (m, J = 11.8 Hz, 3H), 8.05-7.88 (m, 1H), 7.64-7.50 (m, 3H), 7.20 (dd, J = 10.5, 5.2 Hz, 1H), 6.24 (m, 2H), 4.55 (d, J = 6.0 Hz, 2H); 292.2 (M + H⁺). |
| 86 | 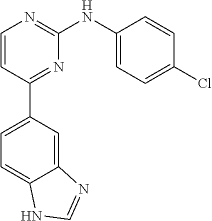 [SMILES: Clc1ccc(Nc2nccc(-c3ccc4[nH]cnc4c3)n2)cc1] | A | ¹H NMR (400 MHz, DMSO-d₆) δ 9.80 (s, 1H), 8.54 (dd, J = 5.2, 1.4 Hz, 1H), 8.45 (s, 1H), 8.36 (s, 1H), 8.07 (m, 1H), 7.94- 7.87 (m, 2H), 7.73 (m, 1H), 7.52 (m, 1H), 7.41-7.34 (m, 2H); 322.2 (M + H⁺). |
| 87 | 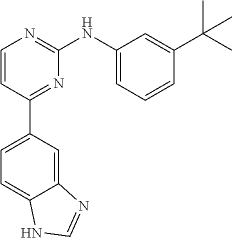 [SMILES: CC(C)(C)c1cccc(Nc2nccc(-c3ccc4[nH]cnc4c3)n2)c1] | A | ¹H NMR (400 MHz, DMSO-d₆) δ 8.29 (dd, J = 5.2, 1.1 Hz, 2H), 8.13 (s, 1H), 7.89 (s, 1H), 7.75 (s, 1H), 7.46 (d, J = 8.9 Hz, 2H), 7.22 (d, J = 5.3 Hz, 1H), 7.03 (td, J = 7.9, 1.0 Hz, 1H), 6.80 (ddt, J = 7.8, 2.1, 1.1 Hz, 1H), 1.11 (d, J = 1.1 Hz, 9H); 344.2 (M + H⁺). |
| 88 | 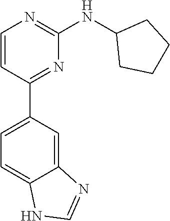 [SMILES: c1cc(-c2ccc3[nH]cnc3c2)nc(NC2CCCC2)n1] | B | ¹H NMR (400 MHz, CD₃OD) δ 8.41 (s, 1H), 8.25 (dd, J = 5.4, 0.6 Hz, 2H), 8.03 (dd, J = 8.6, 1.7 Hz, 1H), 7.68 (d, J = 8.6 Hz, 1H), 7.13 (dd, J = 5.4, 0.7 Hz, 1H), 4.39-4.31 (m, 1H), 2.09 (dt, J = 12.5, 6.1 Hz, 2H), 1.87- 1.52 (m, 6H); 280.24 (M + H⁺). |
| 89 | 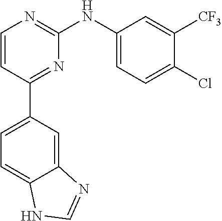 [SMILES: FC(F)(F)c1cc(Nc2nccc(-c3ccc4[nH]cnc4c3)n2)ccc1Cl] | B | ¹H NMR (400 MHz, CDCl₃) δ 8.64-8.57 (m, 2H), 8.30 (d, J = 0.6 Hz, 1H), 8.21 (dd, J = 8.6, 1.7 Hz, 1H), 7.91 (d, J = 8.6 Hz, 1H), 7.79-7.71 (m, 2H), 7.43 (d, J = 0.6 Hz, 1H), 7.39-7.29 (m, 3H); 390.1 (M + H⁺). |
| 90 | 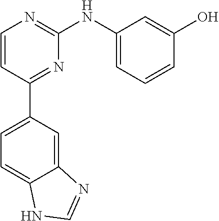 [SMILES: Oc1cccc(Nc2nccc(-c3ccc4[nH]cnc4c3)n2)c1] | * | ¹H NMR (400 MHz, DMSO-d₆) δ 9.27 (s, 1H), 8.49 (d, J = 5.2 Hz, 1H), 8.33 (s, 1H), 8.06 (s, 1H), 7.70 (s, 1H), 7.44 (d, J = 5.3 Hz, 1H), 7.37 (t, J = 2.2 Hz, 1H), 7.27 (d, J = 8.1 Hz, 1H), 7.07 (t, J = 8.1 Hz, 1H), 6.75 (t, J = 8.2 Hz, 1H), 6.37 (ddd, J = 8.1, 2.4, 0.9 Hz, 1H), 5.97 (dd, J = 4.5, 2.5 Hz, 1H); 304.2 (M + H⁺). |
| 91 | 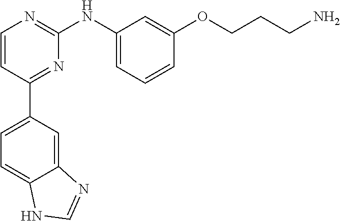 [SMILES: NCCCOc1cccc(Nc2nccc(-c3ccc4[nH]cnc4c3)n2)c1] | A | 361.3 (M + H⁺) |
| 92 | 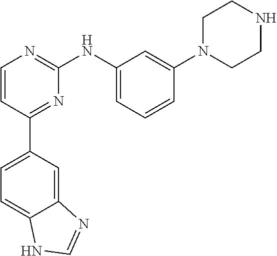 [SMILES: c1cc(Nc2nccc(-c3ccc4[nH]cnc4c3)n2)cc(N2CCNCC2)c1] | B | ¹H NMR (400 MHz, CD₃OD) δ 9.35 (s, 1H), 8.58 (s, 1H), 8.47 (m, 1H), 8.37 (dd, J = 8.7, 1.5 Hz, 1H), 7.92 (d, J = 8.7 Hz, 1H), 7.65 (d, J = 8.1 Hz, 2H), 7.46-7.36 (m, 2H), 7.34-7.21 (m, 1H), 7.25-7.12 (m, 3H), 6.61 (ddd, J = 8.1, 2.6, 0.9 Hz, 1H), 4.10 (t, J = 5.7 Hz, 2H), 3.13 (t, J = 7.3 Hz, 2H), 2.17- 2.06 (m, 2H); 372.3(M + H⁺). |
| 93 | 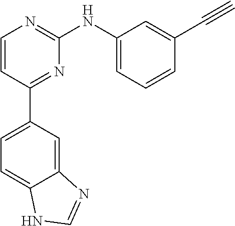 [SMILES: C#Cc1cccc(Nc2nccc(-c3ccc4[nH]cnc4c3)n2)c1] | A | ¹H NMR (400 MHz, CD₃OD) δ 9.50-9.45 (m, 1H), 8.65 (dt, J = 1.6, 0.8 Hz, 1H), 8.51-8.40 (m, 2H), 7.97 (dt, J = 8.8, 0.7 Hz, 1H), 7.77 (t, J = 1.9 Hz, 1H), 7.70-7.60 (m, 3H), 7.38 (t, J = 7.9 Hz, 1H), 7.27 (d, J = 7.7 Hz, 1H), 7.21-7.14 (m, 2H), 3.65-3.41 (m, 1H); 312.2 (M + H⁺). |
| 94 | 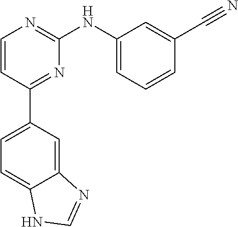 [SMILES: N#Cc1cccc(Nc2nccc(-c3ccc4[nH]cnc4c3)n2)c1] | B | ¹H NMR (400 MHz, DMSO-d₆) δ 10.14 (s, 1H), 9.52 (s, 1H), 8.93 (d, J = 1.3 Hz, 1H), 8.68 (d, J = 5.2 Hz, 2H), 7.71 (m, J = 5.3 Hz, 3H), 7.57 (m, 2H), 7.31 (m, 2H); 313.2 (M + H⁺). |
| 95 | 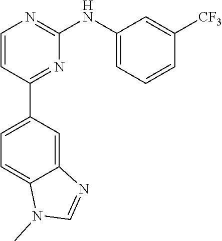 [SMILES: Cn1cnc2cc(-c3ccnc(Nc4cccc(C(F)(F)F)c4)n3)ccc21] | * | ¹H NMR (400 MHz, CD₃OD) δ 8.56-8.45 (m, 2H), 8.35 (s, 1H), 8.23-8.17 (m, 2H), 7.93 (d, J = 8.3 Hz, 1H), 7.72-7.62 (m, 1H), 7.47 (t, J = 7.9 Hz, 1H), 7.41 (d, J = 5.3 Hz, 1H), 7.21 (d, J = 8.1 Hz, 1H), 3.92 (s, 3H). |
| 96 | 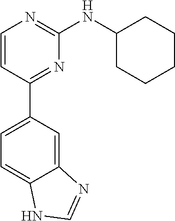 [SMILES: c1cc(-c2ccc3[nH]cnc3c2)nc(NC2CCCCC2)n1] | B | ¹H NMR (400 MHz, CDCl₃) δ 8.15 (s, 1H), 8.06 (d, J = 5.3 Hz, 1H), 7.79 (s, 1H), 7.77- 7.70 (m, 1H), 7.57-7.32 (m, 1H), 6.73 (d, J = 5.3 Hz, 1H), 5.01 (d, J = 8.1 Hz, 1H), 3.69 (d, J = 11.0 Hz, 1H), 1.84 (dd, J = 12.5, 4.4 Hz, 2H), 1.50-1.38 (m, 3H), 1.12-0.8 (m, 6H); 294.24 (M + H⁺). |
| 97 | 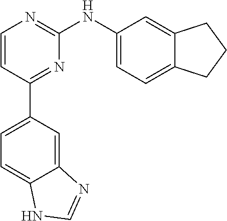 [SMILES: c1cc(-c2ccc3[nH]cnc3c2)nc(Nc2ccc3c(c2)CCC3)n1] | B | ¹H NMR (400 MHz, CD₃OD) δ 8.36 (d, J = 1.7 Hz, 1H), 8.28 (dt, J = 5.3, 1.0 Hz, 1H), 8.21- 8.15 (m, 1H), 7.98 (dt, J = 8.5, 1.4 Hz, 1H), 7.61 (dd, J = 8.6, 1.3 Hz, 1H), 7.51 (s, 1H), 7.38 (ddt, J = 8.0, 2.1, 1.0 Hz, 1H), 7.18 (dt, J = 5.3, 1.0 Hz, 1H), 7.07 (d, J = 8.0 Hz, 1H), 5.45- 5.39 (m, 1H), 2.88-2.75 (m, 4H), 2.09-1.94 (m, 2H); 345.2 (M + H₂O). |
| 98 | 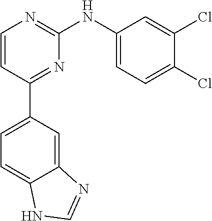 [SMILES: Clc1ccc(Nc2nccc(-c3ccc4[nH]cnc4c3)n2)cc1Cl] | A | ¹H NMR (400 MHz, CD₃OD) δ 8.65 (dd, J = 6.1, 4.1 Hz, 1H), 8.49 (m, 2H), 8.32 (m, 2H), 8.12 (m, 2H), 8.00 (t, J = 5.1 Hz, 2H), 7.74 (m, 2H); 356.08 (M + H⁺). |
| 99 | 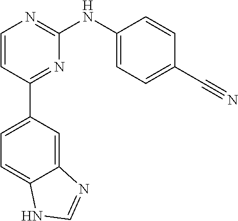 [SMILES: N#Cc1ccc(Nc2nccc(-c3ccc4[nH]cnc4c3)n2)cc1] | A | ¹H NMR (400 MHz, CD₃OD) δ 8.49 (dd, J = 5.3, 1.7 Hz, 1H), 8.25 (s, 1H), 8.08 (d, J = 8.4 Hz, 1H), 8.04-7.96 (m, 2H), 7.71-7.59 (m, 2H), 7.44 (dd, J = 5.3, 1.6 Hz, 1H); 313.2 (M + H⁺). |
| 100 | 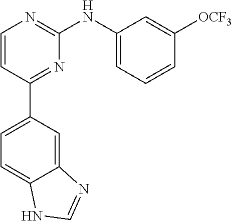 [SMILES: FC(F)(F)Oc1cccc(Nc2nccc(-c3ccc4[nH]cnc4c3)n2)c1] | A | ¹H NMR (400 MHz, CD₃OD) δ 8.65 (dd, J = 5.4, 0.8 Hz, 1H), 8.52-8.42 (m, 2H), 8.30 (d, J = 14.2 Hz, 2H), 8.16-8.08 (m, 2H), 8.05-7.95 (m, 2H), 7.77- 7.71 (m, 2H), 7.44-7.33 (m, 1H); 372.1 (M + H⁺). |
| 101 | 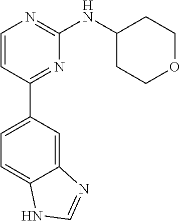 [SMILES: c1cc(-c2ccc3[nH]cnc3c2)nc(NC2CCOCC2)n1] | B | ¹H NMR (400 MHz, CD₃OD) δ 8.34 (s, 1H), 8.23-8.16 (m, 2H), 7.99-7.91 (m, 1H), 7.66- 7.58 (m, 1H), 7.07 (ddd, J = 5.2, 3.2, 1.6 Hz, 1H), 4.06 (s, 1H), 3.93 (dt, J = 11.0, 3.3 Hz, 2H), 3.52 (ddt, J = 14.6, 9.7, 2.0 Hz, 2H), 3.28 (ddd, J = 15.6, 3.2, 1.4 Hz, 2H), 1.98 (d, J = 13.1 Hz, 2H), 1.65-1.49 (m, 2H); 297.26 (M + 2H⁺). |
| 102 | 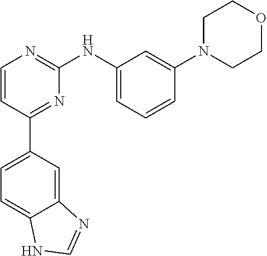 [SMILES: c1cc(Nc2nccc(-c3ccc4[nH]cnc4c3)n2)cc(N2CCOCC2)c1] | * | ¹H NMR (400 MHz, CD₃OD) δ 8.35-8.21 (m, 3H), 8.21-8.11 (m, 2H), 8.00-7.89 (m, 1H), 7.61-7.44 (m, 2H), 7.20-7.01 (m, 3H), 6.50 (q, J = 7.8 Hz, 1H), 3.76-3.64 (m, 4H), 3.05 (m, 4H); 373.5 (M + H⁺). |
| 103 | 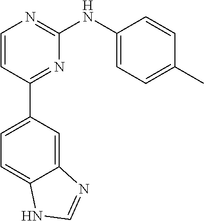 [SMILES: Cc1ccc(Nc2nccc(-c3ccc4[nH]cnc4c3)n2)cc1] | A | ¹H NMR (400 MHz, CDCl₃) δ 8.68 (bs, 2H), 8.40 (d, J = 1.7 Hz, 1H), 8.04 (d, J = 0.7 Hz, 1H), 7.85 (d, J = 2.6 Hz, 1H), 7.76 (dd, J = 8.7, 2.6 Hz, 2H), 7.53-7.38 (m, 2H), 7.27-7.18 (m, 1H), 2.17 (s, 3H); 302.2 (M + H⁺). |
| 104 | 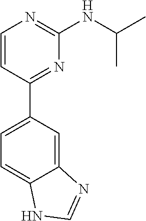 [SMILES: CC(C)Nc1nccc(-c2ccc3[nH]cnc3c2)n1] | B | ¹H NMR (400 MHz, CD₃OD) δ 8.38-8.28 (m, 1H), 8.14 (m, 2H), 7.59 (dd, J = 8.5, 1.9 Hz, 1H), 6.98 (dd, J = 5.4, 2.0 Hz, 1H), 4.20-4.09 (m, 1H), 1.19 (dd, J = 6.6, 2.1 Hz, 6H); 254.2 (M + H⁺). |
| 105 | 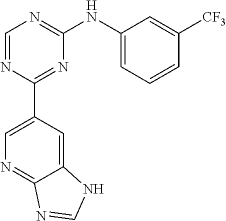 [SMILES: FC(F)(F)c1cccc(Nc2ncnc(-c3cnc4nc[nH]c4c3)n2)c1] | * | ¹H NMR (400 MHz, DMSO- d₆): δ = 10.67 (brs, 1H), 9.39 (s, 1H), 8.93 (s, 1H), 8.86 (s, 1H), 8.59 (s, 1H), 8.40 (brs, 1H), 8.04-8.02 (m, 1H), 7.65-7.63 (m, 1H), 7.47-7.45 (m, 1H); 358.0 (M + H⁺). |
| 106 | 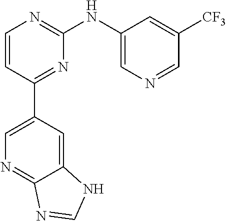 [SMILES: FC(F)(F)c1cncc(Nc2nccc(-c3cnc4nc[nH]c4c3)n2)c1] | * | ¹H NMR (400 MHz, DMSO- d₆): δ = 10.35 (brs, 1H), 9.21 (dd, J = 7.6, 1.2 Hz, 2H), 8.85 (s, 1H), 8.76 (s, 1H), 8.68 (d, J = 5.2 Hz, 1H), 8.58-8.55 (m, 2H), 7.72 (d, J = 5.2 Hz, 1H); 358.0 (M + H⁺). |
| 107 | 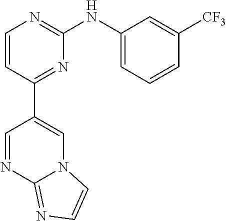 [SMILES: FC(F)(F)c1cccc(Nc2nccc(-c3cnc4nccn4c3)n2)c1] | B | ¹H NMR (400 MHz, DMSO- d₆): δ = 10.12 (s, 1H), 9.42 (s, 1H), 9.22 (s, 1H), 8.71 (d, J = 4.8 Hz, 1H), 8.32 (s, 1H), 8.28 (s, 1H), 8.16 (d, J = 8.0 Hz, 1H), 7.94 (s, 1H), 7.74 (d, J = 4.8 Hz, 1H), 7.60 (t, J = 3.6 Hz, 1H), 7.34 (d, J = 3.6 Hz, 1H); 357.0 (M + H⁺). |
| 108 | 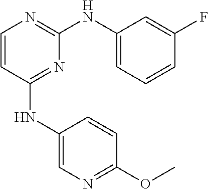 [SMILES: COc1ccc(Nc2ccnc(Nc3cccc(F)c3)n2)cn1] | B | ¹H NMR (400 MHz, DMSO- d₆): δ = 9.39 (s, 1H), 9.33 (s, 1H), 8.50 (d, J = 2.8 Hz, 1H), 8.03 (d, J = 5.6 Hz, 1H)), 7.98 (dd, J = 2.8, 2.8 Hz, 1H), 7.81- 7.77 (m, 1H), 7.42-7.39 (m, 1H), 7.25-7.19 (m, 1H), 6.81 (d, J = 8.8 Hz, 1H), 6.70-6.65 (m, 1H), 6.20 (d, J = 5.6 Hz, 1H), 3.85 (s, 3H); 312.1 (M + H⁺). |
| 109 | 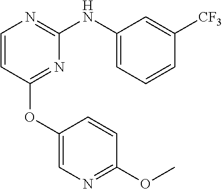 [SMILES: COc1ccc(Oc2ccnc(Nc3cccc(C(F)(F)F)c3)n2)cn1] | B | ¹H NMR (400 MHz, CD₃OD): δ = 8.35 (d, J = 4.0 Hz, 1 H), 8.02 (d, J = 4.0 Hz, 1 H), 7.89 (s, 1 H), 7.63-7.56 (m, 2 H), 7.31- 7.29 (t, J = 8.0 HZ, 1 H), 7.19 (d, J = 8.0 Hz, 1 H), 6.89 (d, J = 8.0 Hz, 1 H), 6.54 (d, J = 4.0 Hz, 1 H), 3.96 (s, 3 H); 363.0 (M + H⁺). |
| 110 | 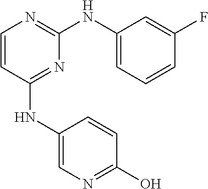 [SMILES: Oc1ccc(Nc2ccnc(Nc3cccc(F)c3)n2)cn1] | B | ¹H NMR (400 MHz, DMSO- d₆): δ = 9.93 (s, 1 H), 8.46 (d, J = 4.0 Hz, 1 H), 7.68-7.56 (m, 3 H), 7.40 (d, J = 8.0 Hz, 1 H), 7.29-7.23 (m, 1 H), 6.79 (t, J = 8.0 Hz, 1 H), 6.61 (d, J = 8.0 Hz, 1 H), 6.52 (d, J = 12.0 Hz, 1 H); 299.0 (M + H⁺). |
| 111 | 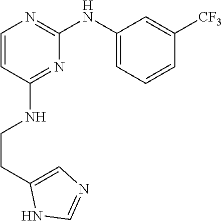 [SMILES: FC(F)(F)c1cccc(Nc2nccc(NCCc3cnc[nH]3)n2)c1] | B | ¹H NMR (400 MHz, DMSO- d₆): δ = 11.82 (brs, 1H), 9.36 (s, 1H), 8.34 (s, 1H), 7.97 (d, J = 5.6 Hz, 1H), 7.84 (s, 1H), 7.56 (s, 1H), 7.42 (t, J = 7.4 Hz, 1H)), 7.35 (s, 1H), 7.17 (d, J = 7.2 Hz, 1H), 6.82 (s, 1H), 6.01 (d, J = 6.0 Hz, 1H), 3.59 (s, 2H), 2.80 (t, J = 7.2 Hz, 2H); 349.1 (M + H⁺). |
| 112 | 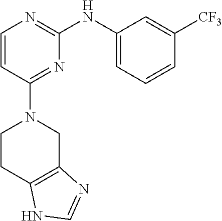 [SMILES: FC(F)(F)c1cccc(Nc2nccc(N3CCc4[nH]cnc4C3)n2)c1] | B | ¹H NMR (400 MHz, DMSO- d₆): δ = 11.86 (brs, 1H), 9.46 (brs, 1H), 8.41 (s, 1H), 8.03 (d, J = 5.6 Hz, 1H), 7.84 (d, J = 7.4 Hz, 1H), 7.50-7.45 (m, 2H), 7.21 (d, J = 7.6 Hz, 1H), 6.43 (d, J = 6.0 Hz, 1H), 4.56 (m, 2H), 3.97 (m, 2H), 2.70 (m, 2H); 361.1 (M + H⁺). |
| 113 | 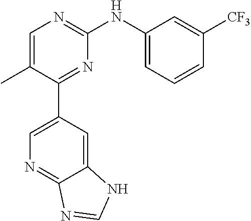 [SMILES: Cc1cnc(Nc2cccc(C(F)(F)F)c2)nc1-c1cnc2nc[nH]c2c1] | B | ¹H NMR (400 MHz, DMSO- d₆): δ = 13.05 (brs, 1H), 9.30 (brs, 1H), 8.93 (s, 1H), 8.76- 8.69 (m, 1H), 8.53-8.37 (m, 3H), 8.09 (s, 1H), 7.65 (t, J = 8.0 Hz, 1H), 7.44 (d, J = 7.6 Hz, 1H), 2.30 (s, 3H); 371.1 (M + H⁺). |
| 114 | 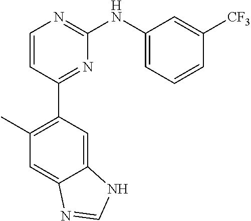 [SMILES: Cc1cc2nc[nH]c2cc1-c1ccnc(Nc2cccc(C(F)(F)F)c2)n1] | B | ¹H NMR (400 MHz, DMSO- d₆): δ = 9.22 (s, 1H), 8.61 (d, J = 4.8 Hz, 1H), 8.32 (s, 1H), 7.92 (s, 1H), 7.88 (d, J = 8.0 Hz, 1H), 7.78 (s, 1H), 7.45 (t, J = 4.8, 2.4 Hz, 1H), 7.25 (d, J = 4.8, 2.4 Hz, 1H), 7.08 (d, J = 5.2 Hz, 1H), 2.63 (s, 3H); 370.1 (M + H⁺). |
| 115 | 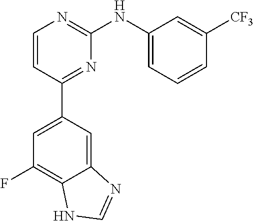 [SMILES: Fc1cc(-c2ccnc(Nc3cccc(C(F)(F)F)c3)n2)cc2nc[nH]c12] | * | ¹H NMR (400 MHz, DMSO- d₆): 8 = 10.55 (s, 1H), 8.60 (d, J = 5.2 Hz, 1H), 8.49 (s, 1H), 8.40 (s, 1H), 8.26 (s, 1H), 7.97 (d, J = 8.0 Hz, 1H), 7.86 (d, J = 12.4 Hz, 1H), 7.58 (d, J = 5.6 Hz, 1H), 7.55 (d, J = 8.4 Hz, 1H), 7.30 (d, J = 7.6 Hz, 1H); 374.0 (M + H⁺). |
| 116 | 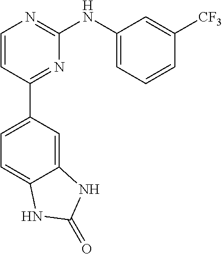 [SMILES: O=c1[nH]c2ccc(-c3ccnc(Nc4cccc(C(F)(F)F)c4)n3)cc2[nH]1] | C | ¹H NMR (400 MHz, DMSO- d₆): δ = 10.92 (s, 2H), 9.98 (s, 1H), 8.54 (d, J = 5.3 Hz, 1H), 8.38 (s, 1H), 8.06 (d, J = 8.2 Hz, 1H), 7.84 (d, J = 8.2 Hz, 1H), 7.76 (s, 1H), 7.54 (t, J = 8.0 Hz, 1H), 7.42 (d, J = 5.3 Hz, 1H), 7.29 (d, J = 7.6 Hz, 1H), 7.06 (d, J = 8.2 Hz, 1H); 372.0 (M + H⁺). |
| 117 | 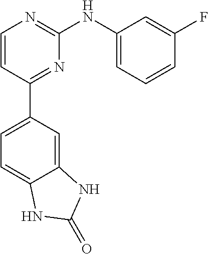 [SMILES: O=c1[nH]c2ccc(-c3ccnc(Nc4cccc(F)c4)n3)cc2[nH]1] | C | ¹H NMR (400 MHz, DMSO- d₆): δ = 10.92 (s, 2H), 9.85 (s, 1H), 8.51 (d, J = 5.3 Hz, 1H), 7.88-7.79 (m, 2H), 7.76 (s, 1H), 7.58 (d, J = 8.0 Hz, 1H), 7.39 (d, J = 5.3 Hz, 1H), 7.32 (dd, J = 15.4, 8.2 Hz, 1H), 7.06 (d, J = 8.2 Hz, 1H), 6.80-6.70 (m, 1H); 321.9 (M + H⁺). |
| 118 | 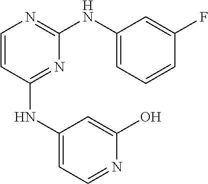 [SMILES: Oc1cc(Nc2ccnc(Nc3cccc(F)c3)n2)ccn1] | C | ¹H NMR(400 MHz, DMSO-d₆): δ = 10.98 (brs, 1H), 9.59 (s, 1H), 9.54 (s, 1H), 8.16 (d, J = 5.6 Hz, 1H), 7.78 (d, J = 12.4 Hz, 1H), 7.50 (d, J = 8.4 Hz, 1H), 7.30-7.27 (m, 1H), 7.24- 7.22 (m, 1H), 6.94 (s, 1H), 6.74-6.70 (m, 1H), 6.43-6.40 (m, 1H), 6.35 (d, J = 5.6 Hz, 1H); 298.1 (M + H⁺). |
| 119 | 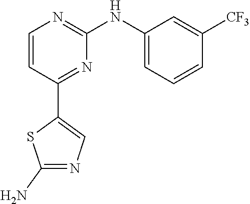 [SMILES: Nc1ncc(-c2ccnc(Nc3cccc(C(F)(F)F)c3)n2)s1] | * | ¹H NMR (400 MHz, DMSO- d₆): δ = 9.82 (s, 1H), 8.35 (s, 1H), 8.33 (s, 1H), 7.99 (s, 1H), 7.94 (d, J = 8.8 Hz, 1H), 7.68 (s, 2H), 7.47 (t, J = 7.6, 3.6 Hz, 1H), 7.24 (d, J = 7.6 Hz, 1H), 7.21 (d, J = 5.6 Hz, 1H); 338.0 (M + H⁺). |
| 120 | 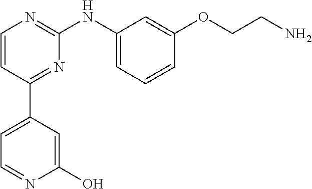 [SMILES: NCCOc1cccc(Nc2nccc(-c3ccnc(O)c3)n2)c1] | B | ¹H NMR (400 MHz, DMSO- d₆): δ = 9.78 (brs, 1H), 8.62 (d, J = 4 Hz, 1H), 7.65-7.63 (m, 1H), 7.56 (t, J = 4 Hz, 1H), 7.41 (t, J = 4 Hz, 1H), 7.31-7.28 (m, 1H), 7.19 (t, J = 8 Hz, 1H), 7.13-7.11 (m, 1H), 6.86-6.84 (m, 1H), 6.58-6.55 (m, 1H), 3.96 (t, J = 4 Hz, 2H), 2.93 (t, J = 4 Hz, 2H), 1.87 (s, 3H); 324.1 (M + H⁺). |
| 121 | 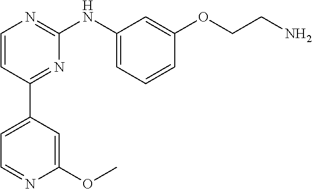 [SMILES: COc1cc(-c2ccnc(Nc3cccc(OCCN)c3)n2)ccn1] | B | ¹H NMR (400 MHz, DMSO- d₆): δ = 9.78 (brs, 1H), 8.65 (d, J = 4 Hz, 1H), 8.36 (d, J = 8 Hz, 1H), 7.71-7.68 (m, 1H), 7.66- 7.64 (m, 1H), 7.56-7.54 (m, 1H), 7.52-7.50 (m, 3H), 7.33- 7.30 (m, 1H), 7.20 (t, J = 8 Hz, 1H), 6.58-6.55 (m, 1H), 3.95- 3.92 (m, 5H), 2.90 (t, J = 4 Hz, 2H); 338.1 (M + H⁺). |
| 122 | 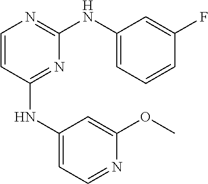 [SMILES: COc1cc(Nc2ccnc(Nc3cccc(F)c3)n2)ccn1] | B | ¹H NMR (400 MHz, DMSO- d₆): δ = 9.77 (s, 1H), 9.57 (s, 1H), 8.16 (d, J = 5.6 Hz, 1H), 7.98 (d, J = 6.0 Hz, 1H)), 7.82- 7.78 (m, 1H), 7.49-7.46 (m, 1H), 7.31 (d, J = 2Hz, 1H), 7.29-7.27 (m, 1H), 7.26-7.24 (m, 1H), 6.76-6.71 (m, 1H), 6.35 (d, J = 6.0 Hz, 1H), 3.83 (s, 3H) 312.1 (M + H⁺). |
| 123 | 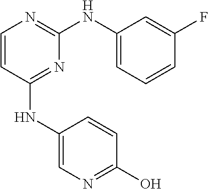 [SMILES: Oc1ccc(Nc2ccnc(Nc3cccc(F)c3)n2)cn1] | C | ¹H NMR(400 MHz, DMSO-d₆): δ = 11.43 (brs, 1H), 9.33 (s, 1H), 8.93 (s, 1H), 7.97 (d, J = 6.0 Hz, 1H), 7.78 (d, J = 12.8 Hz, 1H), 7.62 (s, 1H), 7.58-7.55 (m, 1H), 7.40 (d, J = 8.4 Hz, 1H), 7.20 (m, 1H), 6.66 (m, 1H), 6.38 (d, J = 9.6 Hz, 1H), 6.10 (d, J = 5.6 Hz, 1H); 298.1 (M + H⁺). |
| 124 | 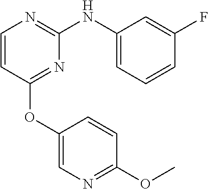 [SMILES: COc1ccc(Oc2ccnc(Nc3cccc(F)c3)n2)cn1] | C | ¹H NMR (400 MHz, CD₃OD): δ = 8.21 (d, J = 8.0 Hz, 1 H), 7.90 (d, J = 4.0 Hz, 1 H), 7.48- 7.45 (m, 1 H), 7.25 (d, J = 12.0 Hz, 1 H), 7.03-6.94 (m, 2 H), 6.80 (d, J = 8.0 Hz, 1 H), 6.50 (t, J = 16.0 Hz, 1 H), 6.41 (d, J = 8.0 Hz, 1 H), 3.85 (s, 3 H); 313.0 (M + H⁺). |
| 125 | 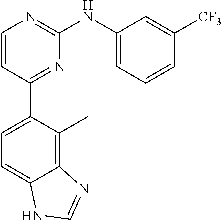 [SMILES: Cc1c(-c2ccnc(Nc3cccc(C(F)(F)F)c3)n2)ccc2[nH]cnc12] | B | ¹H NMR (300 MHz, CD₃OD): δ = 9.44 (s, 1H), 8.59 (d, J = 4.8 Hz, 1H), 8.34 (s, 1H), 7.77-7.86 (m, 3H), 7.43 (t, J = 7.6, 4.8 Hz, 3H), 7.24 (d, J = 6.8 Hz, 1H), 7.08 (d, J = 4.8 Hz, 1H), 2.77 (s, 3H); 370.1 (M + H⁺). |
| 126 | 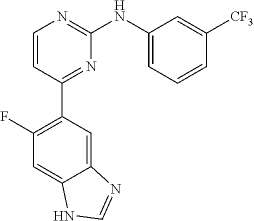 [SMILES: Fc1cc2[nH]cnc2cc1-c1ccnc(Nc2cccc(C(F)(F)F)c2)n1] | A | ¹H NMR (400 MHz, DMSO- d₆): δ = 10.13 (s, 1H), 8.84 (s, 1H), 8.64 (d, J = 6.4 Hz, 1H), 8.39 (s, 1H), 8.31 (d, J = 6.8 Hz, 1H), 8.03 (d, J = 8.4 Hz, 1H), 7.70 (d, J = 11.6 Hz, 1H), 7.52 (t, J = 6.8, 2.4 Hz, 1H), 7.34 (dd, J = 8.0, 2.4 Hz, 1H), 7.29 (d, J = 3.6 Hz, 1H); 374.0 (M + H⁺). |
| 127 | 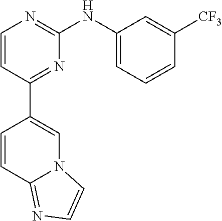 [SMILES: FC(F)(F)c1cccc(Nc2nccc(-c3ccc4nccn4c3)n2)c1] | A | ¹H NMR (400 MHz, DMSO- d₆): δ = 10.11 (s, 1H), 9.43 (s, 1H), 8.65 (d, J = 5.2 Hz, 1H), 8.41 (s, 1H), 8.04-7.98 (m, 3H), 7.70 (t, J = 9.6 Hz, 2H), 7.57 (t, J = 8.0 Hz, 1H), 7.51 (d, J = 5.2 Hz, 1H), 7.32 (d, J = 7.6 Hz, 1H); 355.9 (M + H⁺). |
| 128 | 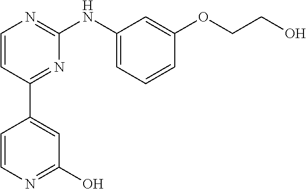 [SMILES: OCCOc1cccc(Nc2nccc(-c3ccnc(O)c3)n2)c1] | B | ¹H NMR (400 MHz, DMSO- d₆): δ = 11.83 (brs, 1H), 9.77 (brs, 1H), 8.62 (d, J = 4 Hz, 1H), 7.59-7.57 (m, 1H), 7.54 (d, J = 8 Hz, 1H), 7.41 (t, J = 8 Hz, 1H), 7.34-7.31 (m, 1H), 7.19 (t, J = 8 Hz, 1H), 7.11-7.09 (m, 1H), 6.87-6.85 (m, 1H), 6.57- 6.55 (m, 1H), 4.88-4.86 (m, 1H), 4.00-3.97 (m, 2H), 3.75- 3.72 (m, 2H); 325.1 (M + H⁺). |
| 129 | 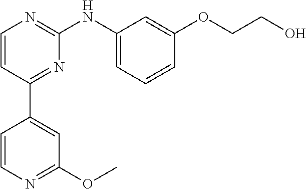 [SMILES: COc1cc(-c2ccnc(Nc3cccc(OCCO)c3)n2)ccn1] | B | ¹H NMR (400 MHz, DMSO- d₆): δ = 9.79 (brs, 1H), 8.68- 8.64 (m 1H), 8.38-8.35 (m, 1H), 7.71-7.68 (m, 1H), 7.63-7.60 (m, 1H), 7.55-7.51 (m, 2H), 7.37-7.34 (m, 1H), 7.22-7.18 (m, 1H), 6.58-6.55 (m, 1H), 4.86-4.86 (m, 1H), 4.01-3.98 (m, 2H), 3.92 (s, 3H), 3.75-3.72 (m, 2H); 339.1 (M + H⁺). |
| 130 | 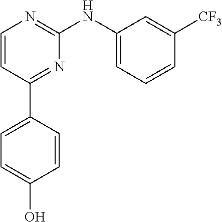 [SMILES: Oc1ccc(-c2ccnc(Nc3cccc(C(F)(F)F)c3)n2)cc1] | A | ¹H NMR (400 MHz, DMSO- d₆): δ = 10.08 (brs, 1H), 9.56 (brs, 1H), 8.52-8.49 (m, 2H), 8.06 (t, J = 8 Hz, 2H), 7.97 (d, J = 8 Hz, 1H), 7.53 (d, J = 8 Hz, 1H), 7.37 (d, J = 4 Hz, 1H), 7.28 (d, J = 4 Hz, 1H), 6.90 (d, J = 8 Hz, 2H); 331.9 (M + H⁺). |
| 131 | 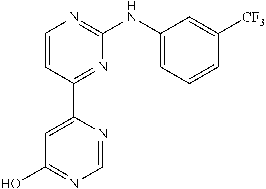 [SMILES: Oc1cc(-c2ccnc(Nc3cccc(C(F)(F)F)c3)n2)ncn1] | B | ¹H NMR (400 MHz, DMSO- d₆): δ = 12.82 (s, 1H), 10.18 (s, 1H), 8.75 (d, 1H), 8.40 (s, J = 5.2 Hz, 1H), 8.35 (s, 1H), 7.94 (d, J = 8.4 Hz, 1H), 7.65 (d, J = 3.2 Hz, 1H), 7.54 (t, J = 8.0, 3.6 Hz, 1H), 7.31 (d, J = 8.0 Hz, 1H), 7.23 (s, 1H); 334.0 (M + H⁺). |
| 132 | 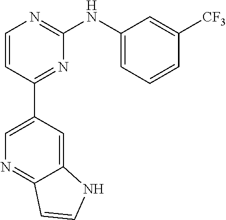 [SMILES: FC(F)(F)c1cccc(Nc2nccc(-c3cnc4cc[nH]c4c3)n2)c1] | A | ¹HNMR (400 MHz, DMSO-d₆): δ = 11.94 (brs, 1H), 10.10 (brs, 1H), 9.21 (s, 1H), 8.73-8.58 (m, 2H), 8.41 (s, 1H), 8.07 (d, J = 7.2 Hz, 1H), 7.98-7.90 (m, 1H), 7.63 (d, J = 5.2, 1H), 7.56 (t, J = 8.0 Hz, 1H), 7.31 (d, J = 7.2 Hz, 1H), 6.72 (s, 1H); 355.9 (M + H⁺). |
| 133 | 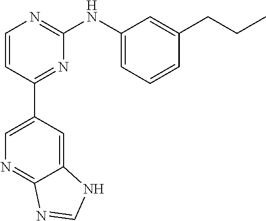 [SMILES: CCCc1cccc(Nc2nccc(-c3cnc4nc[nH]c4c3)n2)c1] | * | ¹H NMR (400 MHz, DMSO- d₆): δ = 9.77 (brs, 1H), 9.37 (s, 1H), 9.29 (s, 1H), 8.92 (s, 1H), 8.62 (d, J = 5.2 Hz, 1H), 7.68 (s, 1H), 7.66-7.63 (m, 2H), 7.38-7.13 (m, 2H), 6.84 (d, J = 7.2 Hz, 1H), 2.57 (t, J = 7.6 Hz, 1H), 1.68-1.59 (m, 2H), 0.93 (t, J = 7.6 Hz, 3H); 331.1 (M + H⁺). |
| 134 | 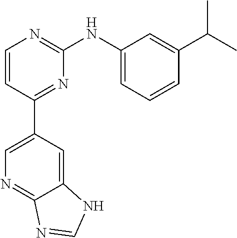 [SMILES: CC(C)c1cccc(Nc2nccc(-c3cnc4nc[nH]c4c3)n2)c1] | * | ¹H NMR (400 MHz, DMSO- d₆): δ = 9.81 (brs, 1H), 9.43- 9.40 (m, 2H), 8.95 (d, J = 1.6 Hz, 1H), 8.63 (d, J = 5.2 Hz, 1H), 7.75 (s, 1H), 7.66-7.62 (m, 2H), 7.28-7.24 (m, 2H), 6.90 (d, J = 7.6 Hz, 1H), 2.90 (t, J = 6.8 Hz, 1H), 1.26 (s, 3H), 1.24 (s, 3H); 331.1 (M + H⁺). |
| 135 | 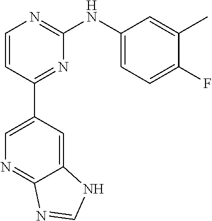 [SMILES: Cc1cc(Nc2nccc(-c3cnc4nc[nH]c4c3)n2)ccc1F] | * | ¹H NMR (400 MHz, DMSO- d₆): δ = 9.79 (brs, 1H), 9.35- 9.33 (m, 2H), 8.91 (d, J = 2.0 Hz, 1H), 8.61 (d, J = 5.2 Hz, 1H), 7.72-7.70 (m, 1H), 7.66- 7.62 (m, 2H), 7.12 (t, J = 9.2 Hz, 1H), 2.26 (d, J = 1.2 Hz, 3H); 321.1 (M + H⁺). |
| 136 | 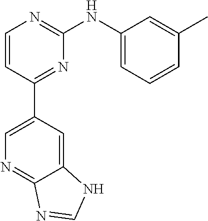 [SMILES: Cc1cccc(Nc2nccc(-c3cnc4nc[nH]c4c3)n2)c1] | * | ¹H NMR (400 MHz, DMSO- d₆): δ = 9.61 (brs, 1H), 9.21 (s, 1H), 8.89 (s, 1H), 8.56 (d, J = 5.2 Hz, 2H), 7.69 (s, 1H), 7.65 (d, J = 8.0 Hz, 1H), 7.56 (d, J = 5.2 Hz, 1H), 7.21 (t, J = 7.6 Hz, 1H), 6.81 (d, J = 7.6 Hz, 1H), 2.33 (s, 3H); 303.1 (M + H⁺). |
| 137 | 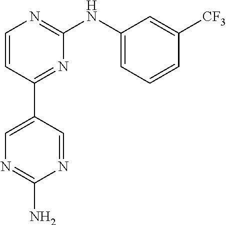 [SMILES: Nc1ncc(-c2ccnc(Nc3cccc(C(F)(F)F)c3)n2)cn1] | B | ¹H NMR (400 MHz, DMSO- d₆): δ = 10.17 (brs, 1H), 9.15 (brs, 2H), 8.59 (d, J = 5.2 Hz, 1H), 8.41 (s, 1H), 7.95 (d, J = 8.0 Hz, 1H), 7.56 (t, J = 8.0 Hz, 1H), 7.49 (d, J = 5.2 Hz, 1H), 7.46-7.20 (m, 2H); 333.0 (M + H⁺). |
| 138 | 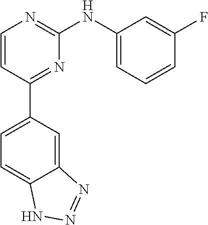 [SMILES: Fc1cccc(Nc2nccc(-c3ccc4[nH]nnc4c3)n2)c1] | B | ¹H NMR (300 MHz, CD₃OD): δ = 8.73 (s, 1H), 8.54 (d, J = 5.2 Hz, 1H), 8.29 (d, J = 8.8 Hz, 1H), 7.97 (d, J = 8.4 Hz, 1H), 7.81 (d, J = 12.4 Hz, 1H), 7.61 (dd, J = 11.6, 7.2 Hz, 1H), 7.55 (d, J = 6.8 Hz, 2H), 7.45 (dd, J = 5.2, 2.4 Hz, 1H), 7.27 (dd, J = 6.8, 2.4 Hz, 1H), 6.84 (dd, J = 5.2, 1.2 Hz, 1H); 306.9 (M + H⁺). |
| 139 | 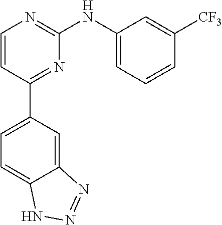 [SMILES: FC(F)(F)c1cccc(Nc2nccc(-c3ccc4[nH]nnc4c3)n2)c1] | A | ¹H NMR (300 MHz, CD₃OD): δ = 8.73 (s, 1H), 8.54 (d, J = 4.8 Hz, 1H), 8.29 (d, J = 4.8 Hz, 1H), 7.97 (d, J = 3.6 Hz, 1H), 7.81 (d, J = 3.6 Hz, 1H), 7.61 (dd, J = 4.8, 3.6 Hz, 1H), 7.55 (d, J = 8.0 Hz, 2H), 7.45 (dd, J = 3.6, 1.2 Hz, 1H), 7.27 (dd, J = 4.8, 1.2 Hz, 1H), 6.84 (dd, J = 2.4, 1.2 Hz, 1H); 357.0 (M + H⁺). |
| 140 | 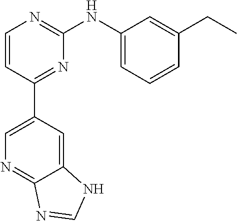 [SMILES: CCc1cccc(Nc2nccc(-c3cnc4nc[nH]c4c3)n2)c1] | * | ¹H NMR (400 MHz, DMSO- d₆): δ = 9.74 (brs, 1H), 9.34 (d, J = 1.6 Hz, 1H), 9.19 (s, 1H), 8.89 (s, 1H), 8.61 (d, J = 5.2 Hz, 1H), 7.72 (s, 1H), 7.64-7.62 (m, 2H), 7.35-7.10 (m, 2H), 6.86 (d, J = 7.2 Hz, 1H), 2.63 (q, J = 7.2 Hz, 2H), 1.23 (t, J = 7.2 Hz, 3H); 317.1 (M + H⁺). |
| 141 | 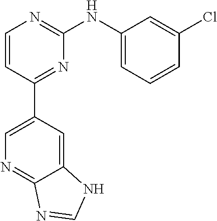 [SMILES: Clc1cccc(Nc2nccc(-c3cnc4nc[nH]c4c3)n2)c1] | * | ¹H NMR (400 MHz, DMSO- d₆): δ = 10.07 (brs, 1H), 9.41- 9.38 (m, 1H), 8.94 (d, J = 2.0 Hz, 1H), 8.68 (d, J = 5.2 Hz, 1H), 8.07 (s, 1H), 7.76 (d, J = 8.4 Hz, 1H), 7.01 (d, J = 5.2 Hz, 1H), 7.39-7.14 (m, 2H), 7.04- 7.02 (m, 1H); 323.0 (M + H⁺). |
| 142 | 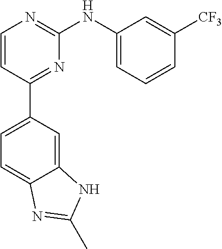 [SMILES: Cc1nc2ccc(-c3ccnc(Nc4cccc(C(F)(F)F)c4)n3)cc2[nH]1] | A | ¹H NMR (400 MHz, DMSO- d₆): δ = 12.45 (brs, 1H), 9.99 (s, 1H), 8.56 (t, J = 5.2 Hz, 1H), 8.50 (s, 1H), 8.31 (s, 1H), 8.02 (t, J = 7.6 Hz, 2H), 7.58-7.52 (m, 3H), 7.29 (d, J = 7.6 Hz, 1H), 2.54 (s, 3H); 370.1 (M + H⁺). |
| 143 | 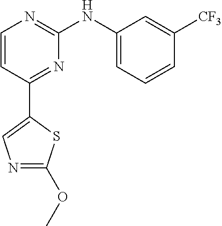 [SMILES: COc1ncc(-c2ccnc(Nc3cccc(C(F)(F)F)c3)n2)s1] | A | ¹H NMR (400 MHz, DMSO- d₆): δ = 10.04 (brs, 1H), 8.52 (d, J = 4.8 Hz, 1H), 8.43 (s, 1H), 8.21 (s, 1H), 7.86 (d, J = 8.4 Hz, 1H), 7.53 (t, J = 8.0 Hz, 1H), 7.41 (d, J = 5.2 Hz, 1H), 7.29 (d, J = 7.6 Hz, 1H), 4.11 (s, 3H); 353.0 (M + H⁺). |
| 144 | 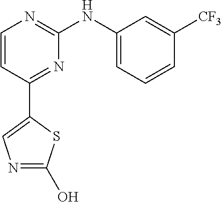 [SMILES: Oc1ncc(-c2ccnc(Nc3cccc(C(F)(F)F)c3)n2)s1] | A | ¹H NMR (400 MHz, CD₃OD): δ = 8.34 (d, J = 6.4 Hz, 1H), 8.31 (s, 1H), 7.20 (s, 1H), 7.84 (d, J = 8.0 Hz, 1H), 7.72 (t, J = 7.6 Hz, 1H), 7.62 (d, J = 7.2 Hz, 1H), 7.48 (d, J = 6.8 Hz, 1H); 339.0 (M + H⁺). |
| 145 | 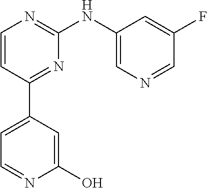 [SMILES: Oc1cc(-c2ccnc(Nc3cncc(F)c3)n2)ccn1] | B | ¹H NMR (400 MHz, DMSO- d₆): δ = 10.47 (brs, 1H), 8.79 (s, 1H), 8.73 (d, J = 4 Hz, 1H), 8.37 (d, J = 4 Hz, 1H), 8.42-8.37 (m, 1H), 8.32-8.30 (m, 1H), 7.62-7.57 (m, 2H), 7.13 (s, 1H), 6.88 (d, J = 4 Hz, 1H); 284.0 (M + H⁺). |
| 146 | 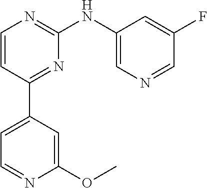 [SMILES: COc1cc(-c2ccnc(Nc3cncc(F)c3)n2)ccn1] | B | ¹H NMR (400 MHz, DMSO- d₆): δ = 10.31 (brs, 1H), 8.79 (s, 1H), 8.73 (d, J = 4 Hz, 1H), 8.37 (d, J = 4 Hz, 1H), 8.33-8.29 (m, 1H), 8.18 (d, J = 4 Hz, 1H), 7.67 (d, J = 4 Hz, 1H), 7.64 (d, J = 4 Hz, 1H), 7.52 (s, 1H), 3.93 (s, 3H); 298.1 (M + H⁺). |
| 147 | 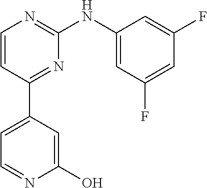 [SMILES: Oc1cc(-c2ccnc(Nc3cc(F)cc(F)c3)n2)ccn1] | B | ¹H NMR (400 MHz, DMSO- d₆): δ = 11.87 (brs, 1H), 10.23 (brs, 1H), 8.70 (d, J = 8 Hz, 1H), 7.60-7.56 (m, 3H), 7.52 (d, J = 8 Hz, 1H), 7.09 (s, 1H), 6.83-6.76 (m, 2H); 301.0 (M + H⁺). |
| 148 | 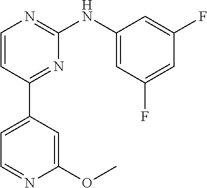 [SMILES: COc1cc(-c2ccnc(Nc3cc(F)cc(F)c3)n2)ccn1] | B | ¹H NMR (400 MHz, DMSO- d₆): δ = 10.28 (brs, 1H), 8.78- 8.75 (m, 1H), 8.43-8.40 (m, 1H), 7.72-7.60 (m, 4H), 7.56- 7.53 (m, 1H), 6.85-6.78 (m, 1H), 3.96 (s, 3H); 315.0 (M + H⁺). |
| 149 | 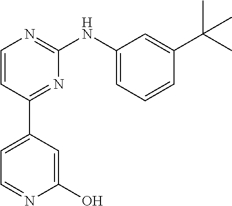 [SMILES: CC(C)(C)c1cccc(Nc2nccc(-c3ccnc(O)c3)n2)c1] | A | ¹H NMR (400 MHz, DMSO- d₆): δ = 11.81 (brs, 1H), 9.68 (brs, 1H), 8.60 (d, J = 8 Hz, 1H), 7.87-7.85 (m, 1H), 7.61 (d, J = 8 Hz, 1H), 7.52 (d, J = 4 Hz, 1H), 7.38 (d, J = 4 Hz, 1H), 7.23 (d, J = 8 Hz, 1H), 7.12- 7.10 (m, 1H), 7.02 (d, J = 8 Hz, 1H), 6.85 (d, J = 8 Hz, 1H), 1.30 (s, 9H); 321.1 (M + H⁺). |
| 150 | 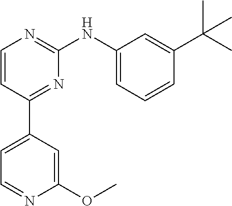 [SMILES: COc1cc(-c2ccnc(Nc3cccc(C(C)(C)C)c3)n2)ccn1] | B | ¹H NMR (400 MHz, DMSO- d₆): δ = 9.71 (brs, 1H), 8.63 (d, J = 8 Hz, 1H), 8.34 (d, J = 8 Hz, 1H), 7.93-7.90 (m, 1H), 7.69 (d, J = 4 Hz, 1H), 7.60 (d, J = 8 Hz, 1H), 7.55 (s, 1H), 7.48 (d, J = 4 Hz, 1H), 7.24 (t, J = 8 Hz, 1H), 7.03 (d, J = 8 Hz, 1H), 3.92 (s, 3H), 1.31 (s, 9H); 335.1 (M + H⁺). |
| 151 | 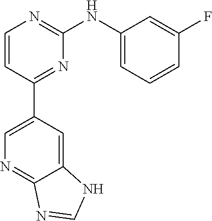 [SMILES: Fc1cccc(Nc2nccc(-c3cnc4nc[nH]c4c3)n2)c1] | * | ¹H NMR (400 MHz, DMSO- d₆): δ = 9.99 (brs, 1H), 9.24 (d, J = 1.2 Hz, 1H), 8.79 (s, 1H), 8.73 (s, 1H), 8.63 (d, J = 5.2 Hz, 1H), 7.91-7.88 (m, 1H), 7.65 (d, J = 5.2 Hz, 1H), 7.58 (d, J = 8.4 Hz, 1H), 7.35 (q, J = 7.6 Hz, 1H), 7.31 (d, J = 7.6 Hz, 1H), 6.81-6.77 (m, 1H); 307.0 (M + H⁺). |
| 152 | 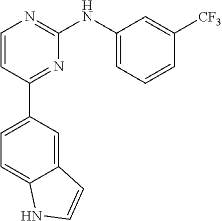 [SMILES: FC(F)(F)c1cccc(Nc2nccc(-c3ccc4[nH]ccc4c3)n2)c1] | B | ¹H NMR(400 MHz, DMSO-d₆): δ = 11.36 (s, 1H), 9.97 (s, 1H), 8.63 (s, 1H), 8.53 (d, J = 5.6 Hz, 1H), 8.50 (s, 1H), 7.99-7.95 (m, 2H), 7.57-7.50 (m, 3H), 7.46-7.44 (m, 1H), 7.30-7.28 (m, 1H), 6.55 (s, 1H); 355.1 (M + H⁺). |
| 153 | 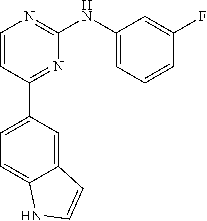 [SMILES: Fc1cccc(Nc2nccc(-c3ccc4[nH]ccc4c3)n2)c1] | B | ¹H NMR(400 MHz, DMSO-d₆): δ = 11.36 (s, 1H), 9.82 (s, 1H), 8.51 (d, J = 5.2 Hz, 1H), 8.45 (s, 1H), 7.98-7.92 (m, 2H), 8.59 (d, J = 8.4 Hz, 1H), 7.53 (d, J = 8.4 Hz, 1H), 7.47-7.43 (m, 2H), 7.34-7.32 (m, 1H), 6.76-6.73 (m, 1H), 6.56 (s, 1H); 305.1 (M + H⁺). |
| 154 | 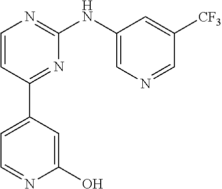 [SMILES: Oc1cc(-c2ccnc(Nc3cncc(C(F)(F)F)c3)n2)ccn1] | A | ¹H NMR (400 MHz, DMSO- d₆): δ = 10.49 (brs, 1H), 9.22- 9.20 (m, 1H), 8.80-8.78 (m, 1H), 8.74 (d, J = 4 Hz, 1H), 8.60-8.58 (m, 1H), 7.61-7.58 (m, 2H), 7.15-7.13 (m, 1H), 6.89-6.86 (m, 1H); 334.0 (M + H⁺). |
| 155 | 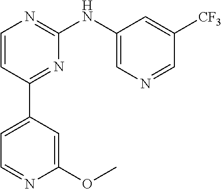 [SMILES: COc1cc(-c2ccnc(Nc3cncc(C(F)(F)F)c3)n2)ccn1] | C | ¹H NMR (400 MHz, DMSO- d₆): δ = 10.44 (brs, 1H), 9.16- 9.14 (m, 1H), 8.83-8.81 (m, 1H), 8.78 (d, J = 4 Hz, 1H), 8.57-8.55 (m, 1H), 8.37 (d, J = 8 Hz, 1H), 7.69-7.68 (m, 2H), 7.53 (m, 1H), 3.93 (s, 3H); 348.0 (M + H⁺). |
| 156 | 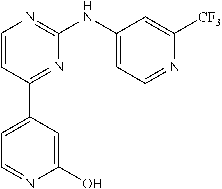 [SMILES: Oc1cc(-c2ccnc(Nc3ccnc(C(F)(F)F)c3)n2)ccn1] | C | ¹H NMR (400 MHz, DMSO- d₆): δ = 10.74 (brs, 1H), 8.80 (d, J = 4 Hz, 1H), 8.57 (d, J = 4 Hz, 1H), 8.43 (s, 1H), 8.38 (d, J = 4 Hz, 1H), 8.03-8.01 (m, 1H), 7.67 (d, J = 4 Hz, 1H), 7.63 (d, J = 8 Hz, 1H), 7.19 (s, 1H), 6.93 (d, J = 8 Hz, 1H); 334.0 (M + H⁺). |
| 157 | 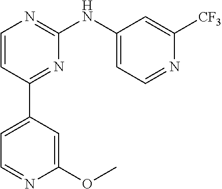 [SMILES: COc1cc(-c2ccnc(Nc3ccnc(C(F)(F)F)c3)n2)ccn1] | C | ¹H NMR (400 MHz, DMSO- d₆): δ = 10.71 (brs, 1H), 8.81 (d, J = 4 Hz, 1H), 8.58 (d, J = 4 Hz, 1H), 8.48 (d, J = 4 Hz, 1H), 8.38 (d, J = 4 Hz, 1H), 8.00- 7.97 (m, 1H), 7.75 (d, J = 4 Hz, 1H), 7.72-7.69 (m, 1H), 7.55 (s, 1H), 3.96 (s, 3H); 348.1 (M + H⁺). |
| 158 | 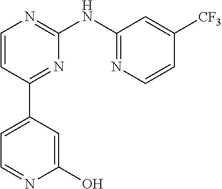 [SMILES: Oc1cc(-c2ccnc(Nc3cc(C(F)(F)F)ccn3)n2)ccn1] | C | ¹H NMR (400 MHz, DMSO- d₆): δ = 11.90 (brs, 1H), 10.63 (brs, 1H), 8.75 (d, J = 4 Hz, 2H), 8.57 (d, J = 4 Hz, 1H), 7.61 (d, J = 4 Hz, 1H), 7.55 (d, J = 4 Hz, 1H), 7.35 (d, J = 4 Hz, 1H), 7.14 (s, 1H), 6.87 (d, J = 4 Hz, 1H); 334.1 (M + H⁺). |
| 159 | 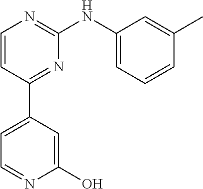 [SMILES: Cc1cccc(Nc2nccc(-c3ccnc(O)c3)n2)c1] | A | ¹H NMR (400 MHz, DMSO- d₆): δ = 11.79 (brs, 1H), 9.68 (brs, 1H), 8.60 (d, J = 8 Hz, 1H), 7.63-7.58 (m, 2H), 7.54 (d, J = 8 Hz, 1H), 7.38 (d, J = 4 Hz, 1H), 7.19 (t, J = 8 Hz, 1H), 7.09 (s, 1H), 6.85-6.79 (m, 2H), 2.30 (s, 3H); 279.1 (M + H⁺). |
| 160 | 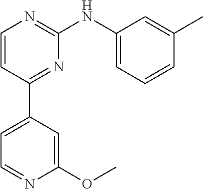 [SMILES: COc1cc(-c2ccnc(Nc3cccc(C)c3)n2)ccn1] | B | ¹H NMR (400 MHz, DMSO- d₆): δ = 9.72 (brs, 1H), 8.63 (d, J = 8 Hz, 1H), 8.36 (d, J = 8 Hz, 1H), 7.69-7.65 (m, 2H), 7.61 (d, J = 4 Hz, 1H), 7.53-7.49 (m, 2H), 7.20 (d, J = 8 Hz, 1H), 6.81 (m, 1H), 3.93 (s, 3H), 3.31 (s, 3H); 293.1 (M + H⁺). |
| 161 | 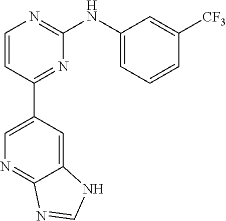 [SMILES: FC(F)(F)c1cccc(Nc2nccc(-c3cnc4nc[nH]c4c3)n2)c1] | * | ¹H NMR (400 MHz, DMSO- d₆): δ = 10.10 (brs, 1H), 9.25 (d, J = 1.6 Hz, 1H), 8.78 (d, J = 1.2 Hz, 1H), 8.69 (s, 1H), 8.64 (d, J = 5.2 Hz, 1H), 8.46 (s, 1H), 8.01 (d, J = 7.6 Hz, 1H), 7.67 (d, J = 5.2 Hz, 1H), 7.56 (t, J = 7.6 Hz, 1H), 7.31 (d, J = 7.6 Hz, 1H), 6.24-6.99 (m, 1H); 357.1 (M + H⁺). |
| 162 | 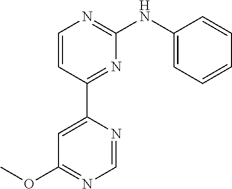 [SMILES: COc1cc(-c2ccnc(Nc3ccccc3)n2)ncn1] | C | ¹H NMR (400 MHz, CDCl₃): δ = 8.88 (s, 1H), 8.62 (d, J = 4.8 Hz, 1H), 7.74 (d, J = 3.6 Hz, 2H), 7.67 (d, J = 8.0 Hz, 2H), 7.37 (t, J = 8.0, 3.6 Hz, 2H), 7.07 (t, J = 4.2, 2.0 Hz, 1H), 4.06 (s, 3H); 280.1 (M + H⁺). |
| 163 | 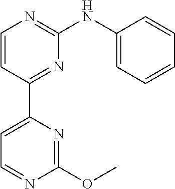 [SMILES: COc1nccc(-c2ccnc(Nc3ccccc3)n2)n1] | C | ¹H NMR (400 MHz, CDCl₃): δ = 8.74 (d, J = 5.2 Hz, 1H), 8.63 (d, J = 5.2 Hz, 1H), 7.97 (d, J = 5.2 Hz, 1H), 7.81 (d, J = 2.8 Hz, 1H), 7.68 (d, J = 8.0 Hz, 2H), 7.37 (dd, J = 7.6, 3.2 Hz, 2H), 7.07 (dd, J = 7.2, 3.2 Hz, 1H), 4.11 (s, 3H); 280.1 (M + H⁺). |
| 164 | 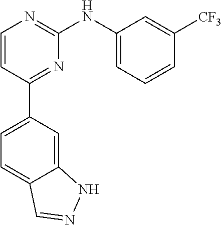 [SMILES: FC(F)(F)c1cccc(Nc2nccc(-c3ccc4cn[nH]c4c3)n2)c1] | B | ¹H NMR (400 MHz, DMSO- d₆): δ = 13.39 (s, 1H), 10.09 (s, 1H), 8.63 (d, J = 5.2, Hz, 1H) 8.442 (s, 1H), 8.35 (s, 1H), 8.17 (s, 1H), 8.06 (d, J = 8.4 Hz, 1H), 7.92 (s, 2H), 7.59-7.53 (m, 2H), 7.30 (d, J = 7.6 Hz, 1H); 356.1 (M + H⁺). |
| 165 | 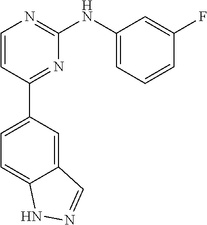 [SMILES: Fc1cccc(Nc2nccc(-c3ccc4[nH]ncc4c3)n2)c1] | B | ¹H NMR (400 MHz, CD₃OD): δ = 12.33 (s, 1H), 9.91 (s, 1H), 8.65 (s, 1H), 8.57 (d, J = 5.2 Hz, 1H), 8.25-8.19 (m, 2H), 7.90 (d, J = 12.8 Hz, 1H), 7.70 (d, J = 8.8 Hz, 1H), 7.60 (d, J = 8.4 Hz, 1H), 7.53 (d, J = 5.2 Hz, 1H), 7.38-7.32 (m, 1H), 6.80- 6.75 (m, 1H); 306.1 (M + H⁺). |
| 166 | 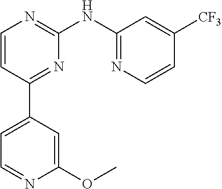 [SMILES: COc1cc(-c2ccnc(Nc3cc(C(F)(F)F)ccn3)n2)ccn1] | C | ¹H NMR (400 MHz, DMSO- d₆): δ = 10.63 (brs, 1H), 8.79- 8.77 (m, 2H), 8.58 (d, J = 4 Hz, 1H), 8.36 (d, J = 4Hz, 1H), 7.72-7.69 (m, 1H), 7.57 (s, 1H), 7.35 (d, J = 4 Hz, 1H), 3.93 (s, 3H); 348.0 (M + H⁺). |
| 167 | 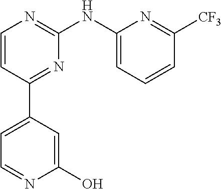 [SMILES: Oc1cc(-c2ccnc(Nc3cccc(C(F)(F)F)n3)n2)ccn1] | C | ¹H NMR (400 MHz, DMSO- d₆): δ = 11.85 (brs, 1H), 10.55 (s, 1H), 8.72 (d, J = 4 Hz, 1H), 8.52 (d, J = 8Hz, 1H), 8.06 (t, J = 8 Hz, 1H), 7.62 (d, J = 4 Hz, 1H), 7.56 (d, J = 4 Hz, 1H), 7.48 (d, J = 4 Hz, 1H), 7.14 (s, 1H), 6.90-6.88 (m, 1H); 334.0 (M + H⁺). |
| 168 | 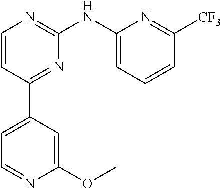 [SMILES: COc1cc(-c2ccnc(Nc3cccc(C(F)(F)F)n3)n2)ccn1] | C | ¹H NMR (400 MHz, DMSO- d₆): δ = 10.52 (brs, 1H), 7.74 (d, J = 4 Hz, 1H), 8.53 (d, J = 8 Hz, 1H), 8.36 (d, J = 4 Hz, 1H), 8.08 (t, J = 4 Hz, 1H), 7.73-7.70 (m, 2H), 7.57 (s, 1H), 7.48 (d, J = 4 Hz, 1H), 3.93 (s, 3H); 348.0 (M + H⁺). |
| 169 | 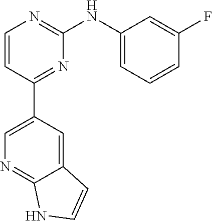 [SMILES: Fc1cccc(Nc2nccc(-c3cnc4[nH]ccc4c3)n2)c1] | C | ¹H NMR (400 MHz, DMSO- d₆): δ = 11.84 (brs, 1H), 9.88 (brs, 1H), 9.21 (d, J = 2.0 Hz, 1H), 8.83 (d, J = 1.2 Hz, 1H), 8.44 (d, J = 5.6 Hz, 1H), 8.00- 7.88 (m, 1H), 7.54 (t, J = 2.8 Hz, 1H), 7.51-7.49 (m, 1H), 7.46-7.40 (m, 1H), 6.90-6.86 (m, 1H), 6.74 (d, J = 4.8 Hz, 1H), 6.59-6.58 (m, 1H); 306.1 (M + H⁺). |
| 170 | 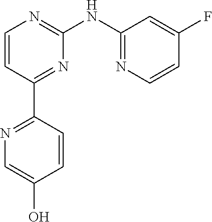 [SMILES: Oc1ccc(-c2ccnc(Nc3cc(F)ccn3)n2)nc1] | B | ¹H NMR (400 MHz, DMSO- d₆): δ = 10.52 (brs, 1H), 9.88 (brs, 1H), 8.58 (d, J = 5.2 Hz, 1H), 8.29-8.25 (m, 1H), 8.86- 7.83 (m, 1H), 7.65 (d, J = 5.2 Hz, 1H), 7.58 (d, J = 4.4 Hz, 1H), 7.39-7.31 (m, 2H), 6.79- 6.74 (m, 1H); 283.0 (M⁺). |
| 171 | 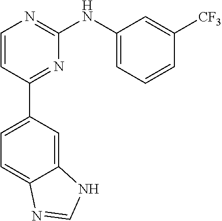 [SMILES: FC(F)(F)c1cccc(Nc2nccc(-c3ccc4nc[nH]c4c3)n2)c1] | * | ¹H NMR(400 MHz, DMSO-d₆): δ = 12.74 (brs, 1H), 10.01 (s, 1H), 8.58 (d, J = 5.6 Hz, 1H), 8.49-8.33 (m, 2H), 8.35 (s, 1H), 8.09 (d, J = 8.0 Hz, 1H), 8.03 (d, J = 8.0 Hz, 1H), 7.71-7.70 (m, 1H), 7.57-7.53 (m, 2H), 7.29 (d, J = 7.8 Hz, 1H); 356.1 (M + H⁺). |
| 172 | 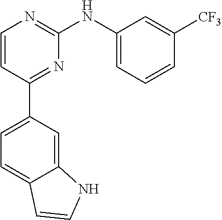 [SMILES: FC(F)(F)c1cccc(Nc2nccc(-c3ccc4cc[nH]c4c3)n2)c1] | B | ¹H NMR(400 MHz, DMSO-d₆): δ = 11.41 (s, 1H), 9.98 (s, 1H), 8.55 (d, J = 5.2 Hz, 1H), 8.46 (s, 1H), 8.26 (s, 1H), 8.07 (d, J = 8.0 Hz, 1H), 7.87-7.85 (m, 1H), 7.67 (d, J = 8.4 Hz, 1H), 7.57-7.45 (m, 2H), 7.29 (d, J = 7.6 Hz, 1H), 6.52 (s, 1H); 355.1 (M + H⁺). |
| 173 | 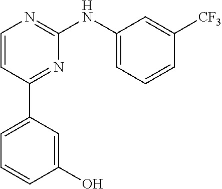 [SMILES: Oc1cccc(-c2ccnc(Nc3cccc(C(F)(F)F)c3)n2)c1] | C | ¹H NMR (400 MHz, DMSO- d₆): δ = 10.00 (s, 1H), 9.54 (s, 1H), 8.50-8.40 (m, 2H), 7.92 (d, J = 9.6 Hz, 1H), 7.81-7.75 (m, 2H), 7.61 (t, J = 8.0 Hz, 1H), 7.37 (d, J = 8.0 Hz, 1H), 7.28 (t, J = 8.0 Hz, 1H), 6.89 (d, J = 1.6 Hz, 1H), 6.77-6.72 (m, 1H); 332.0 (M + H⁺). |
| 174 | 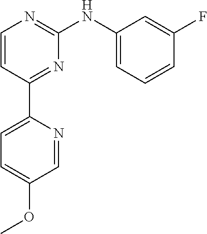 [SMILES: COc1ccc(-c2ccnc(Nc3cccc(F)c3)n2)nc1] | C | ¹H NMR (400 MHz, DMSO- d₆): δ = 9.93 (brs, 1H), 8.62 (d, J = 5.2 Hz, 1H), 8.45 (d, J = 2.8 Hz, 1H), 8.36 (d, J = 8.8 Hz, 1H), 7.89-7.85 (m, 1H), 7.68 (d, J = 5.2 Hz, 1H), 7.65 (dd, J = 8.8, 2.8 Hz, 1H), 7.59-7.57 (m, 1H), 7.34 (q, J = 8.0 Hz, 1H), 6.80-6.75 (m, 1H), 3.93 (s, 3H); 297.1 (M + H⁺). |
| 175 | 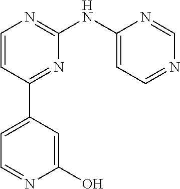 [SMILES: Oc1cc(-c2ccnc(Nc3ccncn3)n2)ccn1] | C | ¹H NMR (400 MHz, DMSO- d₆): δ = 8.80 (brs, 1H), 8.74 (d, J = 4 Hz, 1H), 8.60 (d, J = 4 Hz, 1H), 8.32 (d, J = 4 Hz, 1H), 7.63-7.61 (m, 2H), 6.99 (s, 1H), 6.79 (d, J = 4 Hz, 1H); 267.0 (M + H⁺). |
| 176 | 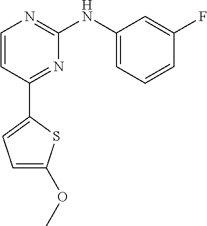 [SMILES: COc1ccc(-c2ccnc(Nc3cccc(F)c3)n2)s1] | B | ¹H NMR (400 MHz, DMSO- d₆): δ = 9.85 (s, 1H), 8.40 (d, J = 5.6 Hz, 1H), 7.87-7.83 (m, 1H), 7.80 (d, J = 4.0 Hz, 1H), 7.50-7.47 (m, 3H), 7.35-7.28 (m, 2H), 7.12-7.10 (m, 2H), 6.79-6.75 (m, 1H), 6.49 (d, J = 4.4 Hz, 1H), 3.96 (s, 3H), 2.29 (s, 3H); 302.0 (M + H⁺). |
| 177 | 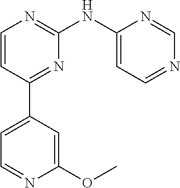 [SMILES: COc1cc(-c2ccnc(Nc3ccncn3)n2)ccn1] | C | ¹H NMR (400 MHz, DMSO- d₆): δ = 10.70 (brs, 1H), 8.82- 8.80 (m, 2H), 8.63 (d, J = 4 Hz, 1H), 8.38 (d, J = 4 Hz, 1H), 8.34 (d, J = 4 Hz, 1H), 7.78 (d, J = 4 Hz, 1H), 7.57 (s, 1H), 3.94 (s, 3H); 281.1 (M + H⁺). |
| 178 | 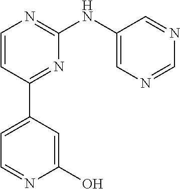 [SMILES: Oc1cc(-c2ccnc(Nc3cncnc3)n2)ccn1] | C | ¹H NMR (400 MHz, DMSO- d₆): δ = 9.25 (s, 2H), 8.78 (s, 1H), 8.82 (s, 1H), 8.57 (d, J = 4 Hz, 1H), 7.74 (d, J = 4 Hz, 1H), 7.35 (d, J = 4 Hz, 1H), 6.63 (s, 1H), 6.57 (d, J = 4 Hz, 1H); 267.0 (M + H⁺). |
| 179 | 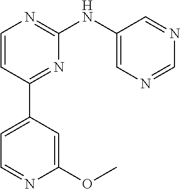 [SMILES: COc1cc(-c2ccnc(Nc3cncnc3)n2)ccn1] | C | ¹H NMR (400 MHz, DMSO- d₆): δ = 10.17 (brs, 1H), 9.22 (s, 2H), 8.82 (s, 1H), 8.72 (d, J = 4 Hz, 1H), 8.38 (d, J = 4 Hz, 1H), 7.67 (d, J = 4 Hz, 1H), 7.63 (d, J = 4 Hz, 1H), 7.52 (s, 1H), 3.92 (s, 3H); 281.1 (M + H⁺). |
| 180 | 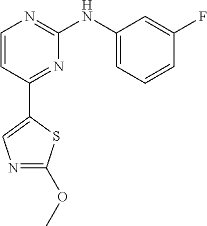 [SMILES: COc1ncc(-c2ccnc(Nc3cccc(F)c3)n2)s1] | B | ¹H NMR (400 MHz, DMSO- d₆): δ = 9.89 (brs, 1H), 8.50 (d, J = 5.2 Hz, 1H), 8.20 (s, 1H), 7.83-7.79 (m, 1H), 7.50-7.48 (m, 1H), 7.38 (d, J = 5.2 Hz, 1H), 7.31(q, J = 7.6 Hz, 1H), 6.79-6.74 (m, 1H), 4.11 (s, 3H); 303.0 (M + H⁺). |
| 181 | 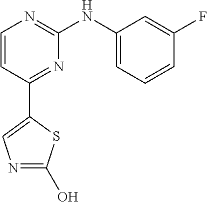 [SMILES: Oc1ncc(-c2ccnc(Nc3cccc(F)c3)n2)s1] | A | ¹H NMR (400 MHz, DMSO- d₆): δ = 11.84 (brs, 1H), 9.83 (brs, 1H), 8.43 (d, J = 5.2 Hz, 1H), 8.07 (s, 1H), 7.85-7.81 (m, 1H), 7.49-7.47 (m, 1H), 7.33- 7.27 (m, 2H), 6.77-6.73 (m, 1H); 289.0 (M + H⁺). |
| 182 | 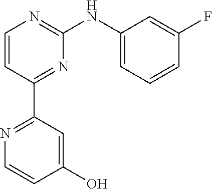 [SMILES: Oc1ccnc(-c2ccnc(Nc3cccc(F)c3)n2)c1] | B | ¹H NMR (400 MHz, DMSO- d₆): δ = 9.85 (brs, 1H), 8.55 (d, J = 5.2 Hz, 1H), 8.04 (d, J = 6.0 Hz, 1H), 7.86-7.83 (m, 1H), 7.69 (d, J = 4.8 Hz, 1H), 7.64 (d, J = 8.4 Hz, 1H), 7.47 (s, 1H), 7.30 (q, J = 8.0 Hz, 1H), 7.74-6.70 (m, 1H), 6.48-6.47 (m, 1H); 283.1 (M + H⁺). |
| 183 | 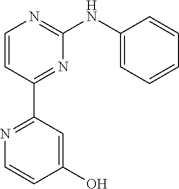 [SMILES: Oc1ccnc(-c2ccnc(Nc3ccccc3)n2)c1] | B | ¹H NMR (400 MHz, CDCl₃): δ = 10.96 (brs, 1H), 9.69 (brs, 1H), 8.60 (d, J = 5.2 Hz, 1H), 8.42 (d, J = 5.2 Hz, 1H), 7.85- 7.83 (m, 2H), 7.67 (d, J = 4.8 Hz, 1H), 7.34 (t, J = 7.6 Hz, 2H), 6.99 (t, J = 7.6 Hz, 1H), 6.93-6.91 (m, 1H); 265.1 (M + H⁺). |
| 184 | 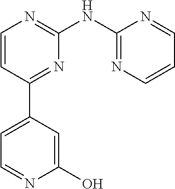 [SMILES: Oc1cc(-c2ccnc(Nc3ncccn3)n2)ccn1] | C | ¹H NMR(400 MHz, DMSO-d₆): δ = 11.83 (brs, 1H), 8.83-8.81 (m, 3H), 7.83 (d, J = 5.6 Hz, 1H), 7.60 (d, J = 6.8 Hz, 1H), 7.32-7.31 (m, 1H), 7. 22 (s, 1H), 6.94-6.92 (m, 1H); 267.0 (M + H⁺). |
| 185 | 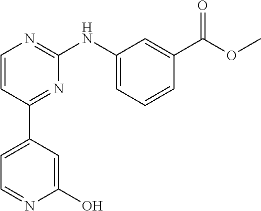 [SMILES: COC(=O)c1cccc(Nc2nccc(-c3ccnc(O)c3)n2)c1] | B | ¹H NMR (400 MHz, DMSO- d₆): δ = 11.83 (brs, 1H), 10.04 (s, 1H), 8.72 (s, 1H), 8.65 (d, J = 5.2 Hz, 1H), 7.93 (dd, J = 8.4, 1.2 Hz, 1H), 7.59-7.44 (m, 4H), 7.16 (s, 1H), 6.91-6.88 (m, 1H), 3.91 (s, 3H); 323.0 (M + H⁺). |
| 186 | 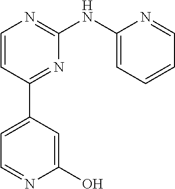 [SMILES: Oc1cc(-c2ccnc(Nc3ccccn3)n2)ccn1] | C | ¹H NMR (400 MHz, DMSO- d₆): δ = 12.28 (brs, 1H), 8.88 (d, J = 4 Hz, 1H), 8.49 (d, J = 8 Hz, 1H), 8.36-8.32 (m, 1H), 7.88 (d, J = 4 Hz, 1H), 7.80 (d, J = 8 Hz, 1H), 7.62 (d, J = 4 Hz, 1H), 7.42 (t, J = 4 Hz, 1H), 7.20-7.18 (m, 1H), 6.89-6.91 (m, 1H); 266.1 (M + H⁺). |
| 187 | 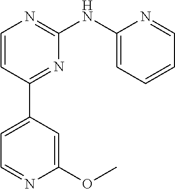 [SMILES: COc1cc(-c2ccnc(Nc3ccccn3)n2)ccn1] | C | ¹H NMR (400 MHz, DMSO- d₆): δ = 9.67 (brs, 1H), 8.71 (d, J = 4 Hz, 1H), 8.36 (d, J = 4 Hz, 1H), 8.33-8.29 (m, 2H), 7.84- 7.81 (m, 1H), 7.72-7.70 (m, 1H), 7.62 (d, J = 4 Hz, 1H), 7.55 (s, 1H), 7.04-7.01 (m, 1H), 3.91 (s, 3H); 280.1 (M + H⁺). |
| 188 | 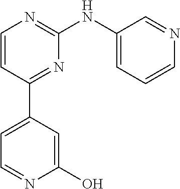 [SMILES: Oc1cc(-c2ccnc(Nc3cccnc3)n2)ccn1] | B | ¹H NMR (400 MHz, DMSO- d₆): δ = 10.74 (brs, 1H), 9.39 (brs, 1H), 8.77 (d, J = 4 Hz, 1H), 8.67-8.65 (m, 1H), 8.50 (d, J = 4 Hz, 1H), 7.98-7.94 (m, 1H), 7.63 (d, J = 4 Hz, 1H), 7.58-7.56 (m, 1H), 7.12 (d, J = 4 Hz, 1H), 6.88-6.86 (m, 1H); 266.1 (M + H⁺). |
| 189 | 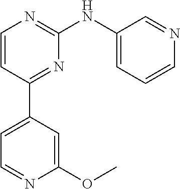 [SMILES: COc1cc(-c2ccnc(Nc3cccnc3)n2)ccn1] | B | ¹H NMR (400 MHz, DMSO- d₆): δ = 10.01 (brs, 1H), 8.97 (d, J = 4 Hz, 1H), 8.69 (d, J = 4 Hz, 1H), 8.36 (d, J = 4 Hz, 1H), 8.25-8.20 (m, 2H), 7.69-7.67 (m, 1H), 7.57 (d, J = 4 Hz, 1H), 7.51 (s, 1H), 7.39-7.35 (m, 1H), 3.93 (s, 3H); 280.1 (M + H⁺). |
| 190 | 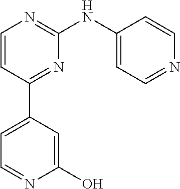 [SMILES: Oc1cc(-c2ccnc(Nc3ccncc3)n2)ccn1] | B | ¹H NMR (400 MHz, DMSO- d₆): δ = 11.84 (brs, 1H), 11.09 (brs, 1H), 8.84 (d, J = 4 Hz, 1H), 8.57-8.55 (m, 2H), 8.11- 8.09 (m, 2H), 7.74 (d, J = 4 Hz, 1H), 7.59 (d, J = 4 Hz, 1H), 7.14 (s, 1H), 6.88 ( d, J = 4 Hz, 1H); 266.1 (M + H⁺). |
| 191 | 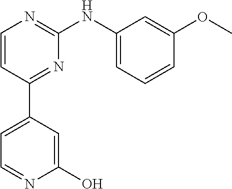 [SMILES: COc1cccc(Nc2nccc(-c3ccnc(O)c3)n2)c1] | B | ¹H NMR (400 MHz, DMSO- d₆): δ = 11.83 (brs, 1H), 9.77 (s, 1H), 8.62 (d, J = 4 Hz, 1H), 7.59-7.57 (m, 2H), 7.55 (d, J = 8 Hz, 1H), 7.41 (d, J = 8 Hz, 1H), 7.33-7.31 (m, 1H), 7.22 (t, J = 8 Hz, 1H), 6.86-6.84 (m, 1H), 6.58-6.55 (m, 1H), 3.76 (s, 3H); 295.1 (M + H⁺). |
| 192 | 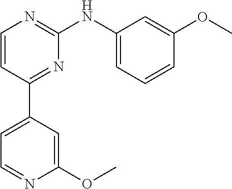 [SMILES: COc1cccc(Nc2nccc(-c3ccnc(OC)c3)n2)c1] | B | ¹H NMR (400 MHz, DMSO- d₆): δ = 9.78 (brs, 1H), 8.65 (d, J = 8 Hz, 1H), 8.35 (d, J = 8 Hz, 1H), 7.69-7.67 (m, 1H), 7.62- 7.60 (m, 1H), 7.54-7.51 (m, 2H), 7.36-7.33 (m, 1H), 7.21 (t, J = 8 Hz, 1H), 6.58-6.56 (m, 1H), 3.93 (s, 3H), 3.77(s, 3H); 309.1 (M + H⁺). |
| 193 | 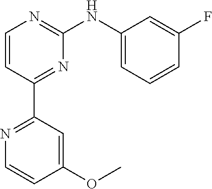 [SMILES: COc1ccnc(-c2ccnc(Nc3cccc(F)c3)n2)c1] | C | ¹H NMR (400 MHz, DMSO- d₆): δ = 10.04 (brs, 1H), 8.68 (d, J = 4.4 Hz, 1H), 8.57 (d, J = 5.2 Hz, 1H), 7.96-7.93 (m, 2H), 7.76 (d, J = 4.8 Hz, 1H), 7.54 (d, J = 7.6 Hz, 1H), 7.38-7.32 (m, 1H), 7.17 (overlap, 1H), 7.69 (t, J = 7.6 Hz, 1H), 3.96 (s, 3H); 297.1 (M + H⁺). |
| 194 | 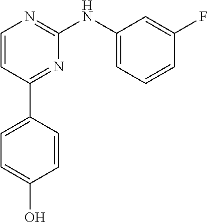 [SMILES: Oc1ccc(-c2ccnc(Nc3cccc(F)c3)n2)cc1] | B | ¹H NMR (400 MHz, CD₃OD): δ = 7.49 (d, J = 5.2 Hz, 1H), 7.14 (d, J = 8.8 Hz, 2H), 6.94-6.91 (m, 1H), 6.52-6.49 (m, 1H), 6.40-6.33 (m, 2H), 6.00 (d, J = 8.8 Hz, 2H), 5.82-5.77 (m, 1H); 282.1 (M + H⁺). |
| 195 | 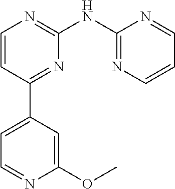 [SMILES: COc1cc(-c2ccnc(Nc3ncccn3)n2)ccn1] | C | ¹H NMR (400 MHz, DMSO- d₆): δ = 10.41 (s, 1H), 8.74 (d, J = 5.6 Hz, 1H), 8.63 (d, J = 5.2 Hz, 1H), 8.34 (d, J = 5.6 Hz, 1H), 7.73-7.70 (m, 2H), 7.58 (s, 1H), 7.08 (t, J = 4.8 Hz, 1H), 3.92 (s, 3H); 281.1 (M + H⁺). |
| 196 | 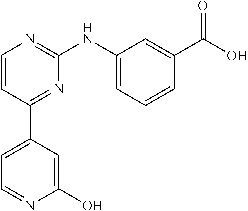 [SMILES: O=C(O)c1cccc(Nc2nccc(-c3ccnc(O)c3)n2)c1] | C | ¹H NMR (400 MHz, DMSO- d₆): δ = 12.10 (brs, 2H), 9.99 (s, 1H), 8.65 (d, J = 5.2 Hz, 1H), 8.55 (s, 1H), 7.98 (d, J = 8.4 Hz, 1H), 7.57-7.42 (m, 4H), 7.12 (s, 1H), 6.94-6.92 (m, 1H); 309.1 (M + H⁺). |
| 197 | 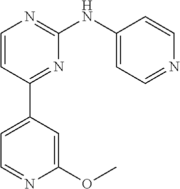 [SMILES: COc1cc(-c2ccnc(Nc3ccncc3)n2)ccn1] | B | 280.1 (M + H⁺) |
| 198 | 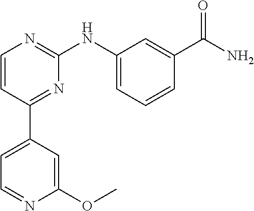 [SMILES: COc1cc(-c2ccnc(Nc3cccc(C(N)=O)c3)n2)ccn1] | B | ¹H NMR (400 MHz, DMSO- d₆): δ = 9.90 (brs, 1H), 8.65 (d, J = 8 Hz, 1H), 8.41-8.39 (m, 1H), 8.33 (d, J = 8 Hz, 1H), 7.90-7.87 (m, 2H).7.74 (d, J = 4 Hz, 1H), 7.55-7.53 (m, 2H), 7.48 (d, J = 8 Hz, 1H), 7.38 (t, J = 8 Hz, 1H), 7.34-7.31 (m, 1H), 3.93 (s, 3H); 322.1 (M + H⁺). |
| 199 | 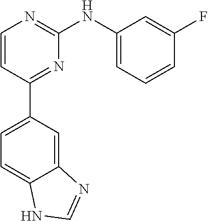 [SMILES: Fc1cccc(Nc2nccc(-c3ccc4[nH]cnc4c3)n2)c1] | * | ¹H NMR (400 MHz, DMSO- d₆): δ = 12.86-12.61 (m, 1H), 9.90 (d, J = 10.4 Hz, 1H), 8.65- 8.26 (m, 3H), 8.15-7.99 (m, 1H), 7.98-7.76 (m, 1H), 7.84- 7.64 (m, 1H), 7.63-7.46 (m, 2H), 7.34 (dd, J = 15.2, 8.0 Hz, 1H), 6.77 (t, J = 7.6 Hz, 1H); 306.1 (M + H⁺). |
| 200 | 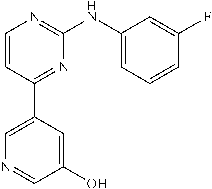 [SMILES: Oc1cncc(-c2ccnc(Nc3cccc(F)c3)n2)c1] | B | ¹H NMR (400 MHz, DMSO- d₆): δ = 10.28 (brs, 1H), 9.97 (brs, 1H), 8.80 (d, J = 1.2 Hz, 1H), 8.62 (d, J = 5.2 Hz, 1H), 8.30 (d, J = 5.2 Hz, 1H), 7.86- 7.82 (m, 2H), 7.58 (dd, J = 8.4, 1.2 Hz, 1H), 7.34 (q, J = 8.0 Hz, 1H), 6.78 (t, J = 8.8 Hz, 1H); 283.0 (M + H⁺). |
| 201 | 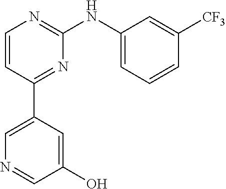 [SMILES: Oc1cncc(-c2ccnc(Nc3cccc(C(F)(F)F)c3)n2)c1] | B | ¹H NMR (400 MHz, DMSO- d6): δ = 10.27 (brs, 1H), 10.10 (brs, 1H), 8.82 (d, J = 1.2 Hz, 1H), 8.64 (d, J = 1.2 Hz, 1H), 8.38 (s, 1H), 8.31 (d, J = 2.8 Hz, 1H), 8.04 (d, J = 8.0 Hz, 1H), 7.86 (t, J = 2.2 Hz, 1H), 7.56 (t, J = 8.0 Hz, 1H), 7.52 (d, J = 5.2 Hz, 1H), 7.30 (d, J = 7.6 Hz, 1H); 333.1 (M + H⁺). |
| 202 | 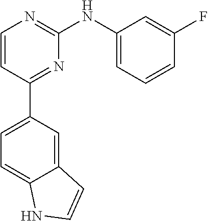 [SMILES: Fc1cccc(Nc2nccc(-c3ccc4[nH]ccc4c3)n2)c1] | C | ¹H NMR (400 MHz, DMSO- d₆): δ = 11.45 (brs, 1H), 9.85 (brs, 1H), 8.53 (d, J = 5.2 Hz, 1H), 8.26 (s, 1H), 7.95-7.87 (m, 1H), 7.83 (d, J = 8.4, 1.6 Hz, 1H), 7.68 (d, J = 8.4 Hz, 1H), 7.62 (dd, J = 8.0, 1.2 Hz, 1H), 7.53 (t, J = 2.8 Hz, 1H), 7.46 (d, J = 5.2 Hz, 1H), 7.33 (dd, J = 15.6, 8.0 Hz, 1H), 6.81-6.72 (m, 1H), 6.55-6.48 (m, 1H); 305.1 (M + H⁺). |
| 203 | 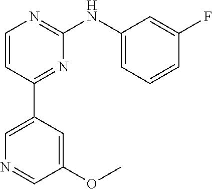 [SMILES: COc1cncc(-c2ccnc(Nc3cccc(F)c3)n2)c1] | A | ¹H NMR (400 MHz, DMSO- d₆): δ = 10.04 (brs, 1H), 8.96 (s, 1H), 8.67 (m, 1H), 8.47 (s, 1H), 8.07 (s, 1H), 7.93-7.90 (m, 1H), 7.62-7.54 (m, 2H), 7.35-7.34 (m, 1H), 6.79 (m, 1H), 3.95 (s, 3H); 297.1 (M + H⁺). |
| 204 | 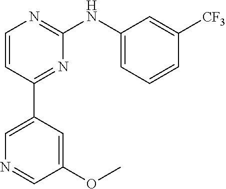 [SMILES: COc1cncc(-c2ccnc(Nc3cccc(C(F)(F)F)c3)n2)c1] | A | ¹H NMR (400 MHz, DMSO- d₆): δ = 10.16 (brs, 1H), 8.96 (d, J = 1.2 Hz, 1H), 8.68 (d, J = 5.2 Hz, 1H), 8.47 (s, 1H), 8.46-8.44 (m, 1H), 8.05 (s, 1H), 7.98 (d, J = 8.0 Hz, 1H), 7.64 (d, J = 4.8 Hz, 1H), 7.56 (t, J = 8.0 Hz, 1H), 7.31 (d, J = 7.6 Hz, 1H), 3.94 (s, 3H); 347.1 (M + H⁺). |
| 205 | 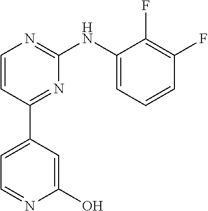 [SMILES: Oc1cc(-c2ccnc(Nc3cccc(F)c3F)n2)ccn1] | B | ¹H NMR (400 MHz, DMSO- d₆): δ = 11.80 (brs, 1H), 9.58 (s, 1H), 8.59 (dd, J = 4.8, 1.2 Hz, 1H), 7.54-7.51 (m, 2H), 7.46- 7.45 (m, 1H), 7.21-7.18 (m, 2H), 7.04 (s, 1H), 6.77 (d, J = 10.0 Hz, 1H); 300.9 (M + H⁺). |
| 206 | 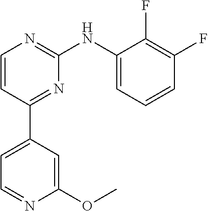 [SMILES: COc1cc(-c2ccnc(Nc3cccc(F)c3F)n2)ccn1] | C | ¹H NMR (400 MHz, DMSO- d₆): δ = 9.58 (s, 1H), 8.62 (d, J = 5.2 Hz, 1H), 8.33 (d, J = 5.2 Hz, 1H), 7.63-7.51 (m, 3H), 7.46 (s, 1H), 7.20 (t, J = 7.2 Hz, 2H), 3.91 (s, 3H); 315.1 (M + H⁺). |
| 207 | 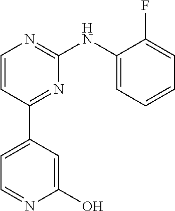 [SMILES: Oc1cc(-c2ccnc(Nc3ccccc3F)n2)ccn1] | C | ¹H NMR (400 MHz, DMSO- d₆): δ = 11.79 (brs, 1H), 9.30 (s, 1H), 8.55 (d, J = 5.6 Hz, 1H), 7.73-7.71 (m, 1H), 7.50 (d, J = 6.4 Hz, 1H), 7.40 (d, J = 5.2 Hz, 1H), 7.25-7.18 (m, 3H), 7.02 (s, 1H), 6.77 (d, J = 5.6 Hz, 1H); 282.9 (M + H⁺). |
| 208 | 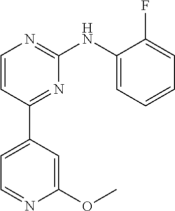 [SMILES: COc1cc(-c2ccnc(Nc3ccccc3F)n2)ccn1] | C | ¹H NMR (400 MHz, DMSO- d₆): δ = 9.30 (s, 1H), 8.58 (d, J = 5.2 Hz, 1H), 8.32 (d, J = 5.2 Hz, 1H), 7.78-7.73 (m, 1H), 7.61 (dd, J = 9.6, 1.6 Hz, 1H), 7.50 (d, J = 5.2 Hz, 1H), 7.45 (s, 1H), 7.30-7.16 (m, 3H), 3.91 (s, 3H); 297.1 (M + H⁺). |
| 209 | 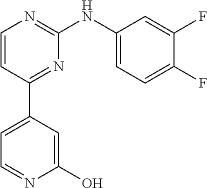 [SMILES: Oc1cc(-c2ccnc(Nc3ccc(F)c(F)c3)n2)ccn1] | B | ¹H NMR (400 MHz, DMSO- d₆): δ = 11.84 (brs, 1H), 10.03 (s, 1H), 8.65 (d, J = 5.6 Hz, 1H), 8.03-7.98 (m, 1H), 7.57- 7.35 (m, 4H), 7.08 (s, 1H), 6.83 (d, J = 7.2 Hz, 1H); 301.0 (M + H⁺). |
| 210 | 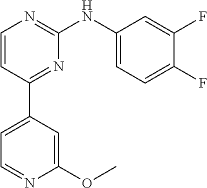 [SMILES: COc1cc(-c2ccnc(Nc3ccc(F)c(F)c3)n2)ccn1] | B | ¹H NMR (400 MHz, DMSO- d₆): δ = 10.02 (s, 1H), 8.68 (d, J = 5.2 Hz, 1H), 8.37 (d, J = 5.2 Hz, 1H), 8.05-8.00 (m, 1H), 7.67 (dd, J = 5.2, 1.6 Hz, 1H), 7.57-7.31 (m, 3H), 7.43-7.35 (m, 1H), 3.93 (s, 3H); 315.1 (M + H⁺). |
| 211 | 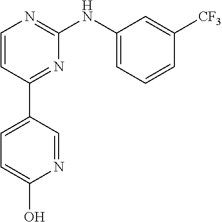 [SMILES: Oc1ccc(-c2ccnc(Nc3cccc(C(F)(F)F)c3)n2)cn1] | A | ¹H NMR (400 MHz, DMSO- d₆): δ = 10.05 (brs, 1H), 8.51 (d, J = 8 Hz, 1H), 8.34-8.31 (m, 2H), 8.21 (dd, J = 8, 4 Hz, 1H), 7.98-7.96 (m, 1H), 7.53 (t, J = 4 Hz, 1H), 7.38 (d, J = 4 Hz, 1H), 7.30 (d, J = 8 Hz, 1H), 6.48 (d, J = 8 Hz, 1H); 333.1 (M + H⁺). |
| 212 | 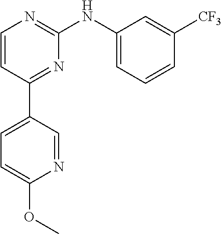 [SMILES: COc1ccc(-c2ccnc(Nc3cccc(C(F)(F)F)c3)n2)cn1] | B | ¹H NMR (400 MHz, DMSO- d₆): δ = 10.05 (brs, 1H), 9.00 (m, 1H), 8.60 (d, J = 8 Hz, 1H), 8.44 (dd, J = 8.0, 4.0 Hz, 1H), 8.40 (m, 1H), 8.01 (d, J = 8 Hz, 1H), 7.57-7.49 (m, 2H), 7.29 (d, J = 8 Hz, 1H), 6.99 (d, J = 8 Hz, 1H), 6.79-6.75 (m, 1H), 3.95 (s, 3H); 347.1 (M + H⁺). |
| 213 | 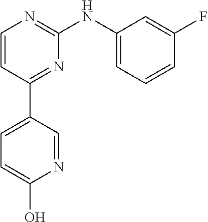 [SMILES: Oc1ccc(-c2ccnc(Nc3cccc(F)c3)n2)cn1] | B | ¹H NMR (400 MHz, DMSO- d₆): δ = 9.95 (brs, 1H), 8.49 (d, J = 4 Hz, 1H), 8.34 (m, 1H), 8.20 (dd, J = 4 Hz, 1H), 7.80- 7.77 (m, 1H), 7.52-7.49 (m, 1H), 7.36-7.31 (m, 2H), 7.08- 7.06 (m, 1H), 6.80-6.76 (m, 1H), 6.50 (d, J = 4 Hz, 1H); 283.1 (M + H⁺) |
| 214 | 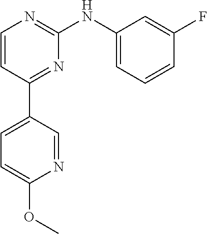 [SMILES: COc1ccc(-c2ccnc(Nc3cccc(F)c3)n2)cn1] | C | ¹H NMR (400 MHz, DMSO- d₆): δ = 9.91 (brs, 1H), 9.00 (d, J = 4 Hz, 1H), 8.58 (d, J = 4 Hz, 1H), 8.43 (dd, J = 4 Hz, 1H), 7.86-7.83 (m, 1H), 7.56 (d, J = 8 Hz, 1H), 7.47 (d, J = 4 Hz, 1H), 7.36-7.38 (m, 1H), 7.00 (d, J = 4 Hz, 1H), 6.79-6.75 (m, 1H), 3.95 (s, 3H); 297.1 (M + H⁺). |
| 215 | 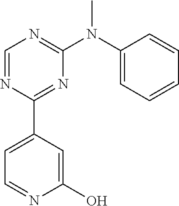 [SMILES: CN(c1ccccc1)c1ncnc(-c2ccnc(O)c2)n1] | C | ¹H NMR (400 MHz, DMSO- d₆): δ = 11.83 (brs, 1H), 8.75 (brs, 1H), 7.58-7.38 (m, 5H), 7.34 (t, J = 7.2 Hz, 1H), 7.29- 7.05 (m, 1H), 7.01-6.56 (m, 1H), 3.56 (s, 3H); 280.1 (M + H⁺). |
| 216 | 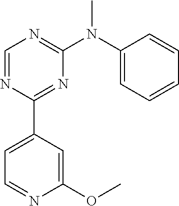 [SMILES: COc1cc(-c2ncnc(N(C)c3ccccc3)n2)ccn1] | C | ¹H NMR (400 MHz, DMSO- d₆): δ = 8.77 (brs, 1H), 8.33 (brs, 1H), 7.71 (brs, 1H), 7.52- 7.39 (m, 5H), 7.35 (t, J = 7.2 Hz, 1H), 3.90 (s, 3H), 3.58 (s, 3H); 294.1 (M + H⁺). |
| 217 | 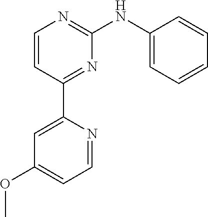 [SMILES: COc1ccnc(-c2ccnc(Nc3ccccc3)n2)c1] | C | ¹H NMR (400 MHz, CDCl₃): δ = 8.56 (d, J = 4.8 Hz, 1H), 8.52 (d, J = 5.6 Hz, 1H), 8.00 (d, J = 2.0 Hz, 1H), 7.76 (d, J = 4.8 Hz, 1H), 7.72 (d, J = 7.6 Hz, 2H), 7.37 (t, J = 7.6 Hz, 2H), 7.22 (overlap, 1H), 7.07 (t, J = 7.2 Hz, 1H), 6.93-6.91 (m, 1H), 3.96 (s, 3H); 279.1 (M + H⁺). |
| 218 | 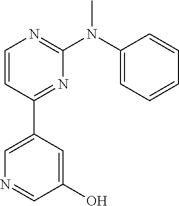 [SMILES: CN(c1ccccc1)c1nccc(-c2cncc(O)c2)n1] | C | ¹H NMR (400 MHz, DMSO- d₆): δ = 10.17 (brs, 1H), 8.70 (d, J = 1.2 Hz, 1H), 8.45 (d, J = 5.2 Hz, 1H), 8.24 (d, J = 2.4 Hz, 1H), 7.75 (t, J = 2.6 Hz, 1H), 7.42-7.40 (m, 4H), 7.34 (d, J = 5.2 Hz, 1H), 7.26-7.21 (m, 1H), 3.56 (s, 3H); 279.1 (M + H⁺). |
| 219 | 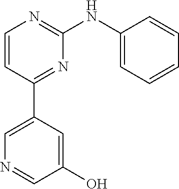 [SMILES: Oc1cncc(-c2ccnc(Nc3ccccc3)n2)c1] | B | ¹H NMR (400 MHz, DMSO- d₆): δ = 9.73 (brs, 1H), 8.79 (d, J = 1.6 Hz, 1H), 8.57 (d, J = 4.8 Hz, 1H), 8.28 (d, J = 2.8 Hz, 1H), 7.87-7.86 (m, 1H), 7.82 (d, J = 7.6 Hz, 2H), 7.43 (d, J = 5.2 Hz, 1H), 7.33 (t, J = 8.0 Hz, 2H), 6.98 (t, J = 7.2 Hz, 1H); 265.1 (M + H⁺). |
| 220 | 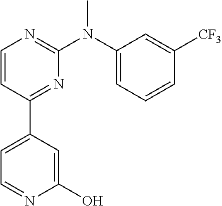 [SMILES: CN(c1cccc(C(F)(F)F)c1)c1nccc(-c2ccnc(O)c2)n1] | C | ¹H NMR (400 MHz, DMSO- d₆): δ = 11.74 (brs, 1H), 8.56 (d, J = 4.8 Hz, 1H), 7.83 (s, 1H), 7.74 (d, J = 8.4 Hz, 1H), 7.65 (t, J = 8.0 Hz, 1H), 7.57 (d, J = 7.2 Hz, 1H), 7.47 (d, J = 6.8 Hz, 1H), 7.41 (d, J = 5.2 Hz, 1H), 7.01 (s, 1H), 6.74 (dd, J = 6.8, 1.2 Hz, 1H), 3.60 (s, 3H); 347.1 (M + H⁺). |
| 221 | 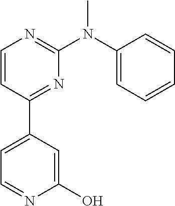 [SMILES: CN(c1ccccc1)c1nccc(-c2ccnc(O)c2)n1] | C | ¹H NMR (400 MHz, DMSO- d₆): δ = 8.45 (d, J = 4.8 Hz, 1H), 7.66-7.60 (m, 2H), 7.44- 7.38 (m, 5H), 7.34 (d, J = 4.8 Hz, 1H), 7.24-7.21 (m, 1H), 7.08-7.06 (m, 1H), 3.56 (s, 3H); 278.1 (M⁺). |
| 222 | 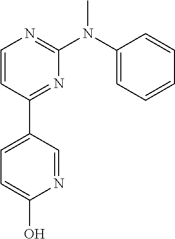 [SMILES: CN(c1ccccc1)c1nccc(-c2ccc(O)nc2)n1] | C | ¹H NMR (400 MHz, DMSO- d₆): δ = 10.47 (brs, 1H), 8.32 (d, J = 4 Hz, 1H), 8.17 (d, J = 4 Hz, 1H), 8.09-8.06 (m, 1H), 7.42- 7.36 (m, 4H), 7.23-7.16 (m, 2H), 6.40 (d, J = 8 Hz, 1H), 3.51 (s, 3H); 279.1 (M + H⁺). |
| 223 | 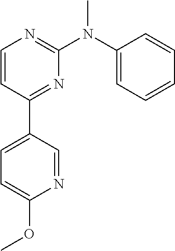 [SMILES: COc1ccc(-c2ccnc(N(C)c3ccccc3)n2)cn1] | C | ¹H NMR (400 MHz, DMSO- d₆): δ = 8.87 (d, J = 4 Hz, 1H), 8.42 (d, J = 4 Hz, 1H), 8.31 (dd, J = 3 Hz, 1H), 7.43-7.39 (m, 4H), 7.31 (d, J = 4 Hz, 1H), 7.25-7.21 (m, 1H), 6.92 (d, J = 4 Hz, 1H), 3.91 (s, 3H), 3.54 (s, 3H); 293.1 (M + H⁺). |
| 224 | 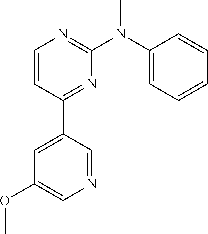 [SMILES: COc1cncc(-c2ccnc(N(C)c3ccccc3)n2)c1] | C | ¹H NMR (400 MHz, DMSO- d₆): δ = 8.85 (d, J = 5.6 Hz, 1H), 8.50 (d, J = 5.6 Hz, 1H), 8.41-8.40 (m, 1H), 7.92-7.91 (m, 1H), 7.46-7.42 (m, 5H), 7.25-7.23 (m, 1H), 3.87 (s, 3H), 3.56 (s, 3H); 293.1 (M + H⁺). |
| 225 | 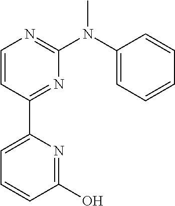 [SMILES: CN(c1ccccc1)c1nccc(-c2cccc(O)n2)n1] | C | ¹H NMR (400 MHz, DMSO- d₆): δ = 10.80 (brs, 1H), 8.51 (d, J = 4.8 Hz, 1H), 7.63 (t, J = 7.8 Hz, 1H), 7.44-7.40 (m, 5H), 7.27-7.23 (m, 2H), 6.60 (d, J = 8.8 Hz, 1H), 3.56 (s, 3H); 279.1 (M + H⁺). |
| 226 | 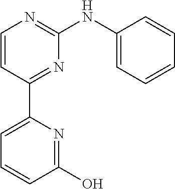 [SMILES: Oc1cccc(-c2ccnc(Nc3ccccc3)n2)n1] | C | ¹H NMR (400 MHz, DMSO- d₆): δ = 11.01 (brs, 1H), 9.79 (s, 1H), 8.63 (d, J = 5.2 Hz, 1H), 7.78 (d, J = 7.6 Hz, 2H), 7.71 (t, J = 7.8 Hz, 1H), 7.54 (d, J = 4.8 Hz, 1H), 7.42 (s, 1H), 7.33 (t, J = 8.0 Hz, 2H), 6.99 (t, J = 7.2 Hz, 1H), 6.66 (d, J = 8.8 Hz, 1H); 265.1 (M + H⁺). |
| 227 | 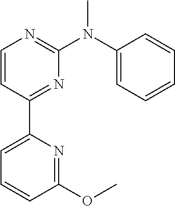 [SMILES: COc1cccc(-c2ccnc(N(C)c3ccccc3)n2)n1] | C | ¹H NMR (400 MHz, DMSO- d₆): δ = 8.53 (d, J = 4.8 Hz, 1H), 7.86-7.82 (m, 2H), 7.62 (d, J = 5.2 Hz, 1H), 7.43-7.42 (m, 4H), 7.24-7.22 (m, 1H), 6.96 (dd, J = 7.2, 1.6 Hz, 1H), 3.97 (s, 3H), 3.57 (s, 3H); 293.1 (M + H⁺). |
| 228 | 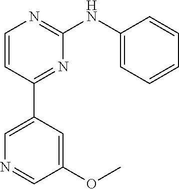 [SMILES: COc1cncc(-c2ccnc(Nc3ccccc3)n2)c1] | B | ¹H NMR (400 MHz, DMSO- d₆): δ = 9.75 (brs, 1H), 8.95 (d, J = 1.6 Hz, 1H), 8.60 (d, J = 5.2 Hz, 1H), 8.45 (d, J = 2.8 Hz, 1H), 8.06-8.04 (m, 1H), 7.82 (d, J = 8.0 Hz, 2H), 7.54 (d, J = 5.2 Hz, 1H), 7.33 (t, J = 8.0 Hz, 2H), 6.98 (t, J = 7.4 Hz, 1H), 3.94 (s, 3H); 279.1 (M + H⁺). |
| 229 | 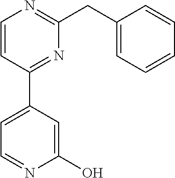 [SMILES: Oc1cc(-c2ccnc(Cc3ccccc3)n2)ccn1] | C | ¹H NMR (400 MHz, DMSO- d₆): δ = 11.79 (brs, 1H), 8.86 (d, J = 5.2 Hz, 1H), 7.93 (d, J = 5.2 Hz, 1H), 7.53 (d, J = 6.4 Hz, 1H), 7.36-7.28 (m, 4H), 7.23- 7.19 (m, 1H), 7.12 (d, J = 1.2 Hz, 1H), 6.87 (dd, J = 6.8, 1.6 Hz, 1H), 4.28 (s, 2H); 264.1 (M + H⁺). |
| 230 | 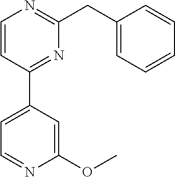 [SMILES: COc1cc(-c2ccnc(Cc3ccccc3)n2)ccn1] | C | ¹H NMR (400 MHz, DMSO- d₆): δ = 8.89 (d, J = 5.2 Hz, 1H), 8.35 (dd, J = 5.2, 1.2 Hz, 1H), 8.02 (d, J = 5.2 Hz, 1H), 7.71 (dd, J = 5.2, 1.2 Hz, 1H), 7.55 (s, 1H), 7.38-7.36 (m, 2H), 7.31 (t, J = 7.6 Hz, 2H), 7.24- 7.20 (m, 1H), 4.30 (s, 2H), 3.92 (s, 3H); 278.1 (M + H⁺). |
| 231 | 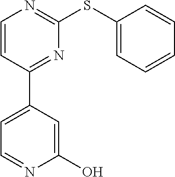 [SMILES: Oc1cc(-c2ccnc(Sc3ccccc3)n2)ccn1] | C | ¹H NMR (400 MHz, DMSO- d₆): δ = 8.73 (d, J = 5.2 Hz, 1H), 7.82 (d, J = 5.2 Hz, 1H), 7.67-7.65 (m, 2H), 7.53-7.47 (m, 4H), 6.96 (d, J = 1.2 Hz, 1H), 6.65 (dd, J = 7.0, 2.0 Hz, 1H ). |
| 232 | 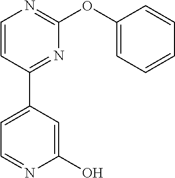 [SMILES: Oc1cc(-c2ccnc(Oc3ccccc3)n2)ccn1] | C | ¹H NMR (400 MHz, DMSO- d₆): δ = 11.84 (brs, 1H), 8.76 (d, J = 5.2 Hz, 1H), 7.86 (d, J = 5.2 Hz, 1H), 7.51 (t, J = 6.8 Hz, 1H), 7.49-7.45 (m, 2H), 7.31- 7.25 (m, 3H), 7.03 (s, 1H), 6.73 (d, J = 6.8 Hz, 1H); 282.0 (M + H⁺). |
| 233 | 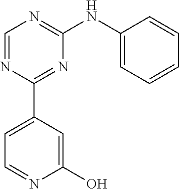 [SMILES: Oc1cc(-c2ncnc(Nc3ccccc3)n2)ccn1] | B | ¹H NMR (400 MHz, DMSO- d₆): δ = 11.80 (brs, 1H), 10.45 (brs, 1H), 8.88 (s, 1H), 7.75 (d, J = 7.6 Hz, 2H), 7.55 (d, J = 6.8 Hz, 1H), 7.39 (t, J = 7.2 Hz, 2H), 7.27 (s, 1H), 7.12 (t, J = 7.2 Hz, 1H), 6.96 (t, J = 6.4 Hz, 1H); 266.0 (M + H⁺). |
| 234 | 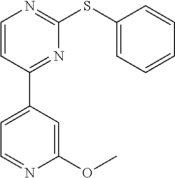 [SMILES: COc1cc(-c2ccnc(Sc3ccccc3)n2)ccn1] | A | ¹H NMR (400 MHz, DMSO- d₆): δ = 8.76 (d, J = 5.2 Hz, 1H), 8.29 (d, J = 5.2 Hz, 1H), 7.92 (d, J = 5.2 Hz, 1H), 7.69- 7.66 (m, 2H), 7.54-7.51 (m, 4H), 7.34 (s, 1H), 3.88 (s, 3H); 296.0 (M + H⁺). |
| 235 | 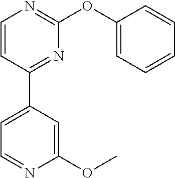 [SMILES: COc1cc(-c2ccnc(Oc3ccccc3)n2)ccn1] | C | ¹H NMR (400 MHz, DMSO- d₆): δ = 8.80 (d, J = 5.2 Hz, 1H), 8.34 (d, J = 5.2 Hz, 1H), 7.96 (d, J = 5.2 Hz, 1H), 7.61- 7.60 (m, 1H), 7.48 (t, J = 8.0 Hz, 2H), 7.44 (s, 1H), 7.32-7.27 (m, 3H), 3.90 (s, 3H); 280.1 (M + H⁺). |
| 236 | 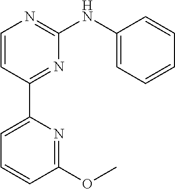 [SMILES: COc1cccc(-c2ccnc(Nc3ccccc3)n2)n1] | C | ¹H NMR (400 MHz, DMSO- d₆): δ = 9.72 (brs, 1H), 8.65 (d, J = 5.2 Hz, 1H), 8.02 (d, J = 4.8 Hz, 1H), 7.94 (t, J = 7.8 Hz, 1H), 7.85 (d, J = 8.0 Hz, 1H), 7.71 (d, J = 8.0 Hz, 1H), 7.33 (t, J = 7.8 Hz, 2H), 7.02-6.96 (m, 2H), 3.99 (s, 3H); 279.1 (M + H⁺). |
| 237 | 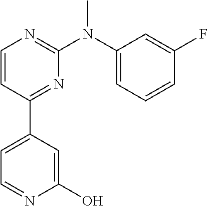 [SMILES: CN(c1cccc(F)c1)c1nccc(-c2ccnc(O)c2)n1] | C | ¹H NMR (400 MHz, DMSO- d₆): δ = 11.72 (brs, 1H), 8.54 (d, J = 4.8 Hz, 1H), 7.48 (d, J = 6.8 Hz, 1H), 7.44 (dd, J = 15.2, 8.0 Hz, 1H), 7.38 (d, J = 5.2 Hz, 1H), 7.35-7.29 (m, 1H), 7.28- 7.24 (m, 1H), 7.09-7.03 (m, 1H), 7.01 (d, J = 1.2 Hz, 1H), 6.75 (dd, J = 6.8, 1.6 Hz, 1H), 3.55 (s, 3H); 297.0 (M + H⁺). |
| 238 | 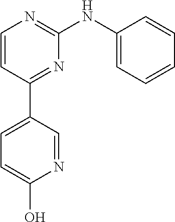 [SMILES: Oc1ccc(-c2ccnc(Nc3ccccc3)n2)cn1] | B | 265.1 (M + H⁺) |
| 239 | 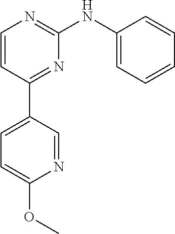 [SMILES: COc1ccc(-c2ccnc(Nc3ccccc3)n2)cn1] | C | ¹H NMR (400 MHz, DMSO- d₆): δ = 12.18 (brs, 1H), 9.69 (s, 1H), 8.44 (d, J = 8 Hz, 1H), 8.32 (d, J = 4 Hz, 1H), 8.20 (d, J = 8 Hz, 1H), 7.75(d, J = 8 Hz, 2H), 7.33-7.28 (m, 3H), 6.98 (t, J = 8 Hz, 1H), 6.48 (d, J = 8 Hz, 1H); 279.1 (M + H⁺). |
| 240 | 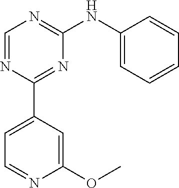 [SMILES: COc1cc(-c2ncnc(Nc3ccccc3)n2)ccn1] | B | ¹H NMR (400 MHz, DMSO- d₆): δ = 10.48 (brs, 1H), 8.90 (s, 1H), 8.40 (d, J = 5.6 Hz, 1H), 7.81 (d, J = 5.2 Hz, 1H), 7.77 (d, J = 8.0 Hz, 2H), 7.62 (s, 1H), 7.46-7.34 (m, 2H), 7.13 (t, J = 7.6 Hz, 1H), 3.93 (s, 3H); 280.1 (M + H⁺). |
| 241 | 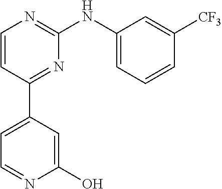 [SMILES: Oc1cc(-c2ccnc(Nc3cccc(C(F)(F)F)c3)n2)ccn1] | A | ¹H NMR (400 MHz, DMSO- d₆): δ = 11.86 (brs, 1H), 10.15 (s, 1H), 8.68 (d, J = 5.2 Hz, 1H), 8.38 (s, 1H), 7.99 (d, J = 8.4 Hz, 1H), 7.59-7.52 (m, 2H), 7.50 (d, J = 5.2 Hz, 1H), 7.31 (d, J = 8.0 Hz, 1H), 7.11 (d, J = 1.2 Hz, 1H), 6.85 (dd, J = 6.8, 1.6 Hz, 1H); 332.9 (M + H⁺). |
| 242 | 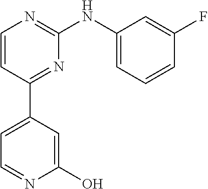 [SMILES: Oc1cc(-c2ccnc(Nc3cccc(F)c3)n2)ccn1] | A | ¹H NMR (400 MHz, DMSO- d₆): δ = 11.85 (brs, 1H), 10.03 (s, 1H), 8.66 (d, J = 5.2 Hz, 1H), 7.87-7.79 (m, 1H), 7.59- 7.55 (m, 1H), 7.54-7.50 (m, 1H), 7.47 (d, J = 5.6 Hz, 1H), 7.33 (dd, J = 15.2, 8.4 Hz, 1H), 7.09 (s, 1H), 6.84 (dd, J = 6.8, 1.2 Hz, 1H), 6.82-6.75 (m, 1H); 283.1 (M + H⁺). |
| 243 | 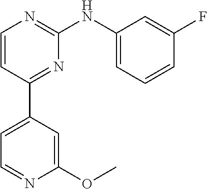 [SMILES: COc1cc(-c2ccnc(Nc3cccc(F)c3)n2)ccn1] | B | ¹H NMR (400 MHz, DMSO- d₆): δ = 10.04 (brs, 1H), 8.69 (d, J = 5.2 Hz, 1H), 8.37 (d, J = 5.2 Hz, 1H), 7.90-7.82 (m, 1H), 7.68 (dd, J = 5.2, 1.2 Hz, 1H), 7.60-7.50 (m, 3H), 7.35 (dd, J = 15.2, 8.0 Hz, 1H), 6.84-6.74 (m, 1H), 3.93 (s, 3H); 296.9 (M + H⁺). |
| 244 | 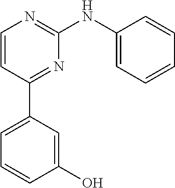 [SMILES: Oc1cccc(-c2ccnc(Nc3ccccc3)n2)c1] | B | ¹H NMR (400 MHz, DMSO- d₆): δ = 9.70 (brs, 1H), 9.62 (brs, 1H), 8.52 (d, J = 4.8 Hz, 1H), 7.84 (d, J = 7.6 Hz, 2H), 7.57-7.56 (m, 2H), 7.36-7.30 (m, 4H), 6.98-6.93 (m, 2H); 264.1 (M + H⁺). |
| 245 | 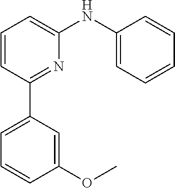 [SMILES: COc1cccc(-c2cccc(Nc3ccccc3)n2)c1] | C | ¹H NMR (400 MHz, CD₃OD): δ = 9.11 (s, 1H), 7.80 (d, J = 7.6 Hz, 2H), 7.68-7.61 (m, 3H), 7.41-7.28 (m, 4H), 6.97-6.99 (m, 1H), 6.91 (t, J = 7.2 Hz, 1H), 6.80 (d, J = 8.0 Hz, 1H), 3.85 (s, 3H); 277.1 (M + H⁺). |
| 246 | 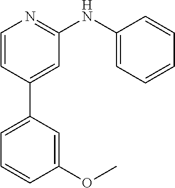 [SMILES: COc1cccc(-c2ccnc(Nc3ccccc3)c2)c1] | C | ¹H NMR (400 MHz, CD₃OD): δ = 8.19 (d, J = 5.6 Hz, 1H), 7.58- 7.56 (m, 2H), 7.46 (t, J = 8.0 Hz, 1H), 7.38-7.34 (m, 2H), 7.31-7.29 (m, 1H), 7.26-7.25 (m, 1H), 7.12 (s, 1H), 7.11-7.03 (m, 3H), 3.93 (s, 3H); 277.1 (M + H⁺). |
| 247 | 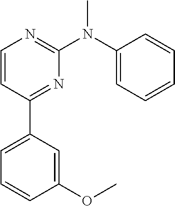 [SMILES: COc1cccc(-c2ccnc(N(C)c3ccccc3)n2)c1] | B | ¹H NMR (400 MHz, DMSO- d₆): δ = 8.44 (d, J = 5.2 Hz, 1H), 7.65-7.60 (m, 2H), 7.43- 7.38 (m, 5H), 7.33 (d, J = 5.2 Hz, 1H), 7.24-7.20 (m, 1H), 7.07 (dd, J = 8.0, 2.4 Hz, 1H), 3.79 (s, 3H), 3.56 (s, 3H); 292.1 (M + H⁺). |
| 248 | 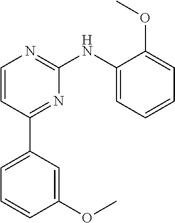 [SMILES: COc1cccc(-c2ccnc(Nc3ccccc3OC)n2)c1] | C | ¹H NMR (400 MHz, DMSO- d₆): δ = 8.53 (d, J = 5.2 Hz, 1H), 8.27 (dd, J = 8.0, 1.2 Hz, 1H), 8.14 (s, 1H), 7.74-7.71 (m, 2H), 7.48-7.44 (m, 2H), 7.13- 6.97 (m, 4H), 3.88 (s, 3H), 3.85 (s, 3H); 308.1 (M + H⁺). |
| 249 | 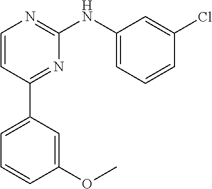 [SMILES: COc1cccc(-c2ccnc(Nc3cccc(Cl)c3)n2)c1] | B | ¹H NMR (400 MHz, DMSO- d6): δ = 9.92 (brs, 1H), 8.60 (d, J = 5.2 Hz, 1H), 8.16 (t, J = 2.0 Hz, 1H), 7.75-7.73 (m, 2H), 7.69 (dd, J = 8.4, 1.2 Hz, 1H), 7.50-7.45 (m, 2H), 7.33 (t, J = 8.0 Hz, 1H), 7.15-7.12 (m, 1H), 7.02-6.99 (m, 1H), 3.88 (s, 3H); 312.0 (M + H⁺). |
| 250 | 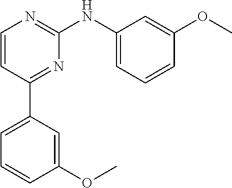 [SMILES: COc1cccc(Nc2nccc(-c3cccc(OC)c3)n2)c1] | B | ¹H NMR (400 MHz, DMSO- d₆): δ = 9.66 (brs, 1H), 8.56 (d, J = 5.6 Hz, 1H), 7.76-7.74 (m, 2H)), 7.67 (t, J = 2.0 Hz, 1H), 7.48-7.43 (m, 2H), 7.35 (dd, J = 8.2, 1.0 Hz, 1H), 7.20 (t, J = 8.2 Hz, 1H), 7.12 (dd, J = 8.0, 2.0 Hz, 1H), 6.55 (dd, J = 8.0, 2.0 Hz, 1H), 3.87 (s, 3H), 3.76 (s, 3H); 308.1 (M + H⁺). |
| 251 | 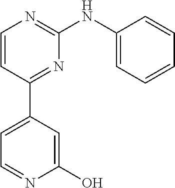 [SMILES: Oc1cc(-c2ccnc(Nc3ccccc3)n2)ccn1] | B | ¹H NMR (400 MHz, DMSO- d₆): δ = 11.79 (brs, 1H), 9.76 (s, 1H), 8.61 (d, J = 5.2 Hz, 1H), 7.78 (d, J = 7.6 Hz, 2H), 7.54 (d, J = 6.8 Hz, 1H), 7.39 (d, J = 5.2 Hz, 1H), 7.31 (t, J = 7.6 Hz, 2H), 7.08 (s, 1H), 6.98 (t, J = 8 Hz, 1H), 6.85-6.83 (m, 1H); 265.1 (M + H⁺). |
| 252 | 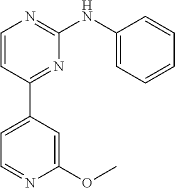 [SMILES: COc1cc(-c2ccnc(Nc3ccccc3)n2)ccn1] | C | ¹H NMR (400 MHz, DMSO- d₆): δ = 9.78 (s, 1H), 8.64 (d, J = 5.2 Hz, 1H), 8.36 (d, J = 5.2 Hz, 1H)), 7.81 (d, J = 7.6 Hz, 2H), 7.68 (dd, J = 5.2, 1.2 Hz, 1H), 7.52-7.50 (m, 2H), 7.33 (t, J = 8 Hz, 2H), 6.99 (t, J = 7.2 Hz, 1H), 3.93 (s, 3H); 279.1 (M + H⁺). |
| 253 | 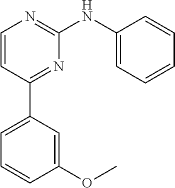 [SMILES: COc1cccc(-c2ccnc(Nc3ccccc3)n2)c1] | C | ¹H NMR (400 MHz, DMSO- d₆): δ = 9.67 (s, 1H), 8.39 (d, J = 5.6 Hz, 1H), 7.95-7.91 (m, 2H), 7.78 (d, J = 7.6 Hz, 2H), 7.45-7.37 (m, 3H), 7.09-7.04 (m, 2H), 6.73 (d, J = 5.6 Hz, 1H), 3.84 (s, 3H); 278.1 (M + H⁺). |
| 254 | 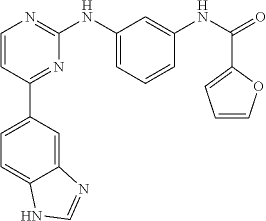 [SMILES: O=C(Nc1cccc(Nc2nccc(-c3ccc4[nH]cnc4c3)n2)c1)c1ccco1] | * | ¹H NMR (400 MHz, CD₃OD) δ 8.56-8.42 (m, 2H), 8.31-8.21 (m, 2H), 8.20-8.08 (m, 1H), 7.78-7.68 (m, 2H), 7.65-7.56 (m, 1H), 7.41-7.25 (m, 4H), 6.65 (ddd, J = 3.5, 1.7, 0.7 Hz, 1H); 397.1 (M + H⁺). |
| 255 | 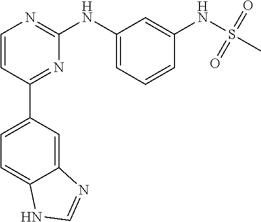 [SMILES: CS(=O)(=O)Nc1cccc(Nc2nccc(-c3ccc4[nH]cnc4c3)n2)c1] | B | ¹H NMR (400 MHz, CD₃OD) δ 9.37 (q, J = 1.0 Hz, 1H), 9.13 (d, J = 2.6 Hz, 1H), 8.67-8.51 (m, 4H), 8.43-8.32 (m, 5H), 8.03-7.82 (m, 1H), 7.62 (ddt, J = 8.3, 4.9, 1.0 Hz, 1H), 7.58- 7.44 (m, 1H); 381.1 (M + H⁺). |
| 256 | 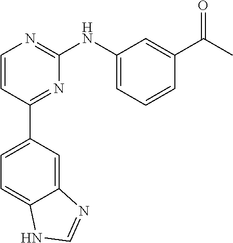 [SMILES: CC(=O)c1cccc(Nc2nccc(-c3ccc4[nH]cnc4c3)n2)c1] | * | ¹H NMR (400 MHz, CD₃OD) δ 8.60 (s, 1H), 8.48-8.40 (m, 2H), 8.26 (d, J = 0.8 Hz, 1H), 8.12 (d, J = 8.6 Hz, 1H), 7.94 (dd, J = 8.1, 2.3 Hz, 1H), 7.70 (d, J = 8.5 Hz, 1H), 7.61 (ddt, J = 7.7, 1.8, 0.9 Hz, 1H), 7.43 (dd, J = 9.0, 6.8 Hz, 1H), 7.38- 7.32 (m, 1H), 2.64-2.59 (s, 3H); 330.1 (M + H⁺). |
| 257 | 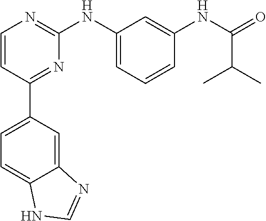 [SMILES: CC(C)C(=O)Nc1cccc(Nc2nccc(-c3ccc4[nH]cnc4c3)n2)c1] | A | ¹H NMR (400 MHz, CD₃OD) δ 8.49-8.35 (m, 2H), 8.25 (s, 1H), 8.16-8.03 (m, 2H), 7.69 (d, J = 8.5 Hz, 1H), 7.53-7.46 (m, 1H), 7.39-7.21 (m, 2H), 7.19-7.00 (m, 1H), 2.70-2.57 (m, 1H), 1.27-1.10 (m, 6H); 373.1 (M + H⁺). |
| 258 | 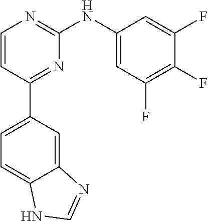 [SMILES: Fc1cc(Nc2nccc(-c3ccc4[nH]cnc4c3)n2)cc(F)c1F] | * | ¹H NMR (400 MHz, CDCl₃) δ 9.52(1H, m), 8.59-8.51 (m, 1H), 8.14 (t, J = 8.0 Hz, 2H), 7.98-7.86 (m, 2H), 7.60 (dd, J = 16.0, 8.7 Hz, 1H), 7.50-7.37 (m, 3H); 342.1 (M + H⁺). |
| 259 | 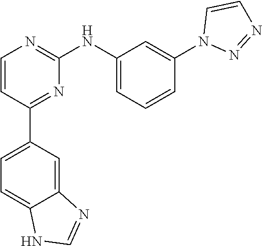 [SMILES: c1cc(Nc2nccc(-c3ccc4[nH]cnc4c3)n2)cc(-n2ccnn2)c1] | * | ¹H NMR (400 MHz, CDCl₃) δ 8.55-8.46 (m, 2H), 8.30 (m, 2H), 7.51-7.42 (m, 3H), 7.27- 7.21 (m, 3H), 4.53 (m, 1H); 355.1 (M + H⁺). |
| 260 | 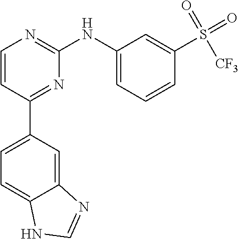 [SMILES: O=S(=O)(c1cccc(Nc2nccc(-c3ccc4[nH]cnc4c3)n2)c1)C(F)(F)F] | * | ¹H NMR (400 MHz, CD₃OD) δ 9.06 (s, 1H), 8.52 (dt, J = 5.3, 1.1 Hz, 1H), 8.45 (s, 1H), 8.27 (s, 1H), 8.19 (d, J = 8.5 Hz, 1H), 8.11 (d, J = 7.8 Hz, 1H), 7.74-7.60 (m, 3H), 7.47 (dd, J = 5.2, 2.0 Hz, 1H); 420.1 (M + H⁺). |
| 261 | 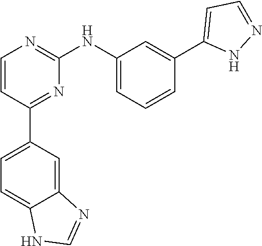 [SMILES: c1cc(Nc2nccc(-c3ccc4[nH]cnc4c3)n2)cc(-c2ccn[nH]2)c1] | * | ¹H NMR (400 MHz, CDCl₃) δ 8.48 (d, J = 5.3 Hz, 1H), 8.16 (s, 1H), 7.87 (m, 1H), 7.65 (m, 3H), 7.5 (m, 3H), 7.40 (m, 2H), 7.20 (m, 2H), 6.67 (m, 1H); 354.2 (M + H⁺). |
| 262 | 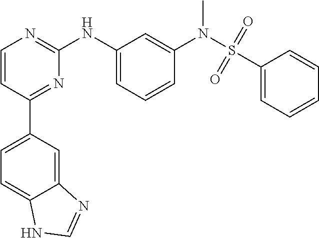 [SMILES: CN(c1cccc(Nc2nccc(-c3ccc4[nH]cnc4c3)n2)c1)S(=O)(=O)c1ccccc1] | B | 457.1 (M + H⁺) |
| 263 | 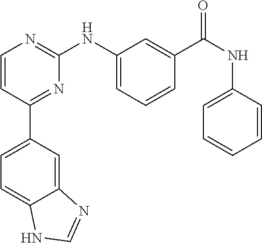 [SMILES: O=C(Nc1ccccc1)c1cccc(Nc2nccc(-c3ccc4[nH]cnc4c3)n2)c1] | * | ¹H NMR (400 MHz, CDCl₃) δ 11.86-11.56 (m, 1H), 9.17 (d, J = 14.2 Hz, 1H), 8.36 (m, 1H), 8.15-7.78 (m, 3H), 7.59 (d, J = 14.7 Hz, 1H), 7.50 (m, 1H), 7.27 (m, 3H), 7.00-6.72 (m, 4H), 6.61-6.54 (m, 1H); 407.2 (M + H⁺). |
| 264 | 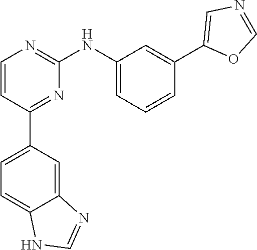 [SMILES: c1cc(Nc2nccc(-c3ccc4[nH]cnc4c3)n2)cc(-c2cnco2)c1] | * | ¹H NMR (400 MHz, CDCl₃) δ 8.49 (d, J = 5.3 Hz, 2H), 8.14 (m, 2H), 7.51 (m, 1H), 7.48 (m, 1H), 7.45-7.31 (m, 3H), 7.28 (m, 2H), 7.27 (m, 2H); 355.1 (M + H⁺). |
| 265 | 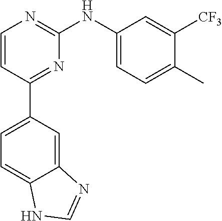 [SMILES: Cc1ccc(Nc2nccc(-c3ccc4[nH]cnc4c3)n2)cc1C(F)(F)F] | * | ¹H NMR (400 MHz, CDCl₃) δ 8.35 (d, J = 5.3 Hz, 1H), 8.19 (m, 1H), 8.03 (m, 1H), 7.95- 7.85 (m, 1H), 7.75 (m, 1H), 7.61 (m, 1H), 7.25 (d, J = 1.0 Hz, 2H), 7.19 (t, J = 6.7 Hz, 2H), 2.38 (d, J = 2.2 Hz, 3H); 370.1 (M + H⁺). |
| 266 | 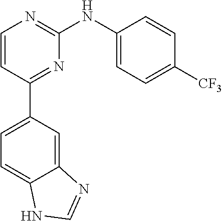 [SMILES: FC(F)(F)c1ccc(Nc2nccc(-c3ccc4[nH]cnc4c3)n2)cc1] | B | ¹H NMR (400 MHz, CDCl₃) δ 8.35 (d, J = 5.3 Hz, 1H), 8.19 (s, 1H), 8.03 (s, 1H), 7.75 (s, 0H), 7.61 (s, 1H), 7.25 (d, J = 1.0 Hz, 2H), 7.19 (t, J = 6.7 Hz, 2H), 2.38 (d, J = 2.2 Hz, 3H); 356.1 (M + H⁺). |
| 267 | 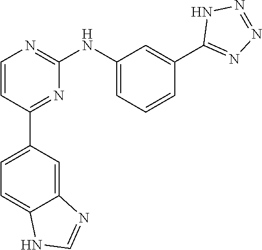 [SMILES: c1cc(Nc2nccc(-c3ccc4[nH]cnc4c3)n2)cc(-c2nnn[nH]2)c1] | * | ¹H NMR (400 MHz, CD₃OD) δ 9.75 (d, J = 1.7 Hz, 1H), 8.76 (s, 1H), 8.50 (d, J = 8.3 Hz, 2H), 8.28 (d, J = 1.8 Hz, 1H), 8.19 (s, 1H), 7.81 (d, J = 8.3 Hz, 1H), 7.73 (s, 1H), 7.54 (dd, J = 8.9, 7.0 Hz, 1H), 7.47-7.40 (m, 2H); 356.1 (M + H⁺). |
| 268 | 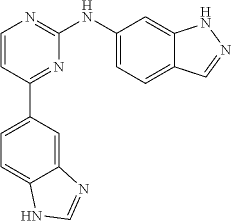 [SMILES: c1cc(-c2ccc3[nH]cnc3c2)nc(Nc2ccc3cn[nH]c3c2)n1] | A | ¹H NMR (400 MHz, CDCl₃) δ 11.85 (s, 1H), 8.66-8.20 (m, 1H), 8.26-7.91 (m, 2H), 7.91- 7.61 (m, 1H), 7.59-7.40 (m, 1H), 7.22-7.05 (m, 3H), 6.98- 6.61 (m, 2H); 328.2 (M + H⁺). |
| 269 | 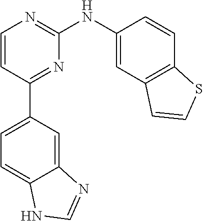 [SMILES: c1cc(-c2ccc3[nH]cnc3c2)nc(Nc2ccc3sccc3c2)n1] | A | ¹H NMR (400 MHz, CD₃OD) δ 8.56-8.49 (m, 3H), 8.36 (d, J = 2.2 Hz, 1H), 8.17 (d, J = 8.3 Hz, 1H), 7.90 (d, J = 8.7 Hz, 1H), 7.75 (m, 2H), 7.64 (t, J = 4.1 Hz, 1H), 7.44 (dd, J = 5.4, 1.7 Hz, 2H); 344.1 (M + H⁺). |
| 270 | 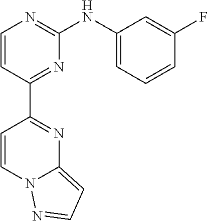 [SMILES: Fc1cccc(Nc2nccc(-c3ccn4nccc4n3)n2)c1] | B | ¹H NMR (400 MHz, CD₃OD) δ 9.35 (dd, J = 12.8, 2.1 Hz, 1H), 9.00 (dd, J = 15.0, 2.1 Hz, 1H), 8.53 (d, J = 5.3 Hz, 1H), 8.25 (s, 1H), 7.81 (dt, J = 12.1, 2.3 Hz, 1H), 7.72-7.59 (m, 1H), 7.48-7.21 (m, 3H), 6.77-6.63 (m, 2H); 307.1 (M + H⁺). |
| 271 | 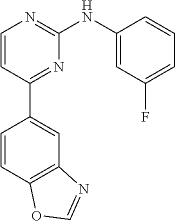 [SMILES: Fc1cccc(Nc2nccc(-c3ccc4ocnc4c3)n2)c1] | B | ¹H NMR (400 MHz, CD₃OD) δ 8.33 (d, J = 5.3 Hz, 1H), 7.77 (dt, J = 12.2, 2.3 Hz, 1H), 7.54 (d, J = 2.2 Hz, 1H), 7.47-7.33 (m, 2H), 7.25 (td, J = 8.2, 6.7 Hz, 1H), 7.15 (d, J = 5.4 Hz, 1H), 6.78 (d, J = 8.3 Hz, 1H), 6.66 (td, J = 8.5, 2.8 Hz, 1H); 338.1 (M + CH₃OH). |
| 272 | 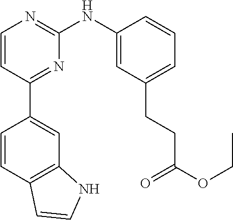 [SMILES: CCOC(=O)CCc1cccc(Nc2nccc(-c3ccc4cc[nH]c4c3)n2)c1] | A | ¹H NMR (400 MHz, CDCl₃) δ 8.48 (s, 1H), 8.38 (d, J = 5.2 Hz, 1H), 8.11 (m, 2H), 7.76-7.66 (m, 2H), 7.27-7.13 (m, 3H), 6.85 (m, 1H), 4.14 (q, J = 7.1 Hz, 2H), 2.96 (t, J = 8.0 Hz, 2H), 2.71 (t, J = 7.9 Hz, 2H), 1.22 (t, J = 7.1 Hz, 3H); 388.2 (M + H⁺). |
| 273 | 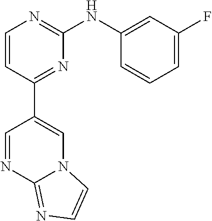 [SMILES: Fc1cccc(Nc2nccc(-c3cnc4nccn4c3)n2)c1] | B | ¹H NMR (400 MHz, CD₃OD) δ 10.61 (s, 1H), 9.21 (d, J = 5.9 Hz, 1H), 8.96 (d, J = 5.7 Hz, 1H), 8.61 (dd, J = 5.9, 1.0 Hz, 1H), 8.29 (q, J = 7.6 Hz, 1H), 8.02-7.80 (m, 4H), 7.42 (td, J = 8.5, 2.5 Hz, 1H); 306.3 (M+). |
| 274 | 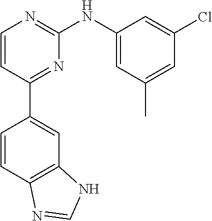 [SMILES: Cc1cc(Cl)cc(Nc2nccc(-c3ccc4nc[nH]c4c3)n2)c1] | * | ¹H NMR (400 MHz, DMSO- D6) δ 12.73 (s, 2H), 8.71 (m, 2H), 8.51 (s, 1H), 8.34 (d, J = 14.5 Hz, 3H), 8.16 (m, 1H), 7.75 (d, J = 8.7 Hz, 1H), 7.64 (d, J = 8.5 Hz, 1H), 2.46 (s, 3H); 336.1 (M + H⁺). |
| 275 | 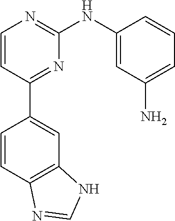 [SMILES: Nc1cccc(Nc2nccc(-c3ccc4nc[nH]c4c3)n2)c1] | * | ¹H NMR (400 MHz, CD₃OD) δ 8.45 (s, 1H), 8.45-8.35 (m, 1H), 8.25 (d, J = 0.9 Hz, 1H), 8.07 (dt, J = 8.6, 1.3 Hz, 1H), 7.69 (d, J = 8.5 Hz, 1H), 7.37- 7.24 (m, 2H), 7.12-7.01 (m, 2H), 6.50-6.32 (m, 1H); 303.2 (M + H⁺). |
| 276 | 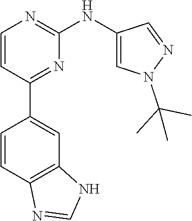 [SMILES: CC(C)(C)n1cc(Nc2nccc(-c3ccc4nc[nH]c4c3)n2)cn1] | A | ¹H NMR (400 MHz, CD₃OD) δ 8.41-8.34 (m, 2H), 8.26 (d, J = 1.3 Hz, 1H), 8.13 (s, 1H), 8.06 (dt, J = 8.4, 1.5 Hz, 1H), 7.69 (d, J = 7.9 Hz, 2H), 7.23 (dd, J = 5.3, 1.4 Hz, 1H), 1.60 (d, J = 1.3 Hz, 9H); 334.2 (M + H⁺). |
| 277A | 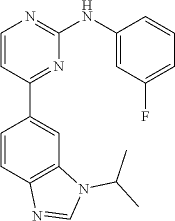 [SMILES: CC(C)n1cnc2ccc(-c3ccnc(Nc4cccc(F)c4)n3)cc21] | * | ¹H NMR (400 MHz, CD₃OD) δ 8.58-8.46 (m, 2H), 8.40 (s, 1H), 8.29 (s, 1H), 8.19 (dd, J = 8.5, 1.8 Hz, 1H), 7.89-7.73 (m, 2H), 7.51-7.39 (m, 2H), 7.35-7.25 (m, 1H), 6.71 (td, J = 8.2, 2.5 Hz, 1H), 1.74-1.63 (m, 6H). MS: m/z 347.8 (M⁺). |
| 277B | 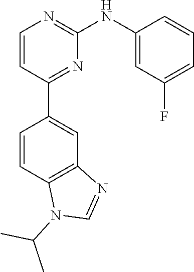 [SMILES: CC(C)n1cnc2cc(-c3ccnc(Nc4cccc(F)c4)n3)ccc21] | | |
| 278 | 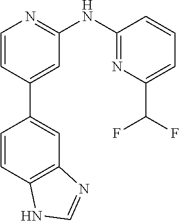 [SMILES: FC(F)c1cccc(Nc2cc(-c3ccc4[nH]cnc4c3)ccn2)n1] | * | ¹H NMR (400 MHz, CD₃OD) δ 8.25 (d, J = 5.7 Hz, 3H), 7.99 (s, 1H), 7.83-7.65 (m, 3H), 7.62 (d, J = 8.6 Hz, 2H), 7.28 (d, J = 5.9 Hz, 2H), 7.15 (d, J = 7.2 Hz, 1H). MS: m/z 338 (M + H). |
| 279 | 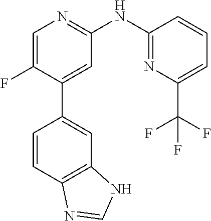 [SMILES: Fc1cnc(Nc2cccc(C(F)(F)F)n2)cc1-c1ccc2nc[nH]c2c1] | * | ¹H NMR (400 MHz, CD₃OD) δ 8.32-8.24 (m, 2H), 8.19 (s, 1H), 7.96 (s, 1H), 7.84-7.70 (m, 2H), 7.61 (t, J = 7.8 Hz, 2H), 7.22 (d, J = 7.4 Hz, 1H). MS: m/z 373.6 (M⁺). |
| 280 | 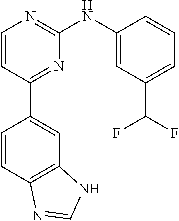 [SMILES: FC(F)c1cccc(Nc2nccc(-c3ccc4nc[nH]c4c3)n2)c1] | * | ¹H NMR (400 MHz, CDCl₃) δ 7.66 (d, J = 5.4 Hz, 1H), 7.36 (d, J = 37.5 Hz, 2H), 7.05 (d, J = 8.2 Hz, 1H), 6.95 (s, 2H), 6.69-6.48 (m, 2H), 6.36 (d, J = 7.6 Hz, 1H), 5.94 (s, 1H), 3.70 (s, 1H). MS: m/z 339.0 (M + H). |
| 281 | 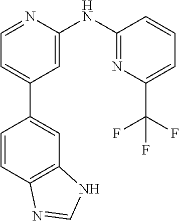 [SMILES: FC(F)(F)c1cccc(Nc2cc(-c3ccc4nc[nH]c4c3)ccn2)n1] | * | ¹H NMR (400 MHz, CD₃OD) δ 6.71-6.62 (m, 3H), 6.50 (s, 1H), 6.37 (s, 1H), 6.21 (t, J = 8.0 Hz, 1H), 6.07 (dd, J = 12.5, 7.1 Hz, 3H), 5.66 (dd, J = 20.2, 6.6 Hz, 2H). MS: m/z 356.1 (M + H). |
| 282 | 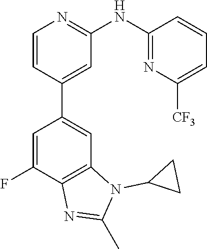 [SMILES: Cc1nc2c(F)cc(-c3ccnc(Nc4cccc(C(F)(F)F)n4)c3)cc2n1C1CC1] | | ¹H NMR (400 MHz, METHANOL-D₃) δ 7.64 (s, 1H), 7.44 (s, 1H), 7.15-6.86 (m, 2H), 6.75 (d, J = 8.3 Hz, 1H), 6.62-6.31 (m, 3H), 1.87 (s, 3H), 0.50 (d, J = 6.7 Hz, 2H), 0.31 (d, J = 3.5 Hz, 2H). MS: m/z 429.2 (M + H) |
| 283 | 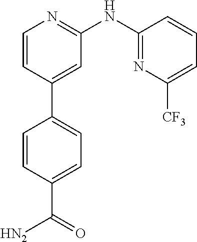 | | ¹H NMR (400 MHz, DMSO- D₆) δ 10.30 (s, 1H), 8.25 (d, J = 15.7 Hz, 2H), 8.09 (s, 1H), 8.02 (t, J = 9.1 Hz, 3H), 7.94 (t, J = 7.9 Hz, 1H), 7.81 (d, J = 8.0 Hz, 2H), 7.48 (s, 1H), 7.35 (t, J = 6.4 Hz, 2H). MS: m/z 359 (M + H) |
| 284 | 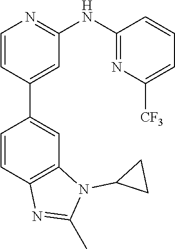 [SMILES: Cc1nc2ccc(-c3ccnc(Nc4cccc(C(F)(F)F)n4)c3)cc2n1C1CC1] | | ¹H NMR (400 MHz, DMSO- D₆) δ 10.23 (s, 1H), 8.36-8.25 (m, 3H), 8.03-7.88 (m, 2H), 7.84 (s, 1H), 7.63 (d, J = 8.2 Hz, 1H), 7.54 (d, J = 8.4 Hz, 1H), 7.34 (d, J = 7.6 Hz, 2H), 2.62 (s, 3H), 1.23 (t, J = 6.3 Hz, 2H), 1.10 (q, J = 3.9 Hz, 2H). MS: m/z 410 (M + H) |
| 285 | 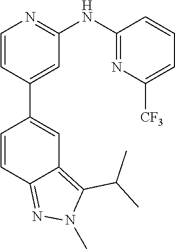 [SMILES: CC(C)c1c2cc(-c3ccnc(Nc4cccc(C(F)(F)F)n4)c3)ccc2nn1C] | | MS: m/z 412 (M + H) |
| 286 | 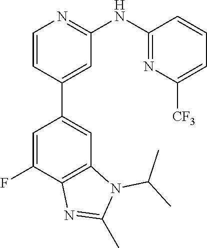 [SMILES: Cc1nc2c(F)cc(-c3ccnc(Nc4cccc(C(F)(F)F)n4)c3)cc2n1C(C)C] | | ¹H NMR (400 MHz, DMSO- D₆) δ 10.22 (s, 1H), 8.37-8.27 (m, 2H), 7.94 (d, J = 6.1 Hz, 2H), 7.80 (s, 1H), 7.51-7.29 (m, 3H), 4.84 (p, J = 7.0 Hz, 1H), 2.63 (s, 3H), 1.63 (d, J = 6.8 Hz, 6H). MS: m/z 430.99 (M + H) |
| 287 | 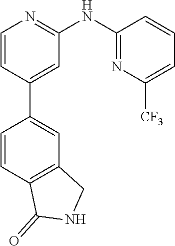 [SMILES: O=C1NCc2cc(-c3ccnc(Nc4cccc(C(F)(F)F)n4)c3)ccc21] | | MS: m/z 371 (M + H) |
| 288 | 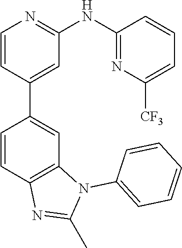 [SMILES: Cc1nc2ccc(-c3ccnc(Nc4cccc(C(F)(F)F)n4)c3)cc2n1-c1ccccc1] | | MS: m/z 446 (M + H) |
| 289 | 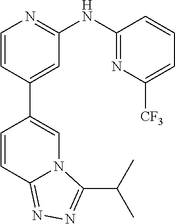 [SMILES: CC(C)c1nnc2ccc(-c3ccnc(Nc4cccc(C(F)(F)F)n4)c3)cn12] | | MS: m/z 400 (M + H) |
| 290 | 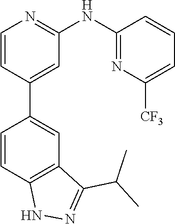 [SMILES: CC(C)c1n[nH]c2ccc(-c3ccnc(Nc4cccc(C(F)(F)F)n4)c3)cc12] | | ¹H NMR (400 MHz, DMSO- D₆) δ 12.82 (s, 1H), 10.22 (s, 1H), 8.52 (s, 1H), 8.38-8.30 (m, 2H), 8.15 (s, 1H), 7.93 (d, J = 4.3 Hz, 2H), 7.75 (dd, J = 19.0, 8.7 Hz, 1H), 7.61 (d, J = 8.7 Hz, 1H), 7.40-7.35 (m, 2H), 3.45 (p, J = 6.9 Hz, 1H), 1.43 (d, J = 6.8 Hz, 6H). MS: m/z 397.9 (M + H) |
| 291 | 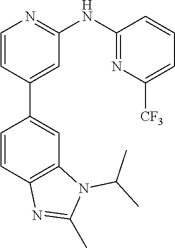 [SMILES: Cc1nc2ccc(-c3ccnc(Nc4cccc(C(F)(F)F)n4)c3)cc2n1C(C)C] | | ¹H NMR (400 MHz, DMSO- D₆) δ 10.20 (s, 1H), 8.32 (d, J = 5.4 Hz, 1H), 8.25 (d, J = 4.9 Hz, 2H), 8.00 (d, J = 8.6 Hz, 1H), 7.96-7.88 (m, 2H), 7.64 (d, J = 8.5 Hz, 1H), 7.53 (d, J = 8.6 Hz, 1H), 7.36 (dd, J = 9.8, 6.3 Hz, 2H), 4.82 (p, J = 7.0 Hz, 1H), 2.60 (s, 3H), 1.62 (d, J = 6.8 Hz, 6H). MS: m/z 412 (M + H) |

### Example 4—Myc Inhibition Assays of Compounds

To identify Myc inhibitors, cells isolated from patient-derived orthotopic xenografts (PDXs) from patients with Myc-driven medulloblastoma were used. Specifically, PDX cells were propagated by transplantation directly into the cerebellum of immunodeficient (NSG) mice and were never passaged in vitro in order to maintain properties of the tumor from which they were derived. Tumor tissues were isolated from tumor-bearing animals, dissociated into a single-cell suspension, resuspended in serum-free media, and immediately plated in 384 well plates. After overnight recovery, compounds were added to each well, and cells were incubated for 4 hours before being fixed and stained with anti-Myc antibodies and with DAPI to label nuclei. Cells were then analyzed using high-content imaging to determine the levels of Myc expression per cell and IC₅₀ values of the compounds.

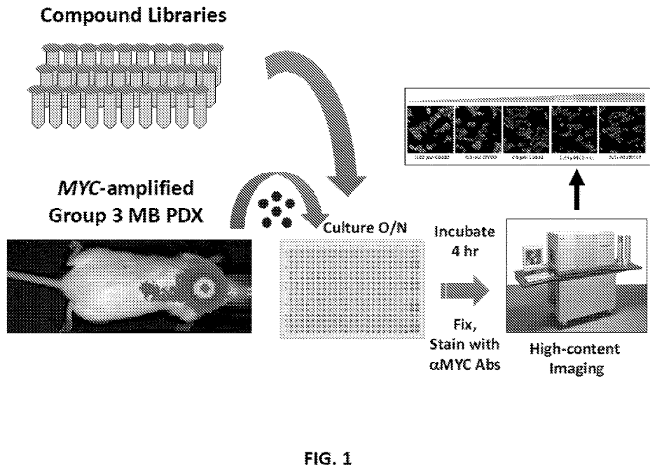

FIG. 1 shows the experimental set-up and workflow of the Myc inhibition assay.

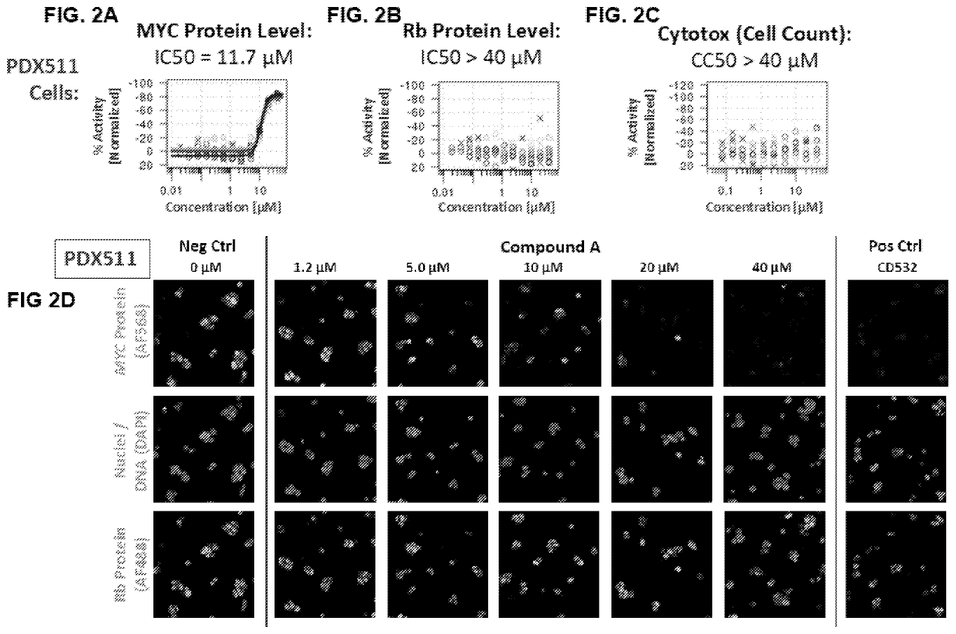

FIGS. 2A-2D shows the compound profile for Compound A as shown below. FIG. 2A shows Myc expression inhibition in PDX511 medulloblastoma cells by Compound A with an IC₅₀ of 11.7 μM. FIG. 2B shows that Compound A had no effect on the expression of the retinoblastoma (Rb) protein in PDX511 medulloblastoma cells. FIG. 2C shows that Compound A had no effect on cell viability of PDX511 medulloblastoma cells at 4 hours. FIG. 2D shows images of immunofluorescence-based detection of Myc expression (top row), cell viability (middle row), and Rb protein expression (bottom row) of PDX511 medulloblastoma cells treated with Compound A at various concentrations.

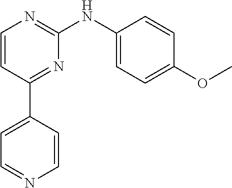 [SMILES: COc1ccc(Nc2nccc(-c3ccncc3)n2)cc1]

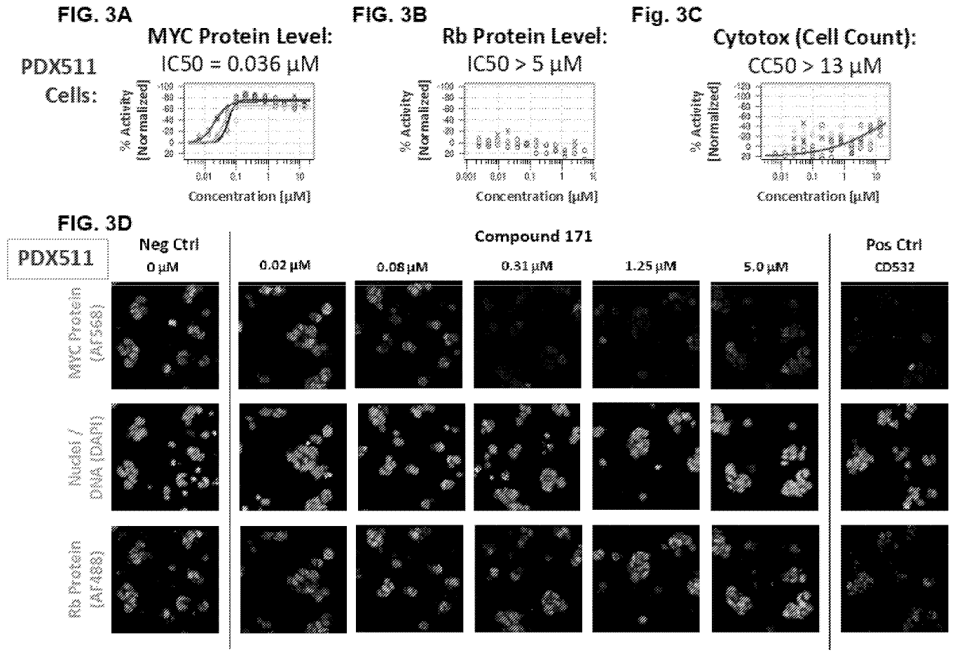

FIGS. 3A-3D show the compound profile for Compound 171. FIG. 3A shows Myc expression inhibition in PDX511 medulloblastoma cells by Compound 171. FIG. 3B shows that Compound 171 had no effect on expression of the retinoblastoma (Rb) protein in PDX511 medulloblastoma cells. FIG. 3C shows that Compound 171 had no effect on cell viability of PDX511 medulloblastoma cells at 4 hours. FIG. 3D shows images of immunofluorescence-based detection of Myc expression (top row), cell viability (middle row), and Rb protein expression (bottom row) of PDX511 medulloblastoma cells treated with Compound 171 at various concentrations.

Table 2 below shows the IC₅₀ value ranges for Myc inhibition assays of compounds described herein, with compounds having *=IC₅₀ less than 0.2 μM; A=IC₅₀ greater than or equal to 0.2 μM and less than 1 μM; B=IC₅₀ greater than or equal to 1 μM and less than 10 μM; and C=IC₅₀ greater than or equal to 10 μM and less than 100 μM.

TABLE 2
IC₅₀ Value Ranges of Compounds in Myc Inhibition Assays

| | MYC | | MYC | | MYC | | MYC |
|---|---|---|---|---|---|---|---|
| Cmpd | Inhibition | Cmpd | Inhibition | Cmpd | Inhibition | Cmpd | Inhibition |
| No. | IC50 (μM) | No. | IC50 (μM) | No. | IC50 (μM) | No. | IC50 (μM) |
| 1 | * | 2 | A | 3 | B | 4 | B |
| 5 | * | 6 | B | 7 | * | 8 | * |
| 9 | A | 10 | B | 11 | A | 12 | A |
| 13 | * | 14 | C | 15 | A | 16 | * |
| 17 | C | 18 | B | 19 | A | 20 | A |
| 21 | A | 22 | C | 23 | B | 24 | A |
| 25 | * | 26 | * | 27 | * | 28 | B |
| 29 | B | 30 | B | 31 | * | 32 | A |
| 33 | A | 34 | B | 35 | * | 36 | A |
| 37 | A | 38 | A | 39 | * | 40 | A |
| 41 | * | 42 | A | 43 | * | 44 | * |
| 45 | * | 46 | * | 47 | A | 48 | A |
| 49 | B | 50 | B | 51 | B | 52 | B |
| 53 | B | 54 | B | 55 | A | 56 | A |
| 57 | B | 58 | B | 59 | A | 60 | A |
| 61 | B | 62 | B | 63 | B | 64 | B |
| 65 | A | 66 | B | 67 | A | 68 | A |
| 69 | * | 70 | * | 71 | A | 72 | B |
| 73 | B | 74 | B | 75 | B | 76 | B |
| 77 | A | 78 | B | 79 | A | 80 | B |
| 81 | A | 82 | B | 83 | B | 84 | B |
| 85 | B | 86 | A | 87 | A | 88 | B |
| 89 | B | 90 | * | 91 | A | 92 | B |
| 93 | A | 94 | B | 95 | * | 96 | B |
| 97 | B | 98 | A | 99 | A | 100 | A |
| 101 | B | 102 | * | 103 | A | 104 | B |
| 105 | * | 106 | * | 107 | B | 108 | B |
| 109 | B | 110 | B | 111 | B | 112 | B |
| 113 | B | 114 | B | 115 | * | 116 | C |
| 117 | C | 118 | C | 119 | * | 120 | B |
| 121 | B | 122 | B | 123 | C | 124 | C |
| 125 | B | 126 | A | 127 | A | 128 | B |
| 129 | B | 130 | A | 131 | B | 132 | A |
| 133 | * | 134 | * | 135 | * | 136 | * |
| 137 | B | 138 | B | 139 | A | 140 | * |
| 141 | * | 142 | A | 143 | A | 144 | A |
| 145 | B | 146 | B | 147 | B | 148 | B |
| 149 | A | 150 | B | 151 | * | 152 | B |
| 153 | B | 154 | A | 155 | C | 156 | C |
| 157 | C | 158 | C | 159 | A | 160 | B |
| 161 | * | 162 | C | 163 | C | 164 | B |
| 165 | B | 166 | C | 167 | C | 168 | C |
| 169 | C | 170 | B | 171 | * | 172 | B |
| 173 | C | 174 | C | 175 | C | 176 | B |
| 177 | C | 178 | C | 179 | C | 180 | B |
| 181 | A | 182 | B | 183 | B | 184 | C |
| 185 | B | 186 | C | 187 | C | 188 | B |
| 189 | B | 190 | B | 191 | B | 192 | B |
| 193 | C | 194 | B | 195 | C | 196 | C |
| 197 | B | 198 | B | 199 | * | 200 | B |
| 201 | B | 202 | C | 203 | A | 204 | A |
| 205 | B | 206 | C | 207 | C | 208 | C |
| 209 | B | 210 | B | 211 | A | 212 | B |
| 213 | B | 214 | C | 215 | C | 216 | C |
| 217 | C | 218 | C | 219 | B | 220 | C |
| 221 | C | 222 | C | 223 | C | 224 | C |
| 225 | C | 226 | C | 227 | C | 228 | B |
| 229 | C | 230 | C | 231 | C | 232 | C |
| 233 | B | 234 | A | 235 | C | 236 | C |
| 237 | C | 238 | B | 239 | C | 240 | B |
| 241 | A | 242 | A | 243 | B | 244 | B |
| 245 | C | 246 | C | 247 | B | 248 | C |
| 249 | B | 250 | B | 251 | B | 252 | C |
| 253 | C | 254 | * | 255 | B | 256 | * |
| 257 | A | 258 | * | 259 | * | 260 | * |
| 261 | * | 262 | B | 263 | * | 264 | * |
| 265 | * | 266 | B | 267 | * | 268 | A |
| 269 | A | 270 | B | 271 | B | 272 | A |
| 273 | B | 274 | * | 275 | * | 276 | A |
| 277 | * | 278 | * | 279 | * | 280 | * |
| 281 | * | | | | | | |

### Example 5—Inhibition of Myc Expression with Treatment of Compound 171 in Medulloblastoma PDX Cells in Mice

### Experimental Procedure

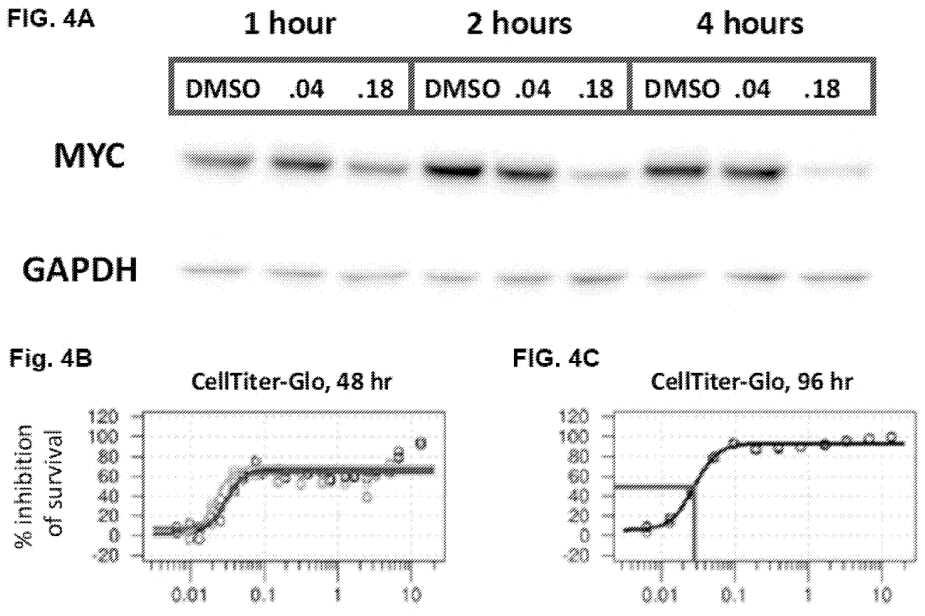

Medulloblastoma PDX cells were harvested from the brains of tumor-bearing mice, cultured overnight, and then treated with Compound 171 for various amounts of time. Cells cultured for time points of 1 hour, 2 hours, and 4 hours were lysed and subjected to Western blotting with antibodies specific for Myc or GAPDH as control, as shown in FIG. 4A. Cells cultured for longer time points of 48 hours and 96 hours were assayed for viability using the CellTiter Glo assay, as shown in FIG. 4B and FIG. 4C.

### Results

Cells exposed to 0.18 μM of Compound 171 showed inhibition of Myc expression as early as 1 hour, and greater inhibition at 2 and 4 hours, as shown in FIG. 4A. Compound 171 led to an 80% reduction in cell viability by 48 hours, and 100% reduction in cell viability by 96 hours, as shown in FIG. 4B and FIG. 4C.

### Conclusion

Compound 171 inhibited Myc expression in medulloblastoma PDX Cells in mice.

### Example 6—Inhibition of Myc Expression with Treatment of Compound 171 in Cell Lines Derived from Various Cancers

### Experimental Procedure

Cell lines derived from acute monocytic leukemia, acute promyelocytic leukemia, pancreatic adenocarcinoma and small cell lung cancer were treated with vehicle (DMSO) or the indicated concentrations of Compound 171 for 4 hours, and then lysed and subjected to Western blotting using antibodies specific for Myc or GAPDH as control.

### Results

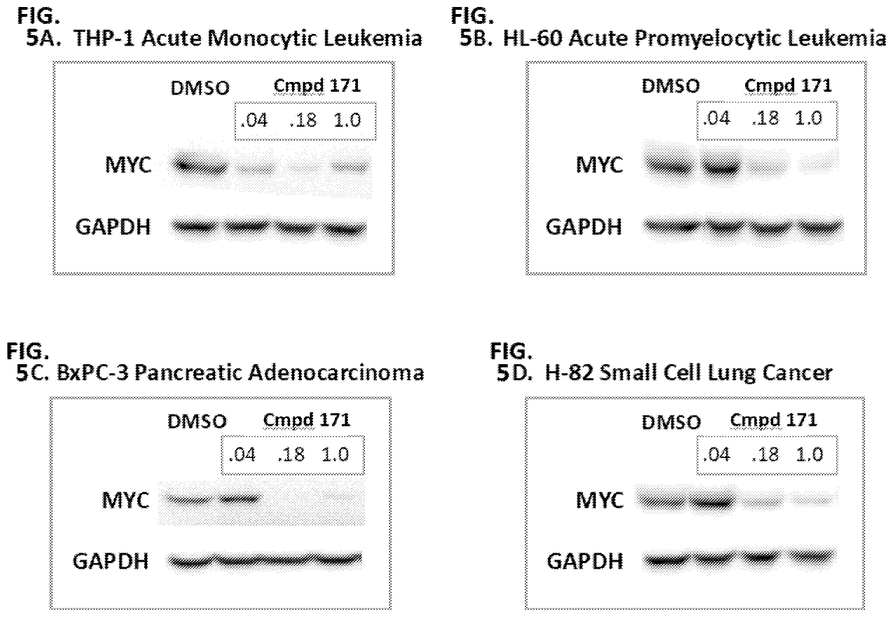

As shown in FIGS. 5A-5D, all cell lines tested showed substantial inhibition of Myc at both 0.18 μM and 1.0 μM concentrations of Compound 171. Specifically, FIG. 5A shows the inhibition result from the THP-1 cell line derived from acute monocytic leukemia. FIG. 5B shows the inhibition result from the HL-60 cell line derived from acute promyelocytic leukemia. FIG. 5C shows the inhibition result from the BxPC-3 cell line derived from pancreatic adenocarcinoma. FIG. 5D shows the inhibition result from the H-82 cell line derived from small cell lung cancer.

### Conclusion

Compound 171 inhibited Myc expression in cell lines derived from acute monocytic leukemia, acute promyelocytic leukemia, pancreatic adenocarcinoma and small cell lung cancer.

### Example 7—Accumulation of Compound 171 in the Brain of Mice Following Oral Administration of the Compound

### Experimental Procedure

Non-tumor-bearing NOD-SCID-IL2Rgamma knockout (NSG) mice were treated with Compound 171 at 100 mg/kg via oral administration. Blood was collected retro-orbitally or by cardiac puncture for measuring compound concentrations. For measurements of compound concentration in the brain, animals were sacrificed, and brain tissue was harvested and homogenized. Compound concentration was determined by liquid chromatography-mass spectrometry (LCMS).

### Results

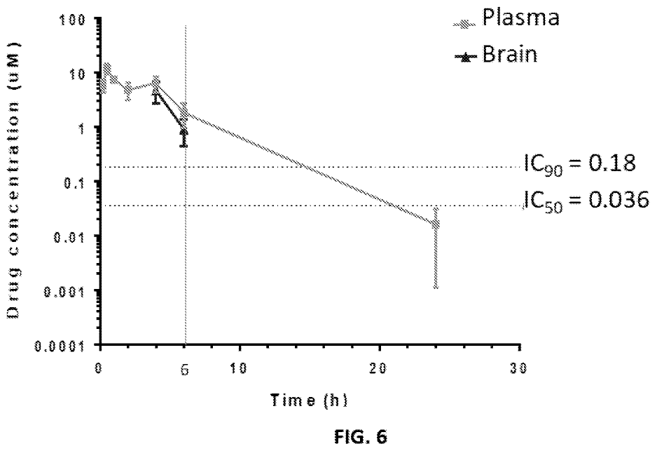

As shown in FIG. 6, concentrations of Compound 171 remained above the IC₉₀ level in both plasma and brain for more than 6 hours following oral administration of the compound.

### Conclusion

Compound 171 demonstrated the ability to cross the blood-brain-barrier with good retention in the brain following oral administration in mice.

Example 8-Reduction of Myc Levels with Oral Administration of Compound 171 in Intracranial Tumors in Mice

### Experimental Procedure

NSG mice transplanted with medulloblastoma PDX cells were treated by oral administration with either vehicle (10% DMSO, 10% Tween 80) or Compound 171 (100 mg/kg). After 5.5 hours, animals were sacrificed, and tumors were harvested, dissociated, lysed and subjected to Western blotting with antibodies specific for Myc or GAPDH as control.

### Results

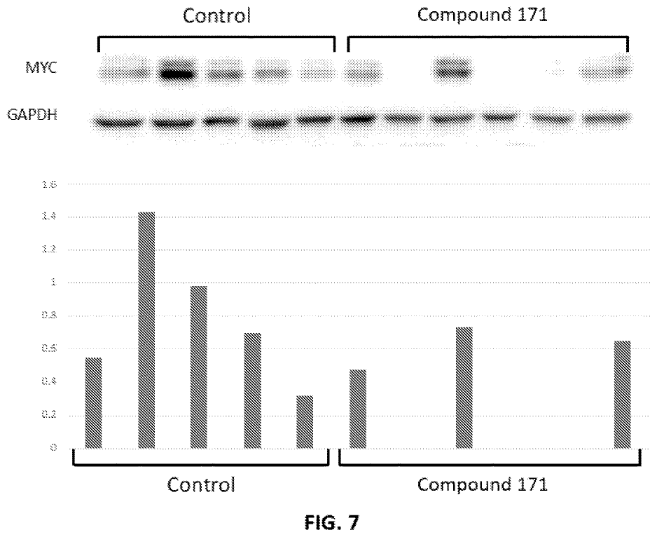

As shown in FIG. 7, Myc expression was undetectable after more than 5 hours in 3 out of 6 mice that were treated with Compound 171 via oral administration.

### Conclusion

Compound 171 inhibited Myc expression in intracranial tumors in mice following oral administration.

The examples and embodiments described herein are for illustrative purposes only and various modifications or changes suggested to persons skilled in the art are to be included within the spirit and purview of this application and scope of the appended claims.

What is claimed is:

1. A compound of Formula (I): 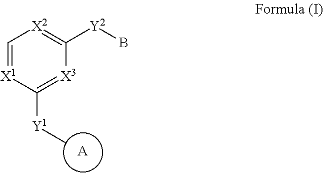
or a pharmaceutically acceptable salt thereof, for use in treating a disease or disorder mediated by a Myc protein,
wherein:
X¹, X², and X³ are each independently N or CR¹, and wherein at least one of X¹, X², and X³ is N;
Y¹ is a bond, —O—, —S—, —CH₂—, or —N(R²)(CH₂)ₙ—;
Y² is —O—, —S—, —CH₂—, or —N(R²)(CH₂)ₙ—; 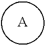
is phenyl, 5-9 membered heteroaryl, 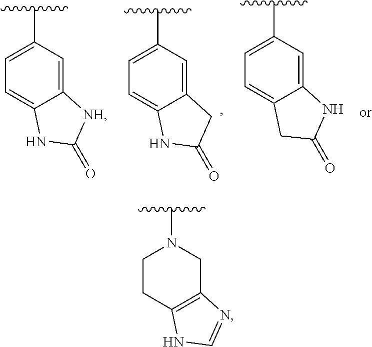
wherein the phenyl and 5-9 membered heteroaryl are each optionally substituted with 1-3 R⁹ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂, with the proviso 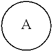
is not 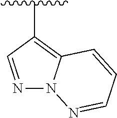 [SMILES: *c1cnn2ncccc12]
optionally substituted with 1-3 R′ groups each independently selected from the group consisting of halogen, C₁-C₅ alkyl, C₂-C₅-alkenyl, C₂-C₅-alkynyl, C₁-C₅ haloalkyl, —(CH₂)ₙOR³, and —NH₂;
B is C₁-C₅ alkyl, aryl, heteroaryl, 5-9 membered heterocycloalkyl, or C₃-C₁₀-cycloalkyl, wherein the aryl, heteroaryl, 5-9 membered heterocycloalkyl, and C₃-C₁₀-cycloalkyl are each optionally substituted with 1-3 R⁸ groups each independently selected from the group consisting of —(CH₂)ₙCN, —(CH₂)ₙOH, —(CH₂)ₙN(R⁴)₂, —(CH₂)ₙC(O)N(R⁴)₂, —(CH₂)ₙNR⁴C(O)R⁴, —(CH₂)ₙNR⁴C(O)N(R⁴)₂, —(CH₂)ₙNR⁴ (C₁-C₅)alkylene(O)R⁴⁴, —(CH₂)ₙC(O)R⁴, —(CH₂)ₙCO₂R⁴, —(CH₂)ₙOC(O)R⁴, —(CH₂)ₙOC(O)OR⁴, —(CH₂)ₙO(C₁-C₅-alkylene)N(R⁴)₂, —(CH₂)ₙSR⁴, —(CH₂)ₙS(O)R⁴, —(CH₂)ₙS(O)₂R⁴, —(CH₂)ₙN(R⁴)S(O)₂R⁴, —(CH₂)ₙS(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo;
R¹ is hydrogen, halogen, or C₁-C₅ alkyl;
R² and R³ are each independently hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl; and
each n is independently 0, 1, 2, 3, or 4.

2. The compound of claim 1, wherein X¹ is CR¹, and X² and X³ are N.

3. The compound of claim 1, wherein X¹ and X² are each independently CR¹, and X₃ is N.

4. The compound of claim 1, wherein X¹ and X³ are each independently CR¹, and X² is N.

5. The compound of claim 1, wherein X¹, X² and X³ are N.

6. The compound of any one of claims 1 to 5, wherein R¹ is hydrogen or —CH₃; and B is aryl or heteroaryl each optionally substituted with 1-3 R⁸ groups each independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, alkylaryl, heteroaryl, 5-9 membered heterocycloalkyl, 5-9-membered cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

7. The compound of any one of claims 1 to 6, wherein 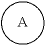
is 5-9-membered heteroaryl or phenyl, wherein the 5-9 membered heteroaryl and the phenyl are each optionally substituted with 1-3 R⁹ groups each independently selected from the group consisting of halogen, C₁-C₅ alkyl, C₁-C₅ haloalkyl, —OR³, and —NH₂.

8. The compound of claim 7, wherein 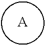
is 5-9 membered heteroaryl or phenyl, wherein the 5-9 membered heteroaryl and the phenyl are each substituted with 1-3 R⁹ groups each independently selected from the group consisting of halogen, —OH, —OCH₃, —NH₂, and —CH₃.

9. The compound of any one of claims 1 to 8, wherein Y¹ is a bond.

10. The compound of claim 9, wherein 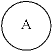
is selected from the group consisting of:

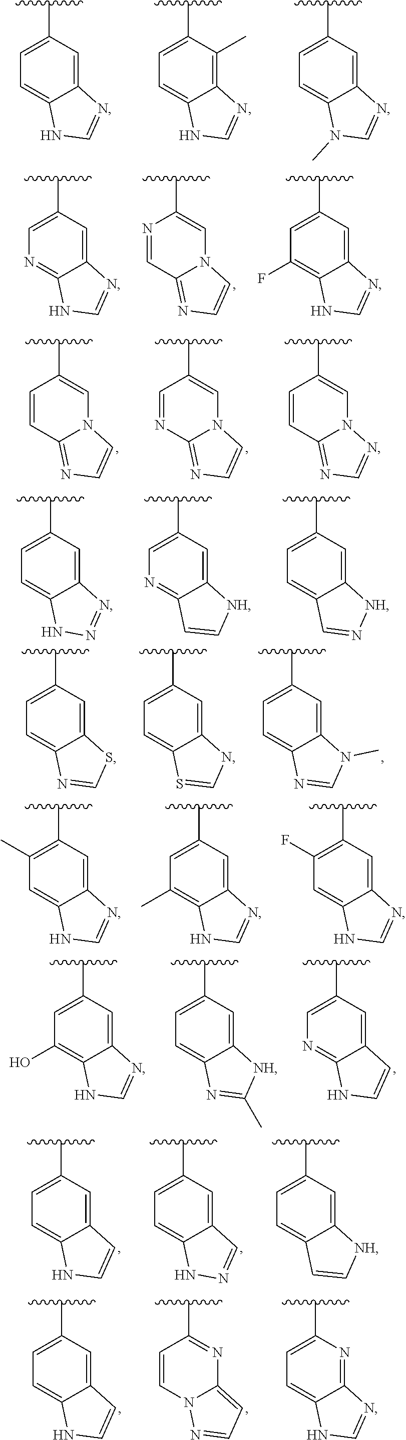

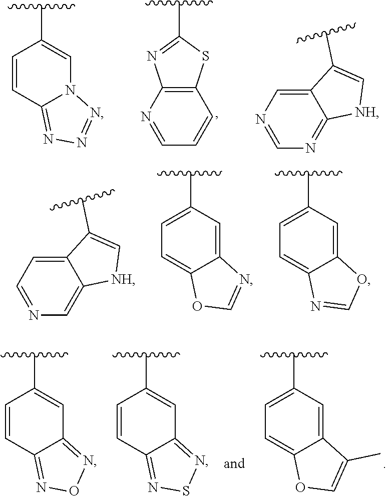

11. The compound of claim 9, wherein 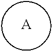
is selected from the group consisting of:

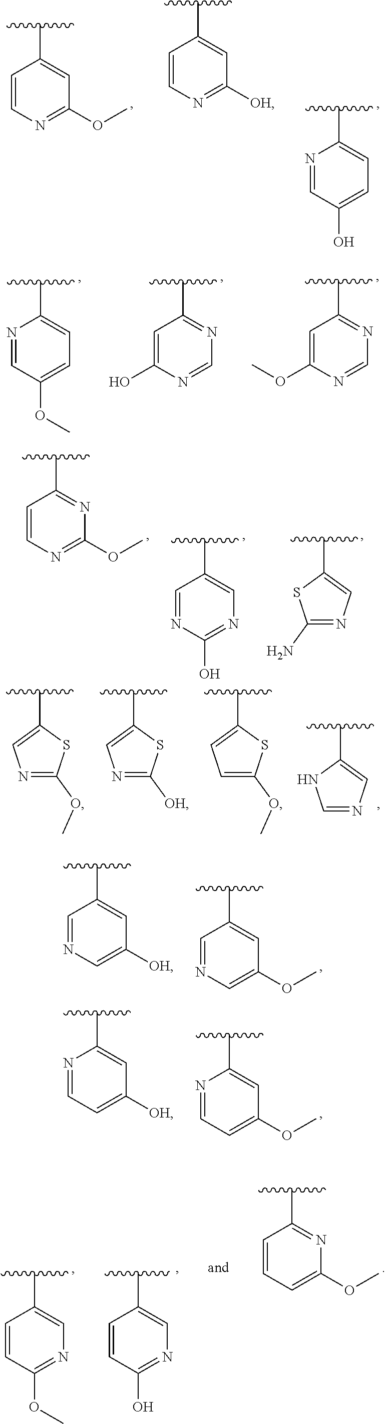

12. The compound of claim 9, wherein 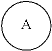
is selected from

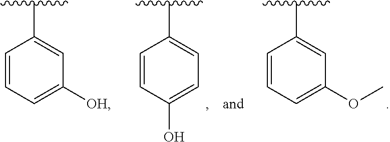

13. The compound of any one of claims 1 to 8, wherein Y¹ is —O—, or —N(R²)(CH₂)ₙ—.

14. The compound of claim 13, wherein 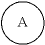
is selected from

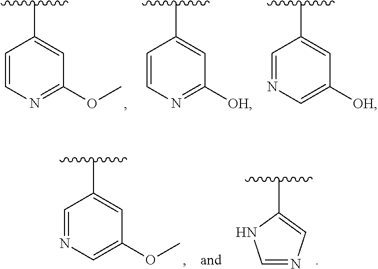

15. The compound of any one of claims 1 to 14, wherein Y² is —N(R²)(CH₂)ₙ—.

16. The compound of any one of claims 1 to 15, wherein B is C₁-C₅ alkyl, C₃-C₉-cycloalkyl, or 5-9 membered heterocycloalkyl.

17. The compound of claim 16, wherein B is selected from

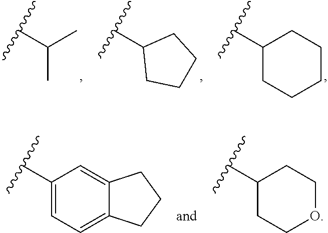

18. The compound of claim 16 or 17, wherein the compound is: 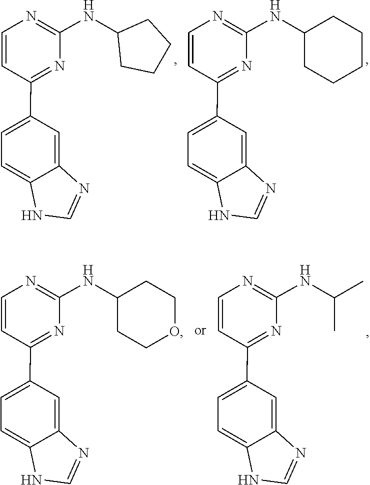
or a pharmaceutically acceptable salt thereof.

19. The compound of any one of claims 1 to 15, wherein B is aryl, and wherein the aryl is optionally substituted with 1-3 R⁸ groups each independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

20. The compound of claim 19, wherein B is aryl substituted with 1 or 2 R⁸ groups each independently selected from the group consisting of halogen, C₁-C₅-alkyl, aryl, C₁-C₅-alkoxy, heteroaryl, C₅-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)₂NR⁴, —NR⁴C(O)R⁴, —NR⁴ (C₁-C₅)alkylene(O)R⁴⁴, and —O(C₁-C₅-alkylene)N(R⁴)₂, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

21. The compound of claim 19 or 20, wherein B is phenyl substituted with 1 or 2 R⁸ groups each independently selected from the group consisting of —F, —Cl, —Br, —CH₃, —CF₃, —CN, —CH(CH₃)₂, —C(CH₃) 3, —CH₂CH₂CH₃, —OCF₃, —OCH₃, —CONH₂, —COOH, —COOCH₃, pyridinyl, phenyl, pyrazolyl, ethynyl, cyclohexyl, cyclopentyl, morpholinyl, tetrahydropyranyl, piperidinyl, piperazinyl, —SO₂NH₂, —SO₂CH₃,

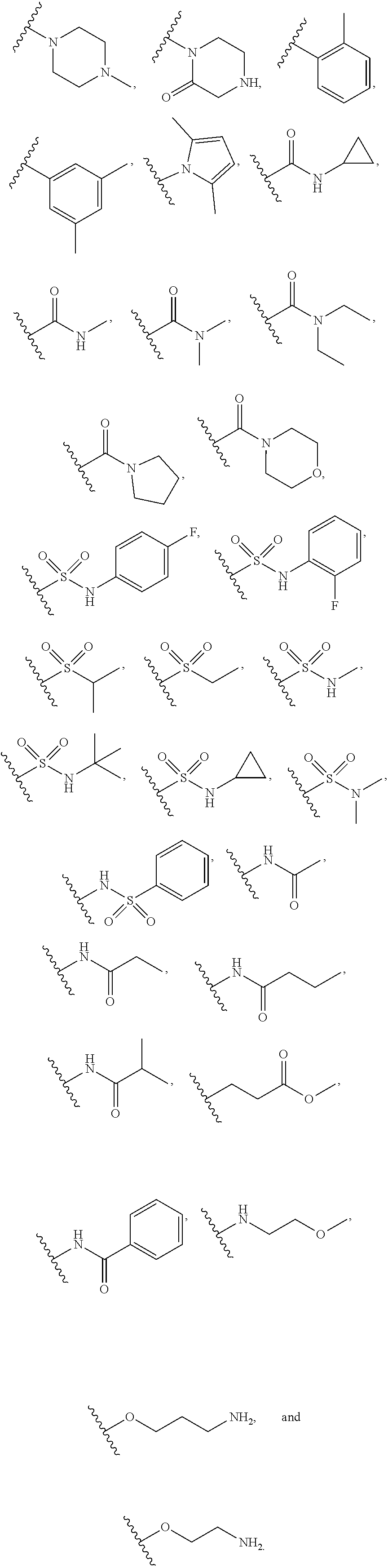

22. The compound of any one of claims 19 to 21, wherein B is aryl substituted with one R⁸ group selected from halogen, C₁-C₅-alkyl, aryl, C₁-C₅-alkoxy, heteroaryl, C₅-C₉-cycloalkyl, 5-9-membered heterocycloalkyl, —CO₂R⁴, —C(O)R⁴, —NR⁴C(O)R⁴, —S(O)₂R⁴, —S(O)₂R⁴, —S(O)₂NR⁴, —NR⁴C(O)R⁴, —NR⁴ (C₁-C₅)alkylene(O)R^{4A}, and —O(C₁-C₅-alkylene)N(R⁴)₂, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

23. The compound of any one of claims 19 to 22, wherein B is selected from the group consisting of:

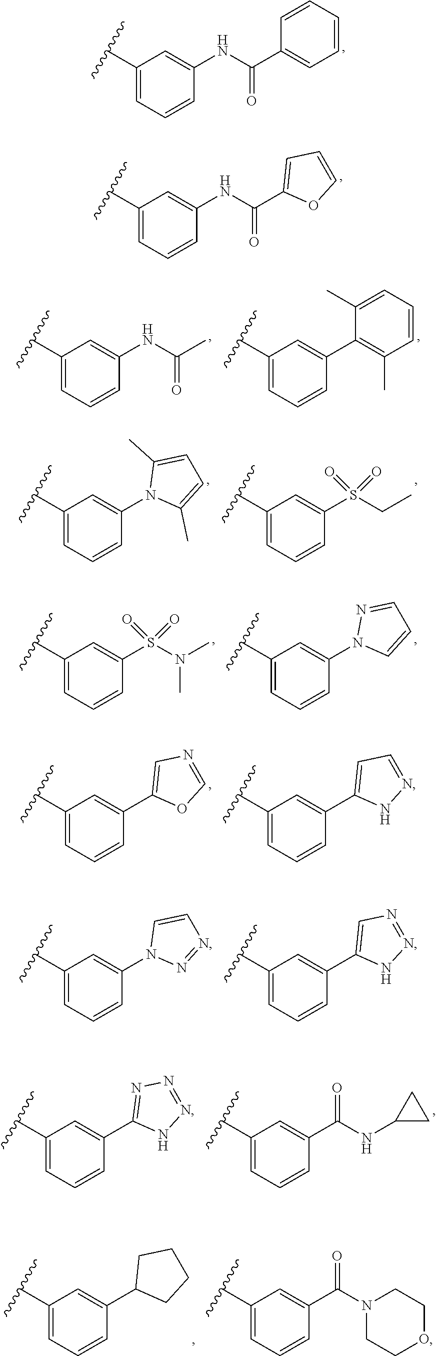

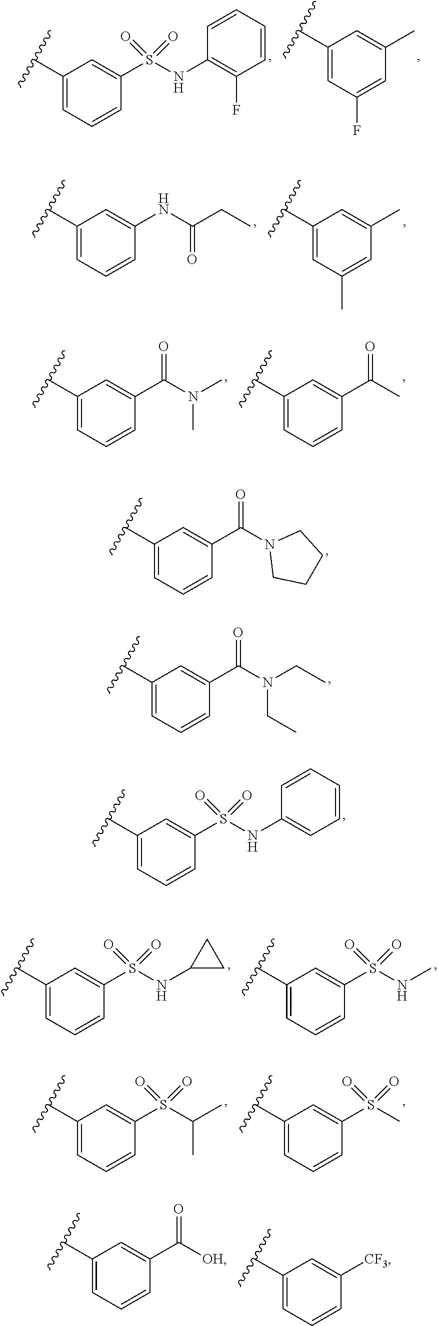

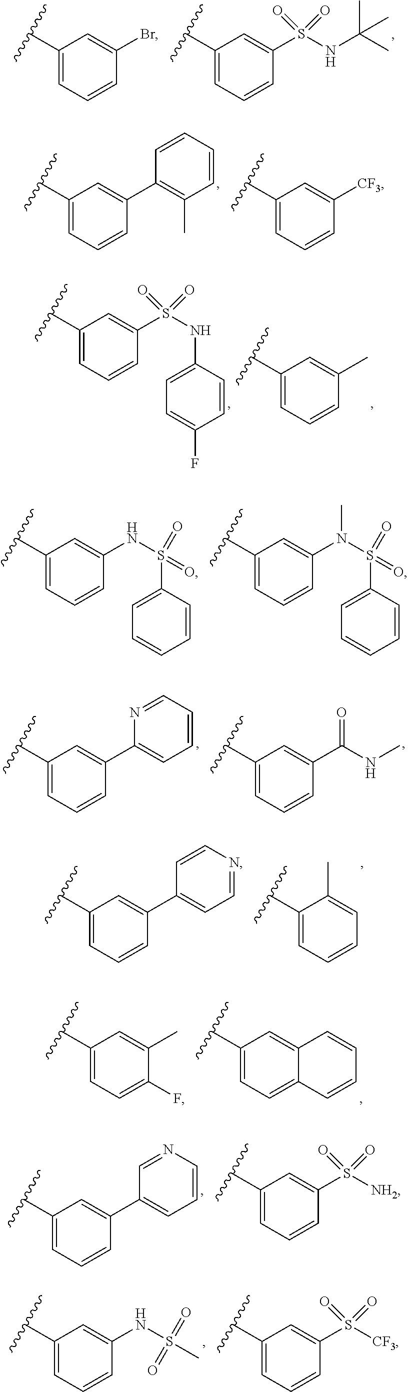

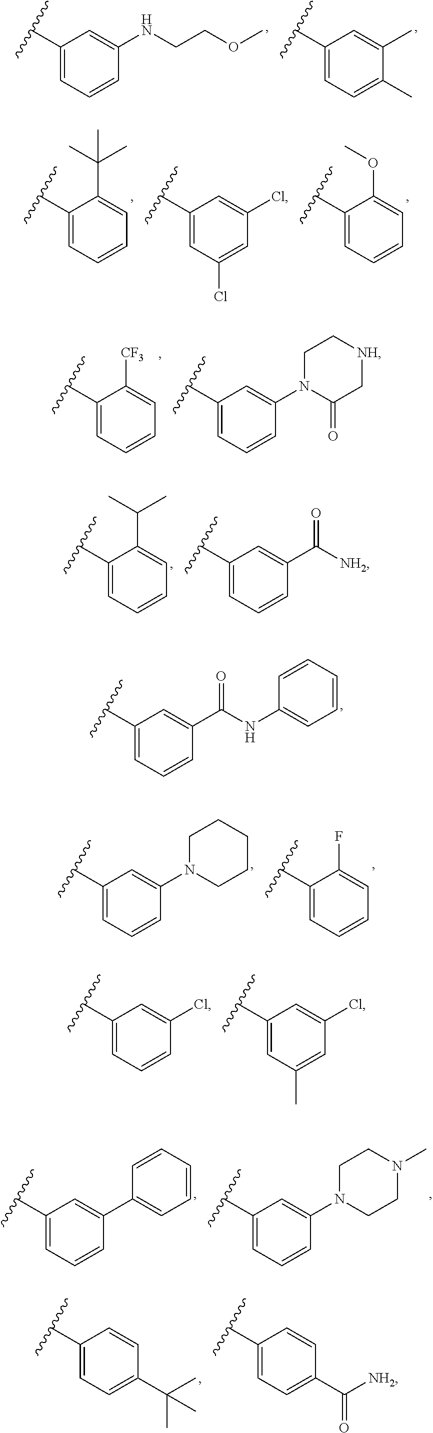

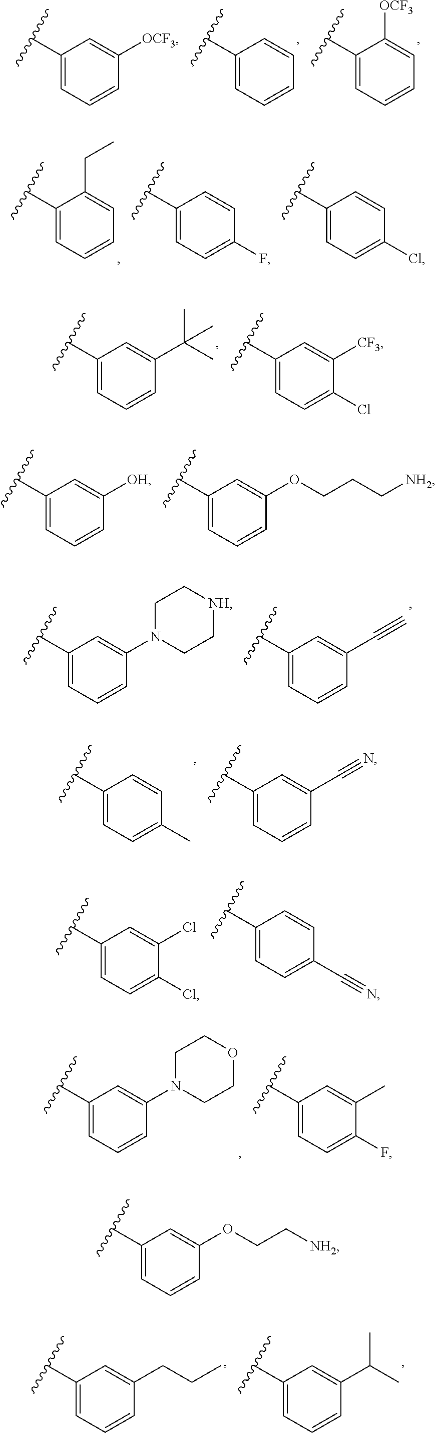

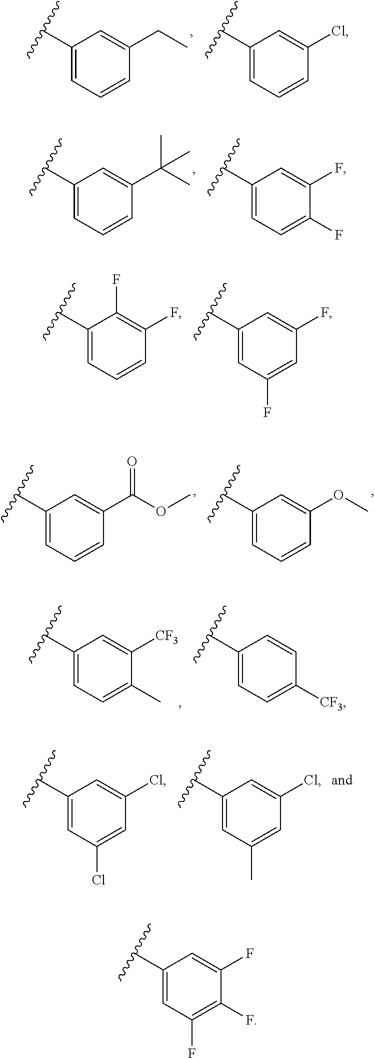

24. The compound of any one of claims 19 to 23, wherein the compound is selected from the group consisting of: 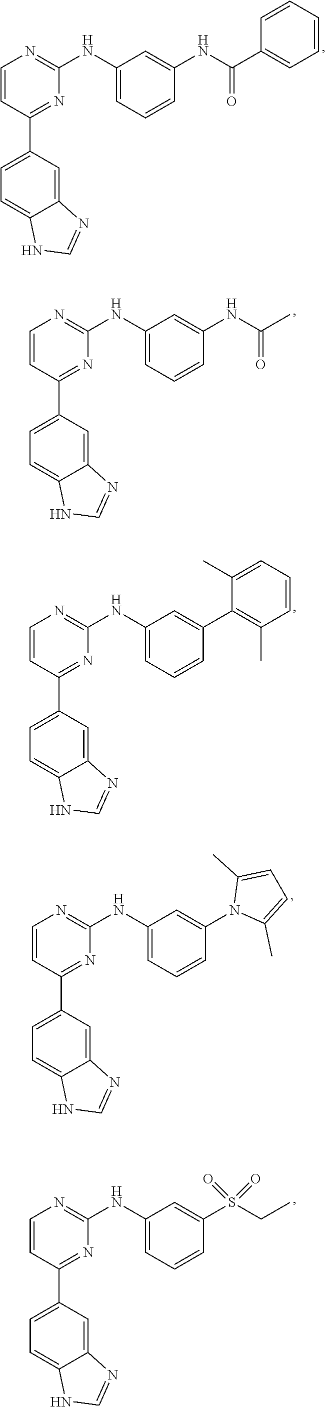 [SMILES: O=C(Nc1cccc(Nc2nccc(-c3ccc4[nH]cnc4c3)n2)c1)c1ccccc1 ; CC(=O)Nc1cccc(Nc2nccc(-c3ccc4[nH]cnc4c3)n2)c1 ; Cc1cccc(C)c1-c1cccc(Nc2nccc(-c3ccc4[nH]cnc4c3)n2)c1 ; Cc1ccc(C)n1-c1cccc(Nc2nccc(-c3ccc4[nH]cnc4c3)n2)c1 ; CCS(=O)(=O)c1cccc(Nc2nccc(-c3ccc4[nH]cnc4c3)n2)c1] 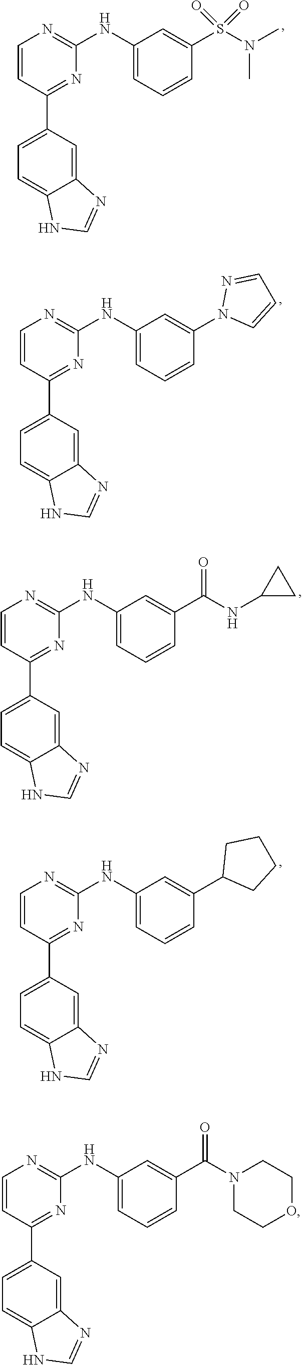 [SMILES: CN(C)S(=O)(=O)c1cccc(Nc2nccc(-c3ccc4[nH]cnc4c3)n2)c1 ; c1cc(Nc2nccc(-c3ccc4[nH]cnc4c3)n2)cc(-n2cccn2)c1 ; O=C(NC1CC1)c1cccc(Nc2nccc(-c3ccc4[nH]cnc4c3)n2)c1 ; c1cc(Nc2nccc(-c3ccc4[nH]cnc4c3)n2)cc(C2CCCC2)c1 ; O=C(c1cccc(Nc2nccc(-c3ccc4[nH]cnc4c3)n2)c1)N1CCOCC1] 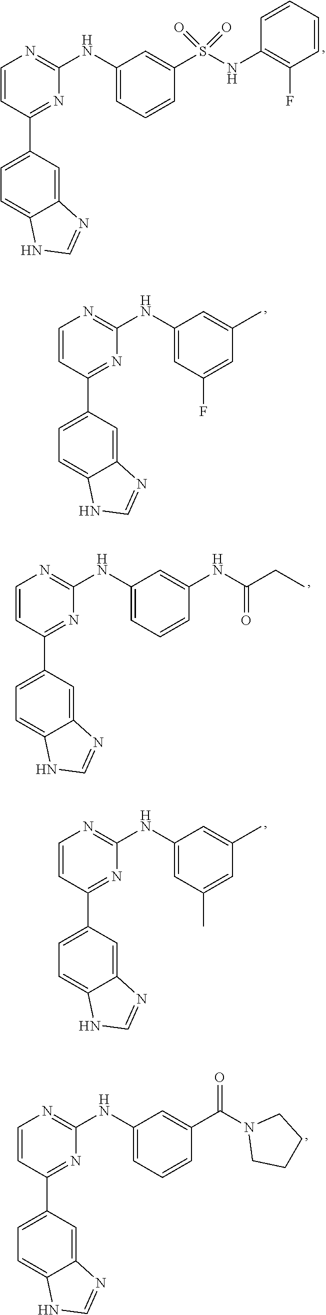 [SMILES: O=S(=O)(Nc1ccccc1F)c1cccc(Nc2nccc(-c3ccc4[nH]cnc4c3)n2)c1 ; Cc1cc(F)cc(Nc2nccc(-c3ccc4[nH]cnc4c3)n2)c1 ; CCC(=O)Nc1cccc(Nc2nccc(-c3ccc4[nH]cnc4c3)n2)c1 ; Cc1cc(C)cc(Nc2nccc(-c3ccc4[nH]cnc4c3)n2)c1 ; O=C(c1cccc(Nc2nccc(-c3ccc4[nH]cnc4c3)n2)c1)N1CCCC1] 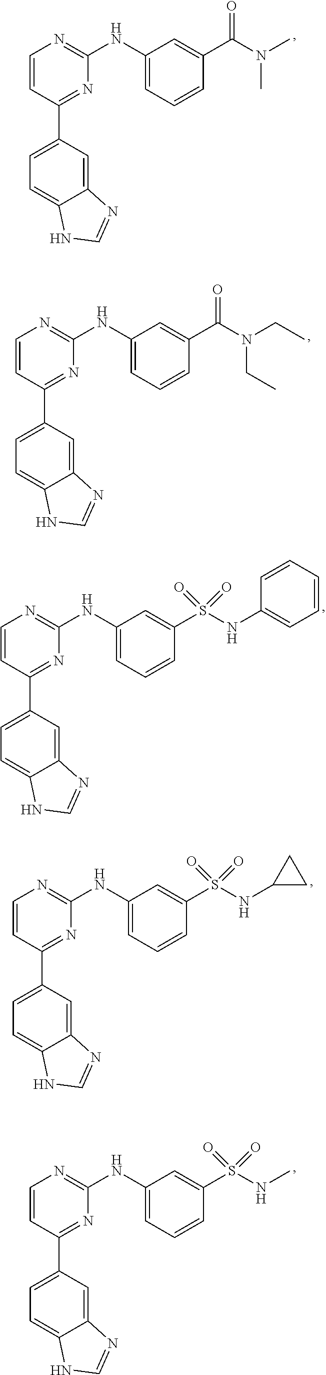 [SMILES: CN(C)C(=O)c1cccc(Nc2nccc(-c3ccc4[nH]cnc4c3)n2)c1 ; CCN(CC)C(=O)c1cccc(Nc2nccc(-c3ccc4[nH]cnc4c3)n2)c1 ; O=S(=O)(Nc1ccccc1)c1cccc(Nc2nccc(-c3ccc4[nH]cnc4c3)n2)c1 ; O=S(=O)(NC1CC1)c1cccc(Nc2nccc(-c3ccc4[nH]cnc4c3)n2)c1 ; CNS(=O)(=O)c1cccc(Nc2nccc(-c3ccc4[nH]cnc4c3)n2)c1] 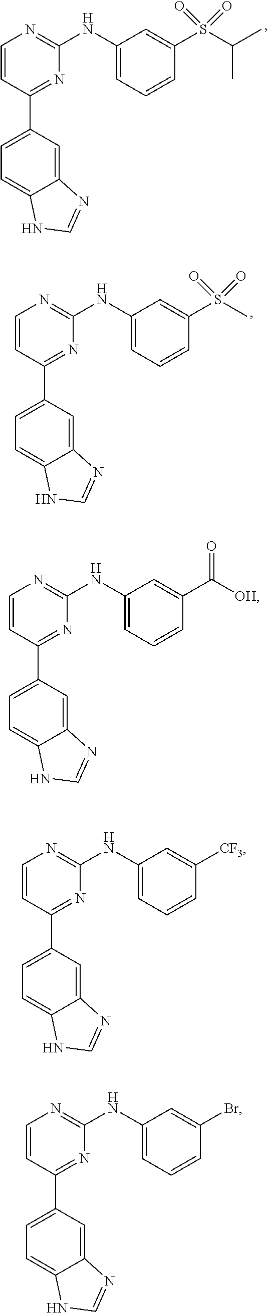 [SMILES: CC(C)S(=O)(=O)c1cccc(Nc2nccc(-c3ccc4[nH]cnc4c3)n2)c1 ; CS(=O)(=O)c1cccc(Nc2nccc(-c3ccc4[nH]cnc4c3)n2)c1 ; O=C(O)c1cccc(Nc2nccc(-c3ccc4[nH]cnc4c3)n2)c1 ; FC(F)(F)c1cccc(Nc2nccc(-c3ccc4[nH]cnc4c3)n2)c1 ; Brc1cccc(Nc2nccc(-c3ccc4[nH]cnc4c3)n2)c1] 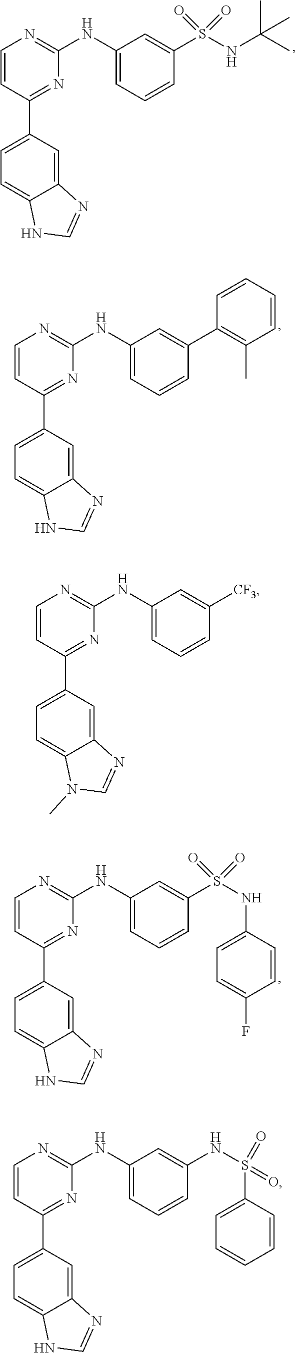 [SMILES: CC(C)(C)NS(=O)(=O)c1cccc(Nc2nccc(-c3ccc4[nH]cnc4c3)n2)c1 ; Cc1ccccc1-c1cccc(Nc2nccc(-c3ccc4[nH]cnc4c3)n2)c1 ; Cn1cnc2cc(-c3ccnc(Nc4cccc(C(F)(F)F)c4)n3)ccc21 ; O=S(=O)(Nc1ccc(F)cc1)c1cccc(Nc2nccc(-c3ccc4[nH]cnc4c3)n2)c1 ; O=S(=O)(Nc1cccc(Nc2nccc(-c3ccc4[nH]cnc4c3)n2)c1)c1ccccc1] 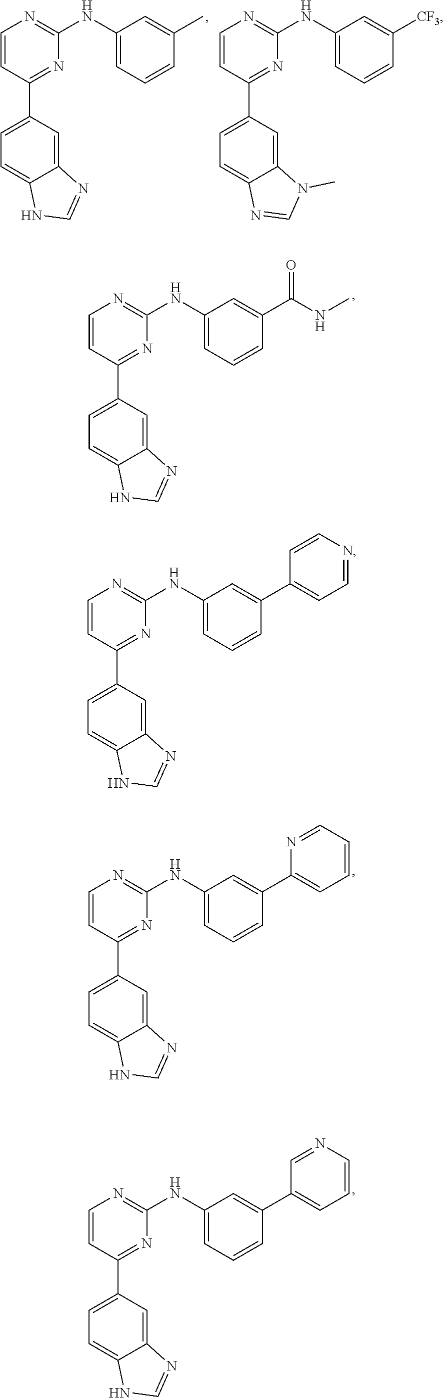 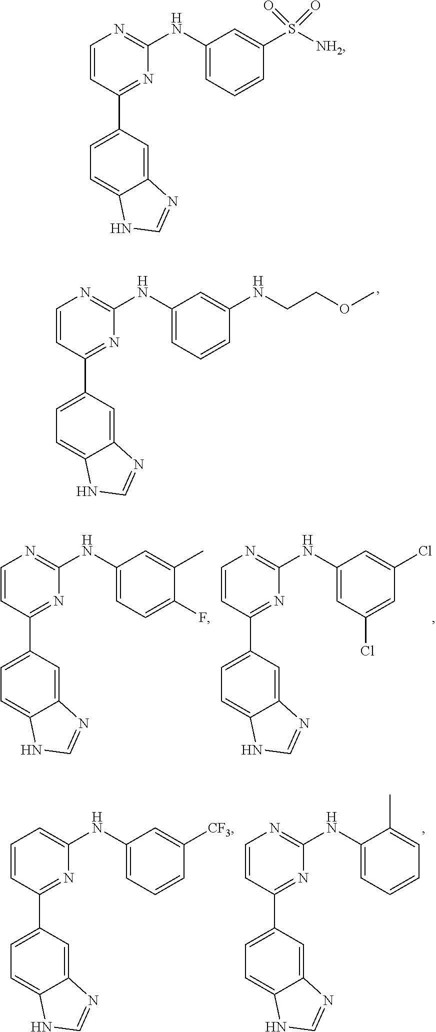 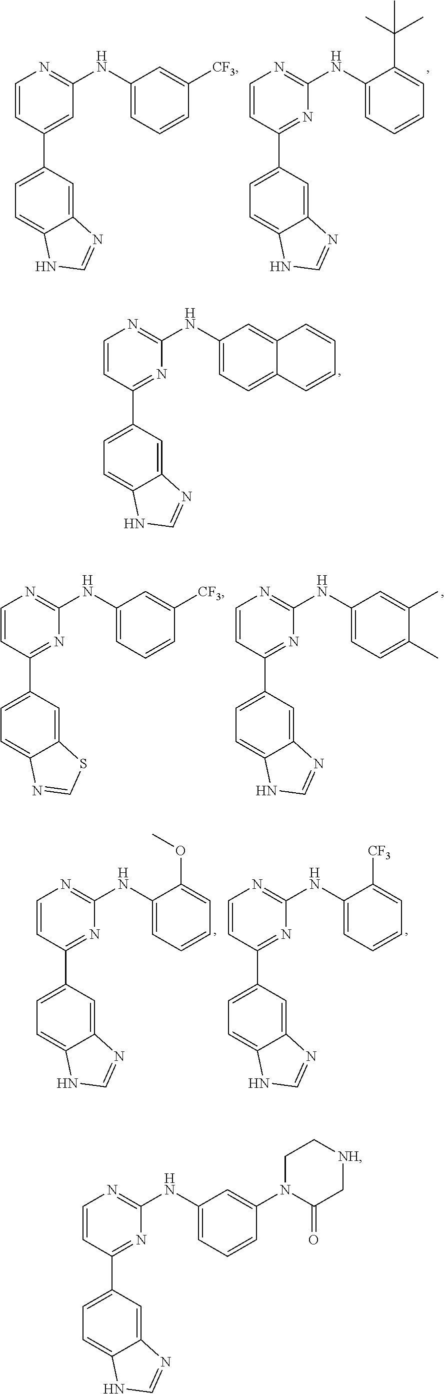 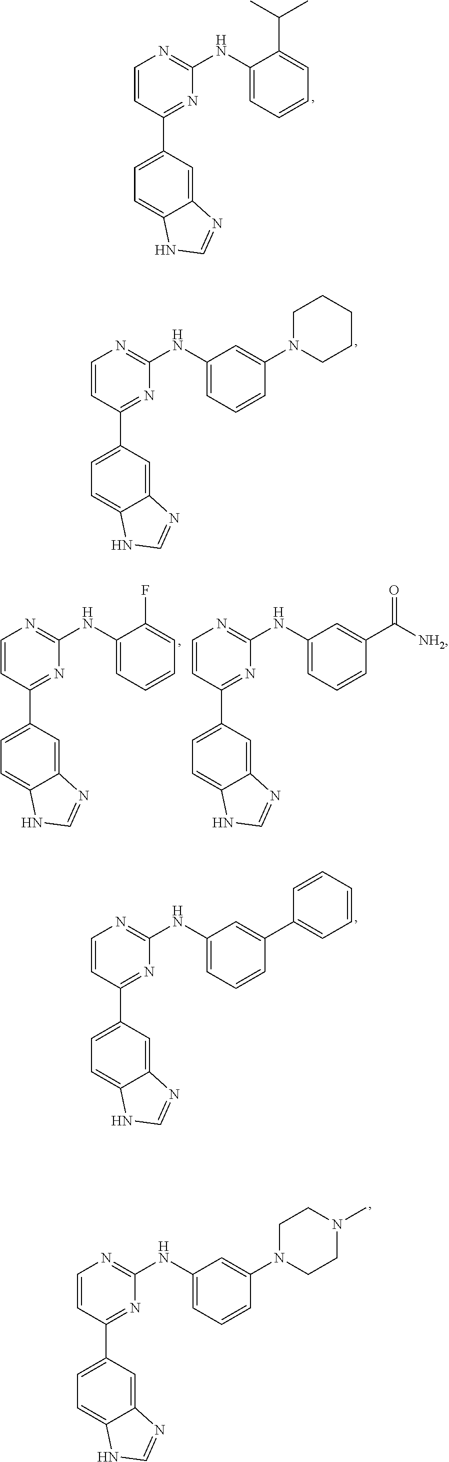 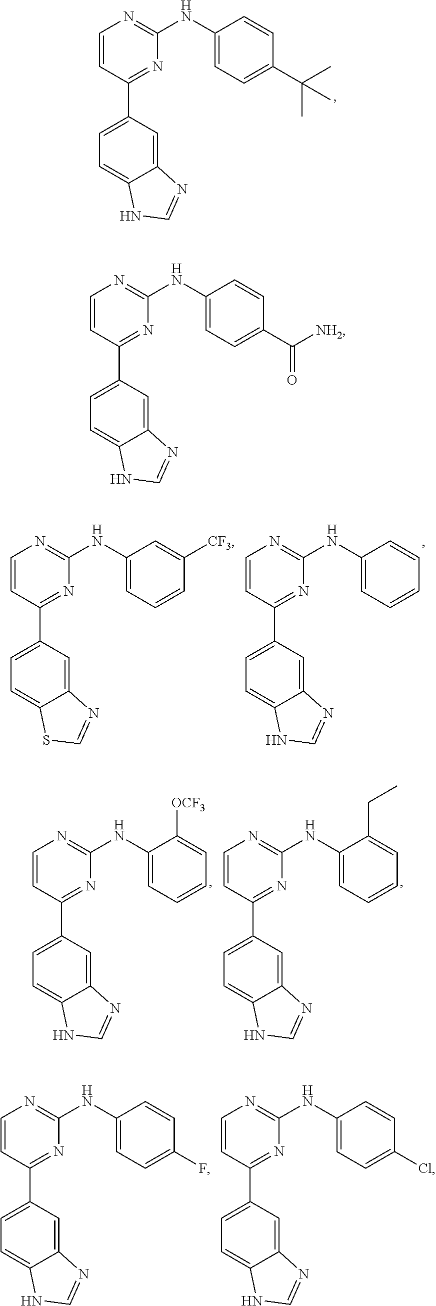 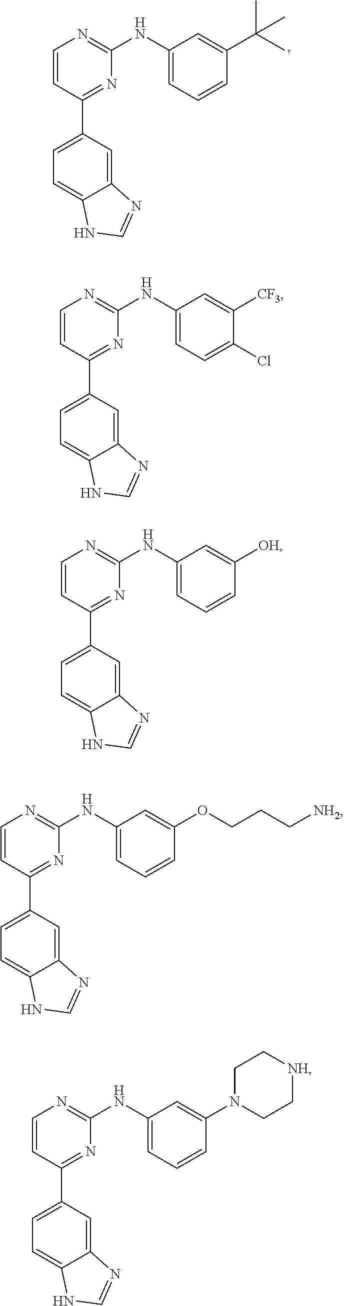 [SMILES: CC(C)(C)c1cccc(Nc2nccc(-c3ccc4[nH]cnc4c3)n2)c1 ; FC(F)(F)c1cc(Nc2nccc(-c3ccc4[nH]cnc4c3)n2)ccc1Cl ; Oc1cccc(Nc2nccc(-c3ccc4[nH]cnc4c3)n2)c1 ; NCCCOc1cccc(Nc2nccc(-c3ccc4[nH]cnc4c3)n2)c1 ; c1cc(Nc2nccc(-c3ccc4[nH]cnc4c3)n2)cc(N2CCNCC2)c1] 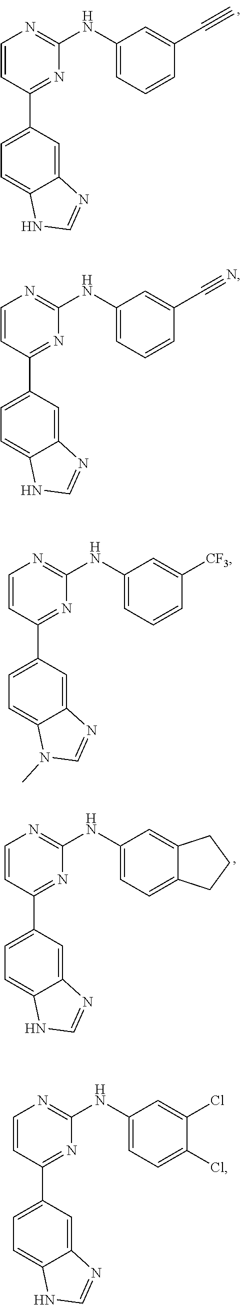 [SMILES: C#Cc1cccc(Nc2nccc(-c3ccc4[nH]cnc4c3)n2)c1 ; N#Cc1cccc(Nc2nccc(-c3ccc4[nH]cnc4c3)n2)c1 ; Cn1cnc2cc(-c3ccnc(Nc4cccc(C(F)(F)F)c4)n3)ccc21 ; c1cc(-c2ccc3[nH]cnc3c2)nc(Nc2ccc3c(c2)CCC3)n1 ; Clc1ccc(Nc2nccc(-c3ccc4[nH]cnc4c3)n2)cc1Cl] 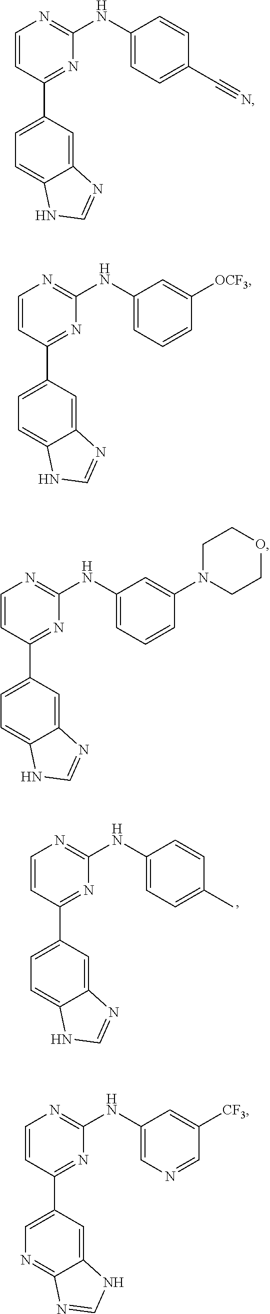 [SMILES: N#Cc1ccc(Nc2nccc(-c3ccc4[nH]cnc4c3)n2)cc1 ; FC(F)(F)Oc1cccc(Nc2nccc(-c3ccc4[nH]cnc4c3)n2)c1 ; c1cc(Nc2nccc(-c3ccc4[nH]cnc4c3)n2)cc(N2CCOCC2)c1 ; Cc1ccc(Nc2nccc(-c3ccc4[nH]cnc4c3)n2)cc1 ; FC(F)(F)c1cncc(Nc2nccc(-c3cnc4nc[nH]c4c3)n2)c1] 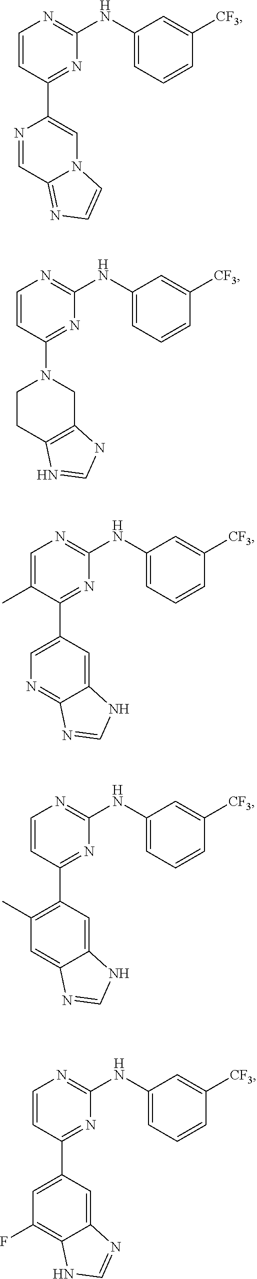 [SMILES: FC(F)(F)c1cccc(Nc2nccc(-c3cn4ccnc4cn3)n2)c1 ; FC(F)(F)c1cccc(Nc2nccc(N3CCc4nc[nH]c4C3)n2)c1 ; Cc1cnc(Nc2cccc(C(F)(F)F)c2)nc1-c1cnc2nc[nH]c2c1 ; Cc1cc2nc[nH]c2cc1-c1ccnc(Nc2cccc(C(F)(F)F)c2)n1 ; Fc1cc(-c2ccnc(Nc3cccc(C(F)(F)F)c3)n2)cc2nc[nH]c12] 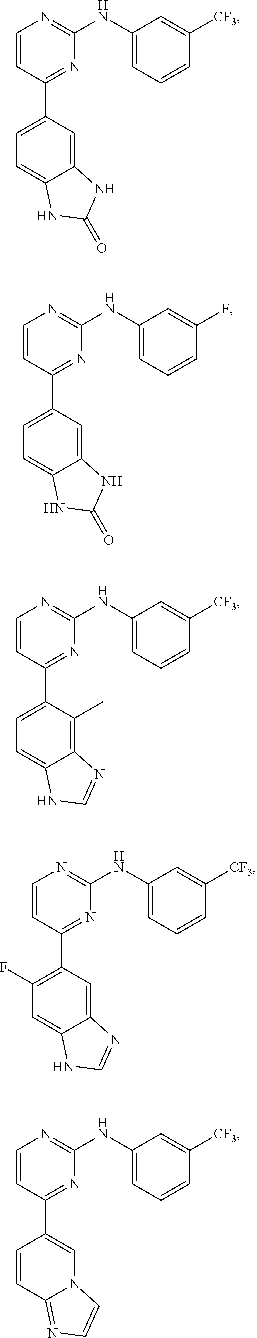 [SMILES: O=c1[nH]c2ccc(-c3ccnc(Nc4cccc(C(F)(F)F)c4)n3)cc2[nH]1 ; O=c1[nH]c2ccc(-c3ccnc(Nc4cccc(F)c4)n3)cc2[nH]1 ; Cc1c(-c2ccnc(Nc3cccc(C(F)(F)F)c3)n2)ccc2[nH]cnc12 ; Fc1cc2[nH]cnc2cc1-c1ccnc(Nc2cccc(C(F)(F)F)c2)n1 ; FC(F)(F)c1cccc(Nc2nccc(-c3ccc4nccn4c3)n2)c1] 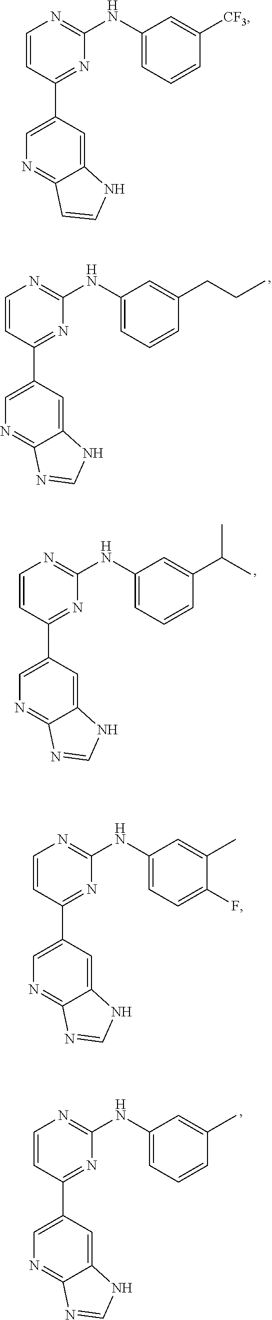 [SMILES: FC(F)(F)c1cccc(Nc2nccc(-c3cnc4cc[nH]c4c3)n2)c1 ; CCCc1cccc(Nc2nccc(-c3cnc4nc[nH]c4c3)n2)c1 ; CC(C)c1cccc(Nc2nccc(-c3cnc4nc[nH]c4c3)n2)c1 ; Cc1cc(Nc2nccc(-c3cnc4nc[nH]c4c3)n2)ccc1F ; Cc1cccc(Nc2nccc(-c3cnc4nc[nH]c4c3)n2)c1] 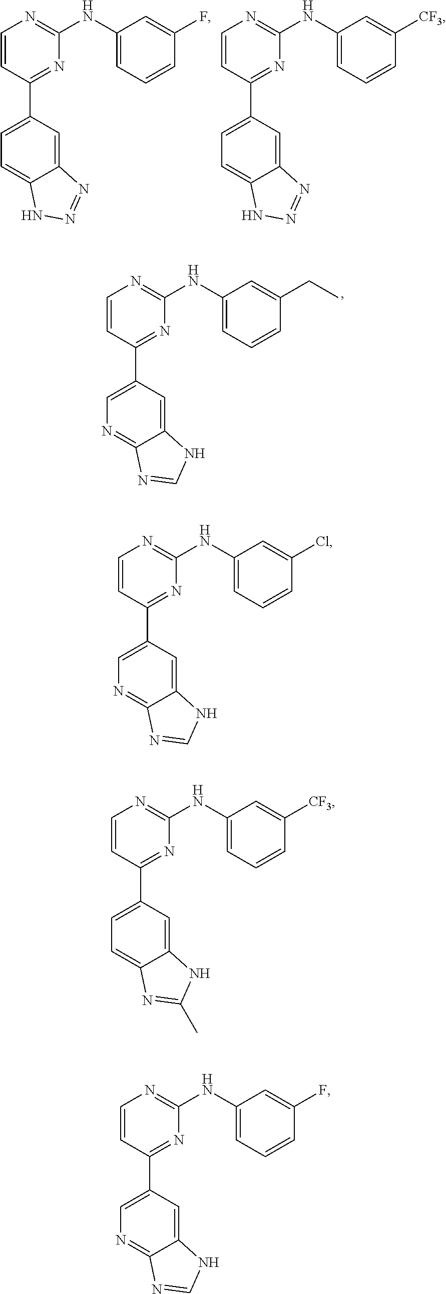 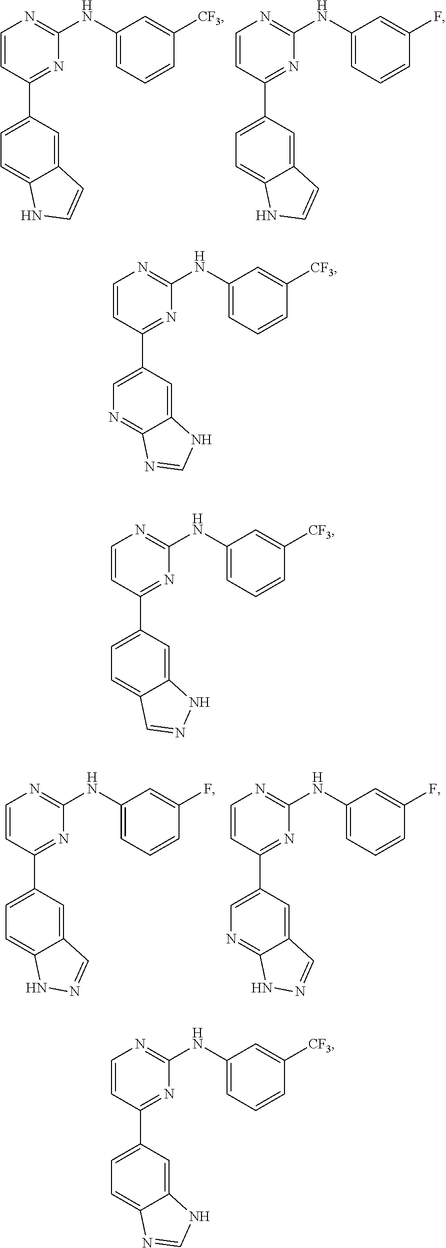 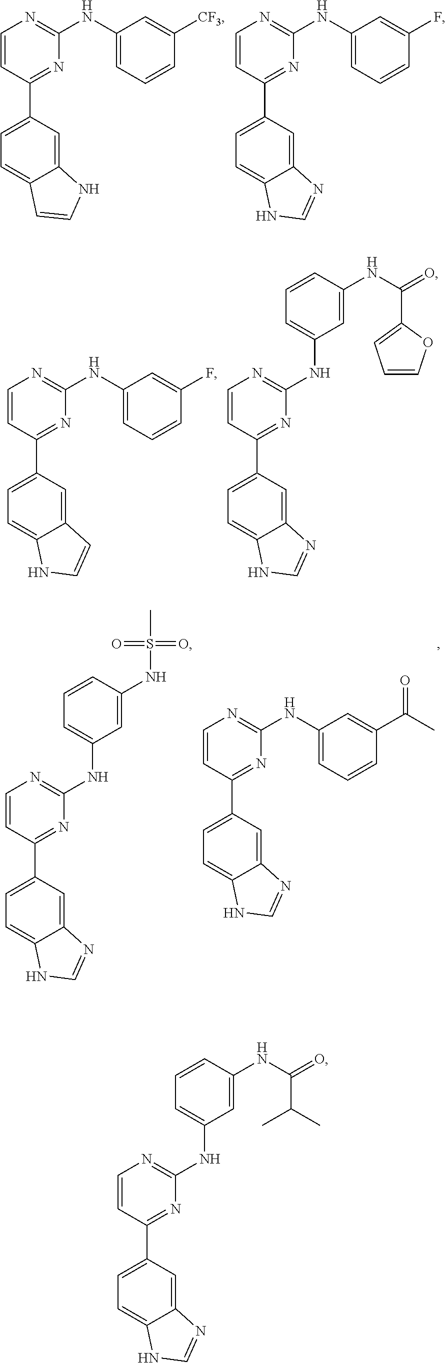 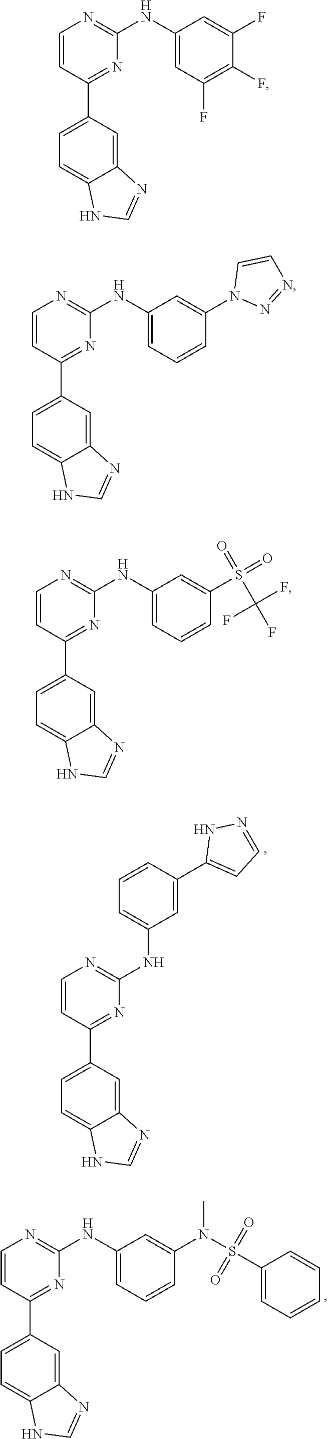 [SMILES: Fc1cc(Nc2nccc(-c3ccc4[nH]cnc4c3)n2)cc(F)c1F ; c1cc(Nc2nccc(-c3ccc4[nH]cnc4c3)n2)cc(-n2ccnn2)c1 ; O=S(=O)(c1cccc(Nc2nccc(-c3ccc4[nH]cnc4c3)n2)c1)C(F)(F)F ; c1cc(Nc2nccc(-c3ccc4[nH]cnc4c3)n2)cc(-c2ccn[nH]2)c1 ; CN(c1cccc(Nc2nccc(-c3ccc4[nH]cnc4c3)n2)c1)S(=O)(=O)c1ccccc1] 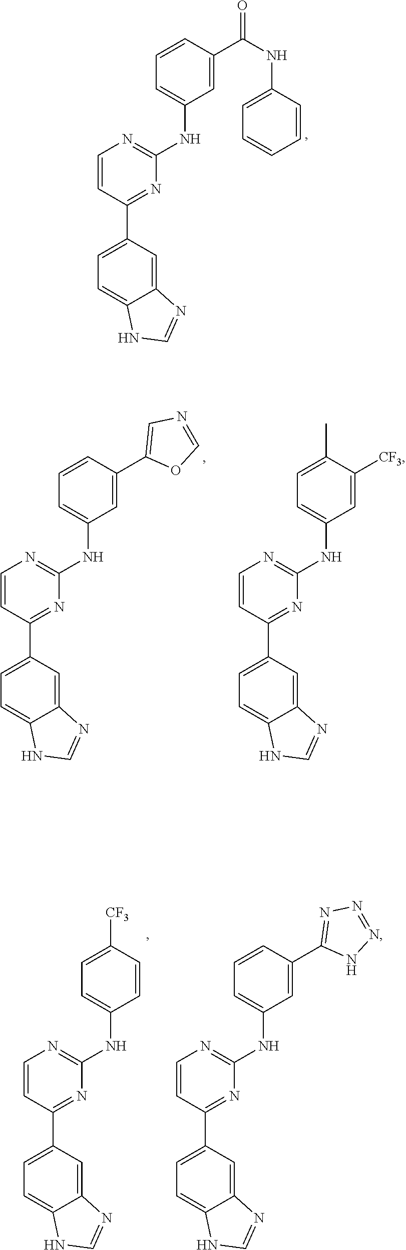 [SMILES: O=C(Nc1ccccc1)c1cccc(Nc2nccc(-c3ccc4[nH]cnc4c3)n2)c1 ; c1cc(Nc2nccc(-c3ccc4[nH]cnc4c3)n2)cc(-c2cnco2)c1 ; Cc1ccc(Nc2nccc(-c3ccc4[nH]cnc4c3)n2)cc1C(F)(F)F ; FC(F)(F)c1ccc(Nc2nccc(-c3ccc4[nH]cnc4c3)n2)cc1 ; c1cc(Nc2nccc(-c3ccc4[nH]cnc4c3)n2)cc(-c2nnn[nH]2)c1] 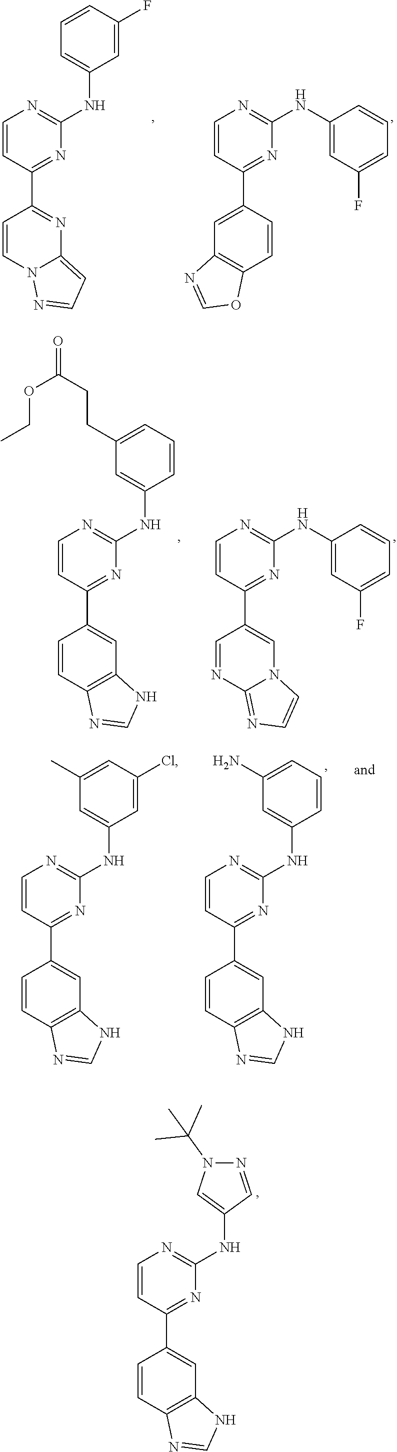
or a pharmaceutically acceptable salt thereof.

25. The compound of any one of claims 19 to 23, wherein the compound is selected from the group consisting of: 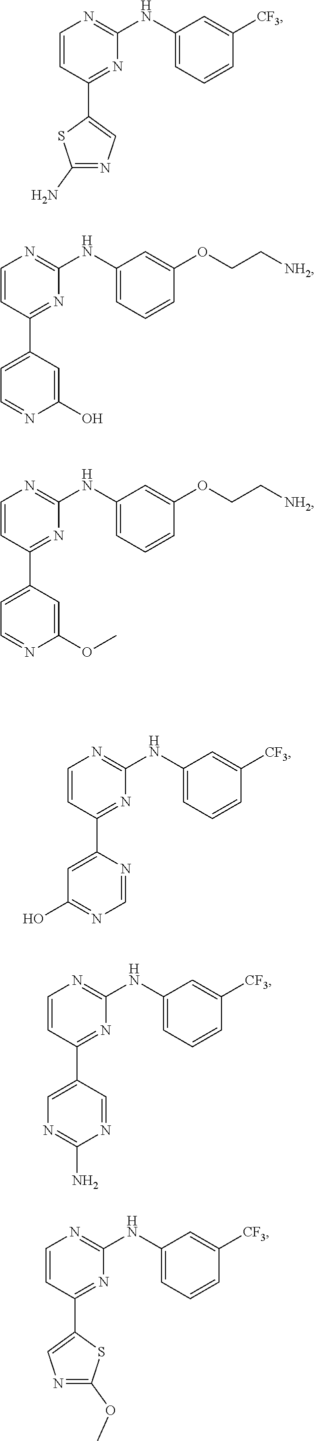 [SMILES: Nc1ncc(-c2ccnc(Nc3cccc(C(F)(F)F)c3)n2)s1 ; NCCOc1cccc(Nc2nccc(-c3ccnc(O)c3)n2)c1 ; COc1cc(-c2ccnc(Nc3cccc(OCCN)c3)n2)ccn1 ; Oc1cc(-c2ccnc(Nc3cccc(C(F)(F)F)c3)n2)ncn1 ; Nc1ncc(-c2ccnc(Nc3cccc(C(F)(F)F)c3)n2)cn1 ; COc1ncc(-c2ccnc(Nc3cccc(C(F)(F)F)c3)n2)s1] 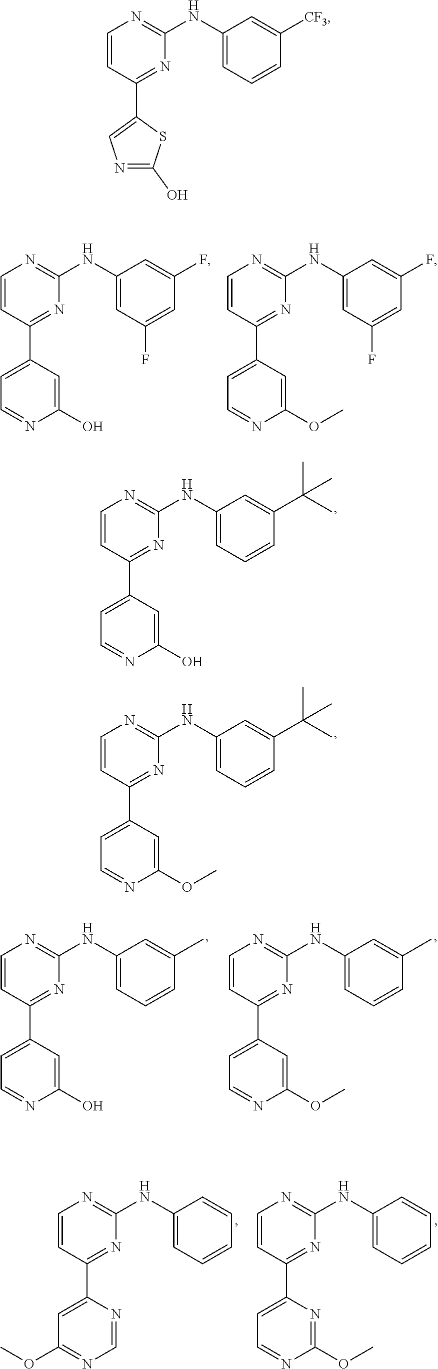 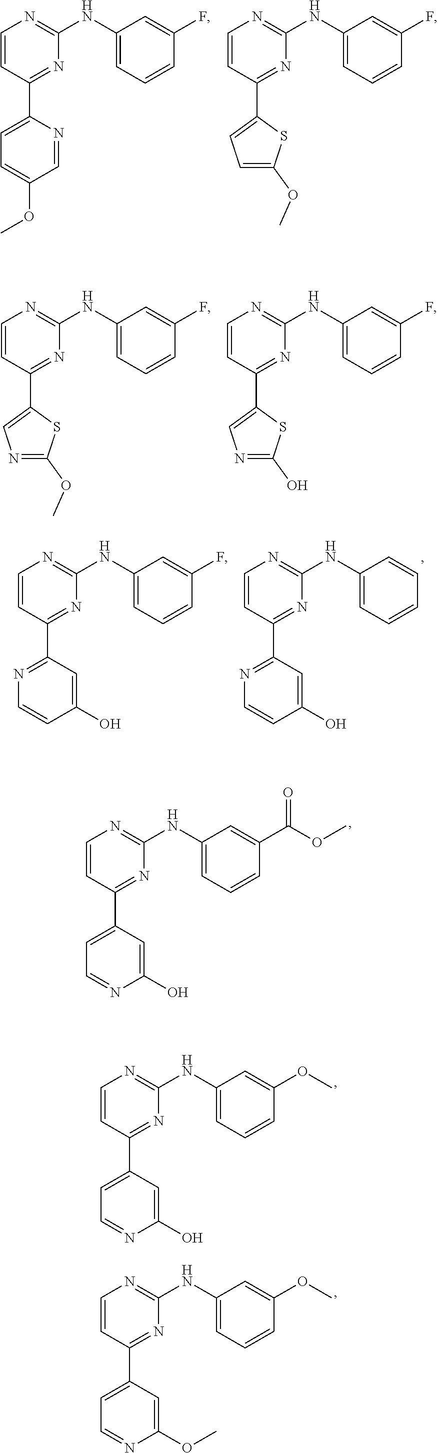 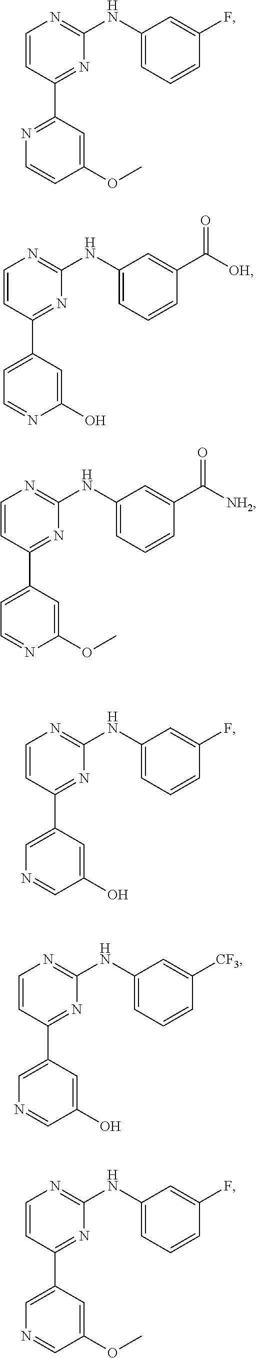 [SMILES: COc1ccnc(-c2ccnc(Nc3cccc(F)c3)n2)c1 ; O=C(O)c1cccc(Nc2nccc(-c3ccnc(O)c3)n2)c1 ; COc1cc(-c2ccnc(Nc3cccc(C(N)=O)c3)n2)ccn1 ; Oc1cncc(-c2ccnc(Nc3cccc(F)c3)n2)c1 ; Oc1cncc(-c2ccnc(Nc3cccc(C(F)(F)F)c3)n2)c1 ; COc1cncc(-c2ccnc(Nc3cccc(F)c3)n2)c1] 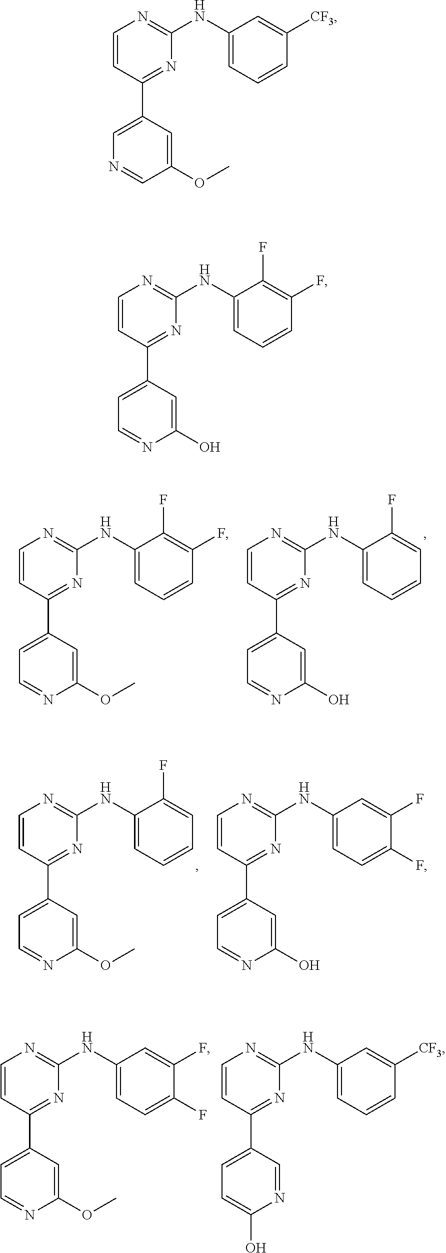 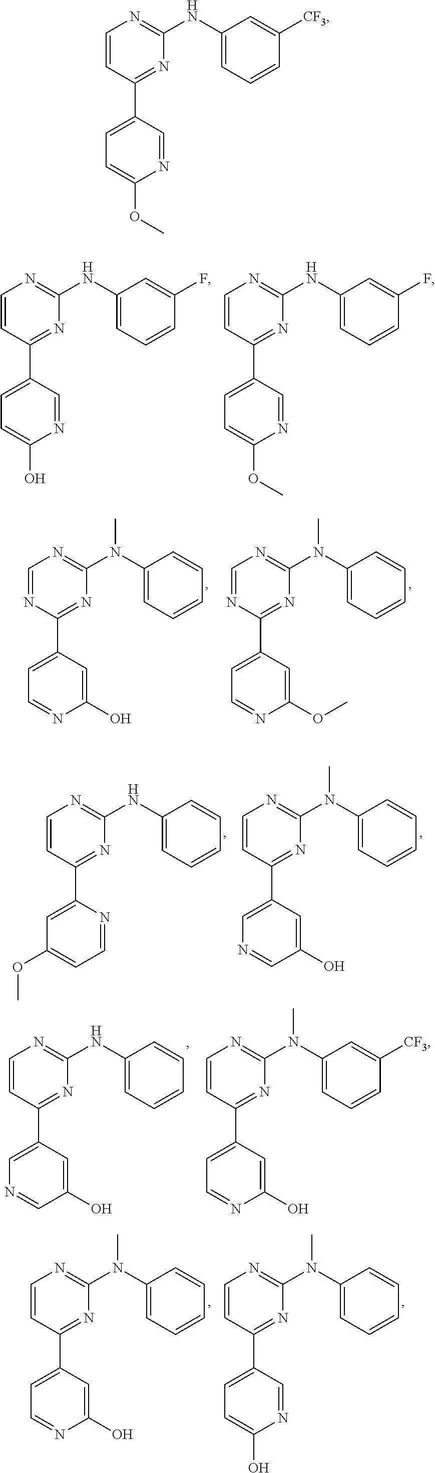 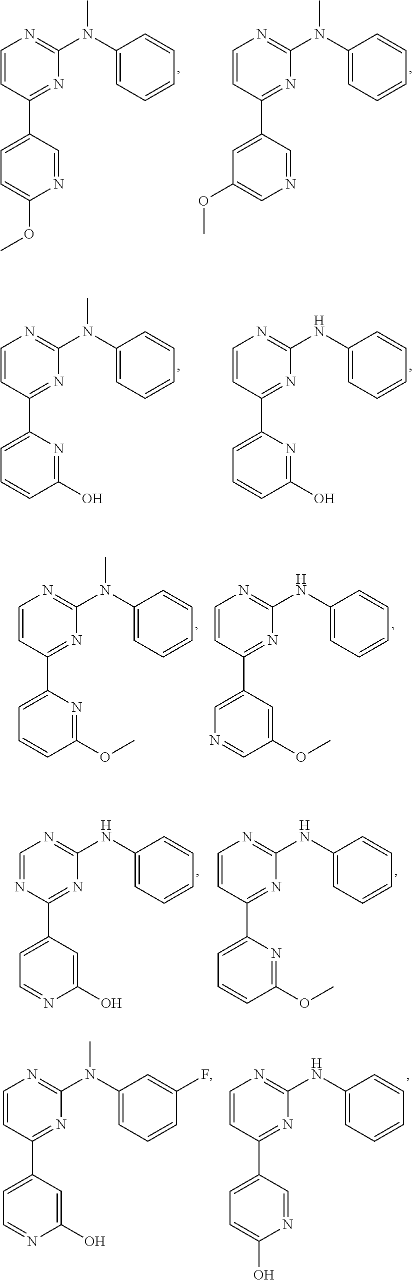 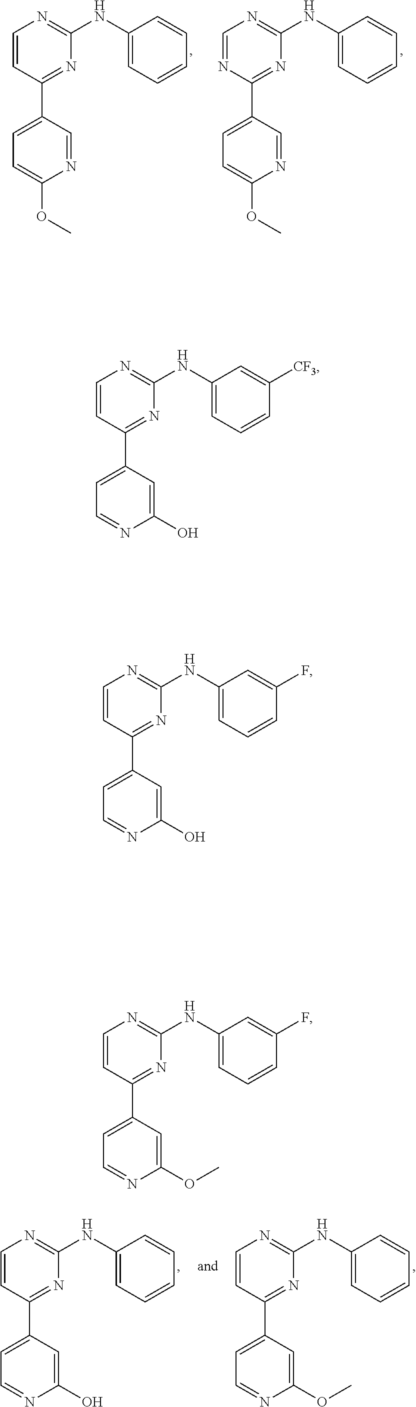
or a pharmaceutically acceptable salt thereof.

26. The compound of any one of claims 19 to 23, wherein the compound is selected from the group consisting of: 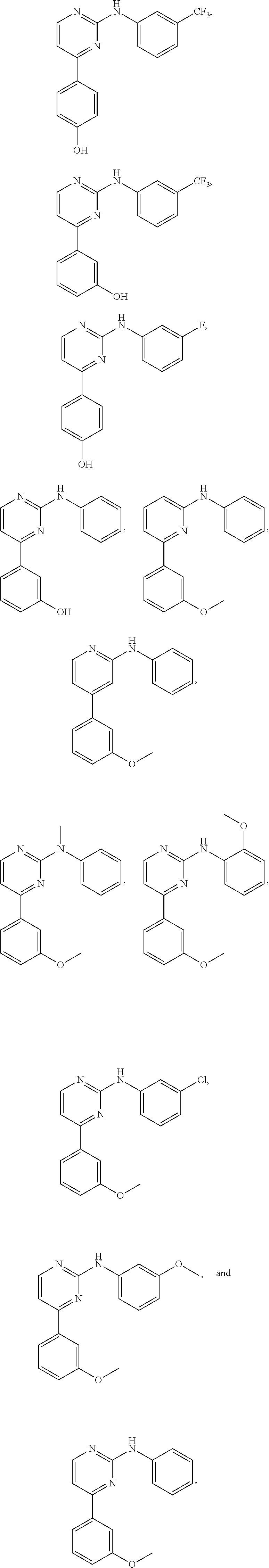
or a pharmaceutically acceptable salt thereof.

27. The compound of any one of claims 19 to 23, wherein the compound is selected from the group consisting of: 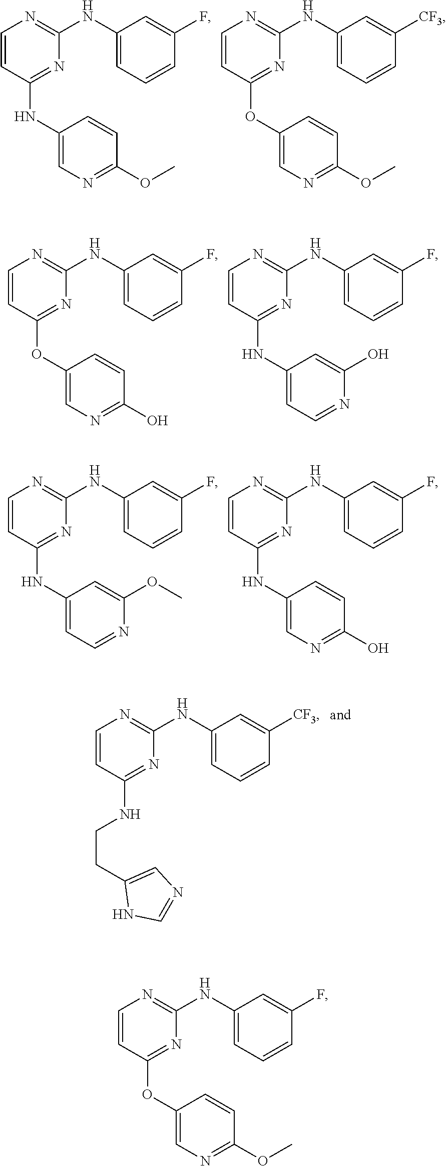
or a pharmaceutically acceptable salt thereof.

28. The compound of any one of claims 1 to 15, wherein B is heteroaryl optionally substituted with 1-3 R⁸ groups each independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R⁴⁴, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₁-C₅-haloalkyl, aryl, heteroaryl, 5-9 membered heterocycloalkyl, C₃-C₅-cycloalkyl, C₁-C₃-alkoxy, and C₁-C₃-haloalkoxy, wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

29. The compound of claim 28, wherein B is heteroaryl substituted with 1-3 R⁸ groups each independently selected from the group consisting of 5-9 membered heterocycloalkyl, C₁-C₅-haloalkyl, C₁-C₅-alkyl, aryl, heteroaryl, and alkylaryl, wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and OXO.

30. The compound of claim 28 or 29, wherein B is heteroaryl substituted with 1 or 2 R⁸ groups each independently —CF₃, —OH, tetrahydropyranyl, isopropyl, pyridinyl, benzyl or phenyl.

31. The compound of any one of claims 28 to 30, wherein B is selected from the group consisting of:

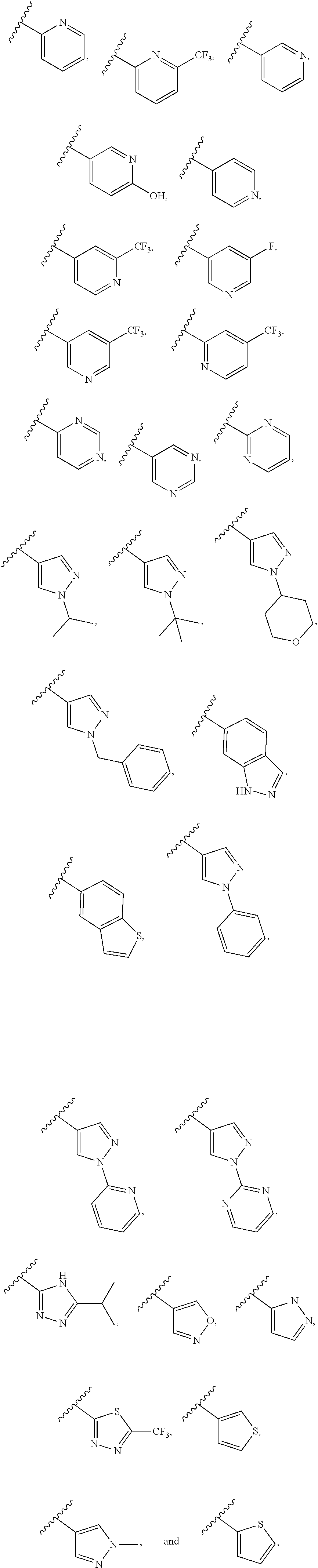

32. The compound of any one of claims 28 to 31, wherein the compound is selected from the group consisting of: 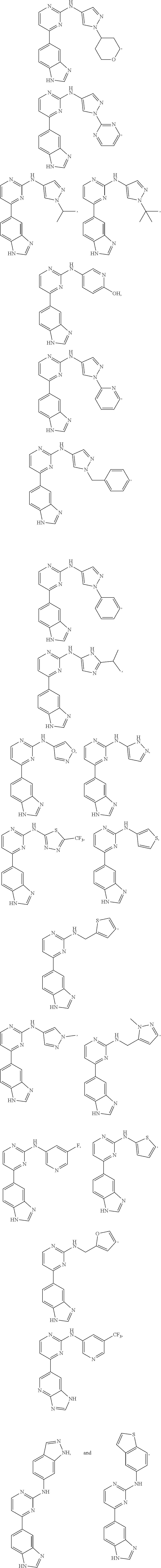
or a pharmaceutically acceptable salt thereof.

33. The compound of any one of claims 28 to 31, wherein the compound is selected from the group consisting of: 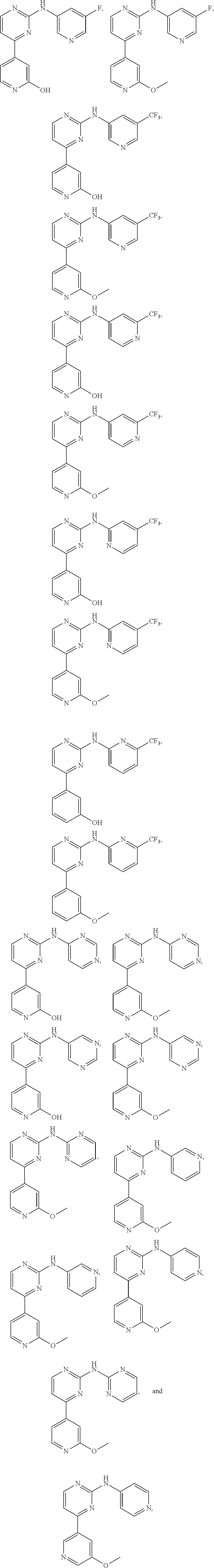
or a pharmaceutically acceptable salt thereof.

34. The compound of claim 1 or 2, wherein Y¹ is —CH₂—, —S—, or —O—.

35. The compound of claim 34, wherein is phenyl optionally substituted with 1-3 R⁸ groups each independently selected from the group consisting of 4-9 membered heterocycloalkyl, C₁-C₅-haloalkyl, C₁-C₅-alkyl, aryl, heteroaryl, and alkylaryl.

36. The compound of claim 34 or 35, wherein 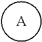
is pyridyl optionally substituted with 1-3 R⁹ groups each independently selected from the group consisting of halogen, C₁-C₃ alkyl, C₁-C₃ haloalkyl, —OR³, and —NH₂.

37. The compound of claim 36, wherein 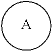
is pyridyl substituted with 1 or 2 R⁹ groups each independently selected from the group consisting of —OH and —OCH₃.

38. The compound of claim 37, wherein the compound is selected from the groups consisting of: 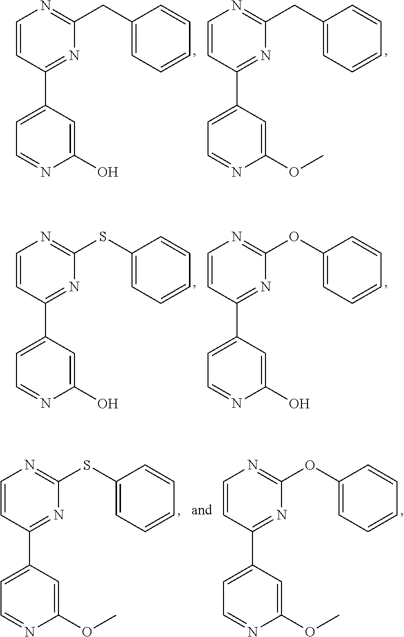
or a pharmaceutically acceptable salt thereof.

39. A compound of Formula (Ia): 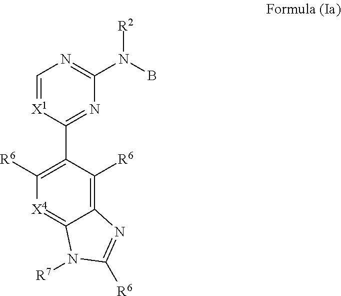
or a pharmaceutically acceptable salt thereof,
wherein:
B is aryl or heteroaryl each optionally substituted with 1-3 R⁸ groups each independently selected from the group consisting of (CH₂)ₘCN, —(CH₂)ₘOH, —(CH₂)ₘN(R⁴)₂, —(CH₂)ₘC(O)N(R⁴)₂, —(CH₂)ₘNR⁴C(O)R⁴, —(CH₂)ₘNR⁴C(O)N(R⁴)₂, —(CH₂)ₘNR⁴ (C₁-C₅)alkylene(O)R⁴⁴, —CH₂)ₘC(O)R⁴, —(CH₂)ₘCO₂R⁴, —(CH₂)ₘOC(O)R⁴, —(CH₂)ₘOC(O)OR⁴, —(CH₂)ₘO(C₁-C₅-alkylene)N(R⁴)₂, —(CH₂)ₘSR⁴, —(CH₂)ₘS(O)R⁴, —(CH₂)ₘS(O)₂R⁴, —(CH₂)ₘN(R⁴)S(O)₂R⁴, —(CH₂)ₘS(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and OXO;
X¹ is N or CR¹;
X⁴ is N or CR⁵;
R¹ is hydrogen, halogen, or C₁-C₅ alkyl;
R² is hydrogen or C₁-C₅ alkyl;
R⁴ is hydrogen, halogen, aryl, heteroaryl, C₁-C₅ alkyl, C₃-C₆-cycloalkyl, or 3-6 membered heterocycloalkyl, wherein the aryl is unsubstituted or substituted with halogen, —OH, or —CN;
R^{4A} is aryl, heteroaryl, C₁-C₅ alkyl, C₃-C₆-cycloalkyl, or 3-6 membered heterocycloalkyl;
R⁵ is hydrogen, halogen, —OH, C₁-C₅ alkoxy, or C₁-C₅ alkyl;
each R⁶ is independently hydrogen, halogen, —OH, C₁-C₅ alkoxy, or C₁-C₅ alkyl;
R⁷ is hydrogen or C₁-C₅ alkyl; and
each m is independently 0, 1, or 2.

40. The compound of claim 39, wherein X¹ is N or CH.

41. The compound of claim 39 or 40, wherein X⁴ is N or CH.

42. The compound of any one of claims 39 to 41, wherein R² is hydrogen or —CH₃.

43. The compound of any one of claims 39 to 42, wherein each R⁶ is independently hydrogen, halogen, or hydroxyl.

44. The compound of any one of claims 39 to 43, wherein R⁷ is hydrogen or —CH₃.

45. The compound of any one of claims 39 to 44, wherein B is phenyl substituted with 1-3 R⁸ groups each independently selected from —OH, —NR⁴C(O)R⁴, —S(O)₂R⁴, —S(O)₂N(R⁴)₂, halogen, C₁-C₅-alkyl, C₁-C₅-haloalkyl, aryl, heteroaryl, and 5-9 membered heterocycloalkyl, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo.

46. The compound of any one of claims 39 to 44, wherein B is pyridyl substituted with 1-3 R⁸ groups each independently selected from halogen, C₁-C₅-alkyl, and C₁-C₅-haloalkyl.

47. The compound of any one of claims 39 to 44, wherein B is pyrazolyl substituted with 1-3 R⁸ groups each independently selected from halogen, C₁-C₅-alkyl, aryl, and heteroaryl, and wherein said aryl and heteroaryl are each optionally substituted with 1 or 2 C₁-C₅-alkyl.

48. The compound of claim 39, wherein the compound is selected from the group consisting of: 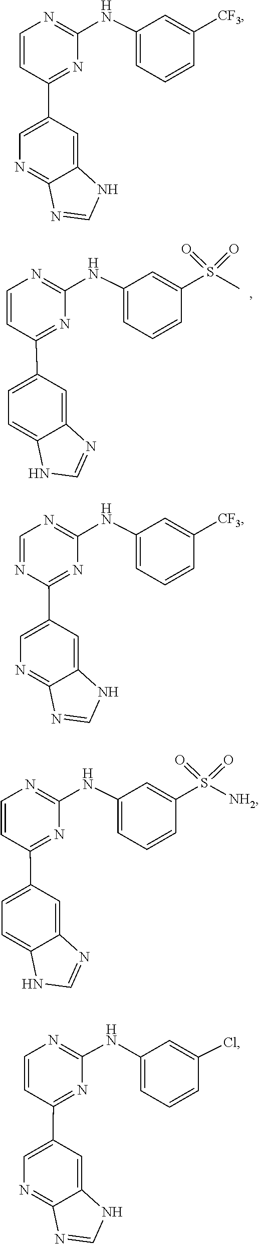 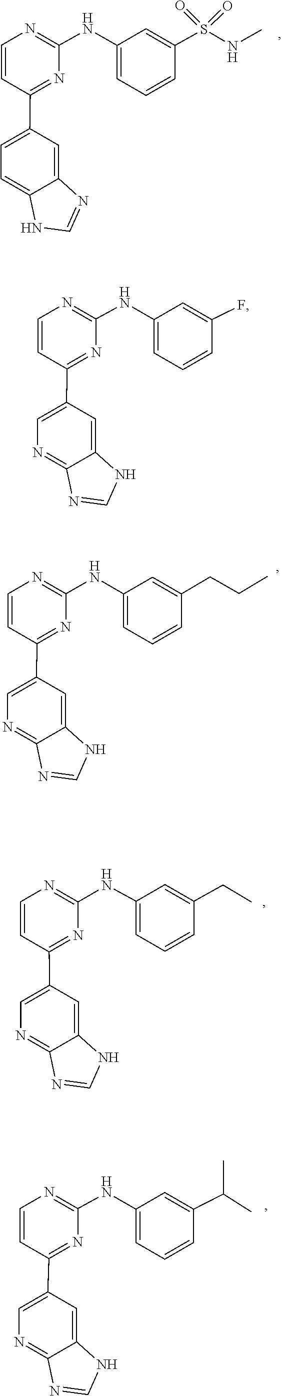 [SMILES: CNS(=O)(=O)c1cccc(Nc2nccc(-c3ccc4[nH]cnc4c3)n2)c1 ; Fc1cccc(Nc2nccc(-c3cnc4nc[nH]c4c3)n2)c1 ; CCCc1cccc(Nc2nccc(-c3cnc4nc[nH]c4c3)n2)c1 ; CCc1cccc(Nc2nccc(-c3cnc4nc[nH]c4c3)n2)c1 ; CC(C)c1cccc(Nc2nccc(-c3cnc4nc[nH]c4c3)n2)c1] 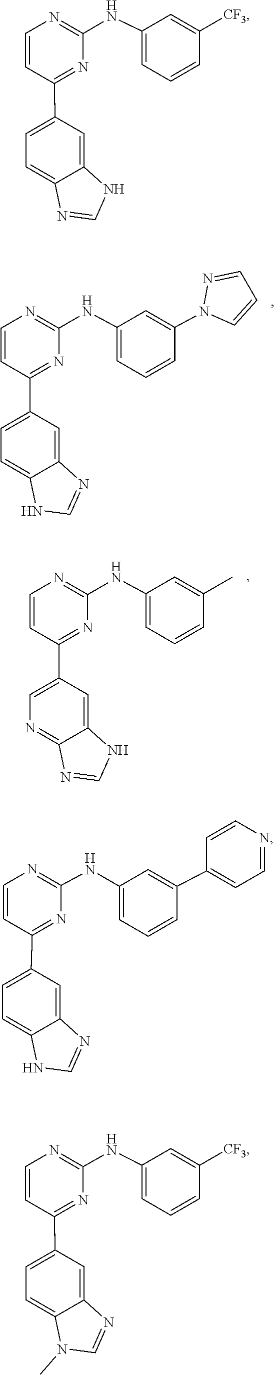 [SMILES: FC(F)(F)c1cccc(Nc2nccc(-c3ccc4nc[nH]c4c3)n2)c1 ; c1cc(Nc2nccc(-c3ccc4[nH]cnc4c3)n2)cc(-n2cccn2)c1 ; Cc1cccc(Nc2nccc(-c3cnc4nc[nH]c4c3)n2)c1 ; c1cc(Nc2nccc(-c3ccc4[nH]cnc4c3)n2)cc(-c2ccncc2)c1 ; Cn1cnc2cc(-c3ccnc(Nc4cccc(C(F)(F)F)c4)n3)ccc21] 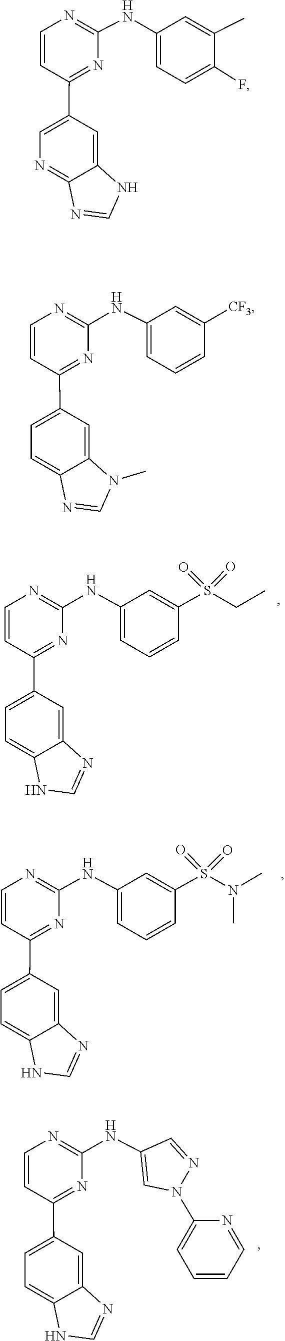 [SMILES: Cc1cc(Nc2nccc(-c3cnc4nc[nH]c4c3)n2)ccc1F ; Cn1cnc2ccc(-c3ccnc(Nc4cccc(C(F)(F)F)c4)n3)cc21 ; CCS(=O)(=O)c1cccc(Nc2nccc(-c3ccc4[nH]cnc4c3)n2)c1 ; CN(C)S(=O)(=O)c1cccc(Nc2nccc(-c3ccc4[nH]cnc4c3)n2)c1 ; c1ccc(-n2cc(Nc3nccc(-c4ccc5[nH]cnc5c4)n3)cn2)nc1] 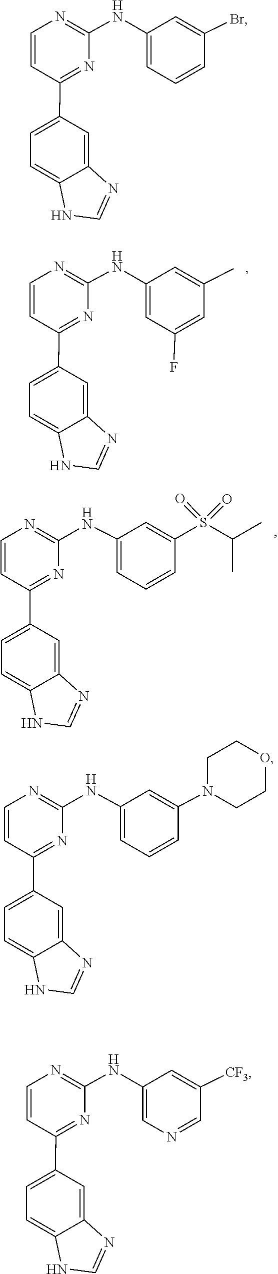 [SMILES: Brc1cccc(Nc2nccc(-c3ccc4[nH]cnc4c3)n2)c1 ; Cc1cc(F)cc(Nc2nccc(-c3ccc4[nH]cnc4c3)n2)c1 ; CC(C)S(=O)(=O)c1cccc(Nc2nccc(-c3ccc4[nH]cnc4c3)n2)c1 ; c1cc(Nc2nccc(-c3ccc4[nH]cnc4c3)n2)cc(N2CCOCC2)c1 ; FC(F)(F)c1cncc(Nc2nccc(-c3ccc4[nH]cnc4c3)n2)c1] 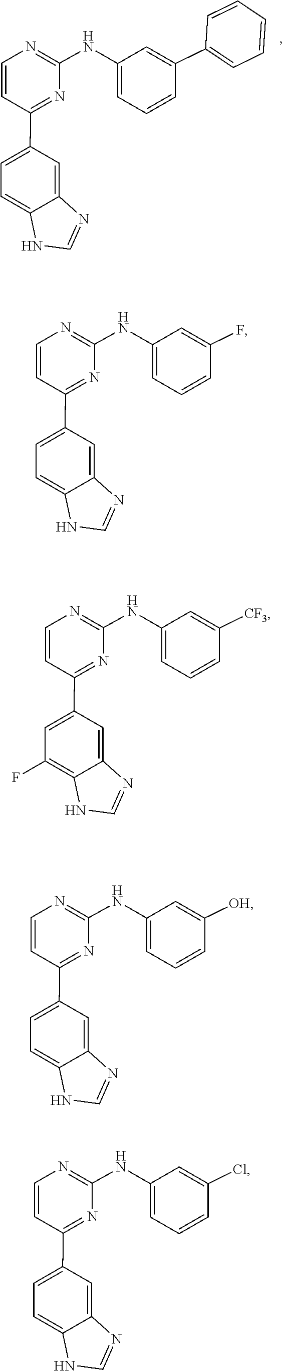 [SMILES: c1ccc(-c2cccc(Nc3nccc(-c4ccc5[nH]cnc5c4)n3)c2)cc1 ; Fc1cccc(Nc2nccc(-c3ccc4[nH]cnc4c3)n2)c1 ; Fc1cc(-c2ccnc(Nc3cccc(C(F)(F)F)c3)n2)cc2nc[nH]c12 ; Oc1cccc(Nc2nccc(-c3ccc4[nH]cnc4c3)n2)c1 ; Clc1cccc(Nc2nccc(-c3ccc4[nH]cnc4c3)n2)c1] 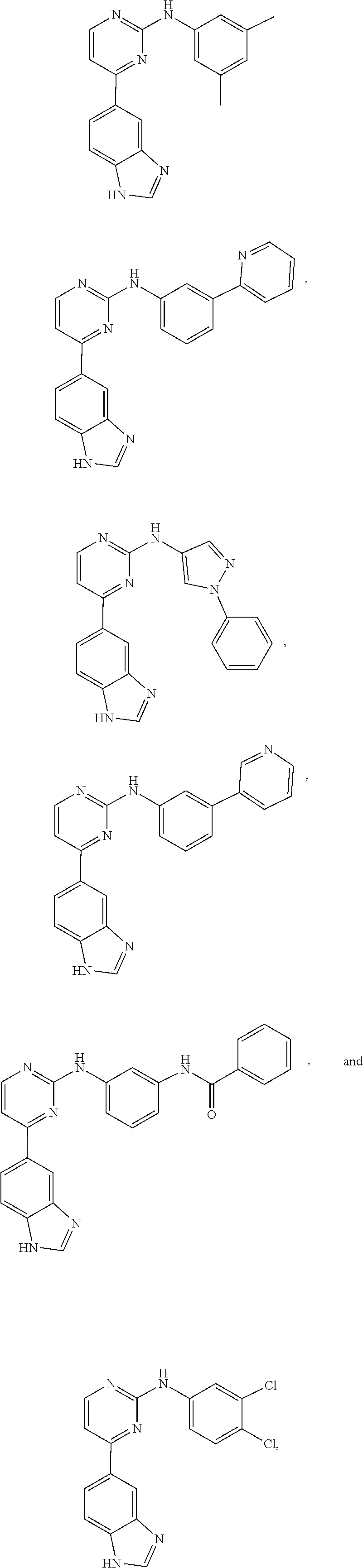 [SMILES: Cc1cc(C)cc(Nc2nccc(-c3ccc4[nH]cnc4c3)n2)c1 ; c1ccc(-c2cccc(Nc3nccc(-c4ccc5[nH]cnc5c4)n3)c2)nc1 ; c1ccc(-n2cc(Nc3nccc(-c4ccc5[nH]cnc5c4)n3)cn2)cc1 ; c1cncc(-c2cccc(Nc3nccc(-c4ccc5[nH]cnc5c4)n3)c2)c1 ; O=C(Nc1cccc(Nc2nccc(-c3ccc4[nH]cnc4c3)n2)c1)c1ccccc1 ; Clc1ccc(Nc2nccc(-c3ccc4[nH]cnc4c3)n2)cc1Cl]
or a pharmaceutically acceptable salt thereof.

49. The compound of claim 39, wherein the compound is of Formula (Ib): 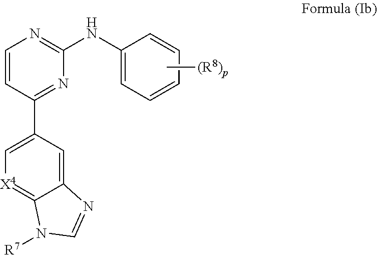
or a pharmaceutically acceptable salt thereof,
wherein:
R⁵ is hydrogen, halogen, —OH, C₁-C₃ alkoxy, or C₁-C₃ alkyl;
R⁷ is hydrogen or C₁-C₃ alkyl;
each R⁸ is independently selected from the group consisting of —CN, —OH, —N(R⁴)₂, —C(O)N(R⁴)₂, —NR⁴C(O)R⁴, —NR⁴C(O)N(R⁴)₂, —NR⁴ (C₁-C₅)alkylene(O)R^{4A}, —C(O)R⁴, —CO₂R⁴, —OC(O)R⁴, —OC(O)OR⁴, —O(C₁-C₅-alkylene)N(R⁴)₂, —SR⁴, —S(O)R⁴, —S(O)₂R⁴, —N(R⁴)S(O)₂R⁴, —S(O)₂N(R⁴)₂, halogen, C₁-C₅-alkyl, C₂-C₅-alkenyl, C₂-C₅-alkynyl, C₁-C₅-haloalkyl, aryl, alkylaryl, heteroaryl, 5-9 membered heterocycloalkyl, C₃-C₉-cycloalkyl, C₁-C₅-alkoxy, and C₁-C₅-haloalkoxy, and wherein said aryl and heteroaryl are each optionally substituted with 1 or 2 C₁-C₅-alkyl, and said 5-9 membered heterocycloalkyl is optionally substituted with 1 or 2 substituents each independently selected from C₁-C₅-alkyl and oxo; and
p is 0, 1, 2, or 3.

50. A compound of Formula (II): 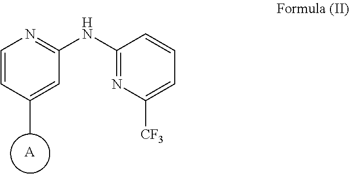
or a pharmaceutically acceptable salt thereof,
wherein: 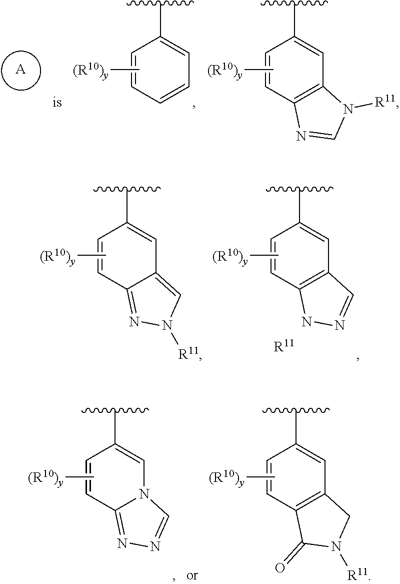
each R¹⁰ is independently halogen, C₁-C₅-alkyl, C₁-C₅-alkoxy, C₁-C₅-haloalkoxy, —CN, —OH, —N(R¹²)₂, —C(O)R¹², —C(O)N(R¹²)₂, —NR¹²C(O)R¹², —CO₂R¹², —OC(O)R¹², —OC(O)OR¹², —SR¹², —S(O)R¹², or —S(O)₂R¹²;
each R¹² is independently hydrogen or C₁-C₅ alkyl;
y is 0, 1, 2, 3, or 4; and
R¹¹ is hydrogen, C₁-C₅ alkyl, C₃-C₆-cycloalkyl, or aryl.

51. The compound of claim 50, or a pharmaceutically acceptable salt thereof, wherein:
each R¹⁰ is independently halogen, C₁-C₅-alkyl, or —C(O)N(R¹²)₂.

52. The compound of any one of claims 50 to 51, or a pharmaceutically acceptable salt thereof, wherein:
each R¹⁰ is independently —F, —Cl, —Br, —CH₃, —CH(CH₃)₂, —C(CH₃) 3, or —CONH₂.

53. The compound of any one of claims 50 to 52, or a pharmaceutically acceptable salt thereof, wherein:
y is 1 or 2.

54. The compound of any one of claims 50 to 53, or a pharmaceutically acceptable salt thereof, wherein:
R¹¹ is hydrogen, C₁-C₃ alkyl, C₃-C₆-cycloalkyl, or phenyl.

55. The compound of any one of claims 50 to 54, or a pharmaceutically acceptable salt thereof, wherein:
R¹¹ is hydrogen, —CH₃, —CH(CH₃)₂, —C(CH₃) 3, cyclopropyl, or phenyl.

56. The compound of any one of claims 50 to 55, or a pharmaceutically acceptable salt thereof, wherein:

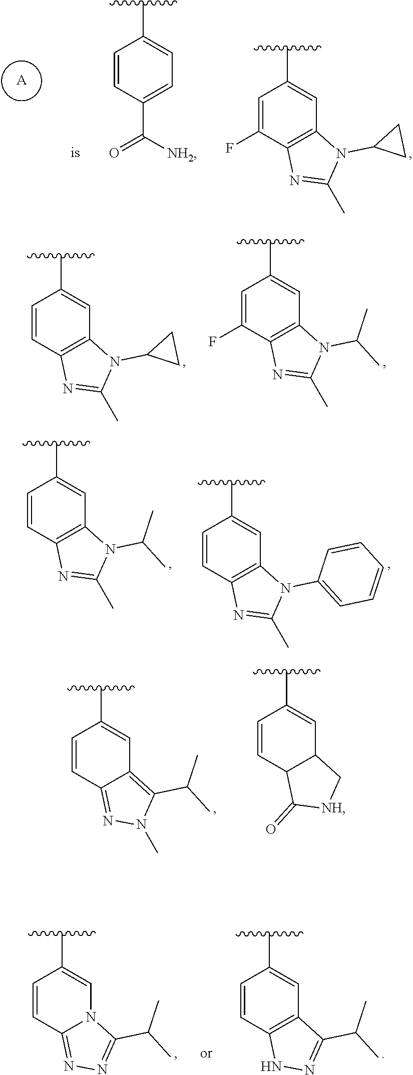

57. The compound of claim 50, or a pharmaceutically acceptable salt thereof, wherein the compound is:

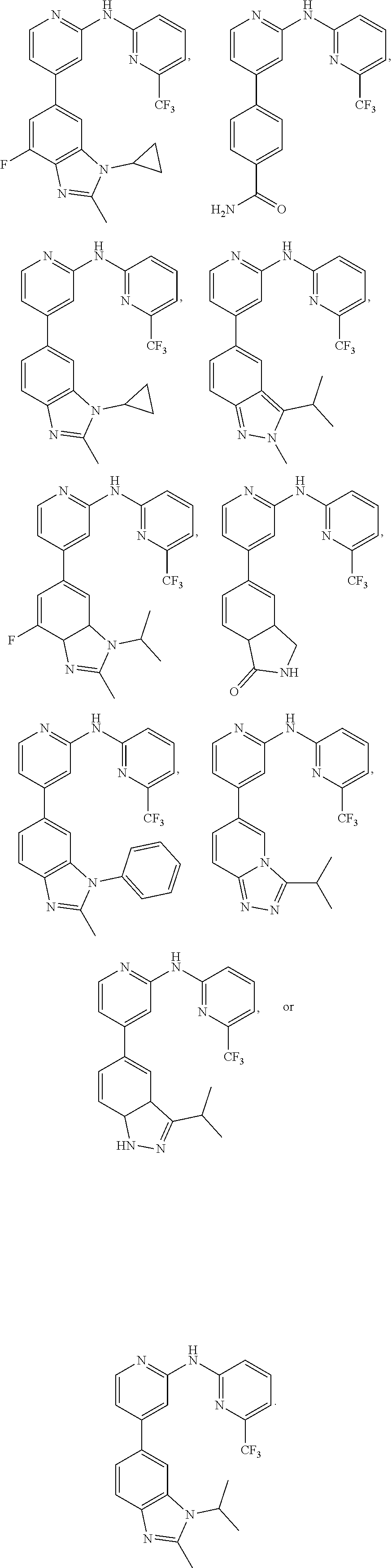

58. A compound, selected from the group consisting of:
N-(3-((4-(1H-benzo[d]imidazol-5-yl)pyrimidin-2-yl)amino)phenyl)benzamide,
N-(3-((4-(1H-benzo[d]imidazol-5-yl)pyrimidin-2-yl)amino)phenyl)acetamide,
4-(1H-benzo[d]imidazol-5-yl)-N-(2′,6′-dimethyl-[1,1′-biphenyl]-3-yl)pyrimidin-2-amine,
4-(1H-benzo[d]imidazol-5-yl)-N-(3-(2,5-dimethyl-1H-pyrrol-1-yl)phenyl)pyrimidin-2-amine,
4-(1H-benzo[d]imidazol-5-yl)-N-(3-(ethylsulfonyl)phenyl)pyrimidin-2-amine,
4-(1H-benzo[d]imidazol-5-yl)-N-(1-(tetrahydro-2H-pyran-4-yl)-1H-pyrazol-4-yl)pyrimidin-2-amine,
3-((4-(1H-benzo[d]imidazol-5-yl)pyrimidin-2-yl)amino)-N,N-dimethylbenzenesulfonamide,
N-(3-(1H-pyrazol-1-yl)phenyl)-4-(1H-benzo[d]imidazol-5-yl)pyrimidin-2-amine,
3-((4-(1H-benzo[d]imidazol-5-yl)pyrimidin-2-yl)amino)-N-cyclopropylbenzamide,
4-(1H-benzo[d]imidazol-5-yl)-N-(3-cyclopentylphenyl)pyrimidin-2-amine,
(3-((4-(1H-benzo[d]imidazol-5-yl)pyrimidin-2-yl)amino)phenyl)(morpholino)methanone,
3-((4-(1H-benzo[d]imidazol-5-yl)pyrimidin-2-yl)amino)-N-(2-fluorophenyl)benzenesulfonamide,
4-(1H-benzo[d]imidazol-5-yl)-N-(3-fluoro-5-methylphenyl)pyrimidin-2-amine,
4-(1H-benzo[d]imidazol-5-yl)-N-(5-(trifluoromethyl)pyridin-3-yl)pyrimidin-2-amine,
N-(3-((4-(1H-benzo[d]imidazol-5-yl)pyrimidin-2-yl)amino)phenyl)propionamide,
4-(1H-benzo[d]imidazol-5-yl)-N-(3,5-dimethylphenyl)pyrimidin-2-amine,
(3-((4-(1H-benzo[d]imidazol-5-yl)pyrimidin-2-yl)amino)phenyl)(pyrrolidin-1-yl)methanone,
3-((4-(1H-benzo[d]imidazol-5-yl)pyrimidin-2-yl)amino)-N,N-dimethylbenzamide,
3-((4-(1H-benzo[d]imidazol-5-yl)pyrimidin-2-yl)amino)-N,N-diethylbenzamide,
4-(1H-benzo[d]imidazol-5-yl)-N-(1-(pyrimidin-2-yl)-1H-pyrazol-4-yl)pyrimidin-2-amine,
4-(1H-benzo[d]imidazol-5-yl)-N-(1-isopropyl-1H-pyrazol-4-yl)pyrimidin-2-amine,
6-((4-(1H-benzo[d]imidazol-5-yl)pyrimidin-2-yl)amino)pyridin-2-ol,
3-((4-(1H-benzo[d]imidazol-5-yl)pyrimidin-2-yl)amino)-N-phenylbenzenesulfonamide,
3-((4-(1H-benzo[d]imidazol-5-yl)pyrimidin-2-yl)amino)-N-cyclopropylbenzenesulfonamide,
3-((4-(1H-benzo[d]imidazol-5-yl)pyrimidin-2-yl)amino)-N-methylbenzenesulfonamide,
4-(1H-benzo[d]imidazol-5-yl)-N-(3-(isopropylsulfonyl)phenyl)pyrimidin-2-amine,
4-(1H-benzo[d]imidazol-5-yl)-N-(3-(methylsulfonyl)phenyl)pyrimidin-2-amine,
3-((4-(1H-benzo[d]imidazol-5-yl)pyrimidin-2-yl)amino)benzoic acid,
5-((4-(1H-benzo[d]imidazol-5-yl)pyrimidin-2-yl)amino)pyridin-2-ol,
4-(1H-pyrrolo[2,3-c]pyridin-3-yl)-N-(3-(trifluoromethyl)phenyl)pyrimidin-2-amine,
4-(1H-benzo[d]imidazol-5-yl)-N-(3-bromophenyl)pyrimidin-2-amine,
3-((4-(1H-benzo[d]imidazol-5-yl)pyrimidin-2-yl)amino)-N-(tert-butyl)benzenesulfonamide,
4-(1H-benzo[d]imidazol-5-yl)-N-(2′-methyl-[1,1′-biphenyl]-3-yl)pyrimidin-2-amine,
4-(1-methyl-1H-benzo[d]imidazol-5-yl)-N-(3-(trifluoromethyl)phenyl)pyrimidin-2-amine,
4-(1H-benzo[d]imidazol-5-yl)-N-(1-(pyridin-2-yl)-1H-pyrazol-4-yl)pyrimidin-2-amine,
3-((4-(1H-benzo[d]imidazol-5-yl)pyrimidin-2-yl)amino)-N-(4-fluorophenyl)benzenesulfonamide,
4-(1H-benzo[d]imidazol-5-yl)-N-(1-benzyl-1H-pyrazol-4-yl)pyrimidin-2-amine,
N-(3-((4-(1H-benzo[d]imidazol-5-yl)pyrimidin-2-yl)amino)phenyl)benzenesulfonamide,
4-(1H-benzo[d]imidazol-5-yl)-N-(1-phenyl-1H-pyrazol-4-yl)pyrimidin-2-amine,
4-(1H-benzo[d]imidazol-5-yl)-N-(m-tolyl)pyrimidin-2-amine,
4-(1-methyl-1H-benzo[d]imidazol-6-yl)-N-(3-(trifluoromethyl)phenyl)pyrimidin-2-amine,
3-((4-(1H-benzo[d]imidazol-5-yl)pyrimidin-2-yl)amino)-N-methylbenzamide,
4-(1H-benzo[d]imidazol-5-yl)-N-(3-(pyridin-4-yl)phenyl)pyrimidin-2-amine,
4-(1H-benzo[d]imidazol-5-yl)-N-(3-(pyridin-2-yl)phenyl)pyrimidin-2-amine,
4-(1H-benzo[d]imidazol-5-yl)-N-(3-(pyridin-3-yl)phenyl)pyrimidin-2-amine,
3-((4-(1H-benzo[d]imidazol-5-yl)pyrimidin-2-yl)amino)benzenesulfonamide,
N¹-(4-(1H-benzo[d]imidazol-5-yl)pyrimidin-2-yl)-N³-(2-methoxyethyl)benzene-1,3-diamine,
4-(1H-benzo[d]imidazol-5-yl)-N-(4-fluoro-3-methylphenyl)pyrimidin-2-amine,
4-(1H-benzo[d]imidazol-5-yl)-N-(3,5-dichlorophenyl)pyrimidin-2-amine,
6-(1H-benzo[d]imidazol-5-yl)-N-(3-(trifluoromethyl)phenyl)pyridin-2-amine,
4-(1H-benzo[d]imidazol-5-yl)-N-(o-tolyl)pyrimidin-2-amine,
4-(1H-benzo[d]imidazol-5-yl)-N-(3-(trifluoromethyl)phenyl)pyridin-2-amine,
4-(1H-benzo[d]imidazol-5-yl)-N-(2-(tert-butyl)phenyl)pyrimidin-2-amine,
4-(1H-benzo[d]imidazol-5-yl)-N-(5-isopropyl-4H-1,2,4-triazol-3-yl)pyrimidin-2-amine,
4-(1H-benzo[d]imidazol-5-yl)-N-(naphthalen-2-yl)pyrimidin-2-amine,
N-(4-(1H-benzo[d]imidazol-5-yl)pyrimidin-2-yl)isoxazol-4-amine,
4-(1H-benzo[d]imidazol-5-yl)-N-(1H-pyrazol-5-yl)pyrimidin-2-amine,
N-(4-(1H-benzo[d]imidazol-5-yl)pyrimidin-2-yl)-5-(trifluoromethyl)-1,3,4-thiadiazol-2-amine,
4-(benzo[d]thiazol-6-yl)-N-(3-(trifluoromethyl)phenyl)pyrimidin-2-amine,
4-(1H-benzo[d]imidazol-5-yl)-N-(3,4-dimethylphenyl)pyrimidin-2-amine,
4-(1H-benzo[d]imidazol-5-yl)-N-(2-methoxyphenyl)pyrimidin-2-amine,
4-(1H-benzo[d]imidazol-5-yl)-N-(2-(trifluoromethyl)phenyl)pyrimidin-2-amine,
1-(3-((4-(1H-benzo[d]imidazol-5-yl)pyrimidin-2-yl)amino)phenyl)piperazin-2-one,
4-(1H-benzo[d]imidazol-5-yl)-N-(2-isopropylphenyl)pyrimidin-2-amine,
4-(1H-benzo[d]imidazol-5-yl)-N-(3-(piperidin-1-yl)phenyl)pyrimidin-2-amine,
4-(1H-benzo[d]imidazol-5-yl)-N-(2-fluorophenyl)pyrimidin-2-amine,
3-((4-(1H-benzo[d]imidazol-5-yl)pyrimidin-2-yl)amino)benzamide,
4-(1H-benzo[d]imidazol-5-yl)-N-(thiophen-3-yl)pyrimidin-2-amine,
4-(1H-benzo[d]imidazol-5-yl)-N-(3-chlorophenyl)pyrimidin-2-amine,
N-([1,1′-biphenyl]-3-yl)-4-(1H-benzo[d]imidazol-5-yl)pyrimidin-2-amine,
4-(1H-benzo[d]imidazol-5-yl)-N-(3-(4-methylpiperazin-1-yl)phenyl)pyrimidin-2-amine,
4-(1H-benzo[d]imidazol-5-yl)-N-(4-(tert-butyl)phenyl)pyrimidin-2-amine,
4-((4-(1H-benzo[d]imidazol-5-yl)pyrimidin-2-yl)amino)benzamide,
5-(2-(4-methylpiperazin-1-yl)pyrimidin-4-yl)-1H-benzo[d]imidazole,
4-(1H-benzo[d]imidazol-5-yl)-N-(thiophen-2-ylmethyl)pyrimidin-2-amine,
4-(benzo[d]thiazol-5-yl)-N-(3-(trifluoromethyl)phenyl)pyrimidin-2-amine,
4-(1H-benzo[d]imidazol-5-yl)-N-(1-methyl-1H-pyrazol-4-yl)pyrimidin-2-amine,
4-(1H-benzo[d]imidazol-5-yl)-N-phenylpyrimidin-2-amine,
4-(1H-benzo[d]imidazol-5-yl)-N-(2-(trifluoromethoxy)phenyl)pyrimidin-2-amine,
4-(1H-benzo[d]imidazol-5-yl)-N-(2-ethylphenyl)pyrimidin-2-amine,
4-(1H-benzo[d]imidazol-5-yl)-N-(4-fluorophenyl)pyrimidin-2-amine,
4-(1H-benzo[d]imidazol-5-yl)-N-((1-methyl-1H-pyrazol-5-yl)methyl)pyrimidin-2-amine,
4-(1H-benzo[d]imidazol-5-yl)-N-(5-fluoropyridin-3-yl)pyrimidin-2-amine,
4-(1H-benzo[d]imidazol-5-yl)-N-(thiophen-2-yl)pyrimidin-2-amine,
4-(1H-benzo[d]imidazol-5-yl)-N-(furan-2-ylmethyl)pyrimidin-2-amine,
4-(1H-benzo[d]imidazol-5-yl)-N-(4-chlorophenyl)pyrimidin-2-amine,
4-(1H-benzo[d]imidazol-5-yl)-N-(3-(tert-butyl)phenyl)pyrimidin-2-amine,
4-(1H-benzo[d]imidazol-5-yl)-N-cyclopentylpyrimidin-2-amine,
4-(1H-benzo[d]imidazol-5-yl)-N-(4-chloro-3-(trifluoromethyl)phenyl)pyrimidin-2-amine,
3-((4-(1H-benzo[d]imidazol-5-yl)pyrimidin-2-yl)amino)phenol,
N-(3-(3-aminopropoxy)phenyl)-4-(1H-benzo[d]imidazol-5-yl)pyrimidin-2-amine,
4-(1H-benzo[d]imidazol-5-yl)-N-(3-(piperazin-1-yl)phenyl)pyrimidin-2-amine,
4-(1H-benzo[d]imidazol-5-yl)-N-(3-ethynylphenyl)pyrimidin-2-amine,
3-((4-(1H-benzo[d]imidazol-5-yl)pyrimidin-2-yl)amino)benzonitrile,
4-(1-methyl-1H-benzo[d]imidazol-5-yl)-N-(3-(trifluoromethyl)phenyl)pyrimidin-2-amine,
4-(1H-benzo[d]imidazol-5-yl)-N-cyclohexylpyrimidin-2-amine,
4-(1H-benzo[d]imidazol-5-yl)-N-(2,3-dihydro-1H-inden-5-yl)pyrimidin-2-amine,
4-(1H-benzo[d]imidazol-5-yl)-N-(3,4-dichlorophenyl)pyrimidin-2-amine,
4-((4-(1H-benzo[d]imidazol-5-yl)pyrimidin-2-yl)amino)benzonitrile,
4-(1H-benzo[d]imidazol-5-yl)-N-(3-(trifluoromethoxy)phenyl)pyrimidin-2-amine,
4-(1H-benzo[d]imidazol-5-yl)-N-(tetrahydro-2H-pyran-4-yl)pyrimidin-2-amine,
4-(1H-benzo[d]imidazol-5-yl)-N-(3-morpholinophenyl)pyrimidin-2-amine,
4-(1H-benzo[d]imidazol-5-yl)-N-(p-tolyl)pyrimidin-2-amine,
4-(1H-benzo[d]imidazol-5-yl)-N-isopropylpyrimidin-2-amine,
4-(1H-imidazo[4,5-b]pyridin-6-yl)-N-(3-(trifluoromethyl)phenyl)-1,3,5-triazin-2-amine,
4-(1H-imidazo[4,5-b]pyridin-6-yl)-N-(5-(trifluoromethyl)pyridin-3-yl)pyrimidin-2-amine,
4-(imidazo[1,2-a]pyrazin-6-yl)-N-(3-(trifluoromethyl)phenyl)pyrimidin-2-amine,
N²-(3-fluorophenyl)-N⁴-(6-methoxypyridin-3-yl)pyrimidine-2,4-diamine,
4-((6-methoxypyridin-3-yl)oxy)-N-(3-(trifluoromethyl)phenyl)pyrimidin-2-amine,
5-((2-((3-fluorophenyl)amino)pyrimidin-4-yl)oxy)pyridin-2-ol,
N⁴-(2-(1H-imidazol-5-yl)ethyl)-N²-(3-(trifluoromethyl)phenyl)pyrimidine-2,4-diamine,
4-(1,4,6,7-tetrahydro-5H-imidazo[4,5-c]pyridin-5-yl)-N-(3-(trifluoromethyl)phenyl)pyrimidin-2-amine,
4-(1H-imidazo[4,5-b]pyridin-6-yl)-5-methyl-N-(3-(trifluoromethyl)phenyl)pyrimidin-2-amine,
4-(5-methyl-1H-benzo[d]imidazol-6-yl)-N-(3-(trifluoromethyl)phenyl)pyrimidin-2-amine,
4-(7-fluoro-1H-benzo[d]imidazol-5-yl)-N-(3-(trifluoromethyl)phenyl)pyrimidin-2-amine,
5-(2-((3-(trifluoromethyl)phenyl)amino)pyrimidin-4-yl)-1,3-dihydro-2H-benzo[d]imidazol-2-one,
5-(2-((3-fluorophenyl)amino)pyrimidin-4-yl)-1,3-dihydro-2H-benzo[d]imidazol-2-one,
4-((2-((3-fluorophenyl)amino)pyrimidin-4-yl)amino)pyridin-2-ol,
5-(2-((3-(trifluoromethyl)phenyl)amino)pyrimidin-4-yl)thiazol-2-amine,
4-(2-((3-(2-aminoethoxy)phenyl)amino)pyrimidin-4-yl)pyridin-2-ol,
N-(3-(2-aminoethoxy)phenyl)-4-(2-methoxypyridin-4-yl)pyrimidin-2-amine,
N²-(3-fluorophenyl)-N⁴-(2-methoxypyridin-4-yl)pyrimidine-2,4-diamine,
5-((2-((3-fluorophenyl)amino)pyrimidin-4-yl)amino)pyridin-2-ol,
N²-(3-fluorophenyl)-N⁴-(6-methoxypyridin-3-yl)pyrimidine-2,4-diamine,
4-(4-methyl-1H-benzo[d]imidazol-5-yl)-N-(3-(trifluoromethyl)phenyl)pyrimidin-2-amine,
4-(6-fluoro-1H-benzo[d]imidazol-5-yl)-N-(3-(trifluoromethyl)phenyl)pyrimidin-2-amine,
4-(imidazo[1,2-a]pyridin-6-yl)-N-(3-(trifluoromethyl)phenyl)pyrimidin-2-amine,
4-(2-((3-(2-hydroxyethoxy)phenyl)amino)pyrimidin-4-yl)pyridin-2-ol,
2-(3-((4-(2-methoxypyridin-4-yl)pyrimidin-2-yl)amino)phenoxy)ethan-1-ol,
4-(2-((3-(trifluoromethyl)phenyl)amino)pyrimidin-4-yl)phenol,
2′-((3-(trifluoromethyl)phenyl)amino)-[4,4′-bipyrimidin]-6-ol,
4-(1H-pyrrolo[3,2-b]pyridin-6-yl)-N-(3-(trifluoromethyl)phenyl)pyrimidin-2-amine,
4-(1H-imidazo[4,5-b]pyridin-6-yl)-N-(3-propylphenyl)pyrimidin-2-amine,
4-(1H-imidazo[4,5-b]pyridin-6-yl)-N-(3-isopropylphenyl)pyrimidin-2-amine,
N-(4-fluoro-3-methylphenyl)-4-(1H-imidazo[4,5-b]pyridin-6-yl)pyrimidin-2-amine,
4-(1H-imidazo[4,5-b]pyridin-6-yl)-N-(m-tolyl)pyrimidin-2-amine,
N²-(3-(trifluoromethyl)phenyl)-[4,5′-bipyrimidine]-2,2′-diamine,
4-(1H-benzo[d][1,2,3]triazol-5-yl)-N-(3-fluorophenyl)pyrimidin-2-amine,
4-(1H-benzo[d][1,2,3]triazol-5-yl)-N-(3-(trifluoromethyl)phenyl)pyrimidin-2-amine,
N-(3-ethylphenyl)-4-(1H-imidazo[4,5-b]pyridin-6-yl)pyrimidin-2-amine,
N-(3-chlorophenyl)-4-(1H-imidazo[4,5-b]pyridin-6-yl)pyrimidin-2-amine,
4-(2-methyl-1H-benzo[d]imidazol-6-yl)-N-(3-(trifluoromethyl)phenyl)pyrimidin-2-amine,
4-(2-methoxythiazol-5-yl)-N-(3-(trifluoromethyl)phenyl)pyrimidin-2-amine,
5-(2-((3-(trifluoromethyl)phenyl)amino)pyrimidin-4-yl)thiazol-2-ol,
4-(2-((5-fluoropyridin-3-yl)amino)pyrimidin-4-yl)pyridin-2-ol,
N-(5-fluoropyridin-3-yl)-4-(2-methoxypyridin-4-yl)pyrimidin-2-amine,
4-(2-((3,5-difluorophenyl)amino)pyrimidin-4-yl)pyridin-2-ol,
N-(3,5-difluorophenyl)-4-(2-methoxypyridin-4-yl)pyrimidin-2-amine,
4-(2-((3-(tert-butyl)phenyl)amino)pyrimidin-4-yl)pyridin-2-ol,
N-(3-(tert-butyl)phenyl)-4-(2-methoxypyridin-4-yl)pyrimidin-2-amine,
N-(3-fluorophenyl)-4-(1H-imidazo[4,5-b]pyridin-6-yl)pyrimidin-2-amine,
4-(1H-indol-5-yl)-N-(3-(trifluoromethyl)phenyl)pyrimidin-2-amine,
N-(3-fluorophenyl)-4-(1H-indol-5-yl)pyrimidin-2-amine,
4-(2-((5-(trifluoromethyl)pyridin-3-yl)amino)pyrimidin-4-yl)pyridin-2-ol,
4-(2-methoxypyridin-4-yl)-N-(5-(trifluoromethyl)pyridin-3-yl)pyrimidin-2-amine,
4-(2-((2-(trifluoromethyl)pyridin-4-yl)amino)pyrimidin-4-yl)pyridin-2-ol,
4-(2-methoxypyridin-4-yl)-N-(2-(trifluoromethyl)pyridin-4-yl)pyrimidin-2-amine,
4-(2-((4-(trifluoromethyl)pyridin-2-yl)amino)pyrimidin-4-yl)pyridin-2-ol,
4-(2-(m-tolylamino)pyrimidin-4-yl)pyridin-2-ol,
4-(2-methoxypyridin-4-yl)-N-(m-tolyl)pyrimidin-2-amine,
4-(1H-imidazo[4,5-b]pyridin-6-yl)-N-(3-(trifluoromethyl)phenyl)pyrimidin-2-amine,
6′-methoxy-N-phenyl-[4,4′-bipyrimidin]-2-amine,
2′-methoxy-N-phenyl-[4,4′-bipyrimidin]-2-amine,
4-(1H-indazol-6-yl)-N-(3-(trifluoromethyl)phenyl)pyrimidin-2-amine,
N-(3-fluorophenyl)-4-(1H-indazol-5-yl)pyrimidin-2-amine,
4-(2-methoxypyridin-4-yl)-N-(4-(trifluoromethyl)pyridin-2-yl)pyrimidin-2-amine,
4-(2-((6-(trifluoromethyl)pyridin-2-yl)amino)pyrimidin-4-yl)pyridin-2-ol,
4-(2-methoxypyridin-4-yl)-N-(6-(trifluoromethyl)pyridin-2-yl)pyrimidin-2-amine,
N-(3-fluorophenyl)-4-(1H-pyrrolo[2,3-b]pyridin-5-yl)pyrimidin-2-amine,
6-(2-((4-fluoropyridin-2-yl)amino)pyrimidin-4-yl)pyridin-3-ol,
4-(1H-benzo[d]imidazol-6-yl)-N-(3-(trifluoromethyl)phenyl)pyrimidin-2-amine,
4-(1H-indol-6-yl)-N-(3-(trifluoromethyl)phenyl)pyrimidin-2-amine,
3-(2-((3-(trifluoromethyl)phenyl)amino)pyrimidin-4-yl)phenol,
N-(3-fluorophenyl)-4-(5-methoxypyridin-2-yl)pyrimidin-2-amine,
4-(2-(pyrimidin-4-ylamino)pyrimidin-4-yl)pyridin-2-ol,
N-(3-fluorophenyl)-4-(5-methoxythiophen-2-yl)pyrimidin-2-amine,
4-(2-methoxypyridin-4-yl)-N-(pyrimidin-4-yl)pyrimidin-2-amine,
4-(2-(pyrimidin-5-ylamino)pyrimidin-4-yl)pyridin-2-ol,
4-(2-methoxypyridin-4-yl)-N-(pyrimidin-5-yl)pyrimidin-2-amine,
N-(3-fluorophenyl)-4-(2-methoxythiazol-5-yl)pyrimidin-2-amine,
5-(2-((3-fluorophenyl)amino)pyrimidin-4-yl)thiazol-2-ol,
2-(2-((3-fluorophenyl)amino)pyrimidin-4-yl)pyridin-4-ol,
2-(2-(phenylamino)pyrimidin-4-yl)pyridin-4-ol,
4-(2-(pyrimidin-2-ylamino)pyrimidin-4-yl)pyridin-2-ol,
methyl 3-((4-(2-hydroxypyridin-4-yl)pyrimidin-2-yl)amino)benzoate,
4-(2-(pyridin-2-ylamino)pyrimidin-4-yl)pyridin-2-ol,
4-(2-methoxypyridin-4-yl)-N-(pyridin-2-yl)pyrimidin-2-amine,
4-(2-(pyridin-3-ylamino)pyrimidin-4-yl)pyridin-2-ol,
4-(2-methoxypyridin-4-yl)-N-(pyridin-3-yl)pyrimidin-2-amine,
4-(2-(pyridin-4-ylamino)pyrimidin-4-yl)pyridin-2-ol,
4-(2-((3-methoxyphenyl)amino)pyrimidin-4-yl)pyridin-2-ol,
N-(3-methoxyphenyl)-4-(2-methoxypyridin-4-yl)pyrimidin-2-amine,
N-(3-fluorophenyl)-4-(4-methoxypyridin-2-yl)pyrimidin-2-amine,
4-(2-((3-fluorophenyl)amino)pyrimidin-4-yl)phenol,
4-(2-methoxypyridin-4-yl)-N-(pyrimidin-2-yl)pyrimidin-2-amine,
3-((4-(2-hydroxypyridin-4-yl)pyrimidin-2-yl)amino)benzoic acid,
4-(2-methoxypyridin-4-yl)-N-(pyridin-4-yl)pyrimidin-2-amine,
3-((4-(2-methoxypyridin-4-yl)pyrimidin-2-yl)amino)benzamide,
4-(1H-benzo[d]imidazol-5-yl)-N-(3-fluorophenyl)pyrimidin-2-amine,
5-(2-((3-fluorophenyl)amino)pyrimidin-4-yl)pyridin-3-ol,
5-(2-((3-(trifluoromethyl)phenyl)amino)pyrimidin-4-yl)pyridin-3-ol,
N-(3-fluorophenyl)-4-(1H-indol-5-yl)pyrimidin-2-amine,
N-(3-fluorophenyl)-4-(5-methoxypyridin-3-yl)pyrimidin-2-amine,
4-(5-methoxypyridin-3-yl)-N-(3-(trifluoromethyl)phenyl)pyrimidin-2-amine,
4-(2-((2,3-difluorophenyl)amino)pyrimidin-4-yl)pyridin-2-ol,
N-(2,3-difluorophenyl)-4-(2-methoxypyridin-4-yl)pyrimidin-2-amine,
4-(2-((2-fluorophenyl)amino)pyrimidin-4-yl)pyridin-2-ol,
N-(2-fluorophenyl)-4-(2-methoxypyridin-4-yl)pyrimidin-2-amine,
4-(2-((3,4-difluorophenyl)amino)pyrimidin-4-yl)pyridin-2-ol,
N-(3,4-difluorophenyl)-4-(2-methoxypyridin-4-yl)pyrimidin-2-amine,
5-(2-((3-(trifluoromethyl)phenyl)amino)pyrimidin-4-yl)pyridin-2-ol,
4-(6-methoxypyridin-3-yl)-N-(3-(trifluoromethyl)phenyl)pyrimidin-2-amine,
5-(2-((3-fluorophenyl)amino)pyrimidin-4-yl)pyridin-2-ol,
N-(3-fluorophenyl)-4-(6-methoxypyridin-3-yl)pyrimidin-2-amine,
4-(4-(methyl(phenyl)amino)-1,3,5-triazin-2-yl)pyridin-2-ol,
4-(2-methoxypyridin-4-yl)-N-methyl-N-phenyl-1,3,5-triazin-2-amine,
4-(4-methoxypyridin-2-yl)-N-phenylpyrimidin-2-amine,
5-(2-(methyl(phenyl)amino)pyrimidin-4-yl)pyridin-3-ol,
5-(2-(phenylamino)pyrimidin-4-yl)pyridin-3-ol,
4-(2-(methyl (3-(trifluoromethyl)phenyl)amino)pyrimidin-4-yl)pyridin-2-ol,
4-(2-(methyl(phenyl)amino)pyrimidin-4-yl)pyridin-2-ol,
5-(2-(methyl(phenyl)amino)pyrimidin-4-yl)pyridin-2-ol,
4-(6-methoxypyridin-3-yl)-N-methyl-N-phenylpyrimidin-2-amine,
4-(5-methoxypyridin-3-yl)-N-methyl-N-phenylpyrimidin-2-amine,
6-(2-(methyl(phenyl)amino)pyrimidin-4-yl)pyridin-2-ol,
6-(2-(phenylamino)pyrimidin-4-yl)pyridin-2-ol,
4-(6-methoxypyridin-2-yl)-N-methyl-N-phenylpyrimidin-2-amine,
4-(5-methoxypyridin-3-yl)-N-phenylpyrimidin-2-amine,
4-(2-benzylpyrimidin-4-yl)pyridin-2-ol,
2-benzyl-4-(2-methoxypyridin-4-yl)pyrimidine,
4-(2-(phenylthio)pyrimidin-4-yl)pyridin-2-ol,
4-(2-phenoxypyrimidin-4-yl)pyridin-2-ol,
4-(4-(phenylamino)-1,3,5-triazin-2-yl)pyridin-2-ol,
4-(2-methoxypyridin-4-yl)-2-(phenylthio)pyrimidine,
4-(2-methoxypyridin-4-yl)-2-phenoxypyrimidine,
4-(6-methoxypyridin-2-yl)-N-phenylpyrimidin-2-amine,
4-(2-((3-fluorophenyl)(methyl)amino)pyrimidin-4-yl)pyridin-2-ol,
5-(2-(phenylamino)pyrimidin-4-yl)pyridin-2-ol,
4-(6-methoxypyridin-3-yl)-N-phenylpyrimidin-2-amine,
4-(2-methoxypyridin-4-yl)-N-phenyl-1,3,5-triazin-2-amine,
4-(2-((3-(trifluoromethyl)phenyl)amino)pyrimidin-4-yl)pyridin-2-ol,
4-(2-((3-fluorophenyl)amino)pyrimidin-4-yl)pyridin-2-ol,
N-(3-fluorophenyl)-4-(2-methoxypyridin-4-yl)pyrimidin-2-amine,
3-(2-(phenylamino)pyrimidin-4-yl)phenol,
6-(3-methoxyphenyl)-N-phenylpyridin-2-amine,
4-(3-methoxyphenyl)-N-phenylpyridin-2-amine,
4-(3-methoxyphenyl)-N-methyl-N-phenylpyrimidin-2-amine,
N-(2-methoxyphenyl)-4-(3-methoxyphenyl)pyrimidin-2-amine,
N-(3-chlorophenyl)-4-(3-methoxyphenyl)pyrimidin-2-amine,
N,4-bis(3-methoxyphenyl)pyrimidin-2-amine,
4-(2-(phenylamino)pyrimidin-4-yl)pyridin-2-ol,
4-(2-methoxypyridin-4-yl)-N-phenylpyrimidin-2-amine,
4-(3-methoxyphenyl)-N-phenylpyrimidin-2-amine,
N-(3-((4-(1H-benzo[d]imidazol-5-yl)pyrimidin-2-yl)amino)phenyl)furan-2-carboxamide,
N-(3-((4-(1H-benzo[d]imidazol-5-yl)pyrimidin-2-yl)amino)phenyl)methanesulfonamide,
1-(3-((4-(1H-benzo[d]imidazol-5-yl)pyrimidin-2-yl)amino)phenyl)ethan-1-one,
N-(3-((4-(1H-benzo[d]imidazol-5-yl)pyrimidin-2-yl)amino)phenyl)isobutyramide,
4-(1H-benzo[d]imidazol-5-yl)-N-(3,4,5-trifluorophenyl)pyrimidin-2-amine,
N-(3-(1H-1,2,3-triazol-1-yl)phenyl)-4-(1H-benzo[d]imidazol-5-yl)pyrimidin-2-amine,
4-(1H-benzo[d]imidazol-5-yl)-N-(3-((trifluoromethyl)sulfonyl)phenyl)pyrimidin-2-amine,
N-(3-(1H-pyrazol-5-yl)phenyl)-4-(1H-benzo[d]imidazol-5-yl)pyrimidin-2-amine,
N-(3-((4-(1H-benzo[d]imidazol-5-yl)pyrimidin-2-yl)amino)phenyl)-N-methylbenzenesulfonamide,
3-((4-(1H-benzo[d]imidazol-5-yl)pyrimidin-2-yl)amino)-N-phenylbenzamide,
4-(1H-benzo[d]imidazol-5-yl)-N-(3-(oxazol-5-yl)phenyl)pyrimidin-2-amine,
4-(1H-benzo[d]imidazol-5-yl)-N-(4-methyl-3-(trifluoromethyl)phenyl)pyrimidin-2-amine,
4-(1H-benzo[d]imidazol-5-yl)-N-(4-(trifluoromethyl)phenyl)pyrimidin-2-amine,
N-(3-(1H-tetrazol-5-yl)phenyl)-4-(1H-benzo[d]imidazol-5-yl)pyrimidin-2-amine,
N-(4-(1H-benzo[d]imidazol-5-yl)pyrimidin-2-yl)-1H-indazol-6-amine,
N-(benzo[b]thiophen-5-yl)-4-(1H-benzo[d]imidazol-5-yl)pyrimidin-2-amine,
N-(3-fluorophenyl)-4-(pyrazolo[1,5-a]pyrimidin-5-yl)pyrimidin-2-amine,
4-(benzo[d]oxazol-5-yl)-N-(3-fluorophenyl)pyrimidin-2-amine,
ethyl 3-(3-((4-(1H-benzo[d]imidazol-6-yl)pyrimidin-2-yl)amino)phenyl)propanoate,
N-(3-fluorophenyl)-4-(imidazo[1,2-a]pyrimidin-6-yl)pyrimidin-2-amine,
4-(1H-benzo[d]imidazol-6-yl)-N-(3-chloro-5-methylphenyl)pyrimidin-2-amine,
N¹-(4-(1H-benzo[d]imidazol-6-yl)pyrimidin-2-yl)benzene-1,3-diamine, and
4-(1H-benzo[d]imidazol-6-yl)-N-(1-(tert-butyl)-1H-pyrazol-4-yl)pyrimidin-2-amine,
or a pharmaceutically acceptable salt thereof.

59. A pharmaceutical composition comprising a compound according to any one of claims 1 to 58, or a pharmaceutically acceptable salt thereof, and at least one diluent or excipient.

60. A method of treating a disease or disorder, comprising administering to a subject in need thereof a therapeutically effective amount of a compound, or a pharmaceutically acceptable salt thereof, according to any one of claims 1 to 58.

61. A method of treating a disease or disorder, comprising administering to a subject in need thereof a therapeutically effective amount of a pharmaceutical composition according to claim 59.

62. The method of claim 60 or 61, wherein the disease or disorder is mediated by a Myc protein.

63. The method of claim 62, wherein the Myc protein is selected from an N-Myc protein, a c-Myc protein, and an L-Myc protein.

64. The method of any one of claims 60 to 63, wherein the disease or disorder comprises cancer.

65. A method of treating cancer, comprising administering to a subject in need thereof a therapeutically effective amount of a compound, or a pharmaceutically acceptable salt thereof, according to any one of claims 1 to 58.

66. A method of treating cancer, comprising administering to a subject in need thereof of a therapeutically effective amount of a pharmaceutical composition according to claim 59.

67. The method of any one of claims 64 to 66, wherein the cancer is selected from head and neck cancer, nervous system cancer, brain cancer, neuroblastoma, medulloblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genitourinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

68. The method of any one of claims 64 to 66, wherein the cancer comprises a medulloblastoma.

69. The method of any one of claims 60 to 68, wherein the subject comprises a human.

70. A method of treating a disease or condition mediated by CDK9 in a subject comprising administering a therapeutically effective amount of a compound of any one of claims 1 to 58, or a pharmaceutically acceptable salt thereof, to the subject.

71. The method of claim 70, wherein the compound comprises a CDK9 inhibitor.

72. The method of claim 70, wherein the compound inhibits activity of CDK9.
